(19) United States
(12) Patent Application Publication
(10) Pub. No.: US 2025/0221901 A1
(43) Pub. Date: Jul. 10, 2025

(54) NEUTRAL ODOR 1,3-BUTYLENE GLYCOL COMPOSITIONS AND METHODS OF USE THEREOF

(71) Applicant: Genomatica, Inc., San Diego, CA (US)

(72) Inventors: Fasil A. Tadesse, San Diego, CA (US); Aakash Parikh, San Diego, CA (US)

(21) Appl. No.: 18/853,340

(22) PCT Filed: Apr. 10, 2023
(86) PCT No.: PCT/US2023/065603

§ 371 Date: Oct. 1, 2024

Related U.S. Application Data
(60) Provisional application No. 63/329,616, filed on Apr. 11, 2022.
(60) Provisional application No. 63/331,605, filed on Apr. 15, 2022.

(51) Int. Cl. A61K 8/34

(52) U.S. Cl. CPC A61K 8/345; A61K 2800/10; A61K 2800/5922

(57) ABSTRACT

The disclosure provides embodiments directed to a 1,3-butylene glycol (BG) product that has a neutral odor, a composition comprising the BG product described here, and methods of using the BG product and compositions comprising the BG products. BG products described here demonstrate exemplary qualities and characteristics of being natural, sustainable, and having a neutral odor for use in cosmetics, cosmeceuticals, and personal care products. Such BG compositions of the description provide a BG product that does not mask or alter the fragrance notes of the cosmetics, cosmeceuticals, and personal care products. The BG products and compositions comprising such BG products as disclosed here are cost-effective while maintaining a neutral odor.

### RELATED APPLICATIONS

This application claims priority to U.S. Provisional Application Ser. No. 63/329,616, filed on Apr. 11, 2022; and U.S. Provisional Application Ser. No. 63/331,605, filed on Apr. 15, 2022; the disclosures of which are hereby incorporated by reference in their entireties.

### FIELD OF DISCLOSURE

The present disclosure is generally related to 1,3-butylene glycol (BG) products and compositions comprising such BG products, and methods and uses thereof, where BG products of the disclosure comprise an essentially neutral odor for use in cosmetics, cosmeceuticals, and personal care products.

### BACKGROUND

The compound BG (also known and used interchangeably here as 1,3-butylene glycol; 1,3-BG; 1,3-butanediol; 1,3-BDO) is a four-carbon diol (C₄H₁₀O₂) traditionally produced from petroleum-derived acetylene through its hydration. The resulting acetaldehyde is then converted to 3-hydroxybutyraldehdye which is subsequently reduced to form a petroleum-derived 1,3-butylene glycol. However, this conventional petrochemical process begins with acetaldehyde, which is a mutagen and carcinogen. Moreover, petroleum-derived BG (petro-BG) is costly, lacks sustainability, has irritant properties, and can contain contaminants or compounds that result in products containing an offensive or undesirable odor.

Therefore, there is a need to develop an alternative to petroleum-derived conventional materials, such as petro-BG, that can be used in cost-efficient compositions where the BG is safe, natural, and sustainable without offensive or undesirable odors.

### SUMMARY

In accordance with the foregoing objectives and others, embodiments of the present disclosure satisfy the aforementioned needs and provide related advantages as well. The present disclosure provides in some aspects, a neutral odor 1,3-butylene glycol (BG) product, a composition comprising such a BG product, including cosmetics, cosmeceuticals, and personal care products, and methods and uses of such BG products and compositions.

Some aspects provide a 1,3-butylene glycol (BG) product comprising one or more compounds selected from the group consisting of: 5-norbornene-2-ol; 2-ethyl-1-hexanol; and salts, derivatives, analogs, or solvates, thereof, or any combinations thereof, where the BG product comprises a bioderived BG (bioBG).

Other aspects provide a BG product comprising one or more compounds characterized by an odor-active region as measured by gas chromatography/mass spectrometry/olfactory (GC/MS/O) having a relative acquisition time selected from a group consisting of: about 0.594 min having a fruity and/or solventy odor; about 0.628 min having a metallic and/or waxy odor; about 0.655 min having a citrus and/or peel odor; about 0.711 min having a solventy and/or acrylic odor; about 1.328 mins having a roasty and/or exhaust odor; and combinations thereof, where the BG product comprises a bioBG, and the bioBG has a relative acquisition time of 1 min.

Additional aspects are directed to a 1,3-butylene glycol (BG) product comprising one or more compounds characterized by an odor-active region as measured by gas chromatography/quadrupole time-of-flight-mass spectrometry (GC/QTOF-MS), where the odor-active region elutes at an acquisition time of about 12.2 mins at 66.0463 m/z, where the BG product comprises bioderived BG (bioBG).

In some aspects, a 1,3-butylene glycol (BG) product comprising one or more compounds comprising a mass spectrum as characterized by quadrupole quadrupole time-of-flight-mass spectrometry electron ionization (QTOF EI) at 70 eV is provided. Such a mass spectrum can comprise peaks: 39.0229 m/z; 43.0707 m/z; 53.0385 m/z; 57.0169 m/z; 66.0463 m/z; 74.0156 m/z; 81.0333 m/z; 85.0284 m/z; 91.0539 m/z; 95.0489 m/z; and 110.0724 m/z; or the mass spectrum comprises peaks: 39.0229 m/z; 43.0707 m/z; 53.0385 m/z; 66.0463 m/z; 81.0333 m/z; 91.0539 m/z; 95.0489 m/z; and 110.0724 m/z.

Other aspects are directed to a 1,3-butylene glycol (BG) product comprising one or more compounds as measured by extracted ion chromatograms selected from the group consisting of: 2-methoxymethyl-2,4,5-trimethyl-1,3-dioxolane having about 0 counts-1.6×10⁶ counts at 59.0489 m/z; 1-octen-3-one having about 0.15×10⁴-about 0.4×10⁴ counts at m/z 70.0411; 3-heptanone having about 0.4×10⁵ counts at m/z 57.0698; N-pentyl-2-butylamine having about 0 counts at m/z 114.0675; 5-norbornene-2-ol having about 0 counts to about 3.75×10⁵ counts at m/z 66.0463; and any combinations thereof, where the BG product comprises bioderived BG (bioBG).

Additional aspects provide any of the disclosed BG products comprising a bioBG, where the BG product further comprises one or more compounds selected from the group consisting of: 3-hydroxy-butanal in an amount of less than 25 ppm; 4-hydroxy-2-butanone in an amount less than 25 ppm; 4-(3-hydroxybutoxy)butan-2-one in an amount less than 25 ppm; 4-((4-hydroxybutan-2-yl)oxy)-butan-2-one in an amount less than 25 ppm; 1,2-propanediol in an amount less than 1 ppm; 1,3-propanediol in an amount less than 10 ppm; 2,3-butanediol in an amount less than 1 ppm; 2,4-dimethyl-6-((1-(4-methyl-1,3-dioxan-2-yl)propan-2-yl)oxy)-1,3-dioxane in an amount less than 2 ppm; 4-(sec-butoxy)-2,6-dimethyl-1,3-dioxane in an amount less than 4 ppm; 2,4-dimethyl-1,3-dioxane in an amount less than 8 ppm; (E)-4-methyl-2-(prop-1-en-1-yl)-1,3-dioxane in an amount less than 8 ppm; 3-hydroxybutyl acetate in an amount less than 8 ppm; 1-(2-methyl-1,3-dioxan-4-yl)propan-2-ol in an amount less than 8 ppm; 2-methyl-4-propyl-1,3-dioxane in an amount less than 8 ppm; tri-substituted 1,3-dioxane

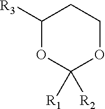

in an amount less than 8 ppm and/or 3-hydroxybutyl carbono-substituted

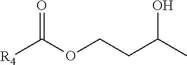

in an amount less than 8 ppm, wherein R1 to R4 are the same or different; a hydrogen atom, a C1-C4 alkyl group optionally substituted by an OH group, or a C2-C4 alkenyl group optionally substituted by OH group; 2,4-dimethyl-2-vinyl-1,3-dioxane; 2-(2,4-dimethyl-1,3-dioxan-2-yl)ethan-1-ol; 2,2,4-trimethyl-1,3-dioxane; 4-methyl-1,3-dioxane, wherein the total of 2,4-dimethyl-2-vinyl-1,3-dioxane; 2-(2,4-dimethyl-1,3-dioxan-2-yl)ethan-1-ol; 2,2,4-trimethyl-1,3-dioxane; 4-methyl-1,3-dioxane in a total amount of less than 34 ppm; (E)-4-methyl-2-(prop-1-en-1-yl)-1,3-dioxane; 1-(4-methyl-1,3-dioxan-2-yl)propan-2-ol; 2,4-dimethyl-1,3-dioxane; 3-hydroxybutyl (E)-but-2-enoate; 3-hydroxybutyl acetate; 3-hydroxybutyl 3-hydroxybutanoate, wherein the total of (E)-4-methyl-2-(prop-1-en-1-yl)-1,3-dioxane; 1-(4-methyl-1,3-dioxan-2-yl)propan-2-ol; 2,4-dimethyl-1,3-dioxane; 3-hydroxybutyl (E)-but-2-enoate; 3-hydroxybutyl acetate; 3-hydroxybutyl 3-hydroxybutanoate in a total amount of less than 63 ppm or wherein the total of 2,4-dimethyl-2-vinyl-1,3-dioxane; 2-(2,4-dimethyl-1,3-dioxan-2-yl)ethan-1-ol; 2,2,4-trimethyl-1,3-dioxane; 4-methyl-1,3-dioxane; (E)-4-methyl-2-(prop-1-en-1-yl)-1,3-dioxane; 1-(4-methyl-1,3-dioxan-2-yl)propan-2-ol; 2,4-dimethyl-1,3-dioxane; 3-hydroxybutyl (E)-but-2-enoate; 3-hydroxybutyl acetate; 3-hydroxybutyl 3-hydroxybutanoate in a total amount of less than 97 ppm; methyl vinyl ketone in an amount less than 8 ppm; 1-hydroxy-3-butanone is in an amount less than 1 ppm; acetone in an amount less than 6 ppm; formaldehyde in an amount less than 2 ppm; crotonaldehyde in an amount less than 1 ppm; acetaldol in an amount less than 1 ppm; acetaldehyde in an amount less than 5 ppm; butyraldehyde in an amount less than 4 ppm; 2-butanol, wherein the total of 2,4-dimethyl-2-vinyl-1,3-dioxane; 2-(2,4-dimethyl-1,3-dioxan-2-yl)ethan-1-ol; 2,2,4-trimethyl-1,3-dioxane; 4-methyl-1,3-dioxane; (E)-4-methyl-2-(prop-1-en-1-yl)-1,3-dioxane; 1-(4-methyl-1,3-dioxan-2-yl)propan-2-ol; 2,4-dimethyl-1,3-dioxane; 3-hydroxybutyl (E)-but-2-enoate; 3-hydroxybutyl acetate; 3-hydroxybutyl 3-hydroxybutanoate; and 2-butanol in a total amount of less than 65 ppm; butyl acrylate; butyl acetate; 4-hydroxy-2-butanone; 2-methoxymethyl-2,4,5-trimethyl-1,3-dioxolane (solventy); 3-heptanone; 1-octen-3-one; N-pentyl-2-butylamine; 3-buten-2-one; 2-butenal; 2,4-dimethyl-1,3-dioxane; 2-ethyl-5-methyl-1,3-dioxane; butyl acrylate; 2-heptyl-1,3-dioxepane; 1-hydroxy-2-propanone; 1,2 propanediol (12PDO); 1,2,3-butanetriol; 1,2,3-propanetriol; 1,3 propanediol (13PDO); 1,4-butanediol (BDO); 2,3-butanediol (23BDO); 2,5-hexanedione; 3-buten-2-ol; cis-cryotyl alcohol; isopropyl alcohol (IPA); n-butane (n-but); dimethylethanolamine (DMAE); toluene; and any combinations thereof.

In some aspects, any of the disclosed BG products comprising a bioBG are provided, where the BG product does not comprise a compound selected from the group consisting of 3-hydroxy-butanal; 4-hydroxy-2-butanone; 4-(3-hydroxybutoxy)butan-2-one; 4-((4-hydroxybutan-2-yl)oxy)-butan-2-one; 1,2-propanediol; 1,3-propanediol; 2,3-butanediol; 2,4-dimethyl-6-((1-(4-methyl-1,3-dioxan-2-yl)propan-2-yl)oxy)-1,3-dioxane; 4-(sec-butoxy)-2,6-dimethyl-1,3-dioxane; 2,4-dimethyl-1,3-dioxane; (E)-4-methyl-2-(prop-1-en-1-yl)-1,3-dioxane; 3-hydroxybutyl acetate; 1-(2-methyl-1,3-dioxan-4-yl)propan-2-ol; 2-methyl-4-propyl-1,3-dioxane; tri-substituted 1,3-dioxane

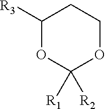

and/or 3-hydroxybutyl carbono-substituted

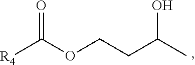

wherein R1 to R4 are the same or different; a hydrogen atom, a C1-C4 alkyl group optionally substituted by an OH group, or a C2-C4 alkenyl group optionally substituted by OH group; 2,4-dimethyl-2-vinyl-1,3-dioxane; 2-(2,4-dimethyl-1,3-dioxan-2-yl)ethan-1-ol; 2,2,4-trimethyl-1,3-dioxane; 4-methyl-1,3-dioxane; (E)-4-methyl-2-(prop-1-en-1-yl)-1,3-dioxane; 1-(4-methyl-1,3-dioxan-2-yl)propan-2-ol; 2,4-dimethyl-1,3-dioxane; 3-hydroxybutyl (E)-but-2-enoate; 3-hydroxybutyl acetate; 3-hydroxybutyl 3-hydroxybutanoate; methyl vinyl ketone; 1-hydroxy-3-butanone; acetone; formaldehyde; crotonaldehyde; acetaldol; acetaldehyde; butyraldehyde; 2-butanol; butyl acrylate; butyl acetate; 4-hydroxy-2-butanone; 2-methoxymethyl-2,4,5-trimethyl-1,3-dioxolane (solventy); 3-heptanone; 1-octen-3-one; N-pentyl-2-butylamine; 3-buten-2-one; 2-butenal; 2,4-dimethyl-1,3-dioxane; 2-ethyl-5-methyl-1,3-dioxane; butyl acrylate; 2-heptyl-1,3-dioxepane; 1-hydroxy-2-propanone; 1,2 propanediol (12PDO); 1,2,3-butanetriol; 1,2,3-propanetriol; 1,3 propanediol (13PDO); 1,4-butanediol (BDO); 2,3-butanediol (23BDO); 2,5-hexanedione; 3-buten-2-ol; cis-cryotyl alcohol; isopropyl alcohol (IPA); n-butane (n-but); dimethylethanolamine (DMAE); toluene; and or any combinations thereof, wherein the BG product comprises bioderived BG (bioBG).

Other aspects are directed to a 1,3-butylene glycol (BG) product, where the BG product comprises a bioderived BG (bioBG), where the BG product further comprises one or more compounds selected from the group consisting of: 3-hydroxy-butanal is in an amount of less than 25 ppm; 4-hydroxy-2-butanone is in an amount less than 25 ppm; 4-(3-hydroxybutoxy)butan-2-one is in an amount less than 25 ppm; 4-((4-hydroxybutan-2-yl)oxy)-butan-2-one is in an amount less than 25 ppm; 1,2-propanediol is in an amount less than 1 ppm; 1,3-propanediol is in an amount less than 10 ppm; 2,3-butanediol is in an amount less than 1 ppm; 2,4-dimethyl-6-((1-(4-methyl-1,3-dioxan-2-yl)propan-2-yl)oxy)-1,3-dioxane is in an amount less than 2 ppm; 4-(sec-butoxy)-2,6-dimethyl-1,3-dioxane is in an amount less than 4 ppm; 2,4-dimethyl-1,3-dioxane is in an amount less than 8 ppm; (E)-4-methyl-2-(prop-1-en-1-yl)-1,3-dioxane is in an amount less than 8 ppm; 3-hydroxybutyl acetate is in an amount less than 8 ppm; 1-(2-methyl-1,3-dioxan-4-yl)propan-2-ol is in an amount less than 8 ppm; 2-methyl-4-propyl-1,3-dioxane is in an amount less than 8 ppm; tri-substituted 1,3-dioxane

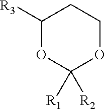

is in an amount less than 8 ppm and/or 3-hydroxybutyl carbono-substituted

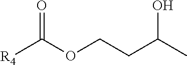

in an amount less than 8 ppm, wherein R1 to R4 are the same or different; a hydrogen atom, a C₁-C₄ alkyl group optionally substituted by an OH group, or a C2-C4 alkenyl group optionally substituted by OH group; 2,4-dimethyl-2-vinyl-1,3-dioxane; 2-(2,4-dimethyl-1,3-dioxan-2-yl)ethan-1-ol; 2,2,4-trimethyl-1,3-dioxane; 4-methyl-1,3-dioxane, wherein the total of 2,4-dimethyl-2-vinyl-1,3-dioxane; 2-(2,4-dimethyl-1,3-dioxan-2-yl)ethan-1-ol; 2,2,4-trimethyl-1,3-dioxane; 4-methyl-1,3-dioxane is in a total amount of less than 34 ppm; (E)-4-methyl-2-(prop-1-en-1-yl)-1,3-dioxane; 1-(4-methyl-1,3-dioxan-2-yl)propan-2-ol; 2,4-dimethyl-1,3-dioxane; 3-hydroxybutyl (E)-but-2-enoate; 3-hydroxybutyl acetate; 3-hydroxybutyl 3-hydroxybutanoate, wherein the total of (E)-4-methyl-2-(prop-1-en-1-yl)-1,3-dioxane; 1-(4-methyl-1,3-dioxan-2-yl)propan-2-ol; 2,4-dimethyl-1,3-dioxane; 3-hydroxybutyl (E)-but-2-enoate; 3-hydroxybutyl acetate; 3-hydroxybutyl 3-hydroxybutanoate is in a total amount of less than 63 ppm or wherein the total of 2,4-dimethyl-2-vinyl-1,3-dioxane; 2-(2,4-dimethyl-1,3-dioxan-2-yl)ethan-1-ol; 2,2,4-trimethyl-1,3-dioxane; 4-methyl-1,3-dioxane; (E)-4-methyl-2-(prop-1-en-1-yl)-1,3-dioxane; 1-(4-methyl-1,3-dioxan-2-yl)propan-2-ol; 2,4-dimethyl-1,3-dioxane; 3-hydroxybutyl (E)-but-2-enoate; 3-hydroxybutyl acetate; 3-hydroxybutyl 3-hydroxybutanoate is in a total amount of less than 97 ppm; methyl vinyl ketone is in an amount less than 8 ppm; 1-hydroxy-3-butanone is in an amount less than 1 ppm; acetone is in an amount less than 6 ppm; formaldehyde is in an amount less than 2 ppm; crotonaldehyde is in an amount less than 1 ppm; acetaldol is in an amount less than 1 ppm; acetaldehyde is in an amount less than 5 ppm; butyraldehyde is in an amount less than 4 ppm; 2-butanol, wherein the total of 2,4-dimethyl-2-vinyl-1,3-dioxane; 2-(2,4-dimethyl-1,3-dioxan-2-yl)ethan-1-ol; 2,2,4-trimethyl-1,3-dioxane; 4-methyl-1,3-dioxane; (E)-4-methyl-2-(prop-1-en-1-yl)-1,3-dioxane; 1-(4-methyl-1,3-dioxan-2-yl)propan-2-ol; 2,4-dimethyl-1,3-dioxane; 3-hydroxybutyl (E)-but-2-enoate; 3-hydroxybutyl acetate; 3-hydroxybutyl 3-hydroxybutanoate; and 2-butanol is in a total amount of less than 65 ppm; butyl acetate; butyl acrylate; 4-hydroxy-2-butanone; 2-methoxymethyl-2,4,5-trimethyl-1,3-dioxolane (solventy); 3-heptanone; 1-octen-3-one; N-pentyl-2-butylamine; 3-buten-2-one; 2-butenal; 2,4-dimethyl-1,3-dioxane; 2-ethyl-5-methyl-1,3-dioxane; butyl acrylate; 2-heptyl-1,3-dioxepane; 1-hydroxy-2-propanone; 1,2 propanediol (12PDO); 1,2,3-butanetriol; 1,2,3-propanetriol; 1,3 propanediol (13PDO); 1,4-butanediol (BDO); 2,3-butanediol (23BDO); 2,5-hexanedione; 3-buten-2-ol; cis-cryotyl alcohol; isopropyl alcohol (IPA); methyl vinyl ketone (MVK); n-butane (n-but); dimethylethanolamine (DMAE); toluene; and any combinations thereof.

In additional aspects, a 1,3-butylene glycol (BG) product is provided, where the BG product comprises a bioderived BG (bioBG), where the BG product does not comprise a compound selected from the group consisting of: 3-hydroxy-butanal; 4-hydroxy-2-butanone; 4-(3-hydroxybutoxy)butan-2-one; 4-((4-hydroxybutan-2-yl)oxy)-butan-2-one; 1,2-propanediol; 1,3-propanediol; 2,3-butanediol; 2,4-dimethyl-6-((1-(4-methyl-1,3-dioxan-2-yl)propan-2-yl)oxy)-1,3-dioxane; 4-(sec-butoxy)-2,6-dimethyl-1,3-dioxane; 2,4-dimethyl-1,3-dioxane; (E)-4-methyl-2-(prop-1-en-1-yl)-1,3-dioxane; 3-hydroxybutyl acetate; 1-(2-methyl-1,3-dioxan-4-yl)propan-2-ol; 2-methyl-4-propyl-1,3-dioxane; tri-substituted 1,3-dioxane

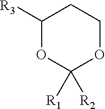

and/or 3-hydroxybutyl carbono-substituted

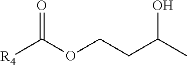

wherein R1 to R4 are the same or different; a hydrogen atom, a C1-C4 alkyl group optionally substituted by an OH group, or a C2-C4 alkenyl group optionally substituted by OH group; 2,4-dimethyl-2-vinyl-1,3-dioxane; 2-(2,4-dimethyl-1,3-dioxan-2-yl)ethan-1-ol; 2,2,4-trimethyl-1,3-dioxane; 4-methyl-1,3-dioxane; (E)-4-methyl-2-(prop-1-en-1-yl)-1,3-dioxane; 1-(4-methyl-1,3-dioxan-2-yl)propan-2-ol; 2,4-dimethyl-1,3-dioxane; 3-hydroxybutyl (E)-but-2-enoate; 3-hydroxybutyl acetate; 3-hydroxybutyl 3-hydroxybutanoate; methyl vinyl ketone; 1-hydroxy-3-butanone; acetone; formaldehyde; crotonaldehyde; acetaldol; acetaldehyde; butyraldehyde; 2-butanol; butyl acrylate; butyl acetate; 4-hydroxy-2-butanone; 2-methoxymethyl-2,4,5-trimethyl-1,3-dioxolane (solventy); 3-heptanone; 1-octen-3-one; N-pentyl-2-butylamine; 3-buten-2-one; 2-butenal; 2,4-dimethyl-1,3-dioxane; 2-ethyl-5-methyl-1,3-dioxane; butyl acrylate; 2-heptyl-1,3-dioxepane; 1-hydroxy-2-propanone; 1,2 propanediol (12PDO); 1,2,3-butanetriol; 1,2,3-propanetriol; 1,3 propanediol (13PDO); 1,4-butanediol (BDO); 2,3-butanediol (23BDO); 2,5-hexanedione; 3-buten-2-ol; cis-cryotyl alcohol; isopropyl alcohol (IPA); n-butane (n-but); dimethylethanolamine (DMAE); toluene; and or any combinations thereof, wherein the BG product comprises bioderived BG (bioBG).

Some aspects provide a BG product, wherein the BG product does not comprise, does not consist essentially of, does not consist of, or minimally comprises one or more compounds characterized by a peak as measured by gas chromatography/quadrupole time-of-flight-mass spectrometry (GC/QTOF-MS), where the peak elutes at an acquisition time selected from a group consisting of: about 12 mins at 59.0489 m/z, about 13 mins at 57.0698 m/z, about 15.5 mins at 70.0411 m/z, about 28.5 mins at 114.0675 m/z, and combinations thereof, wherein the BG product comprises a bioBG.

In some aspects, provided here is a BG product, where the BG product does not comprise, does not consist essentially of, does not consist of, or minimally comprises one or more compounds, where the compound comprises a mass spectrum as characterized by quadrupole time-of-flight-mass spectrometry electron ionization (QTOF EI) at 70 eV. Such a mass spectrum as characterized by QTOF EI comprises an aromatic region, an odor-active region, or a peak, wherein the mass spectrum is selected from the group consisting of: (a) 43.017 m/z, 55.0541 m/z, 73.0283 m/z, 86.0723 m/z, 97.0642 m/z, and 115.0752; (b) 41.0385 m/z, 57.0698 m/z, 68.0259 m/z, 72.0569 m/z, 85.0646 m/z, 99.0801 m/z, and 114.1033 m/z; and (c) 43.0179 m/z, 57.0336 m/z, 72.0206 m/z, 86.0362 m/z, 114.0675 m/z, 128.0830 m/z, 138.0670 m/z, and 155.1060 m/z, where the BG product comprises bioBG.

Some aspects provide a 1,3-butylene glycol (BG) product comprising a bioBG, where the BG product does not comprise one or more compounds comprising an odor-active region as measured by GC/MS that elutes at an acquisition time selected from a group consisting of: about 9.3 mins (toluene); about 9.6 mins (butyl acetate); about 10.5 mins (butyl acrylate); and about 16 mins (4-hydroxy-2-butanone), where bioBG elutes at about 18 mins. Further aspects provide a BG product comprising a bioBG, where the BG product does not comprise one or more compounds comprising an odor-active region as measured by GC/MS that elutes at an acquisition time selected from a group consisting of: about 9.3 mins (toluene); about 9.6 mins (butyl acetate); about 10.5 mins (butyl acrylate); about 11 mins; about 11.7 mins; about 14.2 mins; about 16 mins (4-hydroxy-2-butanone); about 17.7 mins; and any combinations thereof, where bioBG elutes at an acquisition time of about 18 mins.

In additional aspects, a 1,3-butylene glycol (BG) product is provided, where the BG product does not comprise one or more compounds characterized by an odor-active region as measured by gas chromatography/mass spectrometry/olfactory (GC/MS/O) having a relative acquisition time selected from a group consisting of: about 0.517 min (toluene); about 0.533 min (butyl acetate); about 0.583 min (butyl acrylate); about 0.889 min (4-hydroxy-2-butanone); and combinations thereof, where the BG product comprises bioderived BG (bioBG), and the bioBG has a relative acquisition time of 1 min. Further aspects provide a 1,3-butylene glycol (BG) product, where the BG product does not comprise one or more compounds characterized by an odor-active region as measured by gas chromatography/mass spectrometry/olfactory (GC/MS/O) having a relative acquisition time selected from a group consisting of: about 0.517 min (toluene); about 0.533 min (butyl acetate); about 0.583 min (butyl acrylate); about 0.611 min; about 0.65 min; about 0.789 min; about 0.889 min (4-hydroxy-2-butanone); about 0.983 min; and combinations thereof, where the BG product comprises bioderived BG (bioBG), and the bioBG has a relative acquisition time of 1 min.

Additional aspects are directed to a 1,3-butylene glycol (BG) product, where the BG product does not comprise one or more compounds comprising an odor-active region as measured by GC/MS/O that elutes at an acquisition time selected from the group consisting of: ˜2.5 mins (rubber and/or plastic and/or green; 3-buten-2-one); ˜2.7 mins (plastic); ˜2.9 mins (solventy; 2-butenal); ˜4.6 mins (plastic: 2,4-dimethyl-1,3-dioxane); ˜6.2 mins (fruity and/or plastic; 2-ethyl-5-methyl-1,3-dioxane); ˜6.6 mins (plastic and/or pungent and/or mushroom and/or metallic; butyl acrylate); ˜8.2 mins (fruity and/or plastic and/or metal and/or mushroom; 2-heptyl-1,3-dioxepane); about 11.8 mins (waxy and/or fatty); and any combinations thereof, where the BG product comprises bioderived BG (bioBG).

In some aspects, a 1,3-butylene glycol (BG) product is provided, where the BG product does not comprise one or more compounds comprising an odor-active region as measured by GC/MS/O that elutes at an acquisition time selected from the group consisting of: ˜9 mins-˜9.2 mins (relative acquisition times ˜0.5 min, ˜0.506 min, ˜0.511 min, respectively) having a fusel and/or sulfur and/or oil odor; ˜9.3 mins (relative acquisition time 0.517 min; toluene); ˜10.2 mins-˜10.4 mins (relative acquisition times ˜0.567 min, ˜0.572 min, ˜0.578 min, respectively) having a fruity and/or plastic odor; ˜10.5 mins-˜10.7 mins (relative acquisition times ˜0.583 min, ˜0.589 min, ˜0.594 min, respectively) having a fruity and/or floral and/or plastic odor; and ˜13.3 mins-˜13.5 mins (relative acquisition times ˜0.739 min, ˜0.744 min, ˜0.75 min, respectively) having a plastic and/or roasty odor; and any combinations thereof, where the BG product comprises a bioderived BG (bioBG).

In some aspects, a 1,3-butylene glycol (BG) product can be provided, where the BG product does not comprise one or more compounds comprising an odor-active region as measured by GC/MS/O having a relative acquisition time selected from a group consisting of: about 0.417 min (rubber and/or plastic and/or green), about 0.450 min (plastic), about 0.483 min (solventy), about 0.783 min (plastic), about 1.033 mins (fruity and/or plastic), about 1.1 mins (plastic and/or pungent and/or mushroom and/or metallic), about 1.367 mins (fruity and/or plastic and/or metal and/or mushroom), about 1.967 mins (waxy and/or fatty), and combinations thereof, where the BG product comprises bioderived BG (bioBG), and the bioBG has a relative acquisition time of 1 min.

In some aspects, the BG product of the disclosure comprises a bioBG selected from the group consisting of: (R)-1,3-BG; (S)-1,3-BG; and a mixture of (R)-1,3-BG and (S)-1,3-BG. Some aspects provide a BG product comprising a bioBG, where the bioBG comprises (R)-1,3-BG. In other aspects of the disclosure, a BG product comprising a bioBG is provided, where the bioBG comprises (S)-1,3-BG. Some aspects provide a BG product, where the BG product comprises bioderived BG (bioBG) comprising a mixture of (R)-1,3-BG and (S)-1,3-BG. In some aspects, any BG product according to the disclosure provides a neutral odor, where the BG product comprises a bioBG.

In other aspects, a composition, comprises: any of the 1,3-butylene glycol (BG) products disclosed here, and a physiologically acceptable vehicle is provided here. The compositions of the disclosure comprising a BG product described here, in some aspects, provide a BG product comprising a neutral odor. Further aspects of the disclosure provide a composition comprising a BG product, where the BG product comprises a bioderived BG (bioBG) and one or more compounds selected from the group consisting of: 5-norbornene-2-ol; 2-ethyl-1-hexanol; and salts, derivatives, analogs, or solvates thereof; or any combinations thereof. In some aspects, provided here are described compositions comprising a BG product disclosed here comprising bioderived BG (bioBG), where the BG product comprises a property selected from the group consisting of: humectant, fungicide, bactericide, conditioning agent, preservative, viscosity-decreasing agent, solvent, and combinations thereof. Some aspects provide compositions comprising a BG product, where the BG product comprises bioderived BG (bioBG), and where the composition is formulated as a cosmetic, a personal care product, or a cosmeceutical. In further aspects, the composition of the disclosure comprising a BG product, where the BG product comprises bioderived BG (bioBG), can be selected from the group consisting of: eye liner, eye shadow, eye makeup, mascara, brow liner, primer, foundation, concealer, BB cream, CC cream, brighteners, blush, bronzer/highlighter, setting powder/spray, body/facial sprays, facial serums, lipstick, lip gloss, lip balm, lip liner, perfumes/fragrances, creams, lotions, moisturizers, face masks, sunscreens, sunblocks, fingernail polishes, deodorants, anti-perspirants, shampoos, conditioners, hair dyes, hair dye removers, soap, cleansers, aromatherapy compositions, massage oils, products comprising an ingredient selected from the group consisting of: vitamins, botanical or marine extracts, peptides, antioxidants, alpha-hydroxy acids, and combinations thereof.

In some aspects, provided here is a method, comprising applying to a subject in need thereof, a composition of any one described here, where the composition comprises any of the aforementioned BG products, where the BG product comprises a bioBG. The method, in other aspects, provides a composition selected from the group consisting of cosmetics, cosmeceutical, and personal care products. Some aspects provide methods of applying to a subject in need thereof, a composition selected from the group consisting of: eye liner, eye shadow, eye makeup, mascara, brow liner, primer, foundation, concealer, BB cream, CC cream, brighteners, blush, bronzer/highlighter, setting powder/spray, lipstick, lip gloss, lip balm, lip liner, perfumes/fragrances, creams, lotions, moisturizers, face masks, sunscreens, fingernail polishes, shampoos, conditioners, hair dyes, hair dye removers, deodorants, anti-perspirants, soaps, cleansers, and cosmetics with vitamins, botanical or marine extracts, peptides, antioxidants, and alpha-hydroxy acids.

Some aspects provide a use of any of the aforementioned 1,3-butylene glycol (BG) products described here or any of the aforementioned compositions described here for: improving appearance, hygiene, or skincare, or providing a property selected from the group consisting of: humectant, fungicide, bactericide, conditioning agent, preservative, viscosity-decreasing agent, solvent, and combinations thereof, to a subject in need thereof.

### BRIEF DESCRIPTION OF FIGURES

FIG. 1 shows an exemplary block flow diagram for deodorization of bioderived 1,3-butylene glycol (bioBG) using a caustic reaction followed by multi-step distillation to produce bioBG Product 1 (DSP-555-2). Values are normalized to 100 kg of feed.

FIG. 2 shows a GC/O analysis of samples DSP-555-1 to DSP-555-4. X axis=Acquisition time (mins). Y axis=counts (%).

FIG. 3 shows a GC/O analysis of samples DSP-555-5 to DSP-555-8. X axis=Acquisition time (mins). Y axis=counts (%).

FIG. 4 shows a GC/O analysis of samples DSP-555-9 to DSP-555-12. X axis=Acquisition time (mins). Y axis=counts (%).

FIG. 5 shows a GC/O analysis of samples DSP-555-13 to DSP-555-16. X axis=Acquisition time (mins). Y axis=counts (%).

FIG. 6 shows GC/MS-O analysis of a DHS (DB5 column) sample of DSP-555-16, where the sample was diluted 10-fold in water and split 10:1 (upper panel) and the sample was diluted 100-fold in water and split 50:1 (lower panel). 1,3-butylene glycol was expected at an acquisition time of 6 mins. X axis=Acquisition time (mins). Y axis=counts (%).

FIG. 7 shows total ion chromatograms from GC/MS/O analysis of neutral odor bioBG (upper panel) and test (lower panel) sample extracts. Aroma regions are highlighted green and indicated by “2” (detected in neutral odor bioBG only) in the upper panel or highlighted blue and indicated by “1”, “3”, “4”, and “5” (detected in test only) in the lower panel.

FIG. 8 shows total ion chromatograms from GC/QTOF-MS analysis of neutral odor bioBG (upper panel) and test (lower panel) sample extracts. Aroma regions are highlighted green and indicated by “2” (detected in neutral odor bioBG only) or highlighted blue and indicated by “1”, “3”, “4”, and “5” (detected in test only).

FIG. 9 shows extracted ion chromatograms (m/z 70.0411) for 1-octen-3-one (aroma 4) in test sample (upper panel), control sample (center panel), and 2.5 ppm pure standard (lower panel).

FIG. 10 shows extracted ion chromatograms (m/z 59.0489), QTOF EI mass spectra (70 eV), and NIST17 library search results for 2-methoxymethyl-2,4,5-trimethyl-1,3-dioxolane (aroma 1) in test (upper panel) and control (lower panel) samples.

FIG. 11 shows extracted ion chromatograms (m/z 66.0463), QTOF EI mass spectra (70 eV), and NIST17 library search results for 5-norbornene-2-ol (bicyclo[2.2.1]hept-5-en-2-ol) (aroma 2) in test (upper panel) and control (lower panel) samples.

FIG. 12 shows extracted ion chromatograms (m/z 57.0698), QTOF EI mass spectra (70 eV), and NIST17 library search results for 3-heptanone (aroma 3) in test and control samples.

FIG. 13 shows extracted ion chromatograms (m/z 114.0675), QTOF EI mass spectra (70 eV), and NIST17 library search results for N-pentyl-2-butylamine (aroma 5) in test (upper panel) and control (lower panel) samples.

FIG. 14 shows a GC/MS chromatogram for sample DSP-555-8. Y-axis=Counts (%); X-axis=Acquisition Time (mins), where butyl acetate, butyl acrylate, 4-hydroxy-2-butanone, and 1,3-BG are identified.

FIG. 15 shows standard addition curves built in test sample (DSP-650-03; equivalent of DSP-555-6 to DSP-555-12 samples) for selected aroma compounds. Values are represented as average standard deviation of three biological replicates.

FIG. 16 shows standard addition curves built in control sample (DSP-650-04; large scale of DSP-555-4) for selected aroma compounds. Values are represented as average±standard deviation of three biological replicates.

FIG. 17 shows results of a dynamic headspace (DHS) GC/MS analysis of a DSP-722 sample and compounds.

FIG. 18 shows overlaid chromatograms of 100-fold dilution solutions of neutral odor bioBG samples, 1,3-butylene glycol standard solution, and methanol blank.

FIG. 19 shows a zoomed-in portion of the overlaid chromatograms from FIG. 18 of a peak present in the neutral odor bioBG samples and not observed in the 1,3-butylene glycol standard solution or methanol blank.

FIG. 20 shows a NIST library match of the peak present in the neutral odor bioBG samples and not observed in the 1,3-butylene glycol standard solution or methanol blank of FIG. 19.

FIG. 21 shows overlaid chromatograms of headspace of neutral odor bioBG samples and 1,3-butylene glycol standard (1,3-butylene glycol method).

FIG. 22 shows a zoomed-in portion of the overlaid chromatograms from FIG. 21 of a peak present in the neutral odor bioBG samples identified as toluene.

FIG. 23 shows a NIST library match of the peak present in the neutral odor bioBG samples of FIGS. 21-22 identified as toluene.

FIG. 24 shows overlaid chromatograms of headspace of neutral odor bioBG samples, 1,3-butylene glycol standard, and toluene standard (volatiles method).

FIG. 25 shows a zoomed-in portion of the overlaid chromatograms from FIG. 24 of a peak present in the neutral odor bioBG samples and toluene standard and identified as toluene.

FIG. 26 shows a NIST library match of the peak identified as toluene in the neutral odor bioBG samples of FIGS. 24-25.

### DETAILED DESCRIPTION

Detailed embodiments of the present disclosure are disclosed herein; however, it is to be understood that the disclosed embodiments are merely illustrative of the disclosure that may be embodied in various forms. In addition, each of the examples given in connection with the various embodiments of the disclosure is intended to be illustrative, and not restrictive.

All terms used herein are intended to have their ordinary meaning in the art unless otherwise provided. All concentrations are in terms of percentage by weight of the specified component (e.g., fragrance ingredient) relative to the entire weight of the composition, unless otherwise defined.

Demand for neutral odor additives in cosmetics, cosmeceuticals, or personal care products is desirable.

### Butylene Glycol (BG) Product

In some embodiments, a purified 1,3-butylene glycol (synonymous and used interchangeably with BG; 1,3-BG; 1,3-butanediol; 1,3-BDO; butane-1,3-diol; etc.) can be present in a product (BG product) of the disclosure. CAS No. 107-88-0 is assigned to BG having a chemical formula of C₄H₁₀O₂. There are various methods for producing 1,3-butylene glycol. For example, U.S. Pat. No. 6,376,725, which is incorporated herein by reference in its entirety, describes methods of liquid phase hydrogenation of acetaldol (3-hydroxybutanal or aldol) with a Raney nickel catalyst resulting in the production of 1,3-butylene glycol. Another method of producing 1,3-butylene glycol is described as having three steps: aldol condensation of acetaldehyde to aldoxane, then decomposition of the aldoxane resulting in paraldol, which is then hydrogenated to finally produce 1,3-butylene glycol (as described in, e.g., U.S. Pat. Nos. 5,345,004; 5,583,270, which are incorporated herein by reference in their entirety).

Some embodiments of the disclosure are directed to a bioderived BG that has been purified and present in a BG product described here to form a bioderived BG product. BG can be a bioderived 1,3-butylene glycol (bioBG), which can include any type or form of BG, including but not limited to (R)-1,3-BG; (S)-1,3-BG; mixtures of (R)-1,3-BG and (S)-1,3-BG; or any combinations or mixtures of the aforementioned. In some examples, BG is a sustainable, natural bioderived 1,3-BG (bioBG), which can be produced by non-naturally occurring microorganisms that express genes encoding enzymes that catalyze bioBG production. The methods and compositions for producing bioBG through culturing of non-naturally occurring microbial organisms are described in, for example, WO 2010/127319 (e.g., Examples I-III), WO 2011/071682 (e.g., Examples I, III), and WO 2012/177619 (e.g., Examples I-IV, VIII), which disclose methods, vectors, strains, and culturing materials and conditions for producing bioderived 1,3-BG; and WO 2018/183628 (e.g., Example 1) and WO 2018/183664, which specifically disclose engineered organisms and enzymes producing bioderived 1,3-BG, as well as characterization of bioderived 1,3-BG, each of which is incorporated by reference in its entirety.

In some embodiments, the BG product or bioBG product of the disclosure can be purified and obtained by any one of the commonly known and used methods, including but not limited to, distillation, solvent extraction, and enfleurage (e.g., steam distillation, hydrodistillation, cohobation, molecular distillation, cold pressed extraction, and sieving). For example, solvent extraction can be performed by mixing a sample comprising 1,3-BG with a mixture of water and dichloromethane (DCM) (e.g., 1:1:1; 1:2:2). The mixture can be vigorously mixed and then the DCM layer collected. The DCM layer can then be concentrated to a volume (e.g., 200 μL) under nitrogen gas. In some embodiments, the concentrated extract can be measured by gas chromatography/mass spectrometry/olfactory (GC/MS/O) by injecting, for example, 1 μL into the device. The extract can be analyzed by a panel of trained sensory experts to assess differentiating aroma attributes between the concentrated extract and control samples. The chemical profile of the extract can be determined and analyzed by mass spectrometry. For example, control samples can comprise more than 99.7 weight (wt)% 1,3-BG. Some non-limiting samples can comprise 10 wt % 1,3-BG in deionized water, less than 10 wt % 1,3-BG in water that can contain other impurities, or 90 wt % 1,3-BG and other impurities.

As used herein, the term “non-naturally occurring” when used in reference to a microbial organism or microorganism of the disclosure is intended to mean that the microbial organism has at least one genetic alteration not normally found in a naturally occurring strain of the referenced species, including wild-type strains of the referenced species. Genetic alterations include, for example, modifications introducing expressible nucleic acids encoding metabolic polypeptides, other nucleic acid additions, nucleic acid deletions and/or other functional disruption of the microbial organism's genetic material. Such modifications include, but are not limited to, coding regions and functional fragments thereof, for heterologous, homologous, or both heterologous and homologous polypeptides for the referenced species. Additional modifications include, for example, non-coding regulatory regions in which the modifications alter expression of a gene or operon. Exemplary metabolic polypeptides include enzymes or proteins within a 1,3-butanediol biosynthetic pathway.

A metabolic modification refers to a biochemical reaction that is altered from its naturally occurring state. Therefore, non-naturally occurring microorganisms can have genetic modifications to nucleic acids encoding metabolic polypeptides or, functional fragments thereof.

These microorganisms can be supplied a source of carbon via any carbohydrate source. Such sources include, but are not limited to, sugars (e.g., hexoses: glucose, fructose, galactose, mannose; pentoses: xylose, arabinose) and starches. Plant biomass, an exemplary carbohydrate source, is a type of renewable organic material from plants that can be converted into chemicals, biofuels, and other materials. Non-limiting carbohydrate sources include renewable plant biomasses and feedstocks, such as but not limited to, cellulosic biomass, hemicellulosic biomass, and lignin feedstocks or portions of feedstocks.

As used herein, the term “bioderived” means derived from or synthesized by a biological organism and can be considered a renewable resource since a bioderived product can be generated by a biological organism. Such a biological organism, in particular, the microbial organisms of the disclosure described here, can utilize feedstock or biomass, such as, sugars or carbohydrates obtained from an agricultural, plant, bacterial, or animal source. Alternatively, the biological organism can utilize atmospheric carbon. As used here, the term “biobased” means a product as described here that is composed of, in whole or in part, a bioderived compound of the disclosure. A biobased or bioderived product of the disclosure (e.g., bioderived 1,3-butylene glycol or bioBG) is not, and is in contrast to, a “petroleum-derived product,” where such a petroleum-derived product is derived from or synthesized from petroleum or a petrochemical feedstock. A bioderived 1,3-butylene glycol used here can be a natural product as measured by the International Organization for Standardization (ISO). ISO 16128-1:2016 provides definitions for organic and natural cosmetic ingredients, and ISO 16128-2:2017 provides guidelines and criteria for determining natural, organic, or natural and organic origins of cosmetic ingredients and products.

### Deodorization of BG Product

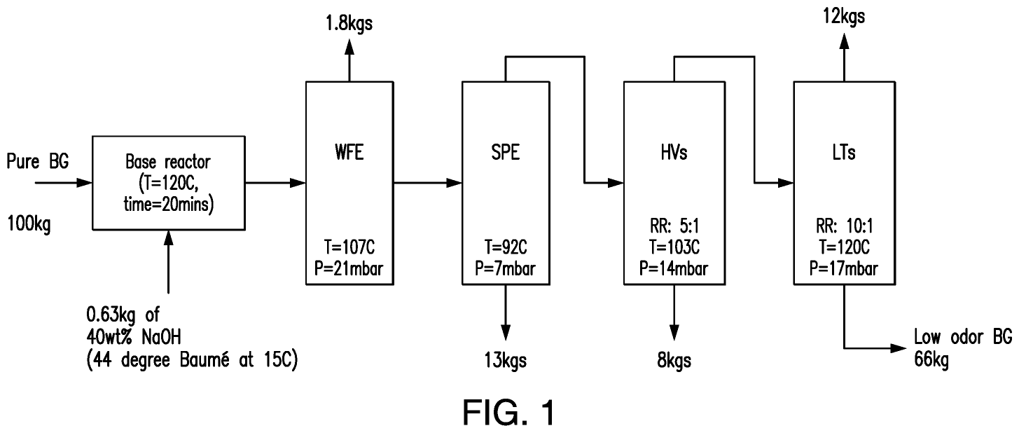

A high odor BG product, such as a bioBG, can be treated to remove or deodorize an undesirable odor using, for example, a distillation process in order to produce a low odor BG or a neutral odor BG. See, EXAMPLE 1; FIG. 1. Briefly, the high odor BG can be treated using a multi-step distillation process after reacting the high odor bioBG of the disclosure with a caustic solution, such as a sodium hydroxide (NaOH) solution of about 40 wt % to about 50 wt %, in order to obtain a final concentration of NaOH of, for example, 0.25 wt % (FIG. 1). The mixture of high odor BG and caustic solution can be reacted in a tank vessel (e.g., base reactor, caustic reactor) resulting in a reaction product. The reaction product can then be fed into multiple distillation columns to ultimately produce a final deodorized BG, low odor BG, or a neutral odor BG (interchangeably used). The distillation process can comprise multiple lights removal and heavies removal. The lights waste can be collected and re-processed.

An exemplary conventional purification method can include a method in which a reaction mixture of, for example, 1,3-butylene glycol (e.g., a bioderived 1,3-BG provided herein) produced by liquid phase hydrogen reduction of acetaldol, is subjected to removal of alcohols, removal of water, removal of salts, and removal of high-boiling materials. Then in a low-boiling material removal distillation tower (product distillation tower), low-boiling materials can be distilled off from the top of the tower and 1,3-butylene glycol (e.g., a neutral odor bioderived BG) can be obtained as a product from the tower bottom. The addition of a base to bioderived 1,3-butylene glycol having a low content of high-boiling materials and heat-treatment of the mixture effectively decreases odor-causing materials and gives rise to a neutral odor bioderived 1,3-butylene glycol or a 1,3-butylene glycol having low to no odor which shows less change with time. The base to be added can include but is not limited to: an alkali metal compound, sodium hydroxide, potassium hydroxide, sodium (bi)carbonate, sodium hydroxide, potassium hydroxide or mixtures thereof. The base can be added in the form of solids as it is or can be added as an aqueous solution for ease of operation and for promoting contact with the target solution. Suitable base reactor and distillation reaction temperatures and conditions are provided in FIG. 1. In some embodiments, a distillation reaction temperature ranges from at least about 90 degrees Celsius (° C.) up to about 140° C. In some embodiments, the reaction retention time is at least about 5 minutes up to about 120 minutes. In some embodiments, the reaction retention time is at least about 10 minutes up to about 30 minutes.

From the top of the tower, bioderived 1,3-butylene glycol containing low-boiling materials is distilled off and is charged to the next product distillation tower. The product distillation tower may be a porous plate tower, a foamed bell tower, etc. The product distillation tower can be a filled tower having a low pressure loss, filled with Sulzer Packing, Melapack™ (both are trade names for products by Sumitomo Heavy Industries, Ltd.), etc., which is more suitable than other fillers. Without being bound by theory, the suitability is because 1,3-butylene glycol is thermally decomposed at 2000° C. or higher which adversely affects odor (See, e.g., U.S. Pat. No. 6,376,725 B1, the contents of which is incorporated herein by reference in its entirety). Accordingly, distillation temperature should be lowered as low as possible. When thermal hysteresis (retention time) to 1,3-butylene glycol is long, similarly influenced. Therefore, the reboiler to be adopted is suitably a thin film evaporator, such as a natural flow-down type thin film evaporator or a forced stirring type thin film evaporator.

The product distillation can depend on the concentration of low-boiling material in the charge stock solution, but when the concentration of low-boiling material in the charge stock solution is 5% or less, it can be one having a theoretical number of daylights of about 10 to 20 (trays). The charge stock solution is fed at a position from the top of the tower to a height 20 to 70% of the height of the tower. At this time, distillation is performed under reduced pressure of 100 torr (133.322 millibars (mbar)) or less, preferably 5 torr to 20 torr (6.66612 mbar-26.6645 mbar), at the top of the tower. For the odor of a bioderived 1,3-butylene glycol to be removed, it is preferred to use a lower distillation temperature. The lower the pressure is, the more suitable. It is desirable to run distillation at a reflux ratio (RR) of at least about 0.5 up to about 15.

The charging into the product distillation tower is carried out by feeding the liquid obtained by concentrating the top of the tower vapor of the dealkalization tower in the condenser. To decrease the calorie for heating the product distillation tower, the top of the tower vapor from the dealkalization tower may be directly fed to the production tower. Bioderived 1,3-butylene glycol product can be obtained from the tower bottom of the product distillation tower. Suitable distillation conditions for generating a low odor bioderived 1,3-bioBG are further described in Example 2.

Alternative distillation protocols known in the art can be used. By way of example only, the treated liquid from an alkali reactor can be fed first into the low-boiling material removal distillation tower where low-boiling materials are distilled off, subsequently the bioderived 1,3-butlene glycol extracted from the gas phase portion in the recover trays or from the tower bottom is distilled or evaporated to remove the base and the resulting high-boiling materials as distillation residues and 1,3-butylene glycol is recovered from the top of the tower or in the midway of concentration trays. The distillate of the above low-boiling materials can further be fed to an additional distillation tower where 1,3-butylene glycol can be distilled off to remove high-boiling materials. Also, a portion of the 1,3-butylene glycol after removal of the above low-boiling materials can be recycled to the dealkalization tower. Alternatively, low-boiling materials containing 1,3-butylene glycol can be recycled to the alkali reactor.

A caustic process can occur in an alkali reactor (see, e.g., U.S. Pat. No. 6,376,725, which is incorporated herein by reference in its entirety) or by simply adding a caustic solution, such as, caustic soda, caustic potash, sodium borohydride, and potassium borohydride during deboiling distillation (see, e.g., JP 3369707 B2, which is incorporated herein by reference in its entirety). Accordingly, some embodiments of the disclosure provide a method of obtaining a neutral odor BG comprising one or more (e.g., 2, 3, 4) distillations for use following any caustic (e.g., alkali) process with or without an alkali reactor.

The final deodorized BG can be used in a neutral odor BG product or composition comprising a neutral odor BG product as described here. A neutral odor, as used here, can be or can essentially be a scentless, an odorless, or an undetectable odor, where a neutral odor can be detected below an odor threshold. In some embodiments, the neutral odor bioBG can comprise, consist essentially of, consist of, or minimally comprise one or more aromatic compounds which can be responsible for an odor region or an odor-active region described here, where the one or more compounds can be present in an amount below its respective odor threshold. In some uses, a composition can have a desired fragrance that is unaffected by the neutral odor components or ingredients described here.

### Neutral Odor BG Product

Embodiments of the disclosure are directed to a 1,3-butylene glycol (1,3-BG; BG) product comprising a bioderived BG (bioBG), where the BG product is removed of or essentially removed of any or all contaminants or compounds, which can have an undesirable or offensive odor, by, for example, the deodorization or distillation process described here. Some embodiments are directed to a BG product of the disclosure, where the BG product comprises a neutral odor. In some embodiments, any of the BG products of the disclosure can comprise a neutral odor bioBG, where the neutral odor bioBG and/or one or more compounds present therein, is present in an amount below its odor threshold. The BG product of the disclosure can be a liquid product or a viscous liquid product. In some embodiments, the BG product can comprise an isolated or a purified bioBG.

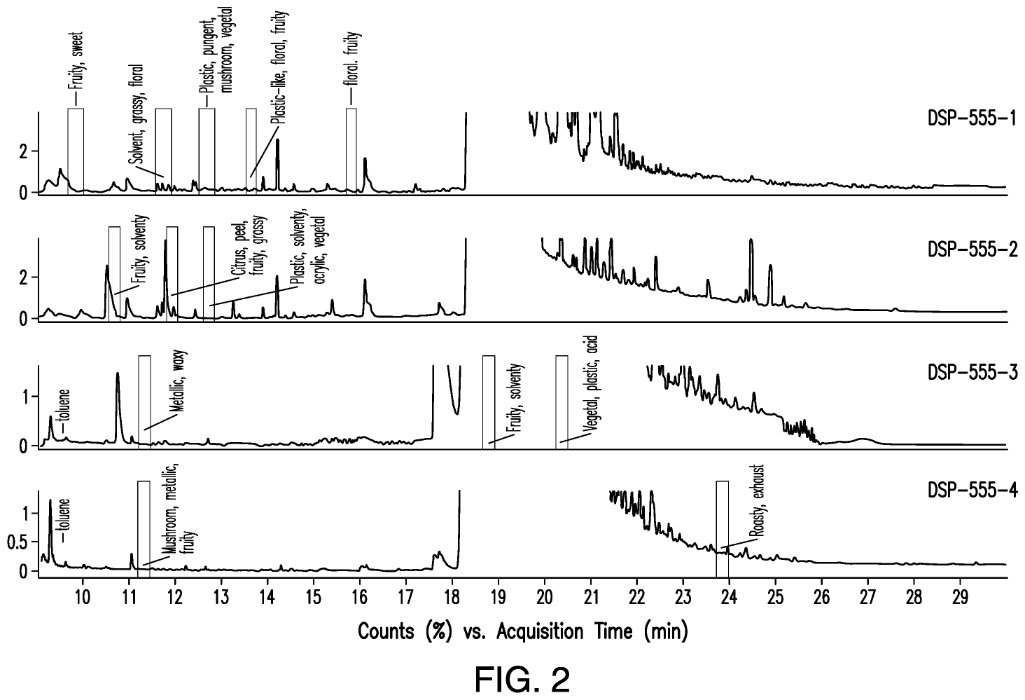

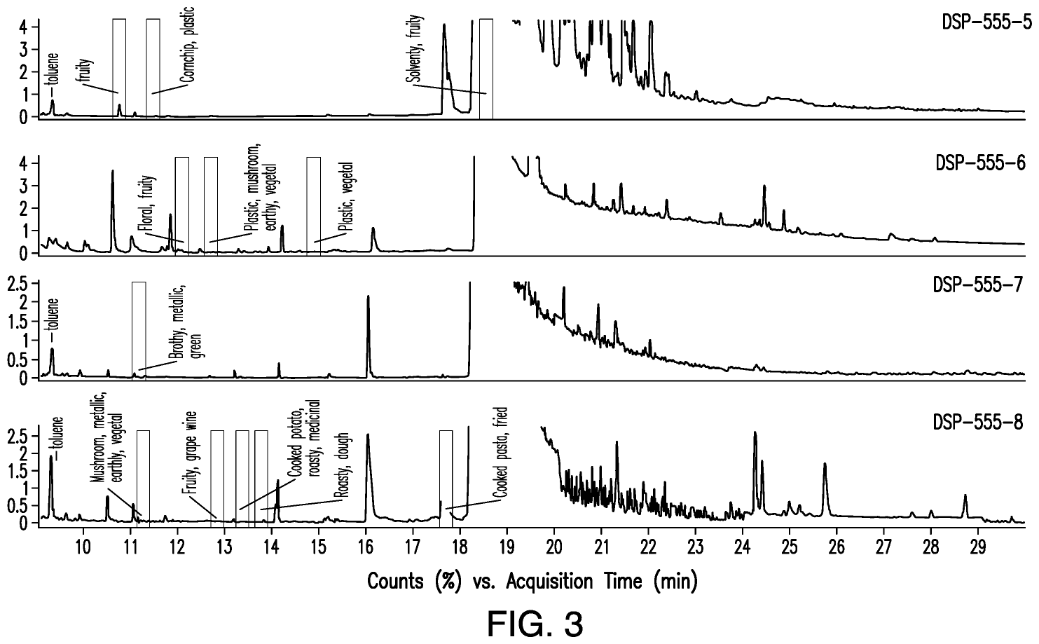

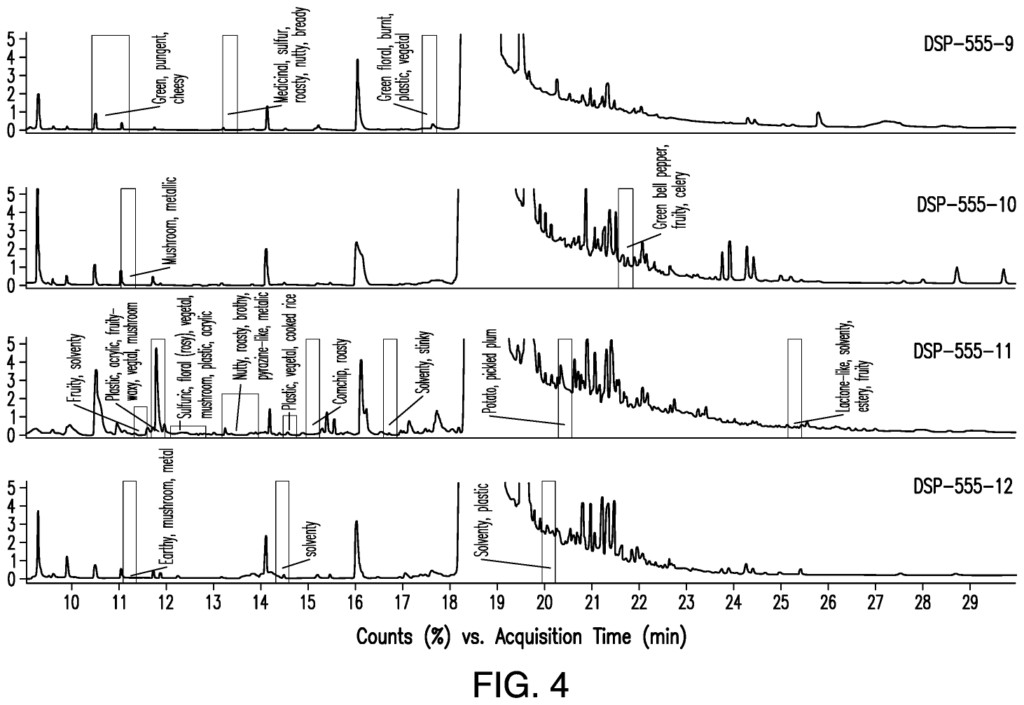

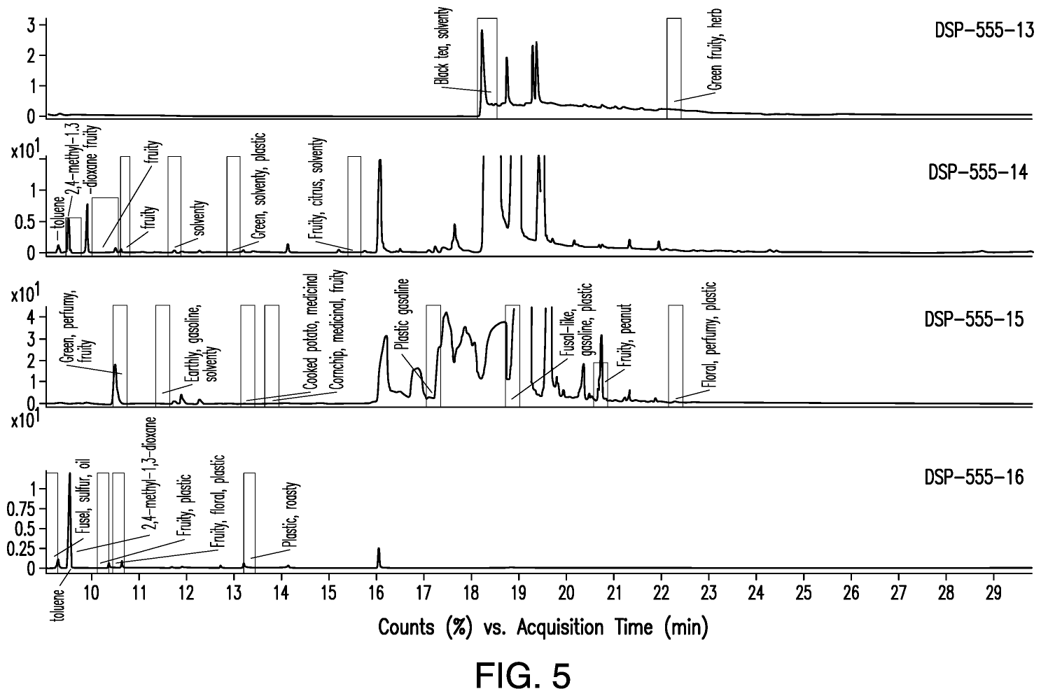

As used herein, the term “odor-active region” is intended to mean an identifiable region within a fractionated sample of 1,3-butylene glycol (BG). The fractionation method can be any method known in the art that separates components based on, for example, chemical and/or physical properties. The fractionation method exemplified in this disclosure is gas chromatography-mass spectrometry (GC/MS) as exemplified in FIGS. 2-5. The odor for the various 1,3-BG fractions is determined by a sensory odor panel composed of trained individuals and referred to herein as GC/O analysis (see, e.g., EXAMPLE 2). In some embodiments, toluene can influence odor, or can be a contributor to “off-odor”, which is an undesirable, malodorous scent. Other embodiments can be directed to crotonaldehyde and/or methyl vinyl ketone as contributors to “off-odor”. In additional embodiments, any of the one or more compounds and/or odor-active regions associated with compounds described here can contribute to “off-odor”.

As used herein, the term “odor threshold” is intended to mean the point at which 50% of a panel of the sensory odor panelists cannot smell a tested odor and corresponds to one (1) odor unit per cubic meter (1 o.u./m³).

As used herein, the term “neutral odor” refers to a non-offensive odor to the olfactory system as determined by a subject, machine, sensory odor panelist, or any combination. A “neutral odor bioderived BG” or a “neutral odor bioBG” and grammatical equivalents refers to a bioderived 1,3 butylene glycol that does not have an offensive odor. For example, a sulfuric odor above the perception threshold may be considered an offensive odor as determined by sensory odor panelists.

As used herein, the term “low odor” and its grammatical equivalents refers to a non-offensive odor to the olfactory system that is below the perception threshold as determined by a subject, machine, or sensory odor panelist The terms “low odor bioderived butylene glycol,” “low odor bioBG,” or “neutral odor BG,” or “neutral odor bioBG” are used interchangeably to refer to a bioderived 1,3-butylene glycol composition that has an odor-active region that is below the perception threshold for the odor associated with the corresponding odor-active region on a GC/MS chromatograph. For example, a bioBG composition of the disclosure can comprise a fruity odor-active region having a relative acquisition time of 0.594 mins that is below the perception threshold for that odor and would not be perceived by a panel of trainer professionals or a machine to have a fruity odor. In some cases, a neutral odor bioBG product is produced by removing one or more chemical compounds from an unpurified, less purified, or crude bioderived BG product described here.

As used herein, the terms “odorous 1,3-bioBG”, “odorous bioBG”, “fragrant 1,3-bioBG”, or “fragrant bioBG” are used interchangeably to refer to a bioderived 1,3-butylene glycol composition that has an odor-active region that is above the perception threshold for the odor associated with the odor-active region. For example, a fragrant bioBG can comprise a fruity odor-active region having a relative acquisition time of 0.594 mins that is above the perception threshold for that odor. In some embodiments, a chemical or fragrance can be added to a neutral odor bioBG composition provided herein to produce a fragrant bioBG composition.

As used herein, the term “fragrance substance” is intended to mean a material that provides or enhances a particular smell. The material can include a solid, powder, a liquid, or a gas, for example. The fragrance substance can be a natural or artificial scent. In some instances, the neutral odor BG product described herein can be a fragrance substance.

As used herein, the term “comprising” is intended to mean that the BG products, compositions comprising such BG products, and methods of using the BG products or compositions thereof as described here include the recited elements, but do not exclude others. “Consisting essentially of” when used to define the BG products, compositions, and methods, shall limit the scope of a claim to the specified materials or steps and those that do not materially affect the basic and novel characteristics of the claimed invention, or affect odor. Thus, a BG product or composition consisting essentially of the elements as defined herein could exclude trace contaminants from the isolation and purification method. “Consisting of” shall exclude any compound, element, or step not specified in the claim. “Minimally comprises” when used here to define the BG products and compositions disclosed, refers to one or more compounds that can contribute to off-odor in an amount or concentration that is, for example, below its odor threshold.

Some embodiments of the disclosure provide a 1,3-butylene glycol (BG) product comprising a bioBG and further comprises, minimally comprises, consists essentially of, or consists of one or more compounds selected from: 5-norbornene-2-ol; 2-ethyl-1-hexanol; and salts, derivatives, analogs, or solvates thereof, or any combinations thereof, wherein the BG product comprises a bioderived BG (bioBG), where, in some embodiments, the one or more compounds are present in an amount or concentration below its odor threshold. Additional embodiments can provide a BG product comprising bioBG and further comprises, minimally comprises, consists essentially of, or consists of one or more compounds selected from the group consisting of: 5-norbornene-2-ol; 2-ethyl-1-hexanol; 3-buten-2-one; 3-heptanone; 1-octen-3-one; styrene; butyl acrylate, HDD; and salts, derivatives, analogs, or solvates thereof, or any combinations thereof. In some embodiments, such one or more compounds can be in an amount or concentration below its odor threshold.

In some embodiments, a BG product comprising a bioBG can comprise, minimally comprise, consist essentially of, or consist of: one or more compounds selected from the group consisting of: 5-norbornene; 2-ethyl-1-hexanol in an amount of ˜0.000001 ppm or greater (e.g., ˜0.000001 ppm-˜10 ppm; ˜0.1 ppm-˜0.2 ppm) 3-buten-2-one in an amount of about 1.75 ppm-2 ppm; styrene in an amount of 0.0005 ppm or less; 3-heptanone in an amount of 0.00005 ppm or less, or in an amount of less than 95 or less than 4 in fold change relative to a reference value or a control as quantified using a dichloromethane extraction method; 1-octen-3-one in an amount of 0.00002 ppm or less, or in an amount of less than 187, 45 or 19 fold change relative to a reference value or a control as quantified using a dichloromethane extraction method; and salts, derivatives, analogs, or solvates, thereof, or any combinations thereof. In some embodiments, such one or more compounds can be in an amount or concentration below its odor threshold.

Additional embodiments provide a BG product of the disclosure comprising a bioBG, and also comprises, consists essentially of, consists of, or minimally comprises one or more compounds as characterized by an aromatic region, an odor-active region, or a peak as measured by extracted ion chromatograms selected from the group consisting of: 2-methoxymethyl-2,4,5-trimethyl-1,3-dioxolane having about 0 counts-1.6×10⁶ counts at 59.0489 m/z; 1-octen-3-one having about 0.15×10⁴-about 0.4×10⁴ counts at m/z 70.0411; 3-heptanone having about 0.4×10⁵ counts at m/z 57.0698; N-pentyl-2-butylamine having about 0 counts at m/z 114.0675; 5-norbornene-2-ol having about 0 counts to about 3.75×10⁵ counts at m/z 66.0463; and any combinations thereof. In some embodiments, such one or more compounds can be in an amount or concentration below its odor threshold.

In some embodiments, a BG product of the disclosure, comprising a bioBG, also comprises, minimally comprises, consists essentially of, or consists of one or more of the compounds disclosed here, where the compound is in an amount of 0 ppm or more (e.g., 0.000001 or more), 10 ppm or less, or 0 ppm-10 ppm, where the compound is selected from the group consisting of: 5-norbornene-2-ol; 2-ethyl-1-hexanol; 3-buten-2-one; 3-heptanone; 1-octen-3-one; styrene; butyl acrylate, HDD; and salts, derivatives, analogs, or solvates thereof, or any combinations thereof. In some embodiments, such one or more compounds can be in an amount or concentration below its odor threshold.

In some embodiments, a disclosed BG product comprising a bioBG comprises, minimally comprises, consists essentially of, or consists of: one or more compounds comprising an aromatic region, an odor-active region, or a peak as measured by GC/MS/O with an odor selected from the group consisting of: (a) fruity; (b) citrus and/or floral and/or oily; (c) rubber and/or plastic and/or green; (d) plastic and/or musty; (e) mushroom and/or earthy, that corresponds to compounds: (a) 5-norbornene-2-ol; (b) 2-ethyl-1-hexanol; (c) 3-buten-2-one; (d) 3-heptanone; (e) 1-octen-3-one, respectively. In some embodiments, such one or more compounds can be in an amount or concentration below its odor threshold.

In some embodiments, the BG product of the disclosure comprising a bioBG, and further comprising, minimally comprising, consisting essentially of, or consisting of one or more compounds selected from the group consisting of: 5-norbornene-2-ol; and 2-ethyl-1-hexanol; 3-buten-2-one; 3-heptanone; 1-octen-3-one; styrene, where the one or more compounds is in an amount of 0 ppm or more (e.g., 0.00001 or more), 10 ppm or less, or 0 ppm-10 ppm. For example, 3-buten-2-one can be in an amount of about 1.75 ppm-2 ppm; or styrene can be in an amount of 0.0005 ppm or less. In some embodiments, such one or more compounds can be in an amount or concentration below its odor threshold.

Additional embodiments are directed to a 1,3-butylene glycol (BG) product comprising one or more compounds characterized by an odor-active region as measured by gas chromatography/mass spectrometry/olfactory (GC/MS/O) having a relative acquisition time selected from a group consisting of: about 0.594 mins (fruity and/or solventy), about 0.628 mins (fruity), about 0.655 mins (citrus and/or peel), about 0.711 mins (solventy), about 1.328 mins (roasty and/or exhaust), and combinations thereof, where the BG product comprises bioderived BG (bioBG), and the bioBG has a relative acquisition time of 1. In some embodiments, such one or more compounds can be in an amount or concentration below its odor threshold.

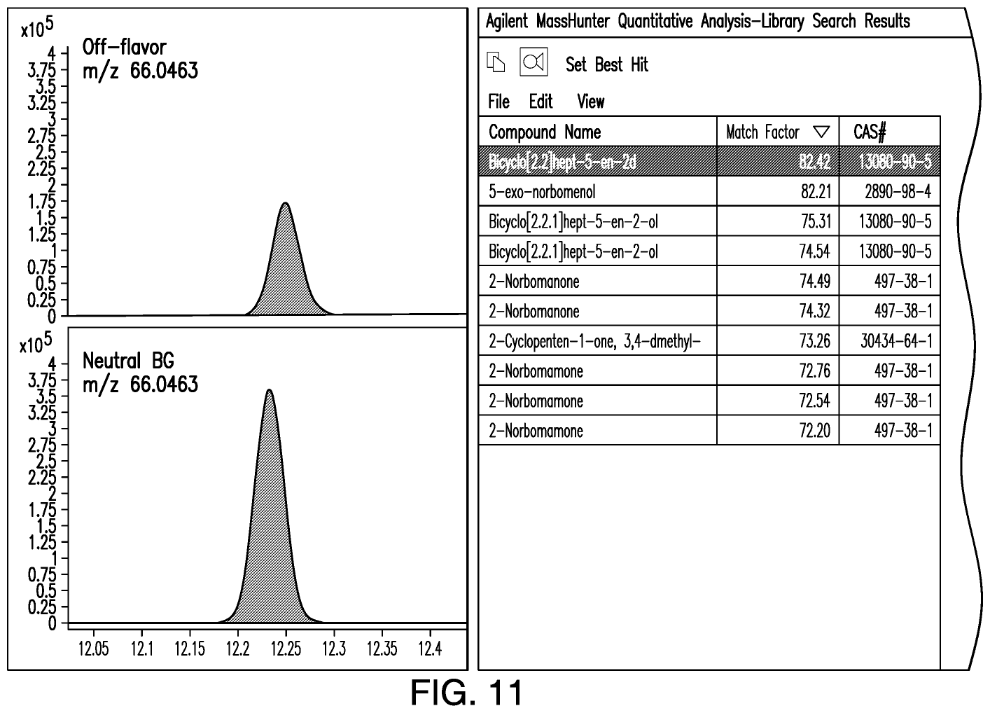

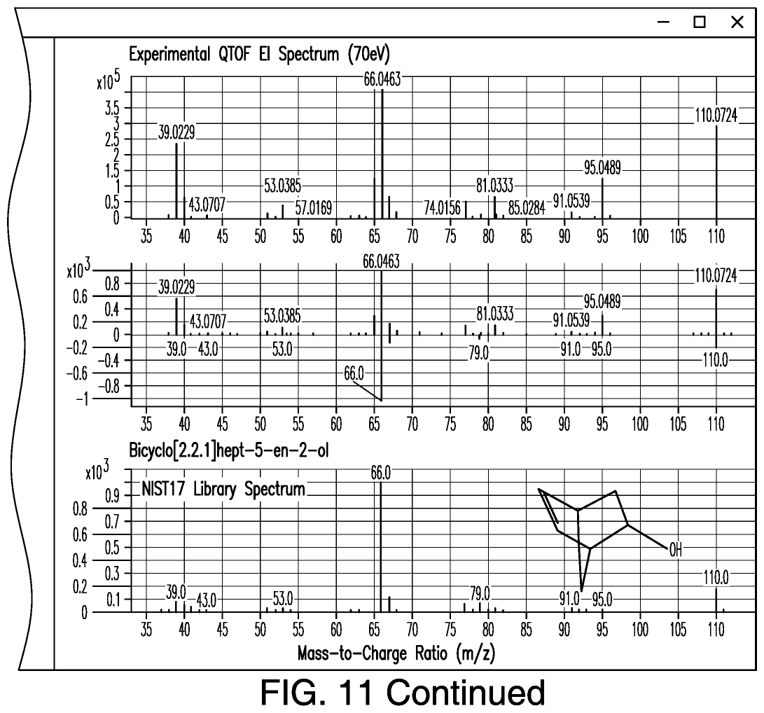

Some embodiments of the disclosure provide a 1,3-butylene glycol (BG) product comprising a bioBG and further comprises, minimally comprises, consists essentially of, or consists of one or more compounds characterized by an aromatic region, an odor-active region, or a peak as measured by gas chromatography/quadrupole time-of-flight-mass spectrometry (GC/QTOF-MS), where the aromatic region, the odor-active region, or the peak elutes at an acquisition time selected from a group consisting of: about 12.2 mins (e.g., 12.18 mins-12.27 mins; 12.18 mins, 12.19 mins, 12.2 mins, 12.21 mins, 12.22 mins, 12.23 mins, 12.24 mins, 12.25 mins, 12.26 mins, 12.27 mins) at 66.0463 m/z. See, FIG. 11. In some embodiments, such one or more compounds can be in an amount or concentration below its odor threshold.

Embodiments of the disclosure can also be directed to a 1,3-butylene glycol (BG) product having a bioBG and further comprises, consists essentially of, or minimally comprises one or more compounds having a mass spectrum as characterized by quadrupole time-of-flight-mass spectrometry electron ionization (QTOF EI) at 70 eV. Such a mass spectrum comprises peaks: 39.0229 m/z; 43.0707 m/z; 53.0385 m/z; 57.0169 m/z; 66.0463 m/z; 74.0156 m/z; 81.0333 m/z; 85.0284 m/z; 91.0539 m/z; 95.0489 m/z; and 110.0724 m/z; or a mass spectrum comprises peaks: 39.0229 m/z; 43.0707 m/z; 53.0385 m/z; 66.0463 m/z; 81.0333 m/z; 91.0539 m/z; 95.0489 m/z; and 110.0724 m/z. See, FIG. 11. In some embodiments, such one or more compounds can be in an amount or concentration below its odor threshold.

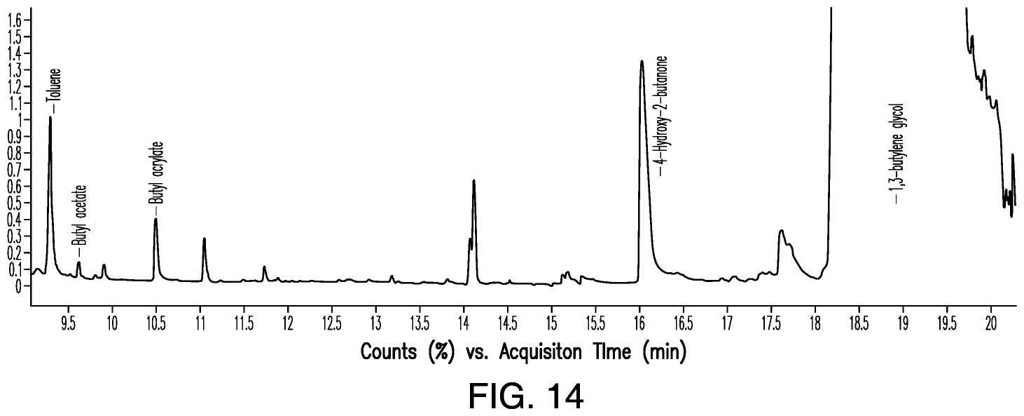

In some embodiments, a BG product, comprising bioBG, does not comprise, does not consist essentially of, does not consist of, or minimally comprises one or more compounds having an aromatic region, an odor-active region, or a peak as measured by GC/MS that elutes at an acquisition time selected from a group consisting of: about 9.3 mins (toluene); about 9.6 mins (butyl acetate); about 10.5 mins (butyl acrylate); about 16 mins (4-hydroxy-2-butanone); and any combinations thereof, where bioBG elutes at an acquisition time of about 18 mins; or an aromatic region, an odor-active region, or a peak as measured by GC/MS that elutes at an acquisition time selected from a group consisting of: about 9.3 mins (toluene); about 9.6 mins (butyl acetate); about 10.5 mins (butyl acrylate); about 11 mins; about 11.7 mins; about 14.2 mins; about 16 mins (4-hydroxy-2-butanone); about 17.7 mins; and any combinations thereof, where bioBG elutes at an acquisition time of about 18 mins. See, FIG. 14. In some embodiments, such one or more compounds can be in an amount or concentration below its odor threshold. Further embodiments provide for such one or more compounds that are contributors to “off-odor”. For example, toluene has an odor threshold ranging from about 0.16 ppm to about 100 ppm, where 50% of the sensory odor panelists cannot smell the odor emitted by toluene.

Additional embodiments are directed to a 1,3-butylene glycol (BG) product, where the BG product does not comprise one or more compounds characterized by an aromatic region, an odor-active region, or a peak as measured by gas chromatography/mass spectrometry/olfactory (GC/MS/O) having a relative acquisition time selected from a group consisting of: about 0.517 mins (toluene); about 0.533 mins (butyl acetate); about 0.583 mins (butyl acrylate); about 0.889 mins (4-hydroxy-2-butanone); and combinations thereof, where the BG product comprises bioderived BG (bioBG), and the bioBG has a relative acquisition time of 1 min; or an aromatic region, an odor-active region, or a peak as measured by gas chromatography/mass spectrometry/olfactory (GC/MS/O) having a relative acquisition time selected from a group consisting of: about 0.517 mins (toluene); about 0.533 mins (butyl acetate); about 0.583 mins (butyl acrylate); about 0.611 mins; about 0.65 mins; about 0.789 mins; about 0.889 mins (4-hydroxy-2-butanone); about 0.983 mins; and combinations thereof, where the BG product comprises bioderived BG (bioBG), and the bioBG has a relative acquisition time of 1 min. In some embodiments, such one or more compounds can be in an amount or concentration below its odor threshold. Further embodiments provide for such one or more compounds that are contributors to “off-odor”.

In further embodiments, provided here is a BG product of the disclosure, where the BG product comprises a neutral odor bioBG and does not comprise, does not consist essentially of, does not consist of, or minimally comprises one or more compounds: 2-methoxymethyl-2,4,5-trimethyl-1,3-dioxolane, toluene, or combinations thereof. In some embodiments, such one or more compounds can be in an amount or concentration below its odor threshold. Further embodiments provide for such one or more compounds that are contributors to “off-odor”.

Some embodiments are directed to a BG product of the disclosure comprising a bioBG and that does not comprise, does not consist essentially of, does not consist of, or minimally comprises any one or more of the compounds selected from the group consisting of: butyl acetate; butyl acrylate; 4-hydroxy-2-butanone; 2-methoxymethyl-2,4,5-trimethyl-1,3-dioxolane; 3-heptanone; 1-octen-3-one; N-pentyl-2-butylamine; 3-buten-2-one; 2-butenal; 2,4-dimethyl-1,3-dioxane; 2-ethyl-5-methyl-1,3-dioxane; butyl acrylate; 2-heptyl-1,3-dioxepane; 1-hydroxy-2-propanone; 1,2 propanediol (12PDO); 1,2,3-butanetriol; 1,2,3-propanetriol; 1,3 propanediol (13PDO); 1,4-butanediol (BDO); 2,3-butanediol (23BDO); 2,5-hexanedione; 3-buten-2-ol; cis-cryotyl alcohol; isopropyl alcohol (IPA); methyl vinyl ketone (MVK); n-butane (n-but); dimethylethanolamine (DMAE); HDD; and any combinations thereof. In some embodiments, such one or more compounds can be in an amount or concentration below its odor threshold. Further embodiments provide for such one or more compounds that are contributors to “off-odor”.

Some embodiments provide a BG product comprising bioBG, where the BG product does not comprise, does not consist essentially of, does not consist of, or minimally comprises one or more compounds selected from the group consisting of: 4-methyl-heptane (cornlike, roasty, nutty, hazelnutty scent); acetone (solvent, ethereal); 2,4-dimethyl-1-heptene (pungent, plastic); 2-methyl-2-propanol (malty); 2,2,4,6,6-pentamethyl-heptane; 2,4-dimethyl-1,3-dioxane (green); cis-2,2,4,5-tetramethyl-1,3-dioxolane; N-formyl-2-hydroxy-3-methyl-2-(1-methylethyl)-butanamide; acetic acid (strong, pungent); 4-hydroxy-2-butanone (sweet, grainy, malty, butterscotch); 2-(2-hydroxyethyl)-2,4-dimethyl-1,3-dioxane (HDD); HDD isomer (e.g., cis-, trans-, BG and 4-hydroxy-2-butanone reaction product); and any combinations thereof. In some embodiments, such one or more compounds can be in an amount or concentration below its odor threshold. Other embodiments are directed to one or more of these compounds as contributors to off-odors in a neutral bioBG product described here.

In additional embodiments, a BG product comprising bioBG is provided, where the BG product does not comprise, does not consist essentially of, does not consist of, or minimally comprises one or more compounds characterized by an odor-active region in a dynamic headspace sample as measured by Gerstel DHS described here, where the compound is selected from the group consisting of and with the indicated elution time: pentane (˜1.6 mins); hexane (˜1.8 mins); 2-methyl-1-pentene (˜1.85 mins); cyclohexane (˜2 mins); 4-methylheptane (˜2.15 mins); 4-methylhexane (˜2.2 mins); acetone (˜2.4 mins); 2,4-dimethyl-1-heptene (˜2.8 mins); 2-butanone (˜2.9 mins); dichloromethane (˜3.1 mins); methyl vinyl ketone (˜3.3 mins); 2-butenal (˜4.1 mins); butyl acetate (˜4.3 mins); 3-hydroxybutanal (˜8.1 mins); 4-hydroxy-2-butanone (˜8.6 mins); and any combinations thereof, where the elution time of BG is ˜10 mins. In some embodiments, such one or more compounds can be in an amount or concentration below its respective odor threshold, where the odor threshold is an amount or concentration of a substance at which 50% of a sensory odor panel cannot smell or perceive an odor.

Other embodiments provide a BG product comprising bioBG, where the BG product comprises, consists essentially of, consists of, or minimally comprises one or more compounds selected from the group consisting of: 4-methyl-heptane; 2,4-dimethyl-1-heptene; 2-methyl-2-propanol; 2,2,4,6,6-pentamethyl-heptane; cis-2,2,4,5-tetramethyl-1,3-dioxolane; N-formyl-2-hydroxy-3-methyl-2-(1-methylethyl)-butanamide; acetic acid; 2-(2-hydroxyethyl)-2,4-dimethyl-1,3-dioxane (HDD); HDD isomer (e.g., cis-, trans-, BG and 4-hydroxy-2-butanone reaction product); and any combinations thereof. In some embodiments, such one or more compounds can be in an amount or concentration below its odor threshold. Other embodiments are directed to one or more of these compounds as contributors to off-odors.

In additional embodiments, a BG product of the disclosure comprising a bioBG and that does not comprise, does not consist essentially of, does not consist of, or minimally comprises any one or more of the compounds selected from the group consisting of: butyl acetate; butyl acrylate; 4-hydroxy-2-butanone; 2-methoxymethyl-2,4,5-trimethyl-1,3-dioxolane; 3-heptanone; 1-octen-3-one; N-pentyl-2-butylamine; 3-buten-2-one; 2-butenal; 2,4-dimethyl-1,3-dioxane; 2-ethyl-5-methyl-1,3-dioxane; 2-heptyl-1,3-dioxepane; 1-hydroxy-2-propanone; 1,2 propanediol (12PDO); 1,2,3-butanetriol; 1,2,3-propanetriol; 1,3 propanediol (13PDO); 1,4-butanediol (BDO); 2,3-butanediol (23BDO); 2,5-hexanedione; 3-buten-2-ol; cis-cryotyl alcohol; isopropyl alcohol (IPA); methyl vinyl ketone (MVK); n-butane (n-but); dimethylethanolamine (DMAE); HDD, and any combinations thereof, wherein the one or more compounds is each in an amount of 10 ppm or less (e.g., 9, 8, 7, 6, 5, 4, 3, 2, 1, 0); 0 ppm or greater (e.g., 0.5, 1.5, 2.5, 3.5, 4.5, 5.5, 6.5, 7.5, 8.5, 9.5); or 0 ppm-10 ppm (e.g., 0.1 ppm-9.8 ppm; 0.2 ppm-9.6 ppm; 0.3 ppm-9.4 ppm; 0.4 ppm-9.2 ppm; 0.5 ppm-9 ppm; 0.6 ppm-8.8 ppm; 0.7 ppm-8.6 ppm; 0.8 ppm-8.4 ppm; 0.9 ppm-8.2 ppm; 1 ppm-8 ppm). In some embodiments, such one or more compounds can be in an amount or concentration below its odor threshold. Further embodiments provide for such one or more compounds that are contributors to “off-odor”.

In some embodiments, the BG product described here comprising a bioBG, further comprises, consists essentially of, consists of, or minimally comprises: 3-hydroxy-butanal in an amount of less than 25 ppm; 4-hydroxy-2-butanone in an amount less than 25 ppm; 4-(3-hydroxybutoxy)butan-2-one in an amount less than 25 ppm; 4-((4-hydroxybutan-2-yl)oxy)-butan-2-one in an amount less than 25 ppm; 1,2-propanediol in an amount less than 1 ppm; 1,3-propanediol in an amount less than 10 ppm; 2,3-butanediol in an amount less than 1 ppm; 2,4-dimethyl-6-((1-(4-methyl-1,3-dioxan-2-yl)propan-2-yl)oxy)-1,3-dioxane in an amount less than 2 ppm; 4-(sec-butoxy)-2,6-dimethyl-1,3-dioxane in an amount less than 4 ppm; 2,4-dimethyl-1,3-dioxane in an amount less than 8 ppm; (E)-4-methyl-2-(prop-1-en-1-yl)-1,3-dioxane in an amount less than 8 ppm; 3-hydroxybutyl acetate in an amount less than 8 ppm; 1-(2-methyl-1,3-dioxan-4-yl)propan-2-ol in an amount less than 8 ppm; 2-methyl-4-propyl-1,3-dioxane in an amount less than 8 ppm; tri-substituted 1,3-dioxane

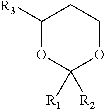

in an amount less than 8 ppm and/or 3-hydroxybutyl carbono-substituted

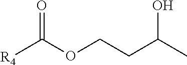

in an amount less than 8 ppm, wherein R1 to R4 are the same or different; a hydrogen atom, a C1-C4 alkyl group optionally substituted by an OH group, or a C2-C4 alkenyl group optionally substituted by OH group; 2,4-dimethyl-2-vinyl-1,3-dioxane; 2-(2,4-dimethyl-1,3-dioxan-2-yl)ethan-1-ol; 2,2,4-trimethyl-1,3-dioxane; 4-methyl-1,3-dioxane, wherein the total of 2,4-dimethyl-2-vinyl-1,3-dioxane; 2-(2,4-dimethyl-1,3-dioxan-2-yl)ethan-1-ol; 2,2,4-trimethyl-1,3-dioxane; 4-methyl-1,3-dioxane is in a total amount of less than 34 ppm; (E)-4-methyl-2-(prop-1-en-1-yl)-1,3-dioxane; 1-(4-methyl-1,3-dioxan-2-yl)propan-2-ol; 2,4-dimethyl-1,3-dioxane; 3-hydroxybutyl (E)-but-2-enoate; 3-hydroxybutyl acetate; 3-hydroxybutyl 3-hydroxybutanoate, wherein the total of (E)-4-methyl-2-(prop-1-en-1-yl)-1,3-dioxane; 1-(4-methyl-1,3-dioxan-2-yl)propan-2-ol; 2,4-dimethyl-1,3-dioxane; 3-hydroxybutyl (E)-but-2-enoate; 3-hydroxybutyl acetate; 3-hydroxybutyl 3-hydroxybutanoate is in a total amount of less than 63 ppm or wherein the total of 2,4-dimethyl-2-vinyl-1,3-dioxane; 2-(2,4-dimethyl-1,3-dioxan-2-yl)ethan-1-ol; 2,2,4-trimethyl-1,3-dioxane; 4-methyl-1,3-dioxane; (E)-4-methyl-2-(prop-1-en-1-yl)-1,3-dioxane; 1-(4-methyl-1,3-dioxan-2-yl)propan-2-ol; 2,4-dimethyl-1,3-dioxane; 3-hydroxybutyl (E)-but-2-enoate; 3-hydroxybutyl acetate; 3-hydroxybutyl 3-hydroxybutanoate is in a total amount of less than 97 ppm; methyl vinyl ketone in an amount less than 8 ppm; 1-hydroxy-3-butanone in an amount less than 1 ppm; acetone in an amount less than 6 ppm; formaldehyde in an amount less than 2 ppm; crotonaldehyde in an amount less than 1 ppm; acetaldol in an amount less than 1 ppm; acetaldehyde in an amount less than 5 ppm; butyraldehyde in an amount less than 4 ppm; 2-butanol, wherein the total of 2,4-dimethyl-2-vinyl-1,3-dioxane; 2-(2,4-dimethyl-1,3-dioxan-2-yl)ethan-1-ol; 2,2,4-trimethyl-1,3-dioxane; 4-methyl-1,3-dioxane; (E)-4-methyl-2-(prop-1-en-1-yl)-1,3-dioxane; 1-(4-methyl-1,3-dioxan-2-yl)propan-2-ol; 2,4-dimethyl-1,3-dioxane; 3-hydroxybutyl (E)-but-2-enoate; 3-hydroxybutyl acetate; 3-hydroxybutyl 3-hydroxybutanoate; and 2-butanol is in a total amount of less than 65 ppm; butyl acetate; butyl acrylate; 4-hydroxy-2-butanone; 2-methoxymethyl-2,4,5-trimethyl-1,3-dioxolane (solventy); 3-heptanone; 1-octen-3-one; N-pentyl-2-butylamine; 3-buten-2-one; 2-butenal; 2,4-dimethyl-1,3-dioxane; 2-ethyl-5-methyl-1,3-dioxane; 2-heptyl-1,3-dioxepane; 1-hydroxy-2-propanone; 1,2 propanediol (12PDO); 1,2,3-butanetriol; 1,2,3-propanetriol; 1,3 propanediol (13PDO); 1,4-butanediol (BDO); 2,3-butanediol (23BDO); 2,5-hexanedione; 3-buten-2-ol; cis-cryotyl alcohol; isopropyl alcohol (IPA); methyl vinyl ketone (MVK); n-butane (n-but); dimethylethanolamine (DMAE); toluene; HDD, and any combinations thereof. In some embodiments, such one or more compounds can be in an amount or concentration below its odor threshold. Further embodiments provide for such one or more compounds that are contributors to “off-odor”.

Other embodiments of the disclosure are directed to a BG product comprising a bioBG, where the BG product does not comprise, does not consist essentially of, does not consist of, or minimally comprises a compound selected from the group consisting of: 3-hydroxy-butanal; 4-hydroxy-2-butanone; 4-(3-hydroxybutoxy)butan-2-one; 4-((4-hydroxybutan-2-yl)oxy)-butan-2-one; 1,2-propanediol; 1,3-propanediol; 2,3-butanediol; 2,4-dimethyl-6-((1-(4-methyl-1,3-dioxan-2-yl)propan-2-yl)oxy)-1,3-dioxane; 4-(sec-butoxy)-2,6-dimethyl-1,3-dioxane; 2,4-dimethyl-1,3-dioxane; (E)-4-methyl-2-(prop-1-en-1-yl)-1,3-dioxane; 3-hydroxybutyl acetate; 1-(2-methyl-1,3-dioxan-4-yl)propan-2-ol; 2-methyl-4-propyl-1,3-dioxane; tri-substituted 1,3-dioxane

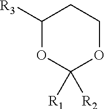

and/or 3-hydroxybutyl carbono-substituted

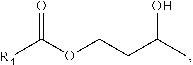

wherein R1 to R4 are the same or different; a hydrogen atom, a C1-C4 alkyl group optionally substituted by an OH group, or a C2-C4 alkenyl group optionally substituted by OH group; 2,4-dimethyl-2-vinyl-1,3-dioxane; 2-(2,4-dimethyl-1,3-dioxan-2-yl)ethan-1-ol; 2,2,4-trimethyl-1,3-dioxane; 4-methyl-1,3-dioxane; (E)-4-methyl-2-(prop-1-en-1-yl)-1,3-dioxane; 1-(4-methyl-1,3-dioxan-2-yl)propan-2-ol; 2,4-dimethyl-1,3-dioxane; 3-hydroxybutyl (E)-but-2-enoate; 3-hydroxybutyl acetate; 3-hydroxybutyl 3-hydroxybutanoate; methyl vinyl ketone; 1-hydroxy-3-butanone; acetone; formaldehyde; crotonaldehyde; acetaldol; acetaldehyde; butyraldehyde; 2-butanol; butyl acrylate; butyl acetate; 4-hydroxy-2-butanone; 2-methoxymethyl-2,4,5-trimethyl-1,3-dioxolane (solventy); 3-heptanone; 1-octen-3-one; N-pentyl-2-butylamine; 3-buten-2-one; 2-butenal; 2,4-dimethyl-1,3-dioxane; 2-ethyl-5-methyl-1,3-dioxane; 2-heptyl-1,3-dioxepane; 1-hydroxy-2-propanone; 1,2 propanediol (12PDO); 1,2,3-butanetriol; 1,2,3-propanetriol; 1,3 propanediol (13PDO); 1,4-butanediol (BDO); 2,3-butanediol (23BDO); 2,5-hexanedione; 3-buten-2-ol; cis-cryotyl alcohol; isopropyl alcohol (IPA); n-butane (n-but); dimethylethanolamine (DMAE); toluene; HDD, and or any combinations thereof, wherein the BG product comprises bioBG. In some embodiments, such one or more compounds can be in an amount or concentration below its odor threshold. Further embodiments provide for such one or more compounds that are contributors to “off-odor”.

Additional embodiments provide for a BG product comprising a bioBG and does not further comprise, does not consist essentially of, does not consist of, or minimally comprises one or more of the compounds selected from the group consisting of: 3-hydroxy-butanal (e.g., less than 25 ppm); 4-hydroxy-2-butanone (e.g., less than 25 ppm); 4-(3-hydroxybutoxy)butan-2-one (e.g., less than 25 ppm); 4-((4-hydroxybutan-2-yl)oxy)-butan-2-one (e.g., less than 25 ppm); 1,2-propanediol (e.g., less than 1 ppm); 1,3-propanediol (e.g., less than 10 ppm); 2,3-butanediol (e.g., less than 1 ppm); 2,4-dimethyl-6-((1-(4-methyl-1,3-dioxan-2-yl)propan-2-yl)oxy)-1,3-dioxane (e.g., less than 2 ppm); 4-(sec-butoxy)-2,6-dimethyl-1,3-dioxane (e.g., less than 4 ppm); 2,4-dimethyl-1,3-dioxane (e.g., less than 8 ppm); (E)-4-methyl-2-(prop-1-en-1-yl)-1,3-dioxane (e.g., less than 8 ppm); 3-hydroxybutyl acetate (e.g., less than 8 ppm); 1-(2-methyl-1,3-dioxan-4-yl)propan-2-ol (e.g., less than 8 ppm); 2-methyl-4-propyl-1,3-dioxane (e.g., less than 8 ppm); tri-substituted 1,3-dioxane

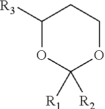

(e.g., less than 8 ppm) and/or 3-hydroxybutyl carbono-substituted)

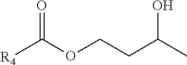

(e.g., less than 8 ppm), wherein R1 to R4 are the same or different; a hydrogen atom, a C1-C4 alkyl group optionally substituted by an OH group, or a C2-C4 alkenyl group optionally substituted by OH group; 2,4-dimethyl-2-vinyl-1,3-dioxane; 2-(2,4-dimethyl-1,3-dioxan-2-yl)ethan-1-ol; 2,2,4-trimethyl-1,3-dioxane; 4-methyl-1,3-dioxane, wherein the total of 2,4-dimethyl-2-vinyl-1,3-dioxane; 2-(2,4-dimethyl-1,3-dioxan-2-yl)ethan-1-ol; 2,2,4-trimethyl-1,3-dioxane; 4-methyl-1,3-dioxane is in a total amount of less than 34 ppm; (E)-4-methyl-2-(prop-1-en-1-yl)-1,3-dioxane; 1-(4-methyl-1,3-dioxan-2-yl)propan-2-ol; 2,4-dimethyl-1,3-dioxane; 3-hydroxybutyl (E)-but-2-enoate; 3-hydroxybutyl acetate; 3-hydroxybutyl 3-hydroxybutanoate, wherein the total of (E)-4-methyl-2-(prop-1-en-1-yl)-1,3-dioxane; 1-(4-methyl-1,3-dioxan-2-yl)propan-2-ol; 2,4-dimethyl-1,3-dioxane; 3-hydroxybutyl (E)-but-2-enoate; 3-hydroxybutyl acetate; 3-hydroxybutyl 3-hydroxybutanoate is in a total amount of less than 63 ppm or wherein the total of 2,4-dimethyl-2-vinyl-1,3-dioxane; 2-(2,4-dimethyl-1,3-dioxan-2-yl)ethan-1-ol; 2,2,4-trimethyl-1,3-dioxane; 4-methyl-1,3-dioxane; (E)-4-methyl-2-(prop-1-en-1-yl)-1,3-dioxane; 1-(4-methyl-1,3-dioxan-2-yl)propan-2-ol; 2,4-dimethyl-1,3-dioxane; 3-hydroxybutyl (E)-but-2-enoate; 3-hydroxybutyl acetate; 3-hydroxybutyl 3-hydroxybutanoate is in a total amount of less than 97 ppm; methyl vinyl ketone (e.g., less than 8 ppm); 1-hydroxy-3-butanone (e.g., less than 1 ppm); acetone (e.g., less than 6 ppm); formaldehyde (e.g., less than 2 ppm); crotonaldehyde (e.g., less than 1 ppm); acetaldol (e.g., less than 1 ppm); acetaldehyde (e.g., less than 5 ppm); butyraldehyde (e.g., less than 4 ppm); 2-butanol, wherein the total of 2,4-dimethyl-2-vinyl-1,3-dioxane; 2-(2,4-dimethyl-1,3-dioxan-2-yl)ethan-1-ol; 2,2,4-trimethyl-1,3-dioxane; 4-methyl-1,3-dioxane; (E)-4-methyl-2-(prop-1-en-1-yl)-1,3-dioxane; 1-(4-methyl-1,3-dioxan-2-yl)propan-2-ol; 2,4-dimethyl-1,3-dioxane; 3-hydroxybutyl (E)-but-2-enoate; 3-hydroxybutyl acetate; 3-hydroxybutyl 3-hydroxybutanoate; and 2-butanol is in a total amount of less than 65 ppm; butyl acrylate (e.g., less than 1.82 mg/L, less than 0.53 mg/L, less than 0.33 mg/L, less than 0.24 mg/L, or less than 0.0001 mg/L); HDD (e.g., less than 215, less than 19.3, less than 4, or less than 2.5 in fold change relative to a reference value or a control as quantified with a dichloromethane extraction method) and any combinations thereof. In some embodiments, such one or more compounds can be in an amount or concentration below its odor threshold. Further embodiments provide for such one or more compounds that are contributors to “off-odor”.

Some embodiments of the disclosure are directed to a BG product, where the BG product is a neutral odor bioBG product that comprises or minimally comprises one or more compounds characterized by an aromatic region, an odor-active region, or a peak, used interchangeably here, as measured by gas chromatography/mass spectrometry/olfactory (GC/MS/O). In some embodiments, the one or more compounds can be aromatic compounds or aromatic compounds minimally having an undesirable or offensive odor as characterized by an aromatic region, an odor-active region, or a peak as measured by gas chromatography/mass spectrometry/olfactory (GC/MS/O). Some embodiments provide a BG product of the disclosure that does not comprise, does not consist essentially of, does not consist of, or minimally comprises one or more compounds having an aromatic region, an odor-active region, or a peak as measured by GC/MS/O that elutes at an acquisition time selected from the group consisting of: ˜2.5 mins (rubber and/or plastic and/or green; 3-buten-2-one); ˜2.7 mins (plastic); ˜2.9 mins (solventy; 2-butenal); ˜4.6 mins (plastic: 2,4-dimethyl-1,3-dioxane); ˜6.2 mins (fruity and/or plastic; 2-ethyl-5-methyl-1,3-dioxane); ˜6.6 mins (plastic and/or pungent and/or mushroom and/or metallic; butyl acrylate);˜8.2 mins (fruity and/or plastic and/or metal and/or mushroom; 2-heptyl-1,3-dioxepane); ˜11.8 mins (waxy and/or fatty); and any combinations thereof. In some embodiments, such one or more compounds can be in an amount or concentration below its odor threshold. Further embodiments provide for such one or more compounds that are contributors to “off-odor”.

In some embodiments, a BG product of the disclosure comprises, consists essentially of, consists of, or minimally comprises one or more compounds, as measured by GC/MS/O, having an aromatic region, an odor-active region, or a peak with a relative acquisition time selected from a group consisting of: about 0.594 (fruity and/or solventy), about 0.628 (fruity), about 0.655 (citrus and/or peel), about 0.711 (solventy), about 1.328 (roasty, exhaust), and combinations thereof, where the BG product comprises bioBG, and the bioBG has a relative acquisition time of 1 min. In some embodiments, such one or more compounds can be in an amount or concentration below its odor threshold. Further embodiments provide for such one or more compounds that are contributors to “off-odor”.

Other embodiments provide a BG product comprising, consisting essentially of, consists of, or minimally comprises one or more compounds, as measured by GC/MS/O, having an aromatic region, an odor-active region, or a peak with a relative acquisition time found in DSP-555-2 and/or DSP-555-4. TABLEs 2-3 show a summary of the GC/MS/O chromatogram results illustrated in FIGS. 2-6. In some embodiments, such one or more compounds can be in an amount or concentration below its odor threshold. Further embodiments provide for such one or more compounds that are contributors to “off-odor”.

In some embodiments, the disclosure provides a 1,3-butylene glycol (BG) product that does not comprise, does not consist essentially of, does not consist of, or minimally comprises one or more compounds characterized by an odor-active region as measured by gas chromatography/mass spectrometry/olfactory (GC/MS/O) having a relative acquisition time selected from a group consisting of: about 0.417 mins (rubber and/or plastic and/or green), about 0.450 mins (plastic), about 0.483 mins (solventy), about 0.783 mins (plastic), about 1.033 mins (fruity and/or plastic), about 1.1 mins (plastic and/or pungent and/or mushroom and/or metallic), about 1.367 mins (fruity and/or plastic and/or metal and/or mushroom), about 1.967 mins (waxy and/or fatty), and any combinations thereof, where the BG product comprises bioderived BG (bioBG), and the bioBG has a relative acquisition time of 1 min. In some embodiments, such one or more compounds can be in an amount or concentration below its odor threshold. Further embodiments provide for such one or more compounds that are contributors to “off-odor”.

Some embodiments of the BG product described here does not comprise, does not consist essentially of, does not consist of, or minimally comprises one or more compounds characterized by an aromatic region, an odor-active region, or a peak as measured by gas chromatography/mass spectrometry/olfactory (GC/MS/O) selected from the group consisting of: a fusel and/or sulfur and/or oil odor at an acquisition time of ˜9 mins-˜9.2 mins (relative acquisition times ˜0.5 mins, ˜0.506 mins, ˜0.511 mins, respectively); toluene at an acquisition time of ˜9.3 mins (relative acquisition time ˜0.517 mins); a fruity and/or plastic odor at an acquisition time of ˜10.2 mins-˜10.4 mins (relative acquisition times ˜0.567 mins, ˜0.572 mins, ˜0.578 mins, respectively); a fruity and/or floral and/or plastic odor at an acquisition time of ˜10.5 mins-˜10.7 mins (relative acquisition times ˜0.583 mins, ˜0.589 mins, ˜0.594 mins, respectively); and a plastic and/or roasty odor at an acquisition time of ˜13.3 mins-˜13.5 mins (relative acquisition times ˜0.739 mins, ˜0.744 mins, ˜0.75 mins, respectively). In some embodiments, such one or more compounds can be in an amount or concentration below its odor threshold. Further embodiments provide for such one or more compounds that are contributors to “off-odor”.

Additional embodiments are directed to BG products comprising bioBG and not comprising, not consisting essentially of, not consisting of, or minimally comprising an odor-active region and/or one or more compounds as measured by gas chromatography/mass spectrometry/olfactory (GC/MS/O) and selected from any of DSP-555-1, DSP-555-3, DSP-555-5, DSP-555-6, DSP-555-7, DSP-555-8, DSP-555-9, DSP-555-10, DSP-555-11, DSP-555-12, DSP-555-13, DSP-555-14, DSP-555-15, DSP-555-16, and any combinations thereof as illustrated in TABLE 2 and/or TABLE 3. In some embodiments, such one or more compounds can be in an amount or concentration below its odor threshold. Further embodiments provide for such one or more compounds that are contributors to “off-odor”.

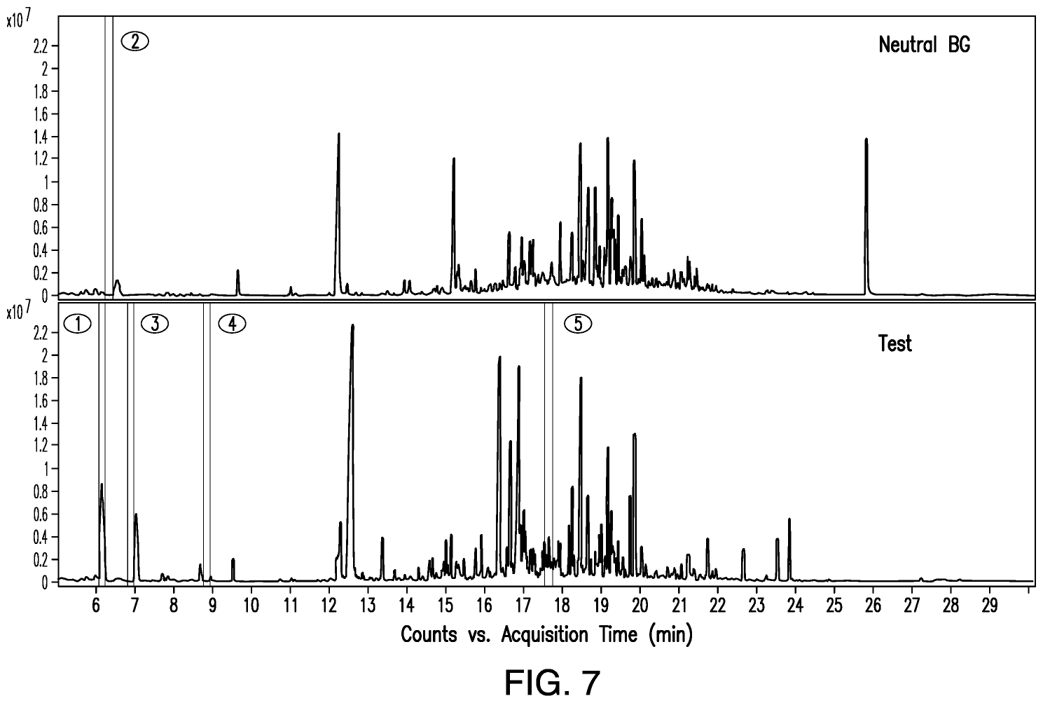

In some embodiments, a BG product of the disclosure comprising bioBG does not comprise, does not consist essentially of, does not consist of, or minimally comprises one or more compounds having an aromatic region, an odor-active region, or a peak as measured by GC/MS/O that elutes at an acquisition time selected from a group consisting of: about 6 mins (solventy), about 7 mins (plastic and/or musty), about 9 mins (mushroom and/or earthy), about 17.5 mins (sulfury and/or urine-like), and combinations thereof. See, FIG. 7 (lower panel). In some embodiments, such one or more compounds can be in an amount or concentration below its odor threshold. Further embodiments provide for such one or more compounds that are contributors to “off-odor”.

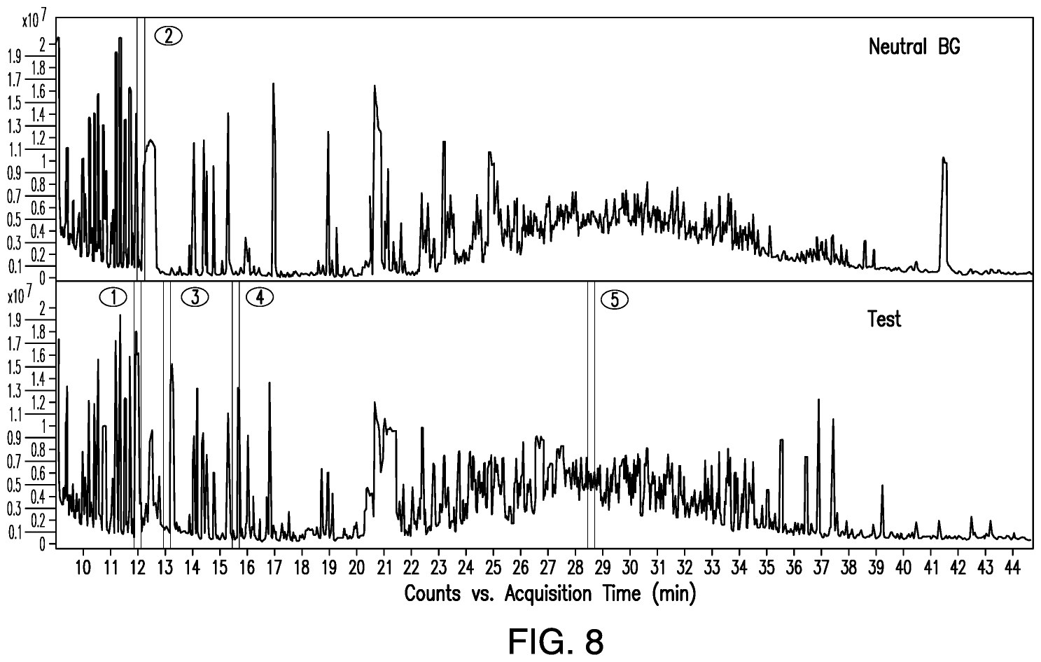

Some embodiments provide a BG product of the disclosure comprising bioBG, does not comprise, consist essentially of, or consist of one or more compounds having an aromatic region, an odor-active region, or a peak as measured by GC/QTOF-MS that elutes at an acquisition time selected from a group consisting of: about 12 mins (solventy), about 13 mins (plastic and/or musty), about 15.5 mins (mushroom and/or earthy), about 28.5 mins (sulfury and/or urine-like), and combinations thereof. See, FIG. 8 (lower panel); TABLE 4. In some embodiments, such one or more compounds can be in an amount or concentration below its odor threshold. Further embodiments provide for such one or more compounds that are contributors to “off-odor”.

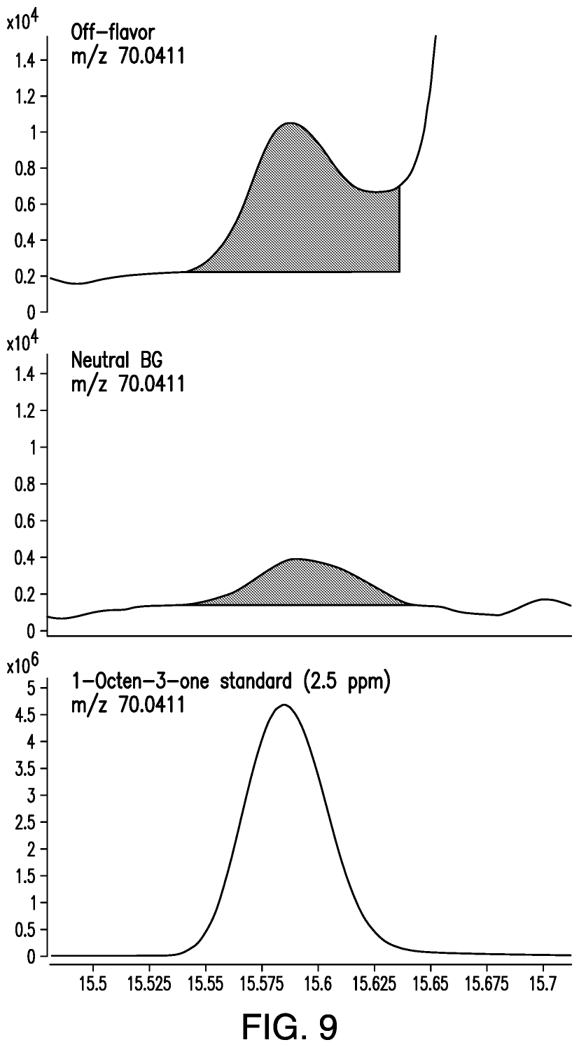

In some embodiments, a BG product of the disclosure does not comprise, does not consist essentially of, does not consist of, or minimally comprises one or more compounds characterized by an aromatic region, an odor-active region, or a peak as measured by gas chromatography/quadrupole time-of-flight-mass spectrometry (GC/QTOF-MS), where the aromatic region, the odor-active region, or the peak elutes at an acquisition time selected from a group consisting of: about 12 mins (e.g., 11.9 mins-12.2 mins; 11.9 mins, 12 mins, 12.1 mins, 12.2 mins) at 59.0489 m/z, about 13 mins (e.g., 12.955 mins-13.15 mins; 12.956 mins, 12.957 mins, 12.958 mins, 12.959 mins, 12.96 mins, 12.961 mins, 12.962 mins, 12.963 mins, 12.964 mins, 12.965 mins, 12.966 mins, 12.967 mins, 12.968 mins, 12.969 mins, 12.97 mins, 12.971 mins, 12.972 mins, 12.973 mins, 12.974 mins, 12.975 mins, 12.976 mins, 12.977 mins, 12.978 mins, 12.979 mins, 12.98 mins, 12.981 mins, 12.982 mins, 12.983 mins, 12.984 mins, 12.985 mins, 12.986 mins, 12.987 mins, 12.988 mins, 12.989 mins, 12.99 mins, 12.991 mins, 12.992 mins, 12.993 mins, 12.994 mins, 12.995 mins, 12.996 mins, 12.997 mins, 12.998 mins, 12.999 mins, 13 mins, 13.01 mins, 13.02 mins, 13.03 mins, 13.04 mins, 13.05 mins, 13.06 mins, 13.07 mins, 13.08 mins, 13.09 mins, 13.1 mins, 13.11 mins, 13.12 mins, 13.13 mins, 13.14 mins, 13.15 mins) at 57.0698 m/z, about 15.5 mins (e.g., 15.55 mins-15.65; 15.55 mins, 15.56 mins, 15.57 mins, 15.58 mins, 15.59 mins, 15.6 mins, 15.61 mins, 15.62 mins, 15.63 mins, 15.64 mins, 15.65 mins) at 70.0411 m/z, about 28.5 mins (e.g., 28.5 mins-28.6 mins) at 114.0675 m/z, and combinations thereof. See, FIGS. 9-10 and 12-13. In some embodiments, such one or more compounds can be in an amount or concentration below its odor threshold. Further embodiments provide for such one or more compounds that are contributors to “off-odor”.

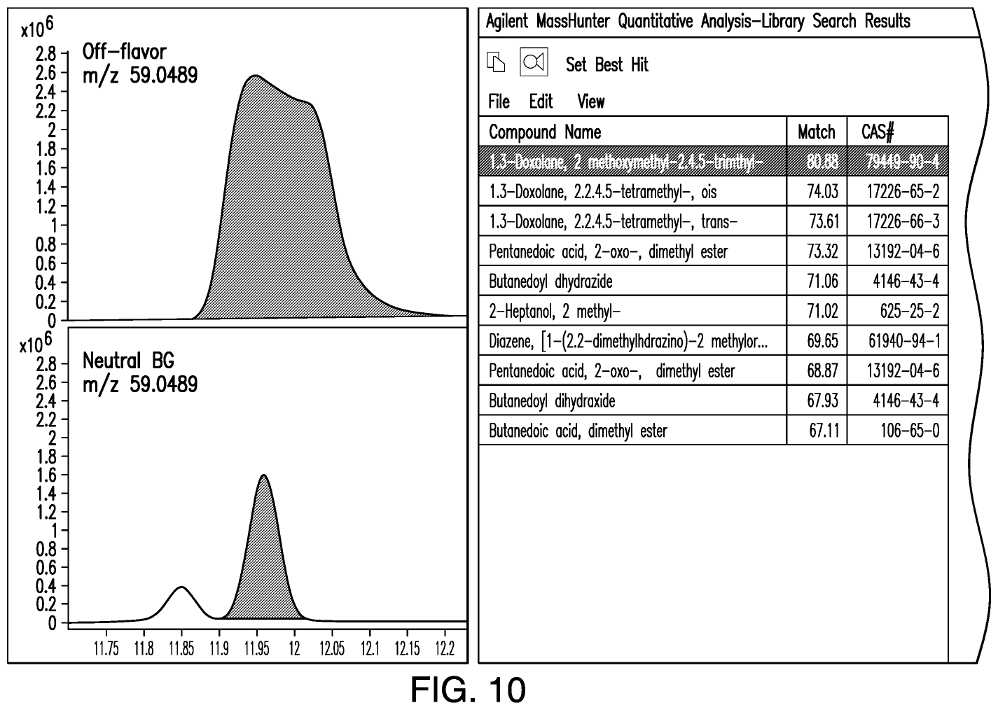

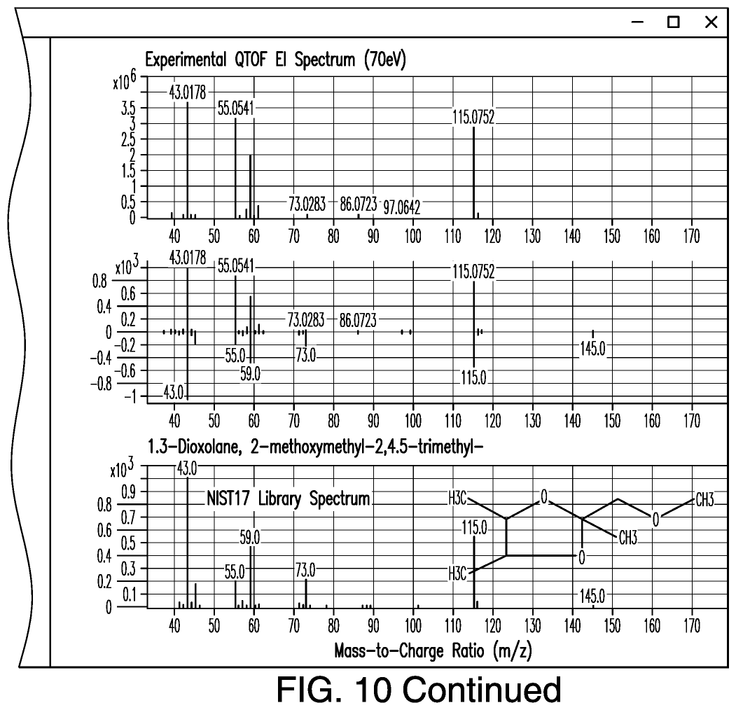

Embodiments of the disclosure can be directed to a 1,3-butylene glycol (BG) product, that does not comprise, does not consist essentially of, or minimally comprises one or more compounds having a mass spectrum as characterized by quadrupole time-of-flight-mass spectrometry electron ionization (QTOF EI) at 70 eV. The mass spectrum comprises peaks, and the mass spectrum is selected from the group consisting of: (a) 43.017 m/z, 55.0541 m/z, 73.0283 m/z, 86.0723 m/z, 97.0642 m/z, and 115.0752; (b) 41.0385 m/z, 57.0698 m/z, 68.0259 m/z, 72.0569 m/z, 85.0646 m/z, 99.0801 m/z, and 114.1033 m/z; and (c) 43.0179 m/z, 57.0336 m/z, 72.0206 m/z, 86.0362 m/z, 114.0675 m/z, 128.0830 m/z, 138.0670 m/z, and 155.1060 m/z. See, FIGS. 10, 12-13. In some embodiments, such one or more compounds can be in an amount or concentration below its odor threshold. Further embodiments provide for such one or more compounds that are contributors to “off-odor”.

In some embodiments, any of the BG products of the disclosure can comprise a neutral odor bioBG, where the neutral odor bioBG and/or one or more compounds present therein, is present in an amount below its odor threshold. Further embodiments described here can be directed to a BG product of the disclosure, such as an isolated or purified BG product, where the BG product comprises a bioBG, wherein the BG product comprises, consists essentially of, or consists of any one or more of the compounds disclosed here in an amount of: 0 parts per million (ppm) or more (e.g., 0.00001 ppm, 0.000015 ppm, 0.00002 ppm, 0.000025 ppm, 0.00005 ppm, 0.0001 ppm, 0.0002 ppm, 0.0003 ppm, 0.0004 ppm, 0.0005 ppm, 0.0006 ppm, 0.0007 ppm, 0.0008 ppm, 0.0009 ppm, 0.001 ppm, 0.002 ppm, 0.003 ppm, 0.004 ppm, 0.005 ppm, 0.006 ppm, 0.007 ppm, 0.008 ppm, 0.009 ppm, 0.01 ppm, 0.02 ppm, 0.03 ppm, 0.04 ppm, 0.05 ppm, 0.06 ppm, 0.07 ppm, 0.08 ppm, 0.09 ppm, 0.1 ppm, 0.2 ppm, 0.3 ppm, 0.4 ppm, 0.5 ppm, 0.6 ppm, 0.7 ppm, 0.8 ppm, 0.9 ppm, 1 ppm, 10 ppm, 100 ppm, 1000 ppm, 10,000 ppm); or 10,000 ppm or less (e.g., 9,500 ppm, 9,000 ppm, 8,500 ppm, 8,000 ppm, 7,500 ppm, 7,000 ppm, 6,500 ppm, 6,000 ppm, 5,500 ppm, 5,000 ppm, 4,500 ppm, 4,000 ppm, 3,500 ppm, 3,000 ppm, 2,500 ppm, 2,000 ppm, 1,500 ppm, 1,000 ppm, 500 ppm, 50 ppm, 5 ppm, 0.75 ppm, 0.5 ppm, 0.25 ppm, 0.2 ppm, 0.190 ppm, 0.180 ppm, 0.170 ppm, 0.160 ppm, 0.150 ppm, 0.140 ppm, 0.130 ppm, 0.120 ppm, 0.110 ppm, 0.1 ppm, 0.09 ppm, 0.08 ppm, 0.07 ppm, 0.06 ppm, 0.05 ppm, 0.04 ppm, 0.03 ppm, 0.02 ppm, 0.01 ppm, 0.009 ppm, 0.008 ppm, 0.007 ppm, 0.006 ppm, 0.005 ppm, 0.004 ppm, 0.003 ppm, 0.002 ppm, 0.001 ppm, 0.0009 ppm, 0.0008 ppm, 0.0007 ppm, 0.0006 ppm, 0.0005 ppm, 0.0004 ppm, 0.0003 ppm, 0.0002 ppm, 0.0001 ppm, 0.00005 ppm, 0.00001); or 0 ppm-10,000 ppm (e.g., 0.000015 ppm-9,000 ppm; 0.00005 ppm-8,000 ppm; 0.0001 ppm-7,000 ppm; 0.01 ppm-6,000 ppm; 0.05 ppm-5,000 ppm; 0.1 ppm-4,000 ppm; 0.5 ppm-3,000 ppm; 0.2 ppm-2,000 ppm; 0.3 ppm-2,000 ppm; 0.4 ppm-1,000 ppm; 0.5 ppm-750 ppm; 0.6 ppm-500 ppm; 0.7 ppm-250 ppm; 0.8 ppm-200 ppm; 0.9 ppm-175 ppm; 1 ppm-150 ppm; 5 ppm-125 ppm; 10 ppm-100 ppm; 20 ppm-75 ppm; 30 ppm-50 ppm; 35 ppm-45 ppm). In some embodiments, the BG product comprising, consisting essentially of, or consisting of one or more of the compounds disclosed here, where the compound is in an amount of 0 ppm or more (e.g., 0.00001 or more), 10 ppm or less, or 0 ppm-10 ppm. In some embodiments, such one or more compounds can be in an amount or concentration below its odor threshold. Further embodiments provide for such one or more compounds that are contributors to “off-odor”.

In some embodiments, the BG product comprising a bioBG and does not comprise, does not consist essentially of, or consist of one or more compounds characterized by an aromatic region, odor-active region, or a peak as measured by gas chromatography/mass spectrometry/olfactory (GC/MS/O), where the aromatic region, odor-active region, or the peak elutes at an acquisition time selected from a group consisting of: ˜2.5 mins (rubber and/or plastic and/or green; 3-buten-2-one); ˜2.7 mins (plastic); ˜2.9 mins (solventy; 2-butenal); ˜6.2 mins (fruity and/or plastic); ˜8.2 mins (fruity and/or plastic and/or metal and/or mushroom; 2-heptyl-1,3-dioxepane); ˜10.7 mins (fruity and/or solventy); ˜11.3 mins (fruity); ˜11.8 mins (citrus and/or peel and/or fatty); ˜12.8 mins (solventy); ˜23.9 mins (roasty and/or exhaust); and any combinations thereof, or about 6 mins (solventy), about 7 mins (plastic and/or musty), about 9 mins (mushroom and/or earthy), about 17.5 mins (sulfury and/or urine-like), and combinations thereof. In some embodiments, such one or more compounds can be in an amount or concentration below its odor threshold. Further embodiments provide for such one or more compounds that are contributors to “off-odor”. The one or more compounds comprising the aromatic region, the odor-active region, or the peak having the acquisition times (about 6 mins, about 7 mins, about 9 mins, about 17.5 mins, and combinations thereof) can be associated with an odor selected from the group consisting of: solventy; plastic and/or musty; mushroom and/or earthy; and sulfury and/or urine-like, respectively, where, in some embodiments, the one or more compounds are present in an amount or concentration below its odor threshold. Further embodiments provide for such one or more compounds that are contributors to “off-odor”. The one or more compounds comprising the aromatic region, the odor-active region, or the peak as measured by GC/MS/O with an odor selected from the group consisting of: (a) solventy; (b) plastic and/or musty; (c) mushroom and/or earthy; and (d) sulfury and/or urine-like, respectively, and correspond to the compounds: (a) 2-methoxymethyl-2,4,5-trimethyl-1,3-dioxolane; (b) 3-heptanone; (c) 1-octen-3-one; and (d) N-pentyl-2-butylamine, respectively, where the one or more compounds can be in an amount of 0 ppm or more (e.g., 0.00001 or more), 10 ppm or less, or 0 ppm-10 ppm. In some embodiments, such one or more compounds can be in an amount or concentration below its odor threshold. Further embodiments provide for such one or more compounds that are contributors to “off-odor”.

Some embodiments of the disclosure are directed to a BG product having a neutral odor, where the BG product of the disclosure does not comprise, does not consist essentially of, does not consist of, or minimally comprises one or more compounds characterized by an aromatic region, an odor-active region, or a peak as measured by gas chromatography/mass spectrometry/olfactory (GC/MS/O), where the aromatic region, the odor-active region, or the peak has a relative acquisition time selected from a group consisting of: about 0.417 mins (rubber and/or plastic and/or green), about 0.450 mins (plastic), about 0.483 mins (solventy), about 0.783 mins (plastic), about 1.033 mins (fruity and/or plastic), about 1.1 mins (plastic and/or pungent and/or mushroom and/or metallic), about 1.367 mins (fruity and/or plastic and/or metal and/or mushroom), about 1.967 mins (waxy and/or fatty), and combinations thereof, wherein the BG product comprises bioBG, wherein the BG has a relative acquisition time of 1 min, and the one or more compounds is in an amount of 0 ppm or more (e.g., 0.00001 or more), 10 ppm or less, or 0 ppm-10 ppm. In some embodiments, such one or more compounds can be in an amount or concentration below its odor threshold. Further embodiments provide for such one or more compounds that are contributors to “off-odor”.

Some embodiments provide the BG product having a bioBG and further not comprising, not consisting essentially of, or not consisting of one or more of the compounds disclosed here, where the compound is in an amount of 0 ppm or more (e.g., 0.00001 or more), 10 ppm or less, or 0 ppm-10 ppm, where the one or more of the compounds can be characterized by an aromatic region, an odor-active region, or a peak as measured by gas chromatography/quadrupole time-of-flight-mass spectrometry (GC/QTOF-MS). The aromatic region, the odor-active region, or the peak can elute at an acquisition time selected from a group consisting of: about 12 mins (e.g., 11.9 mins-12.2 mins) at 59.0489 m/z, about 13 mins (e.g., 12.955 mins-13.15 mins) at 57.0698 m/z, about 15.5 mins (e.g., 15.55 mins-15.65) at 70.0411 m/z, about 28.5 mins (e.g., 28.5 mins-28.6 mins) at 114.0675 m/z, and combinations thereof. In some embodiments, such one or more compounds can be in an amount or concentration below its odor threshold. Further embodiments provide for such one or more compounds that are contributors to “off-odor”.

In some embodiments, the BG product comprising bioBG, and further does not comprise, does not consist essentially of, or does not consist of one or more of the compounds disclosed here, where the compound is in an amount of 0 ppm or more (e.g., 0.00001 or more), 10 ppm or less, or 0 ppm-10 ppm, where the compound comprises a mass spectrum as characterized by quadrupole time-of-flight-mass spectrometry electron ionization (QTOF EI) at 70 eV, wherein the mass spectrum comprises peaks, wherein the mass spectrum is selected from the group consisting of: (a) 43.017 m/z, 55.0541 m/z, 73.0283 m/z, 86.0723 m/z, 97.0642 m/z, and 115.0752; (b) 41.0385 m/z, 57.0698 m/z, 68.0259 m/z, 72.0569 m/z, 85.0646 m/z, 99.0801 m/z, and 114.1033 m/z; and (c) 43.0179 m/z, 57.0336 m/z, 72.0206 m/z, 86.0362 m/z, 114.0675 m/z, 128.0830 m/z, 138.0670 m/z, and 155.1060 m/z. In some embodiments, such one or more compounds can be in an amount or concentration below its odor threshold. Further embodiments provide for such one or more compounds that are contributors to “off-odor”.

In some embodiments, the BG product does not comprise, does not consist essentially of, does not consist of, or minimally comprises any one or more of the compounds characterized by an aromatic region or a peak as measured by gas chromatography/mass spectrometry/olfactory (GC/MS/O), where the aromatic region or the peak elutes at an acquisition time selected from a group consisting of: about 6 mins, about 7 mins, about 9 mins, about 17.5 mins, and combinations thereof. In some embodiments, such one or more compounds can be in an amount or concentration below its odor threshold. Further embodiments provide for such one or more compounds that are contributors to “off-odor”.

Some embodiments provide a BG product that does not comprise, does not consist essentially of, does not consist of, or minimally comprises any one or more of the compounds disclosed here characterized by an aromatic region or a peak as measured by gas chromatography/quadrupole time-of-flight-mass spectrometry (GC/QTOF-MS), where the aromatic region or the peak elutes at an acquisition time selected from a group consisting of: about 12 mins (e.g., 11.9 mins-12.2 mins) at 59.0489 m/z, about 13 mins (e.g., 12.955 mins-13.15 mins) at 57.0698 m/z, about 15.5 mins (e.g., 15.55 mins-15.65) at 70.0411 m/z, about 28.5 mins (e.g., 28.5 mins-28.6 mins) at 114.0675 m/z, and combinations thereof. In some embodiments, such one or more compounds can be in an amount or concentration below its odor threshold. Further embodiments provide for such one or more compounds that are contributors to “off-odor”.

In some embodiments, a BG product of the disclosure does not comprise, does not consist essentially of, does not consist of, or minimally comprises any one or more of the compounds disclosed here comprising a mass spectrum characterized by quadrupole time-of-flight-mass spectrometry electron ionization (QTOF EI) at 70 eV, wherein the mass spectrum comprises peaks, wherein the mass spectrum is selected from the group consisting of: (a) 43.017 m/z, 55.0541 m/z, 73.0283 m/z, 86.0723 m/z, 97.0642 m/z, and 115.0752; (b) 41.0385 m/z, 57.0698 m/z, 68.0259 m/z, 72.0569 m/z, 85.0646 m/z, 99.0801 m/z, and 114.1033 m/z; and (c) 43.0179 m/z, 57.0336 m/z, 72.0206 m/z, 86.0362 m/z, 114.0675 m/z, 128.0830 m/z, 138.0670 m/z, and 155.1060 m/z. In some embodiments, such one or more compounds can be in an amount or concentration below its odor threshold. Further embodiments provide for such one or more compounds that are contributors to “off-odor”.

Some embodiments are directed to a BG product that does not comprise, does not consist essentially of, does not consist of, or minimally comprises any one or more of the compounds selected from the group consisting of: butyl acetate; butyl acrylate; 4-hydroxy-2-butanone; 2-methoxymethyl-2,4,5-trimethyl-1,3-dioxolane (solventy); 3-heptanone; 1-octen-3-one; N-pentyl-2-butylamine (sulfury and/or urine-like); 3-buten-2-one; 2-butenal; 2,4-dimethyl-1,3-dioxane; 2-ethyl-5-methyl-1,3-dioxane; 2-heptyl-1,3-dioxepane; 1-hydroxy-2-propanone; 1,2 propanediol (12PDO); 1,2,3-butanetriol; 1,2,3-propanetriol; 1,3 propanediol (13PDO); 1,4-butanediol (BDO); 2,3-butanediol (23BDO); 2,5-hexanedione; 3-buten-2-ol; cis-cryotyl alcohol; isopropyl alcohol (IPA); methyl vinyl ketone (MVK); n-butane (n-but); dimethylethanolamine (DMAE); toluene; HDD; and any combinations thereof, where the compound is in an amount of 0 ppm or more (e.g., 0.00001 or more), 10 ppm or less, or 0 ppm-10 ppm. In some embodiments, such one or more compounds can be in an amount or concentration below its odor threshold. Further embodiments provide for such one or more compounds that are contributors to “off-odor”.

In other embodiments, a BG product of the disclosure does not comprise, does not consist essentially of, does not consist of, or minimally comprises any one or more of the compounds selected from the group consisting of: 3-buten-2-one; 3-heptanone; 1-octen-3-one; styrene; butyl acetate; butyl acrylate; 4-hydroxy-2-butanone; 2-methoxymethyl-2,4,5-trimethyl-1,3-dioxolane (solventy); N-pentyl-2-butylamine (sulfury and/or urine-like); 3-buten-2-one; 2-butenal; 2,4-dimethyl-1,3-dioxane; 2-ethyl-5-methyl-1,3-dioxane; 2-heptyl-1,3-dioxepane; 1-hydroxy-2-propanone; 1,2 propanediol (12PDO); 1,2,3-butanetriol; 1,2,3-propanetriol; 1,3 propanediol (13PDO); 1,4-butanediol (BDO); 2,3-butanediol (23BDO); 2,5-hexanedione; 3-buten-2-ol; cis-cryotyl alcohol; isopropyl alcohol (IPA); methyl vinyl ketone (MVK); n-butane (n-but); dimethylethanolamine (DMAE); toluene; 2-methoxymethyl-2,4,5-trimethyl-1,3-dioxolane, HDD, or any combinations thereof. Additional embodiments provide for such compounds in an amount of 10 ppm or more. In some embodiments, such one or more compounds can be in an amount or concentration below its odor threshold. Further embodiments provide for such one or more compounds that are contributors to “off-odor”.

Further embodiments can be directed to a BG product comprising a bioBG, and further comprising, minimally comprising, consisting essentially of, consisting of, or minimally comprises one or more compounds selected from the group consisting of: 5-norbornene-2-ol; 2-ethyl-1-hexanol; 3-buten-2-one; 3-heptanone; 1-octen-3-one; styrene; butyl acrylate; and salts, derivatives, analogs, or solvates, thereof, or any combinations thereof as measured by Dynamic Headspace (DHS) and tandem GC/MS using Agilent Triple Quadrupole Mass Spectrometer (DHS-GC/MS-QQQ). See, e.g., EXAMPLE 6, TABLE 11. In some embodiments, such one or more compounds can be in an amount or concentration below its odor threshold. An “odor threshold” is the amount or concentration of a substance at which 50% of sensory odor panelists cannot smell a tested odor. Further embodiments provide for such one or more compounds that are contributors to “off-odor”.

Other embodiments provide for a BG product comprising a bioBG, and further comprising, minimally comprising, consisting essentially of, or consisting of one or more compounds selected from the group consisting of: 5-norbornene-2-ol; 2-ethyl-1-hexanol; 3-buten-2-one; 3-heptanone; 1-octen-3-one; styrene; butyl acrylate; HDD; and salts, derivatives, analogs, or solvates, thereof, or any combinations thereof, where the one or more compounds comprises a fold change over its respective odor threshold, where the fold change is 0.1× or greater (e.g., 0.2×, 0.3×, 0.4×, 0.5×, 0.6×, 0.7×, 0.8×, 0.9×, 1×, 2×, 3×, 4×, 5×, 6×, 7×, 8×, 9×, 10×, 15×, 20×, 25×, 30×); 50× or less (e.g., 45×, 40×, 35×, 30×, 25×, 20×, 15×, 10×, 9.5×, 8.5×, 7.5×, 6.5×, 5.5×, 4.5×, 3.5×, 2.5×, 1.5×, 0.95×, 0.85×, 0.75×, 0.65×, 0.55×, 0.45×, 0.35×, 0.25×, 0.15×); or 0×-50× (e.g., 0.1×-49.5×; 0.2×-48.5×; 0.3×-47.5×; 0.4×-46.5×; 0.5×-45.5×; 0.6×-44.5×; 0.7×-43.5×; 0.8×-42.5×; 0.9×-41.5×; 1×-40.5×; 1.2×-39.5×; 1.4×-38.5×; 1.6×-37.5×; 1.8×-36.5×; 2×-35.5×; 2.2×-34.5×; 2.4×-33.5×; 2.6×-32.5×; 2.8×-31.5×; 3×-30.5×; 3.2×-29.5×; 3.4×-28.5×; 3.6×-27.5×; 3.8×-26.5×; 4×-25.5×; 4.2×-24.5×; 4.4×-23.5×; 4.6×-22.5×; 4.8×-21.5×; 5×-20.5×; 5.2×-19.5×; 5.4×-18.5×; 5.6×-17.5×; 5.8×-16.5×; 6×-15.5×; 6.2×-14.5×; 6.4×-13.5×; 6.6×-12.5×; 6.8×-11.5×; 7×-10.5×; 7.2×-9.5×; 7.4×-8.5×). In some embodiments, such one or more compounds can be in an amount or concentration below its odor threshold. Further embodiments provide for such one or more compounds that are contributors to “off-odor”.

In some embodiments, a BG product of the disclosure comprises a bioderived BG (bioBG). Some embodiments of the disclosure are directed to a BG product comprising a BG selected from the group consisting of: (R)-1,3-BG; (S)-1,3-BG; a mixture of (R)-1,3-BG and (S)-1,3-BG. Some embodiments provide for a mixture that comprises 75% or greater of (R)-1,3-BG (e.g., 80%, 85%, 90%, 95%, 97%, 99%, 99.5%, 100%); or the mixture comprises 25% or less of (S)-1,3-BG (e.g., 20%, 15%, 10%, 5%, 4%, 3%, 2%, 1%, 0.5%, 0.1%, 0.05%, 0.001%; 0%).

Some embodiments of the disclosure are directed to a BG product comprising bioBG, where the BG product is substantially or essentially non-odorous or neutral, and does not mask an odor, add an odor, or alter a scent profile of the BG product or of a composition comprising the BG product of the disclosure. Further embodiments provide an essentially non-odorous or odorless BG product, including for example, bioBG, having an improved odor characteristic compared to crude or unpurified 1,3-BG. Furthermore, a bioBG product or a composition comprising the bioBG product of the disclosure has several properties advantageous for consumer use or application. For example, the bioBG product described here is non-irritating, non-sensitizing, non-allergenic, non-greasy, or has any combinations of the aforementioned properties. Compositions of the disclosure comprising a bioBG product described here are also non-irritating, non-sensitizing, non-allergenic, non-greasy, or any combinations of the aforementioned properties.

Another embodiment of the disclosure provides a bioderived 1,3-butylene glycol (bioBG), comprising (R)-1,3-butylene glycol (CAS No. 6290-03-5); (S)-1,3-butylene glycol (CAS No. 24621-61-2); or combinations or mixtures of (R)-1,3-butylene glycol and (S)-1,3-butylene glycol. In other embodiments, the bioBG consists essentially of: (R)-1,3-butylene glycol; (S)-1,3-butylene glycol; or combinations or mixtures of (R)-1,3-butylene glycol and (S)-1,3-butylene glycol. In some embodiments, the bioBG is a racemate or a mixture of (R)- and (S)-enantiomers of bioBG. Some embodiments provide for a bioBG racemate having more (R)-1,3-BG than (S)-1,3-BG. Further embodiments provide for bioBG comprising or consisting essentially of: (R)-1,3-butylene glycol. Some embodiments provide for a bioBG racemate having more (R)-1,3-BG than (S)-1,3-BG. In another embodiment, the bioBG comprises (R)-1,3-BG, where (R)-1,3-butylene glycol is present in bioBG in more than or equal to 75% (e.g., 80%, 85%, 90%, 95%; 96%; 97%; 98%; 99%; 99.5%; 99.9%; 100%); where (R)-1,3-butylene glycol is present in bioBG in less than or equal to 100% (e.g., 99.8%, 99.3%, 98.5%, 97.5%, 96.5%, 95.5%, 93%, 87%, 83%, 77%); or (R)-1,3-butylene glycol, where (R)-1,3-butylene glycol is present in bioBG in 75%-100% (e.g., 80%-99%; 85%-95%; 87%-93%) as detected by mechanical separation, reaction with enzymes, formation of diastereomers, or chromatographic methods (e.g., chiral chromatography, gas chromatography, GC/MS, LC-MS, MD-GC, TLC). In another embodiment, a bioBG comprises (S)-1,3-butylene glycol, where (S)-1,3-butylene glycol is present in bioBG in more than or equal to 0% (e.g., 0.001%, 0.01%, 0.05%, 0.1%, 0.15%, 0.2%, 0.5%, 1%, 2%, 3%, 4%, 5%, 10%, 20%, 25%, 30%); (S)-1,3-butylene glycol, where (S)-1,3-butylene glycol is present in bioBG in less than or equal to 25% (e.g., 20%, 15%, 12%, 8%, 5.5%, 4.5%, 3.5%, 2.5%, 1.5%, 1.2%, 0.8%, 0.3%, 0.1%, 0.08%, 0.02%, 0.005%, 0.001%); or (R)-1,3-butylene glycol, where (R)-1,3-butylene glycol is present in 1,3-BG or bioBG in 0%-25% (e.g., 1%-22%; 2%-12%; 3%-10%; 4%-8%).

In some embodiments, the bioderived butylene glycol (bioBG) provided here can have one or more asymmetric carbon atoms and can exist in the form of optically pure enantiomers, mixtures of enantiomers such as racemates, optically pure diastereoisomers, mixtures of diastereoisomers, diastereoisomeric racemates or mixtures of diastereoisomeric racemates. The optically active forms can be obtained for example by resolution of the racemates, by asymmetric synthesis or asymmetric chromatography (chromatography with a chiral adsorbent or eluant). That is, the 1,3-butylene glycol may exist in various stereoisomeric forms. “Stereoisomers” are compounds that differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms and represent the configuration of substituents around one or more chiral carbon atoms. Enantiomers of a compound can be prepared, for example, by separating an enantiomer from a racemate using one or more well-known techniques and methods, such as chiral chromatography and separation methods based thereon. The appropriate technique and/or method for separating an enantiomer of a compound described herein from a racemic mixture can be readily determined by those of skill in the art. “Racemate” or “racemic mixture” means a mixture containing two enantiomers, wherein such mixtures exhibit no optical activity; i.e., they do not rotate the plane of polarized light. “Geometric isomer” means isomers that differ in the orientation of substituent atoms (e.g., to a carbon-carbon double bond, to a cycloalkyl ring, to a bridged bicyclic system). Atoms (other than H) on each side of a carbon-carbon double bond may be in an E (substituents are on opposite sides of the carbon-carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in atropisomeric forms. Atropisomers are stereoisomers resulting from hindered rotation about single bonds where the steric strain barrier to rotation is high enough to allow for the isolation of the conformers. The compounds disclosed herein may be prepared or obtained as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods. The disclosure embraces all of these forms.

In some embodiments, the BG product of the disclosure can be obtained by any one of the commonly known and used methods, including but not limited to, distillation, solvent extraction, and enfleurage (e.g., steam distillation, hydrodistillation, cohobation, molecular distillation, cold pressed extraction, and sieving). For example, for solvent extraction, a sample comprising BG can be extracted with a mixture of water and dichloromethane (DCM). The mixture can be vigorously mixed and then the DCM layer collected. DCM can then be concentrated to a volume under nitrogen gas. See, e.g., EXAMPLE 2.

In further embodiments, any BG product described here comprising a neutral odor bioBG can comprise, consist essentially of, consist of, or minimally comprise one or more aromatic compounds which can be responsible for an odor region or an odor-active region described here, where the one or more compounds can be present in an amount below its respective odor threshold.

### Neutral Odor BG Composition

Some embodiments of the disclosure are directed to compositions or formulations comprising a BG product as described here, where the BG product comprises a neutral odor, and the composition of the disclosure further comprises a vehicle (e.g., a carrier, a diluent, an excipient, or any combinations thereof). In some embodiments, the compositions or formulations comprising a neutral odor BG product (e.g., bioBG) can further comprise, minimally comprise, consist essentially of, or consist of one or more compounds selected from: 5-norbornene-2-ol; 2-ethyl-1-hexanol; and salts, derivatives, analogs, or solvates, thereof, or any combinations thereof, wherein the BG product comprises a bioderived BG (bioBG), and wherein the composition comprises a vehicle (e.g., a carrier, a diluent, an excipient, or any combinations thereof). In some embodiments, such one or more compounds can be in an amount or concentration below its odor threshold.

Additional embodiments can provide a composition or formulation comprising a BG product comprising bioBG which further comprises, minimally comprises, consists essentially of, or consists of one or more compounds selected from the group consisting of: 5-norbornene-2-ol; 2-ethyl-1-hexanol; 3-buten-2-one; 3-heptanone; 1-octen-3-one; styrene; butyl acrylate; HDD; and salts, derivatives, analogs, or solvates thereof, or any combinations thereof, where the composition comprises a vehicle (e.g., a carrier, a diluent, an excipient, or any combinations thereof). In some embodiments, such one or more compounds can be in an amount or concentration below its odor threshold.

In some embodiments, a BG composition or formulation of the disclosure comprises a BG product comprising a bioBG where the BG product can further comprise, consist essentially of, consist of, or minimally comprise: one or more compounds selected from: 5-norbornene; 2-ethyl-1-hexanol in an amount of ˜0.000001 ppm or greater (e.g., ˜0.000001 ppm-˜10 ppm; ˜0.1 ppm-˜0.2 ppm) 3-buten-2-one in an amount of about 1.75 ppm-2 ppm; styrene in an amount of 0.0005 ppm or less; 3-heptanone in an amount of 0.00005 ppm or less; 1-octen-3-one in an amount of 0.00002 or less, or in an amount of less than 187, 45 or 19 fold change relative to a reference value or a control as quantified using a dichloromethane extraction method; and salts, derivatives, analogs, or solvates, thereof, or any combinations thereof, where the composition further comprises a vehicle (e.g., a carrier, a diluent, an excipient, or any combinations thereof). In some embodiments, such one or more compounds can be in an amount or concentration below its odor threshold.

In some embodiments, a BG composition or formulation of the disclosure comprises a BG product described here comprising a bioBG, where the BG product further comprises, minimally comprises, consists essentially of, or consists of one or more of the compounds disclosed here, where the compound is in an amount of 0 ppm or more (e.g., 0.00001 or more), 10 ppm or less, or 0 ppm-10 ppm, where the compound is selected from the group consisting of: 5-norbornene-2-ol; 2-ethyl-1-hexanol; 3-buten-2-one; 3-heptanone; 1-octen-3-one; styrene; butyl acrylate; HDD; and salts, derivatives, analogs, or solvates thereof, or any combinations thereof, and the composition comprises a vehicle (e.g., a carrier, a diluent, an excipient, or any combinations thereof). In some embodiments, such one or more compounds can be in an amount or concentration below its odor threshold.

Some embodiments provide a BG composition or formulation comprising a BG product disclosed here, where the BG product comprises a bioBG, the BG product comprises, minimally comprises, consists essentially of, or consists of one or more compounds comprising an aromatic region, an odor-active region, or a peak as measured by GC/MS/O with an odor selected from the group consisting of: ((a) fruity; (b) citrus and/or floral and/or oily; (c) rubber and/or plastic and/or green; (d) plastic and/or musty; (e) mushroom and/or earthy, that corresponds to compounds: (a) 5-norbornene-2-ol; (b)2-ethyl-1-hexanol; (c) 3-buten-2-one; (d) 3-heptanone; (e) 1-octen-3-one, respectively, and the composition comprises a vehicle (e.g., a carrier, a diluent, an excipient, or any combinations thereof. In some embodiments, such one or more compounds can be in an amount or concentration below its odor threshold.

In some embodiments, a composition or formulation comprising a BG product of the disclosure comprises a bioBG, and further comprises, minimally comprises, consists essentially of, or consists of one or more compounds selected from the group consisting of: 5-norbornene-2-ol; and 2-ethyl-1-hexanol; 3-buten-2-one; 3-heptanone; 1-octen-3-one; styrene, where the one or more compounds is in an amount of 0 ppm or more (e.g., 0.00001 or more), 10 ppm or less, or 0 ppm-10 ppm, where the composition comprises a vehicle (e.g., a carrier, a diluent, an excipient, or any combinations thereof). For example, 3-buten-2-one can be in an amount of about 1.75 ppm-2 ppm; or styrene can be in an amount of 0.0005 ppm or less. In some embodiments, such one or more compounds can be in an amount or concentration below its odor threshold.

Additional embodiments are directed to a composition or formulation comprising a 1,3-butylene glycol (BG) product, where the BG product comprises a bioderived (bioBG) and further comprises, consists essentially of, consists of, or minimally comprises one or more compounds characterized by an odor-active region as measured by gas chromatography/mass spectrometry/olfactory (GC/MS/O) having a relative acquisition time selected from a group consisting of: about 0.594 min (fruity and/or solventy), about 0.628 min (fruity), about 0.655 min (citrus and/or peel), about 0.711 min (solventy), about 1.328 mins (roasty and/or exhaust), and combinations thereof, where the BG product comprises bioderived BG (bioBG), and the bioBG has a relative acquisition time of 1 min, and where the composition comprises a vehicle (e.g., a carrier, a diluent, an excipient, or any combinations thereof. In some embodiments, such one or more compounds can be in an amount or concentration below its odor threshold.

Some embodiments of the disclosure provide a BG composition or formulation comprising a bioBG product and further comprises, minimally comprises, consists essentially of, or consists of one or more compounds characterized by an aromatic region, an odor-active region, or a peak as measured by gas chromatography/quadrupole time-of-flight-mass spectrometry (GC/QTOF-MS), where the aromatic region, the odor-active region, or the peak elutes at an acquisition time selected from a group consisting of: about 12.2 mins (e.g., 12.18 mins-12.27 mins; 12.18 mins, 12.19 mins, 12.2 mins, 12.21 mins, 12.22 mins, 12.23 mins, 12.24 mins, 12.25 mins, 12.26 mins, 12.27 mins) at 66.0463 m/z, where the composition comprises a vehicle (e.g., a carrier, a diluent, an excipient, or any combinations thereof. In some embodiments, such one or more compounds can be in an amount or concentration below its odor threshold.

In further embodiments, provided here are BG compositions or formulations comprising a BG product with a bioBG and further comprising, minimally comprising, consisting essentially of, or consisting of one or more compounds having a mass spectrum as characterized by quadrupole time-of-flight-mass spectrometry electron ionization (QTOF EI) at 70 eV. Such a mass spectrum comprises peaks: 39.0229 m/z; 43.0707 m/z; 53.0385 m/z; 57.0169 m/z; 66.0463 m/z; 74.0156 m/z; 81.0333 m/z; 85.0284 m/z; 91.0539 m/z; 95.0489 m/z; and 110.0724 m/z; or a mass spectrum comprises peaks: 39.0229 m/z; 43.0707 m/z; 53.0385 m/z; 66.0463 m/z; 81.0333 m/z; 91.0539 m/z; 95.0489 m/z; 110.0724 m/z, where the composition comprises a vehicle (e.g., a carrier, a diluent, an excipient, or any combinations thereof. In some embodiments, such one or more compounds can be in an amount or concentration below its odor threshold.

In some embodiments, a composition or formulation of the disclosure comprises a BG product comprising a bioBG, where the BG product does not comprise, does not consist essentially of, does not consist of, or minimally comprises one or more compounds having an aromatic region, an odor-active region, or a peak as measured by GC/MS that elutes at an acquisition time selected from a group consisting of: about 9.3 mins (toluene); about 9.6 mins (butyl acetate); about 10.5 mins (butyl acrylate); and about 16 mins (4-hydroxy-2-butanone); and any combinations thereof, where bioBG elutes at an acquisition time of about 18 mins; or an aromatic region, an odor-active region, or a peak as measured by GC/MS that elutes at an acquisition time selected from a group consisting of: about 9.3 mins (toluene); about 9.6 mins (butyl acetate); about 10.5 mins (butyl acrylate); about 11 mins; about 11.7 mins; about 14.1 mins; about 16 mins (4-hydroxy-2-butanone); and about 17.7 mins; and any combinations thereof, where bioBG elutes at an acquisition time of about 18 mins, and where the composition comprises a vehicle (e.g., a carrier, a diluent, an excipient, or any combinations thereof. In some embodiments, such one or more compounds can be in an amount or concentration below its odor threshold. Further embodiments provide for such one or more compounds that are contributors to “off-odor”.

Additional embodiments are directed to composition or formulation of the disclosure comprising a 1,3-butylene glycol (BG) product, where the BG product does not comprise one or more compounds characterized by an aromatic region, an odor-active region, or a peak as measured by gas chromatography/mass spectrometry/olfactory (GC/MS/O) having a relative acquisition time selected from a group consisting of: about 0.517 mins (toluene); about 0.533 mins (butyl acetate); about 0.583 mins (butyl acrylate); about 0.889 mins (4-hydroxy-2-butanone); and combinations thereof, where the BG product comprises bioderived BG (bioBG), and the bioBG has a relative acquisition time of 1 min; or an aromatic region, an odor-active region, or a peak as measured by gas chromatography/mass spectrometry/olfactory (GC/MS/O) having a relative acquisition time selected from a group consisting of: about 0.517 mins (toluene); about 0.533 mins (butyl acetate); about 0.583 mins (butyl acrylate); about 0.611 mins; about 0.65 mins; about 0.789 mins; about 0.889 mins (4-hydroxy-2-butanone); about 0.983 mins; and combinations thereof, where the BG product comprises bioderived BG (bioBG), and the bioBG has a relative acquisition time of 1 min, and the composition comprises a vehicle (e.g., a carrier, a diluent, an excipient, or any combinations thereof. In some embodiments, such one or more compounds can be in an amount or concentration below its odor threshold. Further embodiments provide for such one or more compounds that are contributors to “off-odor”.

In further embodiments, provided here is a composition or formulation containing a BG product of the disclosure, where the BG product comprises a neutral odor bioBG and does not comprise, does not consist essentially of, does not consist of, or minimally comprises one or more compounds: 2-methoxymethyl-2,4,5-trimethyl-1,3-dioxolane, toluene, or combinations thereof, and comprises a vehicle (e.g., a carrier, a diluent, an excipient, or any combinations thereof. In some embodiments, such one or more compounds can be in an amount or concentration below its odor threshold. Further embodiments provide for such one or more compounds that are contributors to “off-odor”.

Some embodiments are directed to a composition or formulation comprising a BG product of the disclosure, where the BG product comprises a bioBG and does not comprise, does not consist essentially of, does not consist of, or minimally comprises any one or more of the compounds selected from the group consisting of: butyl acetate; butyl acrylate; 4-hydroxy-2-butanone; 2-methoxymethyl-2,4,5-trimethyl-1,3-dioxolane; 3-heptanone; 1-octen-3-one; N-pentyl-2-butylamine; 3-buten-2-one; 2-butenal; 2,4-dimethyl-1,3-dioxane; 2-ethyl-5-methyl-1,3-dioxane; 2-heptyl-1,3-dioxepane; 1-hydroxy-2-propanone; 1,2 propanediol (12PDO); 1,2,3-butanetriol; 1,2,3-propanetriol; 1,3 propanediol (13PDO); 1,4-butanediol (BDO); 2,3-butanediol (23BDO); 2,5-hexanedione; 3-buten-2-ol; cis-cryotyl alcohol; isopropyl alcohol (IPA); methyl vinyl ketone (MVK); n-butane (n-but); dimethylethanolamine (DMAE); HDD; and any combinations thereof, where the composition comprises a vehicle (e.g., a carrier, a diluent, an excipient, or any combinations thereof). In some embodiments, such one or more compounds can be in an amount or concentration below its odor threshold. Further embodiments provide for such one or more compounds that are contributors to “off-odor”.

In additional embodiments, a composition or formulation comprising a BG product of the disclosure, where the BG product comprises a bioBG and does not comprise, does not consist essentially of, does not consist of, or minimally comprises any one or more of the compounds selected from the group consisting of: butyl acetate; butyl acrylate; 4-hydroxy-2-butanone; 2-methoxymethyl-2,4,5-trimethyl-1,3-dioxolane; 3-heptanone; 1-octen-3-one; N-pentyl-2-butylamine; 3-buten-2-one; 2-butenal; 2,4-dimethyl-1,3-dioxane; 2-ethyl-5-methyl-1,3-dioxane; 2-heptyl-1,3-dioxepane; 1-hydroxy-2-propanone; 1,2 propanediol (12PDO); 1,2,3-butanetriol; 1,2,3-propanetriol; 1,3 propanediol (13PDO); 1,4-butanediol (BDO); 2,3-butanediol (23BDO); 2,5-hexanedione; 3-buten-2-ol; cis-cryotyl alcohol; isopropyl alcohol (IPA); methyl vinyl ketone (MVK); n-butane (n-but); dimethylethanolamine (DMAE); HDD; and any combinations thereof, where the one or more compounds is each in an amount of 10 ppm or less (e.g., 9, 8, 7, 6, 5, 4, 3, 2, 1, 0); 0 ppm or greater (e.g., 0.5, 1.5, 2.5, 3.5, 4.5, 5.5, 6.5, 7.5, 8.5, 9.5); or 0 ppm-10 ppm (e.g., 0.1 ppm-9.8 ppm; 0.2 ppm-9.6 ppm; 0.3 ppm-9.4 ppm; 0.4 ppm-9.2 ppm; 0.5 ppm-9 ppm; 0.6 ppm-8.8 ppm; 0.7 ppm-8.6 ppm; 0.8 ppm-8.4 ppm; 0.9 ppm-8.2 ppm; 1 ppm-8 ppm), where the composition comprises a vehicle (e.g., a carrier, a diluent, an excipient, or any combinations thereof). In some embodiments, such one or more compounds can be in an amount or concentration below its odor threshold. Further embodiments provide for such one or more compounds that are contributors to “off-odor”.

In some embodiments, a composition or formulation comprising the BG product described here comprising a bioBG, and does not further comprise, does not consist essentially of, does not consist of, or minimally comprises one or more of the compounds selected from the group consisting of: 3-hydroxy-butanal (e.g., less than 25 ppm); 4-hydroxy-2-butanone (e.g., less than 25 ppm); 4-(3-hydroxybutoxy)butan-2-one (e.g., less than 25 ppm); 4-((4-hydroxybutan-2-yl)oxy)-butan-2-one (e.g., less than 25 ppm); 1,2-propanediol (e.g., less than 1 ppm); 1,3-propanediol (e.g., less than 10 ppm); 2,3-butanediol (e.g., less than 1 ppm); 2,4-dimethyl-6-((1-(4-methyl-1,3-dioxan-2-yl)propan-2-yl)oxy)-1,3-dioxane (e.g., less than 2 ppm); 4-(sec-butoxy)-2,6-dimethyl-1,3-dioxane (e.g., less than 4 ppm); 2,4-dimethyl-1,3-dioxane (e.g., less than 8 ppm); (E)-4-methyl-2-(prop-1-en-1-yl)-1,3-dioxane (e.g., less than 8 ppm); 3-hydroxybutyl acetate (e.g., less than 8 ppm); 1-(2-methyl-1,3-dioxan-4-yl)propan-2-ol (e.g., less than 8 ppm); 2-methyl-4-propyl-1,3-dioxane (e.g., less than 8 ppm); tri-substituted 1,3-dioxane

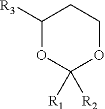

(e.g., less than 8 ppm) and/or 3-hydroxybutyl carbono-substituted) (e.g., less than 8 ppm), wherein R1 to R4 are the same or different; a hydrogen atom, a C1-C4 alkyl group optionally substituted by an OH group, or a C2-C4 alkenyl group optionally substituted by OH group; 2,4-dimethyl-2-vinyl-1,3-dioxane; 2-(2,4-dimethyl-1,3-dioxan-2-yl)ethan-1-ol; 2,2,4-trimethyl-1,3-dioxane; 4-methyl-1,3-dioxane, wherein the total of 2,4-dimethyl-2-vinyl-1,3-dioxane; 2-(2,4-dimethyl-1,3-dioxan-2-yl)ethan-1-ol; 2,2,4-trimethyl-1,3-dioxane; 4-methyl-1,3-dioxane is in a total amount of less than 34 ppm; (E)-4-methyl-2-(prop-1-en-1-yl)-1,3-dioxane; 1-(4-methyl-1,3-dioxan-2-yl)propan-2-ol; 2,4-dimethyl-1,3-dioxane; 3-hydroxybutyl (E)-but-2-enoate; 3-hydroxybutyl acetate; 3-hydroxybutyl 3-hydroxybutanoate, wherein the total of (E)-4-methyl-2-(prop-1-en-1-yl)-1,3-dioxane; 1-(4-methyl-1,3-dioxan-2-yl)propan-2-ol; 2,4-dimethyl-1,3-dioxane; 3-hydroxybutyl (E)-but-2-enoate; 3-hydroxybutyl acetate; 3-hydroxybutyl 3-hydroxybutanoate is in a total amount of less than 63 ppm or wherein the total of 2,4-dimethyl-2-vinyl-1,3-dioxane; 2-(2,4-dimethyl-1,3-dioxan-2-yl)ethan-1-ol; 2,2,4-trimethyl-1,3-dioxane; 4-methyl-1,3-dioxane; (E)-4-methyl-2-(prop-1-en-1-yl)-1,3-dioxane; 1-(4-methyl-1,3-dioxan-2-yl)propan-2-ol; 2,4-dimethyl-1,3-dioxane; 3-hydroxybutyl (E)-but-2-enoate; 3-hydroxybutyl acetate; 3-hydroxybutyl 3-hydroxybutanoate is in a total amount of less than 97 ppm; methyl vinyl ketone (e.g., less than 8 ppm); 1-hydroxy-3-butanone (e.g., less than 1 ppm); acetone (e.g., less than 6 ppm); formaldehyde (e.g., less than 2 ppm); crotonaldehyde (e.g., less than 1 ppm); acetaldol (e.g., less than 1 ppm); acetaldehyde (e.g., less than 5 ppm); butyraldehyde (e.g., less than 4 ppm); 2-butanol, wherein the total of 2,4-dimethyl-2-vinyl-1,3-dioxane; 2-(2,4-dimethyl-1,3-dioxan-2-yl)ethan-1-ol; 2,2,4-trimethyl-1,3-dioxane; 4-methyl-1,3-dioxane; (E)-4-methyl-2-(prop-1-en-1-yl)-1,3-dioxane; 1-(4-methyl-1,3-dioxan-2-yl)propan-2-ol; 2,4-dimethyl-1,3-dioxane; 3-hydroxybutyl (E)-but-2-enoate; 3-hydroxybutyl acetate; 3-hydroxybutyl 3-hydroxybutanoate; and 2-butanol is in a total amount of less than 65 ppm; butyl acrylate (e.g., in an amount of less than 1.82 mg/L, less than 0.53 mg/L, less than 0.33 mg/L, less than 0.24 mg/L, or less than 0.0001 mg/L); HDD (e.g., in an amount less than 215, less than 19.3, less than 4, or less than 2.5 in fold change relative to a reference value or a control as quantified with a dichloromethane extraction method); and any combinations thereof, where the composition comprises a vehicle (e.g., a carrier, a diluent, an excipient, or any combinations thereof. In some embodiments, such one or more compounds can be in an amount or concentration below its odor threshold. Further embodiments provide for such one or more compounds that are contributors to “off-odor”.

In some embodiments of the disclosure, a composition or formulation comprises a BG product, where the BG product is a neutral odor bioBG product that comprises, minimally comprises, consists essentially of, or consists of one or more compounds characterized by an aromatic region, an odor-active region, or a peak, used interchangeably here, as measured by gas chromatography/mass spectrometry/olfactory (GC/MS/O). Some embodiments provide for a composition or formulation comprising a BG product, which comprises a bioBG and can comprise one or more compounds, such as aromatic compounds or aromatic compounds minimally having an undesirable or offensive odor as characterized by the aromatic region, the odor-active region, or the peak as measured by gas chromatography/mass spectrometry/olfactory (GC/MS/O). Some embodiments provide a composition or formulation comprising a BG product of the disclosure that does not comprise, does not consist essentially of, does not consist of, or minimally comprises one or more compounds having an aromatic region, an odor-active region, or a peak of the GC/MS/O that elutes at an acquisition time selected from the group consisting of: ˜2.5 mins (rubber and/or plastic and/or green; 3-buten-2-one); ˜2.7 mins (plastic); ˜2.9 mins (solventy; 2-butenal); ˜6.2 mins (fruity and/or plastic); ˜8.2 mins (fruity and/or plastic and/or metal and/or mushroom; 2-heptyl-1,3-dioxepane); ˜10.7 mins (fruity and/or solventy); ˜11.3 mins (fruity); A11.8 mins (citrus and/or peel and/or fatty); ˜12.8 mins (solventy); ˜23.9 mins (roasty and/or exhaust); and any combinations thereof, where the composition comprises a vehicle (e.g., a carrier, a diluent, an excipient, or any combinations thereof. In some embodiments, such one or more compounds can be in an amount or concentration below its odor threshold. Further embodiments provide for such one or more compounds that are contributors to “off-odor”.

Additional embodiments are directed to a composition or formulation comprising a BG product of the disclosure comprising a bioBG, and does not comprise, does not consist essentially of, does not consist of, or minimally comprises one or more compounds characterized by an aromatic region, an odor-active region, or a peak as measured by gas chromatography/mass spectrometry/olfactory (GC/MS/O), where the aromatic region, the odor-active region, or the peak elutes at an acquisition time selected from a group consisting of: ˜2.5 mins (rubber and/or plastic and/or green; 3-buten-2-one); ˜2.7 mins (plastic); ˜2.9 mins (solventy; 2-butenal); ˜6.2 mins (fruity and/or plastic); ˜8.2 mins (fruity and/or plastic and/or metal and/or mushroom; 2-heptyl-1,3-dioxepane); ˜10.7 mins (fruity and/or solventy); ˜11.3 mins (fruity); ˜11.8 mins (citrus and/or peel and/or fatty); ˜12.8 mins (solventy); ˜23.9 mins (roasty and/or exhaust); and any combinations thereof, wherein the compound is in an amount of 0 ppm or more (e.g., 0.00001 or more), 10 ppm or less, or 0 ppm-10 ppm, where the composition comprises a vehicle (e.g., a carrier, a diluent, an excipient, or any combinations thereof. In some embodiments, such one or more compounds can be in an amount or concentration below its odor threshold. Further embodiments provide for such one or more compounds that are contributors to “off-odor”.

In some embodiments, a composition or formulation of the disclosure comprises a BG product described here, where the BG product comprises, consists essentially of, consists of, or minimally comprises one or more compounds, as measured by GC/MS/O, having an aromatic region, an odor-active region, or a peak with a relative acquisition time selected from a group consisting of: about 0.594 min (fruity and/or solventy), about 0.628 min (fruity), about 0.655 min (citrus and/or peel), about 0.711 min (solventy), about 1.328 min (roasty and/or exhaust), and any combinations thereof, where the BG product comprises bioBG, and the BG has a relative acquisition time of 1 min found in DSP-555-2 and/or DSP-555-4, where the composition comprises a vehicle (e.g., a carrier, a diluent, an excipient, or any combinations thereof. In some embodiments, such one or more compounds can be in an amount or concentration below its odor threshold.

In some embodiments, the disclosure provides a composition or formulation comprising a BG product and that does not comprise, does not consist essentially of, minimally comprises, or does not consist of one or more compounds characterized by an odor-active region as measured by gas chromatography/mass spectrometry/olfactory (GC/MS/O) having a relative acquisition time selected from a group consisting of: about 0.417 mins (rubber and/or plastic and/or green), about 0.450 mins (plastic), about 0.483 mins (solventy), about 0.783 mins (plastic), about 1.033 mins (fruity and/or plastic), about 1.1 mins (plastic and/or pungent and/or mushroom and/or metallic), about 1.367 mins (fruity and/or plastic and/or metal and/or mushroom), about 1.967 mins (waxy and/or fatty), and any combinations thereof, where the BG product comprises bioderived BG (bioBG), and the bioBG has a relative acquisition time of 1 min, where the composition comprises a vehicle (e.g., a carrier, a diluent, an excipient, or any combinations thereof. In some embodiments, such one or more compounds can be in an amount or concentration below its odor threshold. Further embodiments provide for such one or more compounds that are contributors to “off-odor”.

Some embodiments of the disclosure provide a composition or formulation comprising a BG product described here, where the BG product does not comprise, does not consist essentially of, minimally comprises, or does not consist of one or more compounds characterized by an aromatic region, an odor-active region, or a peak as measured by gas chromatography/mass spectrometry/olfactory (GC/MS/O) selected from the group consisting of: a fusel and/or sulfur and/or oil odor at an acquisition time of ˜9 mins-˜9.2 mins (relative acquisition times ˜0.5 mins, ˜0.506 mins, ˜0.511 mins, respectively); ˜9.3 mins (relative acquisition time 0.517; toluene); a fruity and/or plastic odor at an acquisition time of ˜10.2 mins-˜10.4 mins (relative acquisition times ˜0.567 mins, ˜0.572 mins, ˜0.578 mins, respectively); a fruity and/or floral and/or plastic odor at an acquisition time of ˜10.5 mins-˜10.7 mins (relative acquisition times ˜0.583 mins, ˜0.589 mins, ˜0.594 mins, respectively); and a plastic and/or roasty odor at an acquisition time of ˜13.3 mins-˜13.5 mins (relative acquisition times ˜0.739 mins, ˜0.744 mins, ˜0.75 mins, respectively); and any combinations thereof, where the composition comprises a vehicle (e.g., a carrier, a diluent, an excipient, or any combinations thereof. In some embodiments, such one or more compounds can be in an amount or concentration below its odor threshold. Additional embodiments are directed to a composition or formulation comprising a BG product comprising bioBG, where the BG product does not comprise, does not consist essentially of, minimally comprises, or does not consist of an odor-active region and/or one or more compounds as measured by gas chromatography/mass spectrometry/olfactory (GC/MS/O) and selected from any of DSP-555-1, DSP-555-3, DSP-555-5, DSP-555-6, DSP-555-7, DSP-555-8, DSP-555-9, DSP-555-10, DSP-555-11, DSP-555-12, DSP-555-13, DSP-555-14, DSP-555-15, DSP-555-16, and any combinations thereof as found in TABLE 2 and/or TABLE 3, where the composition comprises a vehicle (e.g., a carrier, a diluent, an excipient, or any combinations thereof. In some embodiments, such one or more compounds can be in an amount or concentration below its odor threshold. Further embodiments provide for such one or more compounds that are contributors to “off-odor”.

In some embodiments, a composition or formulation comprising a BG product of the disclosure comprising bioBG does not comprise, does not consist essentially of, does not consist of, or minimally comprises one or more compounds having an aromatic region, an odor-active region, or a peak as measured by GC/MS/O that elutes at an acquisition time selected from a group consisting of: about 6 mins (solventy), about 7 mins (plastic and/or musty), about 9 mins (mushroom and/or earthy), about 17.5 mins (sulfury and/or urine-like), and combinations thereof, where the composition comprises a vehicle (e.g., a carrier, a diluent, an excipient, or any combinations thereof). In some embodiments, such one or more compounds can be in an amount or concentration below its odor threshold. Further embodiments provide for such one or more compounds that are contributors to “off-odor”.

Some embodiments provide a composition or formulation comprising a BG product of the disclosure comprising bioBG, and does not comprise, does not consist essentially of, does not consist of, or minimally comprises one or more compounds having an aromatic region, an odor-active region, or a peak as measured by GC/Q-TOF-MS that elutes at an acquisition time selected from a group consisting of: about 12 mins (solventy), about 13 mins (plastic and/or musty), about 15.5 mins (mushroom and/or earthy), about 28.5 mins (sulfury and/or urine-like), and combinations thereof, where the composition comprises a vehicle (e.g., a carrier, a diluent, an excipient, or any combinations thereof. In some embodiments, such one or more compounds can be in an amount or concentration below its odor threshold. Further embodiments provide for such one or more compounds that are contributors to “off-odor”.

In some embodiments, a composition or formulation described here comprises a BG product of the disclosure and does not comprise, does not consist essentially of, does not consist of, or minimally comprises one or more compounds characterized by an aromatic region, an odor-active region, or a peak as measured by gas chromatography/quadrupole time-of-flight-mass spectrometry (GC/QTOF-MS), where the aromatic region, the odor-active region, or the peak elutes at an acquisition time selected from a group consisting of: about 12 mins (e.g., 11.9 mins-12.2 mins; 11.9 mins, 12 mins, 12.1 mins, 12.2 mins) at 59.0489 m/z, about 13 mins (e.g., 12.955 mins-13.15 mins; 12.956 mins, 12.957 mins, 12.958 mins, 12.959 mins, 12.96 mins, 12.961 mins, 12.962 mins, 12.963 mins, 12.964 mins, 12.965 mins, 12.966 mins, 12.967 mins, 12.968 mins, 12.969 mins, 12.97 mins, 12.971 mins, 12.972 mins, 12.973 mins, 12.974 mins, 12.975 mins, 12.976 mins, 12.977 mins, 12.978 mins, 12.979 mins, 12.98 mins, 12.981 mins, 12.982 mins, 12.983 mins, 12.984 mins, 12.985 mins, 12.986 mins, 12.987 mins, 12.988 mins, 12.989 mins, 12.99 mins, 12.991 mins, 12.992 mins, 12.993 mins, 12.994 mins, 12.995 mins, 12.996 mins, 12.997 mins, 12.998 mins, 12.999 mins, 13 mins, 13.01 mins, 13.02 mins, 13.03 mins, 13.04 mins, 13.05 mins, 13.06 mins, 13.07 mins, 13.08 mins, 13.09 mins, 13.1 mins, 13.11 mins, 13.12 mins, 13.13 mins, 13.14 mins, 13.15 mins) at 57.0698 m/z, about 15.5 mins (e.g., 15.55 mins-15.65; 15.55 mins, 15.56 mins, 15.57 mins, 15.58 mins, 15.59 mins, 15.6 mins, 15.61 mins, 15.62 mins, 15.63 mins, 15.64 mins, 15.65 mins) at 70.0411 m/z, about 28.5 mins (e.g., 28.5 mins-28.6 mins) at 114.0675 m/z, and combinations thereof, where the composition comprises a vehicle (e.g., a carrier, a diluent, an excipient, or any combinations thereof. In some embodiments, such one or more compounds can be in an amount or concentration below its odor threshold. Further embodiments provide for such one or more compounds that are contributors to “off-odor”.

Embodiments of the disclosure can be directed to a composition or formulation comprising a 1,3-butylene glycol (BG) product, that does not comprise, does not consist essentially of, does not consist of, or minimally comprises one or more compounds having a mass spectrum as characterized by quadrupole time-of-flight-mass spectrometry electron ionization (QTOF EI) at 70 eV. The mass spectrum comprises peaks, and the mass spectrum is selected from the group consisting of: (a) 43.017 m/z, 55.0541 m/z, 73.0283 m/z, 86.0723 m/z, 97.0642 m/z, and 115.0752; (b) 41.0385 m/z, 57.0698 m/z, 68.0259 m/z, 72.0569 m/z, 85.0646 m/z, 99.0801 m/z, and 114.1033 m/z; and (c) 43.0179 m/z, 57.0336 m/z, 72.0206 m/z, 86.0362 m/z, 114.0675 m/z, 128.0830 m/z, 138.0670 m/z, and 155.1060 m/z, where the composition comprises a vehicle (e.g., a carrier, a diluent, an excipient, or any combinations thereof. In some embodiments, such one or more compounds can be in an amount or concentration below its odor threshold. Further embodiments provide for such one or more compounds that are contributors to “off-odor”.

Some embodiments can provide a BG composition or a formulation comprising a neutral odor BG product (bioBG) of the disclosure, where the BG product comprises, consists essentially of, or consists of one or more of the compounds disclosed here in an amount of: 0 parts per million (ppm) or more (e.g., 0.00001 ppm, 0.000015 ppm, 0.00002 ppm, 0.000025 ppm, 0.00005 ppm, 0.0001 ppm, 0.0002 ppm, 0.0003 ppm, 0.0004 ppm, 0.0005 ppm, 0.0006 ppm, 0.0007 ppm, 0.0008 ppm, 0.0009 ppm, 0.001 ppm, 0.002 ppm, 0.003 ppm, 0.004 ppm, 0.005 ppm, 0.006 ppm, 0.007 ppm, 0.008 ppm, 0.009 ppm, 0.01 ppm, 0.02 ppm, 0.03 ppm, 0.04 ppm, 0.05 ppm, 0.06 ppm, 0.07 ppm, 0.08 ppm, 0.09 ppm, 0.1 ppm, 0.2 ppm, 0.3 ppm, 0.4 ppm, 0.5 ppm, 0.6 ppm, 0.7 ppm, 0.8 ppm, 0.9 ppm, 1 ppm, 10 ppm, 100 ppm, 1000 ppm, 10,000 ppm); or 10,000 ppm or less (e.g., 9,500 ppm, 9,000 ppm, 8,500 ppm, 8,000 ppm, 7,500 ppm, 7,000 ppm, 6,500 ppm, 6,000 ppm, 5,500 ppm, 5,000 ppm, 4,500 ppm, 4,000 ppm, 3,500 ppm, 3,000 ppm, 2,500 ppm, 2,000 ppm, 1,500 ppm, 1,000 ppm, 500 ppm, 50 ppm, 5 ppm, 0.75 ppm, 0.5 ppm, 0.25 ppm, 0.2 ppm, 0.190 ppm, 0.180 ppm, 0.170 ppm, 0.160 ppm, 0.150 ppm, 0.140 ppm, 0.130 ppm, 0.120 ppm, 0.110 ppm, 0.1 ppm, 0.09 ppm, 0.08 ppm, 0.07 ppm, 0.06 ppm, 0.05 ppm, 0.04 ppm, 0.03 ppm, 0.02 ppm, 0.01 ppm, 0.009 ppm, 0.008 ppm, 0.007 ppm, 0.006 ppm, 0.005 ppm, 0.004 ppm, 0.003 ppm, 0.002 ppm, 0.001 ppm, 0.0009 ppm, 0.0008 ppm, 0.0007 ppm, 0.0006 ppm, 0.0005 ppm, 0.0004 ppm, 0.0003 ppm, 0.0002 ppm, 0.0001 ppm, 0.00005 ppm, 0.00001); or 0 ppm-10,000 ppm (e.g., 0.00001 ppm-9,500 ppm; 0.000015 ppm-9,000 ppm; 0.00005 ppm-8,000 ppm; 0.0001 ppm-7,000 ppm; 0.01 ppm-6,000 ppm; 0.05 ppm-5,000 ppm; 0.1 ppm-4,000 ppm; 0.5 ppm-3,000 ppm; 0.2 ppm-2,000 ppm; 0.3 ppm-2,000 ppm; 0.4 ppm-1,000 ppm; 0.5 ppm-750 ppm; 0.6 ppm-500 ppm; 0.7 ppm-250 ppm; 0.8 ppm-200 ppm; 0.9 ppm-175 ppm; 1 ppm-150 ppm; 5 ppm-125 ppm; 10 ppm-100 ppm; 20 ppm-75 ppm; 30 ppm-50 ppm; 35 ppm-45 ppm), and a vehicle (e.g., a carrier, a diluent, an excipient, or any combinations thereof). In some embodiments, such one or more compounds can be in an amount or concentration below its odor threshold. Further embodiments provide for such one or more compounds that are contributors to “off-odor”.

In other embodiments, a BG composition or formulation comprises a neutral odor BG product (e.g., bioBG) of the disclosure, where the BG product comprises, consists essentially of, or consists of one or more of the compounds disclosed here, where the compound is in an amount of 0 ppm or more (e.g., 0.00001 or more), 10 ppm or less, or 0 ppm-10 ppm, where the composition comprises a vehicle (e.g., a carrier, a diluent, an excipient, or any combinations thereof. In some embodiments, such one or more compounds can be in an amount or concentration below its odor threshold. Further embodiments provide for such one or more compounds that are contributors to “off-odor”.

In some embodiments, provided here is a composition or formulation of the disclosure comprising a BG product described here comprising a bioBG and the composition does not comprise, does not consist essentially of, does not consist of, or minimally comprises one or more compounds characterized by an aromatic region, an odor-active region, or a peak as measured by gas chromatography/mass spectrometry/olfactory (GC/MS/O), where the aromatic region, the odor-active region, or the peak elutes at an acquisition time selected from a group consisting of: ˜2.5 mins (rubber and/or plastic and/or green; 3-buten-2-one); ˜2.7 mins (plastic); ˜2.9 mins (solventy; 2-butenal); ˜6.2 mins (fruity and/or plastic); ˜8.2 mins (fruity and/or plastic and/or metal and/or mushroom; 2-heptyl-1,3-dioxepane); ˜10.7 mins (fruity and/or solventy); ˜11.3 mins (fruity); ˜11.8 mins (citrus and/or peel and/or fatty); ˜12.8 mins (solventy); ˜23.9 mins (roasty and/or exhaust); and any combinations thereof; or about 6 mins (solventy), about 7 mins (plastic and/or musty), about 9 mins (mushroom and/or earthy), about 17.5 mins (sulfury and/or urine-like), and combinations thereof, and the composition comprises a vehicle (e.g., a carrier, a diluent, an excipient, or any combinations thereof). In some embodiments, such one or more compounds can be in an amount or concentration below its odor threshold. The one or more compounds comprising the aromatic region, the odor-active region, or the peak having the acquisition times (about 6 mins, about 7 mins, about 9 mins, about 17.5 mins, and combinations thereof) can be associated with an odor selected from the group consisting of: solventy; plastic and/or musty; mushroom and/or earthy; and sulfury and/or urine-like, respectively, where, in some embodiments, the one or more compounds can be present in an amount or concentration below its odor threshold. The one or more compounds comprising the aromatic region, the odor-active region, or the peak as measured by GC/MS/O with an odor selected from the group consisting of: (a) solventy; (b) plastic and/or musty; (c) mushroom and/or earthy; and (d) sulfury and/or urine-like, respectively, and correspond to the compounds: (a) 2-methoxymethyl-2,4,5-trimethyl-1,3-dioxolane; (b) 3-heptanone; (c) 1-octen-3-one; and (d) N-pentyl-2-butylamine, respectively, where the one or more compounds can be in an amount of 0 ppm or more (e.g., 0.00001 or more), 10 ppm or less, or 0 ppm-10 ppm. In some embodiments, such one or more compounds can be in an amount or concentration below its odor threshold. Further embodiments provide for such one or more compounds that are contributors to “off-odor”.

Some embodiments of the disclosure are directed to a BG composition or formulation comprising a vehicle (e.g., a carrier, a diluent, an excipient, or any combinations thereof) and a BG product having a neutral odor, where the BG product of the disclosure does not comprise, does not consist essentially of, does not consist of, or minimally comprises one or more compounds characterized by an aromatic region, an odor-active region, or a peak as measured by gas chromatography/mass spectrometry/olfactory (GC/MS/O), where the aromatic region, the odor-active region, or the peak has a relative acquisition time selected from a group consisting of: about 0.417 mins (rubber and/or plastic and/or green), about 0.450 mins (plastic), about 0.483 mins (solventy), about 0.783 mins (plastic), about 1.033 mins (fruity and/or plastic), about 1.1 mins (plastic and/or pungent and/or mushroom and/or metallic), about 1.367 mins (fruity and/or plastic and/or metal and/or mushroom), about 1.967 mins (waxy and/or fatty), and combinations thereof, wherein the BG product comprises bioBG, wherein the BG has a relative acquisition time of 1 min, and the one or more compounds is in an amount of 0 ppm or more (e.g., 0.00001 or more), 10 ppm or less, or 0 ppm-10 ppm, and where the composition comprises a vehicle (e.g., a carrier, a diluent, an excipient, or any combinations thereof. In some embodiments, such one or more compounds can be in an amount or concentration below its odor threshold. Further embodiments provide for such one or more compounds that are contributors to “off-odor”.

Some embodiments provide a BG composition or formulation comprising a vehicle (e.g., a carrier, a diluent, an excipient, or any combinations thereof) and a neutral odor BG product (e.g., bioBG) of the disclosure, where the BG product does not comprise, does not consist essentially of, does not consist of, or minimally comprises one or more of the compounds disclosed here, where the compound is in an amount of 0 ppm or more (e.g., 0.00001 or more), 10 ppm or less, or 0 ppm-10 ppm, where the one or more of the compounds can be characterized by an aromatic region or a peak as measured by gas chromatography/quadrupole time-of-flight-mass spectrometry (GC/QTOF-MS), where the aromatic region or the peak elutes at an acquisition time selected from a group consisting of: about 12 mins (e.g., 11.9 mins-12.2 mins) at 59.0489 m/z, about 13 mins (e.g., 12.955 mins-13.15 mins) at 57.0698 m/z, about 15.5 mins (e.g., 15.55 mins-15.65) at 70.0411 m/z, about 28.5 mins (e.g., 28.5 mins-28.6 mins) at 114.0675 m/z, and combinations thereof, where, in some embodiments, the one or more compounds can be present in an amount or concentration below its odor threshold. Further embodiments provide for such one or more compounds that are contributors to “off-odor”.

In some embodiments, a BG composition or formulation comprises a BG product of the disclosure, where the BG product comprises, consists essentially of, or consists of one or more of the compounds disclosed here, where the one or more compounds is in an amount of 0 ppm or more (e.g., 0.00001 or more), 10 ppm or less, or 0 ppm-10 ppm, where the compound comprises a mass spectrum as characterized by quadrupole time-of-flight-mass spectrometry electron ionization (QTOF EI) at 70 eV, where the mass spectrum comprises peaks, wherein the mass spectrum is selected from the group consisting of: (a) 43.017 m/z, 55.0541 m/z, 73.0283 m/z, 86.0723 m/z, 97.0642 m/z, and 115.0752; (b) 41.0385 m/z, 57.0698 m/z, 68.0259 m/z, 72.0569 m/z, 85.0646 m/z, 99.0801 m/z, and 114.1033 m/z; and (c) 43.0179 m/z, 57.0336 m/z, 72.0206 m/z, 86.0362 m/z, 114.0675 m/z, 128.0830 m/z, 138.0670 m/z, and 155.1060 m/z, and a vehicle (e.g., a carrier, a diluent, an excipient, or any combinations thereof), where, in some embodiments, the one or more compounds are present in an amount or concentration below its odor threshold. Further embodiments provide for such one or more compounds that are contributors to “off-odor”.

Some embodiments are directed to a BG composition or formulation comprising a BG product (e.g., bioBG) of the disclosure, where the BG product does not comprise, does not consist essentially of, does not consist of, or minimally comprises one or more of the compounds disclosed here, where the compound is in an amount of 0 ppm or more (e.g., 0.00001 or more), 10 ppm or less, or 0 ppm-10 ppm, where the compound is selected from the group consisting of: butyl acetate; butyl acrylate; 4-hydroxy-2-butanone; 2-methoxymethyl-2,4,5-trimethyl-1,3-dioxolane (solventy); 3-heptanone; 1-octen-3-one; N-pentyl-2-butylamine (sulfury and/or urine-like); 3-buten-2-one; 2-butenal; 2,4-dimethyl-1,3-dioxane; 2-ethyl-5-methyl-1,3-dioxane; 2-heptyl-1,3-dioxepane; 1-hydroxy-2-propanone; 1,2 propanediol (12PDO); 1,2,3-butanetriol; 1,2,3-propanetriol; 1,3 propanediol (13PDO); 1,4-butanediol (BDO); 2,3-butanediol (23BDO); 2,5-hexanedione; 3-buten-2-ol; cis-cryotyl alcohol; isopropyl alcohol (IPA); methyl vinyl ketone (MVK); n-butane (n-but); dimethylethanolamine (DMAE); toluene; HDD; and any combinations thereof, where the compound is in an amount of 0 ppm or more (e.g., 0.00001 or more), 10 ppm or less, or 0 ppm-10 ppm, where, in some embodiments, the one or more compounds are present in an amount or concentration below its odor threshold. Further embodiments provide for such one or more compounds that are contributors to “off-odor”.

In some embodiments, a composition or formulation comprising a BG product of the disclosure and does not further comprise, does not consist essentially of, does not consist of, or minimally comprises any one or more of the compounds selected from the group consisting of: 3-buten-2-one; 3-heptanone; 1-octen-3-one; styrene; butyl acetate; butyl acrylate; 4-hydroxy-2-butanone; 2-methoxymethyl-2,4,5-trimethyl-1,3-dioxolane (solventy); N-pentyl-2-butylamine (sulfury and/or urine-like); 3-buten-2-one; 2-butenal; 2,4-dimethyl-1,3-dioxane; 2-ethyl-5-methyl-1,3-dioxane; 2-heptyl-1,3-dioxepane; 1-hydroxy-2-propanone; 1,2 propanediol (12PDO); 1,2,3-butanetriol; 1,2,3-propanetriol; 1,3 propanediol (13PDO); 1,4-butanediol (BDO); 2,3-butanediol (23BDO); 2,5-hexanedione; 3-buten-2-ol; cis-cryotyl alcohol; isopropyl alcohol (IPA); methyl vinyl ketone (MVK); n-butane (n-but); dimethylethanolamine (DMAE); toluene, HDD; or any combinations thereof, where the compound is in an amount of 0 ppm or more (e.g., 0.00001 or more), 10 ppm or less, or 0 ppm-10 ppm; and the composition comprises a vehicle (e.g., a carrier, a diluent, an excipient, or any combinations thereof). In some embodiments, such one or more compounds can be in an amount or concentration below its odor threshold. Further embodiments provide for such one or more compounds that are contributors to “off-odor”.

Further embodiments can be directed to BG compositions comprising a vehicle, a bioBG, and further comprising, minimally comprising, consisting essentially of, or consisting of one or more compounds selected from the group consisting of: 5-norbornene-2-ol; 2-ethyl-1-hexanol; 3-buten-2-one; 3-heptanone; 1-octen-3-one; styrene; butyl acrylate; HDD; and salts, derivatives, analogs, or solvates, thereof, or any combinations thereof, in an amount below a known odor threshold for each of the one or more compounds as measured by Dynamic Headspace (DHS) and tandem GC/MS using Agilent Triple Quadropole Mass Spectrometer (DHS-GC/MS-QQQ). Other embodiments provide for BG compositions comprising a vehicle, a bioBG, and further comprising, minimally comprising, consisting essentially of, or consisting of one or more compounds selected from the group consisting of: 5-norbornene-2-ol; 2-ethyl-1-hexanol; 3-buten-2-one; 3-heptanone; 1-octen-3-one; styrene; butyl acrylate; HDD; and salts, derivatives, analogs, or solvates, thereof, or any combinations thereof, where the one or more compounds comprises a fold change over its respective odor threshold, where the fold change is 0.1× or greater (e.g., 0.2×, 0.3×, 0.4×, 0.5×, 0.6×, 0.7×, 0.8×, 0.9×, 1×, 2×, 3×, 4×, 5×, 6×, 7×, 8×, 9×, 10×, 15×, 20×, 25×, 30×); 50× or less (e.g., 45×, 40×, 35×, 30×, 25×, 20×, 15×, 10×, 9.5×, 8.5×, 7.5×, 6.5×, 5.5×, 4.5×, 3.5×, 2.5×, 1.5×, 0.95×, 0.85×, 0.75×, 0.65×, 0.55×, 0.45×, 0.35×, 0.25×, 0.15×); or 0×-50× (e.g., 0.1×-49.5×; 0.2×-48.5×; 0.3×-47.5×; 0.4×-46.5×; 0.5×-45.5×; 0.6×-44.5×; 0.7×-43.5×; 0.8×-42.5×; 0.9×-41.5×; 1×-40.5×; 1.2×-39.5×; 1.4×-38.5×; 1.6×-37.5×; 1.8×-36.5×; 2×-35.5×; 2.2×-34.5×; 2.4×-33.5×; 2.6×-32.5×; 2.8×-31.5×; 3×-30.5×; 3.2×-29.5×; 3.4×-28.5×; 3.6×-27.5×; 3.8×-26.5×; 4×-25.5×; 4.2×-24.5×; 4.4×-23.5×; 4.6×-22.5×; 4.8×-21.5×; 5×-20.5×; 5.2×-19.5×; 5.4×-18.5×; 5.6×-17.5×; 5.8×-16.5×; 6×-15.5×; 6.2×-14.5×; 6.4×-13.5×; 6.6×-12.5×; 6.8×-11.5×; 7×-10.5×; 7.2×-9.5×; 7.4×-8.5×). In some embodiments, such one or more compounds can be in an amount or concentration below its odor threshold.

Some embodiments of the disclosure provide BG compositions or formulations comprising a BG product (e.g., bioBG) described here, where the BG product comprises a property selected from: a humectant, a conditioning agent, a preservative, a viscosity-controlling agent, a fragrance enhancer, a fungicide, a bactericide, a solvent, and any combinations thereof. Certain commonly used compositions comprising a petroleum-derived BG, or an unpurified BG, or an agent that performs similar functions or has similar properties as the BG product described here (e.g., a humectant, a conditioning agent, a preservative, a viscosity-controlling agent, a fragrance enhancer, a fungicide, a bactericide, a solvent) can comprise an undesirable odor. In order to mask such undesirable odors in those compositions, more fragrance ingredients can be required to hide or reduce the undesirable odor. Fragrances or fragrance ingredients are expensive. Cosmetics, cosmeceuticals, and personal care products typically have some fragrance, which may be used to mask malodorous ingredients. Since the BG product (e.g., bioBG) of the disclosure has a neutral or minimally-detectable odor, compositions comprising the BG product described here can have a percentage of total fragrance ingredients that is less than what is typically used in compositions without the neutral odor BG product or with other products which contain an undesirable odor. Since, the bioBG product or composition comprising the bioBG product of the disclosure is substantially or essentially non-odorous, and does not mask or suppress an odor, add an odor, or alter a fragrance profile of components of a composition of the disclosure, the amount of any fragrance ingredients can be reduced, yet still provide a strong level of a desirable or pleasant odor or scent without affecting the fragrance profile. An advantage of using fewer fragrance ingredients or less amounts of fragrance ingredients is the cost savings to the composition manufacturer, and potentially to the consumer, since the process of obtaining the fragrance ingredients and/or the fragrance ingredients themselves is costly.

A person of ordinary skill in the art, for example, a formulator, would understand how to incorporate the BG product described here into a cosmetic, cosmeceutical, or personal care product without affecting its desired function. Cosmetics as used here are intended to be rubbed, poured, sprinkled, or sprayed on, introduced into, or otherwise applied to a mammal body or any part thereof for cleansing, beautifying, promoting attractiveness, or altering the appearance.

In some embodiments, a composition of the disclosure comprising a bioBG product described here can be manufactured or sold with the approval of a governmental regulatory agency as part of a safety review for use in or on a mammal. Compositions can be formulated, for example, for topical administration (e.g., an extract or parfum, eau de parfum, eau de toilette, eau de cologne, eau de fraiche, aftershave, cream, gel, lotion, ointment, oil (e.g., essential oil, massage oil, aromatherapy), shampoo, hair conditioner, shower gel, soap, cleanser, deodorant, anti-perspirant). The neutral odor bioBG product of the disclosure can be a multifunctional ingredient, which can be used in a variety of personal care products and cosmetics, including serums, body scrubs, masks, cleansers, and the like. BG compositions comprising the neutral odor BG product disclosed here include, but are not limited to, eye liner, eye shadow, eye makeup, mascara, brow liner, primer, foundation, concealer, BB cream, CC cream, brighteners, blush, bronzer/highlighter, setting powder/spray, body/facial sprays, facial serums, lipstick, lip gloss, lip balm, lip liner, perfumes/fragrances, creams, lotions, moisturizers, face masks, sunscreens, sunblocks, fingernail polishes, deodorants, anti-perspirants, shampoos, conditioners, hair dyes, hair dye removers, soap, cleansers, body washes, products comprising an ingredient selected from: vitamins, botanical or marine extracts, peptides, antioxidants, and alpha-hydroxy acids, or aromatherapy compositions (e.g., aromatherapy oils, bath aromatherapy compositions, aromatherapy chest rubs), massage oils, compresses, room sprays, clothing/linen sprays, office stationery sprays, or any compositions that comprise a BG product of the disclosure for use of its properties (e.g., a humectant, a conditioning agent, a preservative, a viscosity-controlling agent, a fragrance enhancer, a fungicide, a bactericide, a solvent).

### Methods and Uses of Using Neutral Odor BG Compositions

A consumer of cosmetics, cosmeceuticals, and personal care products would know and understand how to use of any of the neutral odor BG compositions disclosed here. Some embodiments of the disclosure provide a method of applying to a subject in need thereof, any of the BG compositions described here, where the BG compositions comprising BG products with bioBG include cosmetics, cosmeceutical, and personal care products. The subject in need thereof includes those who are in need of improving appearance, hygiene (e.g., cleanliness, smell), skincare, and the like, or combinations thereof. As used herein, the term “subject” or “consumer” refers to any organism to which a composition in accordance with the disclosure may be applied or administered, e.g., for experimental, diagnostic, organoleptic, or cosmetic purposes. Typical subjects include any animal (e.g., mammals such as humans, mice, rats, rabbits, and non-human primates, etc.). A subject or consumer in need thereof is typically a subject who uses cosmetics, personal care products, cosmeceuticals. For example, a subject in need thereof can use an eye liner, an eye shadow, an eye makeup, a mascara, a brow liner, a primer, a foundation, a concealer, a BB cream, a CC cream, a brightener, a blush, a bronzer/highlighter, a setting powder/spray, a lipstick, a lip gloss, a lip balm, a lip liner, a perfume/fragrance, a cream, a lotion, a moisturizer, a face mask, a sunscreen, a sunblock, a fingernail polish, a shampoo, a conditioner, a hair dye, a hair dye remover, a deodorant, an anti-perspirant, a soap, a cleanser, and a cosmetic with vitamins, botanical or marine extracts, peptides, antioxidants, and alpha-hydroxy acids.

Some embodiments provide methods of using a neutral odor BG composition comprises applying a neutral odor BG composition comprising a bioBG product to a subject (e.g., face, body, eyelashes, eyebrows, lips, fingernails, hair), where non-limiting examples of a BG composition includes: eye liner, eye shadow, eye makeup, mascara, brow liner, primer, foundation, concealer, BB cream, CC cream, brighteners, blush, bronzer/highlighter, setting powder/spray, lipstick, lip gloss, lip balm, lip liner, perfumes/fragrances, creams, lotions, moisturizers, face masks, sunscreens, fingernail polishes, shampoos, conditioners, hair dyes, hair dye removers, deodorants, anti-perspirants, soaps, cleansers, and products (e.g., cosmetics; lotions, moisturizers, face masks, shampoos, conditions, hair dyes, hair dye removers) with vitamins, botanical, marine extracts, peptides, antioxidants, and alpha-hydroxy acids.

In some embodiments, a use of the 1,3-butylene glycol (BG) product or BG composition comprising a BG product described here for providing a property selected from the group consisting of: humectant, fungicide, bactericide, conditioning agent, preservative, viscosity-decreasing agent, solvent, and combinations thereof, to a subject in need thereof is provided.

Some embodiments provide uses of the BG product or BG composition comprising a BG product described here for improving appearance, hygiene (e.g., cleanliness, smell), skincare, and the like, or combinations thereof, of a subject in need thereof.

### Additional Components

Some embodiments of the disclosure are directed to compositions or formulations comprising a neutral odor BG product described here, where the BG product comprises bioBG, and a physiologically acceptable vehicle (e.g., a carrier, a diluent, an excipient, or combinations thereof). Non-limiting examples of physiologically acceptable vehicles suitable for use in the compositions described here include: water, phosphate buffered saline (e.g., pH 7.4), aqueous sodium chloride solution (e.g., 0.15M), dextrose, glycerol, ethanol, and the like, and mixtures thereof. Additional components can include, but are not limited to, polyethylene glycol, proteins, saccharides, amino acids, inorganic acids, organic acids, stabilizers, lubricants, binding agents, dyes, wetting agents, and the like which may be used cither on their own or in combination. Those of ordinary skill in the art would know and understand the amounts or quantities, as well as the routes of administration, used, which can correspond to the amounts used in similar types of applications or indications.

Some embodiments of the disclosure provide compositions comprising multifunctional bioBG, which may be used as a physiologically acceptable vehicle (e.g., a carrier, a solvent, a diluent (used interchangeably here)) for solubilizing or diluting additional ingredients, while also functioning as a humectant, a conditioning agent, a preservative, a viscosity-controlling agent, a fragrance enhancer, a fungicide, a bactericide, a solvent, or any combinations thereof.

In some embodiments of the disclosure, compositions comprising bioBG or a bioBG product and one or more compounds, can further include additional ingredients depending on the application or use of the composition. These additional ingredients can include, but are not limited to, any and all diluents or solvents, dispersion media, antibacterial and antifungal agents, isotonic and absorption delaying or prolonging agents, and the like that are commonly known and used in such compositions and formulations. The physiologically acceptable vehicle (e.g., carrier, solvent, diluent) does not inhibit or mask the activity or purpose of the composition (e.g., cleansing, improving appearance, hygiene) and is non-toxic when applied in amounts sufficient to deliver the desired effect(s).

As used herein, the phrase “physiologically acceptable” means generally safe for contact with biologic tissues, particularly at the levels employed. Physiologically acceptable is used interchangeably with physiologically compatible. The compositions of the disclosure comprise physiologically acceptable or compatible components, including but not limited to, active agents, fragrance ingredients, diluents, solvents, detergents, dyes, solubilizers, humectants, and the like.

As used herein, all ranges of numeric values include the endpoints and all possible values disclosed between the disclosed values. The exact values of all half-integral numeric values are also contemplated as specifically disclosed and as limits for all subsets of the disclosed range. For example, a range of from 0.1% to 3% specifically discloses a percentage inclusive of the lowermost and uppermost range, and integrals therebetween, such as for example, 0.1%, 1%, 1.5%, 2.0%, 2.5%, and 3%. Additionally, a range of 0.1 to 3% includes, for example, subsets of the original range including from 0.5% to 2.5%, from 1% to 3%, from 0.1% to 2.5%, etc. It will be understood that the sum of all weight percents (weight %) of individual components will not exceed 100%.

Throughout this description, various components may be identified having specific values or parameters, however, these items are provided as exemplary embodiments. Indeed, the exemplary embodiments do not limit the various aspects and concepts of the present disclosure as many comparable parameters, sizes, ranges, and/or values may be implemented.

### EXAMPLES

The following examples illustrate specific aspects of the instant description. The examples should not be construed as limiting, as the example merely provides specific understanding and practice of the embodiments and its various aspects.

### Example 1: Deodorization of Bioderived 1,3-Butylene Glycol

### BG Deodorization Method to Produce Neutral Odor Sample DSP-555-2

Bioderived 1,3-butylene glycol (bioBG) was produced and deodorized using a caustic treatment followed by distillation described here to produce a bioBG product 1 of the disclosure (DSP-555-2). The block flow diagram which describes the process implemented to produce 800 kgs of deodorized bioBG. See, FIG. 1.

Odorous bioBG was treated with a caustic solution (˜40 wt %-˜50 wt % NaOH), such that the final concentration of NaOH in the reaction solution was 0.25 wt %. This mixture was then reacted in a tank vessel (base reactor) at 120° C. for 20 mins while stirring and as a result, impurities, such as ketones and aldehydes, convert to other less volatile materials. Alternatively, the continuous flow reactor with heat exchanger could be used to carry out the reaction.

The reaction product was then fed into a WFE (wiped film evaporator) for degassing and water removal. This helps in reaching low vacuum in subsequent steps. The bottom stream from the WFE was then fed into a SPE (short pass evaporator) for base removal along with some heavies. This step was typically run at a relatively milder temperature (˜90° C.) and a short residence time (in seconds) to avoid formation of degradation products.

The lights vapor stream from the SPE was then condensed and fed into the 1st distillation column, equipped with a wiped film evaporator (WFE) as a reboiler, where residual heavies are removed as bottoms under vacuum (10-20 torr) at 100° C.-110° C. The tops from this step was sent to a 2^{nd} distillation column of the same size where the lights components were distilled off and a low odor BG (DSP-555-2) was drawn from the bottom. The pressure in this column was low, 10 torr-20 torr at 120° C.; however, had a higher reflux ratio than the heavies removal step for better separation.

### BG Deodorization Method to Produce Neutral Odor Sample DSP-555-4

Crude high odor bioderived 1,3-butylene glycol (bioBG) was produced and deodorized using the caustic distillation process described here to produce a bioBG Product 2 (DSP-555-4).

### Crude Analysis

The crude, BIO-BG Natural Butylene Glycol (D2413) was received in 4×500 mL glass bottles. The odor of the crude was noticeable. This high odor bioBG was deodorized as described here:

| TEST | RESULT |
|---|---|
| Water | 0.04% |
| 1,3-butanediol | 99.6781%* |
| GC Lights | 0.1456%* |
| GC Heavies | 0.1336%* |
| Acidity (as acetic acid) | 0.0027% |

*GC results normalized for water and acidity

### Distillation Details

1,3-Butanediol (1,731.8 g) (bioBG) was charged to a 2 L 3 necked flask and placed under an 24 theoretical plate column fitted with a poly(tetrafluorethylene) (PTFE) (0.25 mm ID) nitrogen sparge. The temperature was monitored using a PTFE thermocouple in the base and a stainless steel thermocouple in the side stream and overheads which were connected to a RS52 2channel K handheld thermometer. After 18 theoretical plates, a side stream was fitted and was connected to a Perkin triangle and 250 ml round bottom flask. A reflux controller and Perkin triangle fitted with a 250 ml round bottom flask were used as overheads. A vacuum of 20 mmHg was applied to the system using a Javac DS75 high vacuum pump. The system was brought to a boil and held at a total reflux for ˜15 minutes. After this, because the temperatures at the side stream and at the column top were similar, the top reflux ratio was set to 1:1 to remove any light boilers for ˜45 minutes. The side stream was then set to take off 1,3-butanediol at 1:1 and the reflux ratio adjusted to ˜10:1. The purity and the odor of the side stream was unacceptable, so the side stream take off was stopped while more lights were removed from the column top at a reflux ratio of ˜15:1 or ˜10:1. On start up the following day, the side stream was set to take off 1,3-butanediol at 1:1 and the top reflux ratio set to take off at 5:1. The reflux ratio at the column top was varied slightly through the distillation to determine if the odor and purity of the side stream improved with an increased reflux ratio. The rest of the distillation was run with a top reflux ratio of ˜5:1 and a side stream take off of 1:1. Towards the end of the distillation, the GC heavies in the side stream began to increase and knocked the purity of the side stream below the specification of a 99.5% minimum.

### Distillation Run Sheet

The middle temperature (MT) read low during the run sheet until Apr. 7, 2021 (07/04/2021), which was due to a lack of lagging on the side stream portion of the column.

| | | | | | | | | |
|---|---|---|---|---|---|---|---|---|
| | BT | MT | TT | Middle | Top | Pressure | | Observations/ |
| Time | (° C.) | (° C.) | (° C.) | RR | RR | (mmHg) | Fraction | Comments |

30 Mar. 2021

| | | | | | | | | |
|---|---|---|---|---|---|---|---|---|
| 08:30 | 16.9 | 17.9 | 18.3 | Total | Total | 21 | | Heat on. |
| 09:33 | 93.1 | 18.3 | 18.4 | Total | Total | 20 | | |
| 10:24 | 121.5 | 85.4 | 19.4 | Total | Total | 20 | | |
| 10:54 | 122.8 | 108.7 | 86.0 | Total | Total | 20 | | |
| 11:03 | 123.0 | 111.3 | 118.9 | Total | Total | 20 | | |
| 11:14 | 122.6 | 110.9 | 119.7 | Total | Total | 20 | | TR → 2:1 |
| 12:00 | 121.8 | 114.2 | 119.3 | Total | 1:1 | 20 | T1 (22.9 g) | |
| 12:41 | 122.1 | 113.9 | 119.2 | Total | 2:1 | 20 | T2 (24.3 g) | SS flush |
| 12:49 | 122.4 | 113.9 | 119.0 | Total | 2:1 | 20 | SS1 (9.8 g) | MR → 2:1 |
| 13:34 | 121.7 | 113.2 | 118.5 | 2:1 | 2:1 | 20 | SS2 (16.5 g) | TR → 10:1 |
| | | | | | | | T3 (28.0 g) | |
| 14:23 | 121.2 | 112.4 | 117.1 | 2:1 | 13:1 | 19 | SS3 (13.0 g) | MR → Total |
| | | | | | | | | Take more |
| | | | | | | | | tops |
| 14:35 | 120.7 | 112.8 | 117.4 | Total | 10:1 | 19 | T4 (13.1 g) | |
| 15:17 | 120.4 | 112.1 | 116.8 | Total | 6:1 | 19 | | TR → 3:1 |
| 15:56 | 120.4 | 113.1 | 117.2 | Total | 3:1 | 19 | T5 (26.7 g) | Heat off. |

31 Mar. 2021

| | | | | | | | | |
|---|---|---|---|---|---|---|---|---|
| 07:54 | 25.3 | 36.3 | 30.6 | Total | Total | | | Heat & |
| | | | | | | | | vacuum on. |
| 08:49 | 121.6 | 113.6 | 20.6 | Total | Total | 20 | | |
| 09:02 | 122.2 | 116.7 | 119.4 | Total | Total | 20 | | Refluxing. |
| 09:26 | 122.4 | 117.4 | 119.6 | Total | Total | 20 | | TR → 5:1 |
| 10:26 | 123.6 | 119.2 | 120.9 | Total | 2:1 | 20 | SS4 (5.1 g) | SS flush (SS4) |
| | | | | | | | | MR → 2:1 |
| 11:15 | 123.1 | 117.8 | 119.7 | 1:1 | 5:1 | 20 | SS5 (16.9 g) | |
| 12:03 | 128.3 | 123.6 | 119.6 | 1:1 | 5:1 | 20 | SS6 (13.4 g) | |
| | | | | | | | T6 (58.3 g) | |
| 13:02 | 126.2 | 120.9 | 119.6 | 1:1 | 3:1 | 20 | SS7 (25.2 g) | |
| 13:32 | 124.3 | 119.2 | 119.5 | 1:1 | 2:1 | 20 | T7 (40.1 g) | |
| 13:47 | 123.9 | 118.8 | 119.7 | 1:1 | 3:1 | 20 | SS8 (12.4 g) | MR → Total |
| | | | | | | | | TR → 2:1 |
| 14:10 | 127.4 | 121.8 | 120.0 | Total | 2:1 | 20 | T8 (22.2 g) | |
| 14:46 | 126.2 | 121.3 | 120.1 | Total | 2:1 | 20 | T9 (23.2 g) | |
| 15:36 | 127.6 | 122.3 | 120.0 | Total | 2:1 | 20 | T10 (33.8 g) | |
| 16:02 | 126.2 | 121.1 | 120.2 | Total | 2:1 | 20 | | MR → 1:1 |
| 16:35 | 127.7 | 121.9 | 120.0 | 1:1 | 2:1 | 20 | SS9 (24.9 g) | Odour better, |
| | | | | | | | | purity 99.6% |
| | | | | | | | | TR → 10:1 |
| 17:08 | 127.8 | 122.2 | 119.4 | 1:1 | 10:1 | 22 | SS10 (34.4 g) | |
| 17:31 | 124.2 | 118.2 | 119.6 | 1:1 | 10:1 | 22 | SS11 (30.5 g) | Heat off. |
| | | | | | | | T11 (50.8 g) | |

1 Apr. 2021

BS1 (46.2 g)
Taken for
GC, odor
analysis and
potential
chemical
treatment
for odor
removal.

6 Apr. 2021

| | | | | | | | | |
|---|---|---|---|---|---|---|---|---|
| 09:07 | 13.9 | 16.1 | 16.4 | Total | Total | | | Heat & vacuum |
| | | | | | | | | on. |
| 10:09 | 124.1 | 115.9 | 18.9 | Total | Total | 20 | | |
| 10:19 | 124.4 | 117.2 | 121.7 | Total | Total | 20 | | Refluxing. |
| 10:37 | 127.1 | 119.9 | 121.6 | Total | Total | 20 | | TR → 10:1 |
| 11:06 | 131.6 | 123.6 | 121.7 | Total | 10:1 | 20 | | MR → 1:1 |
| 11:46 | 134.1 | 127.2 | 121.7 | 1:1 | 8:1 | 20 | SS12 (27.4 g) | |
| 12:17 | 125.6 | 118.7 | 121.5 | 1:1 | 5:1 | 20 | SS13 (36.7 g) | |
| 13:39 | 125.0 | 117.8 | 121.4 | 1:1 | 5:1 | 20 | SS14 (87.2 g) | |
| 14:39 | 125.5 | 118.5 | 121.5 | 1:1 | 5:1 | 20 | SS15 (106.6 g) | |
| | | | | | | | T12 (50.0 g) | |
| 16:02 | 124.7 | 117.6 | 121.2 | 1:1 | 5:1 | 20 | SS16 (149.1 g) | Heat off. |

7 Apr. 2021

| | | | | | | | | |
|---|---|---|---|---|---|---|---|---|
| 09:28 | 14.4 | 17.2 | 17.7 | Total | Total | 20 | | Heat on. |
| 10:46 | 122.5 | 115.2 | 119.7 | Total | Total | 20 | | Refluxing. |
| 10:59 | 122.8 | 115.9 | 120.3 | Total | Total | 20 | | TR → 10:1 |
| | | | | | | | | MR → 1:1 |
| 11:25 | 122.8 | 115.9 | 120.3 | 1:1 | 10:1 | 20 | SS17 (34.3 g) | Lagging added |
| | | | | | | | | to SS. |
| 12:18 | 127.2 | 123.1 | 119.7 | 1:1 | 10:1 | 20 | SS18 (100.4 g) | |
| 13:03 | 127.2 | 122.3 | 119.7 | 1:1 | 5:1 | 20 | SS19 (114.2 g) | MR → Total |
| | | | | | | | | SS19 purity |
| | | | | | | | | 99.2% |
| 13:46 | 126.8 | 122.3 | 119.7 | Total | 5:1 | 20 | T13 (42.4 g) | MR → 1:1 |
| 15:17 | 124.9 | 119.6 | 119.6 | 1:1 | 5:1 | 20 | SS20 (204.1 g) | Heat off. |

BT—bottom temperature;
MT—middle temperature;
TT—top temperature;
RR—reflux ratio;
SS—side stream;
T—tops

### Fraction Analysis as GC Area (%)

1,3-Butanediol is quite viscous and significant variation was seen in the repeat injection of the crude sample. For this reason, all GC analysis was performed as a 50:50 mixture with water.

Analysis of the crude and composite was performed neat. The syringe was thoroughly washed with water between each run.

| | Mass | % GC | % 1,3- | % GC | |
|---|---|---|---|---|---|
| Fraction | (g) | Lights | Butanediol | Heavies | Odour |
| Crude * | 1731.8 | 0.0521 | 98.5535 | 1.3517 | |
| SS1 | 9.8 | 0.1769 | 99.4564 | 0.3667 | smelly |
| SS2 | 16.5 | 0.0129 | 99.1592 | 0.8279 | smelly |
| SS3 | 13.0 | 0.0106 | 99.3232 | 0.6662 | smelly |
| SS4 | 5.1 | 0.0163 | 99.6661 | 0.3176 | smelly |
| SS5 | 16.9 | 0.0061 | 99.6748 | 0.3191 | smelly |
| SS6 | 13.4 | 0.0046 | 99.1464 | 0.8490 | smelly |
| SS7 | 25.2 | 0.0131 | 98.3314 | 1.6555 | smelly |
| SS8 | 12.4 | 0.0088 | 99.1968 | 0.7944 | milder |
| SS9 | 24.9 | 0.0065 | 99.6453 | 0.3482 | milder |
| SS10 | 34.4 | 0.0048 | 99.5572 | 0.4380 | mild |
| SS11 | 30.5 | 0.0044 | 99.6370 | 0.3586 | mild |
| SS12 | 27.4 | 0.0097 | 99.4827 | 0.5076 | mild |
| SS13 | 36.7 | 0.0026 | 99.5393 | 0.4581 | very low |
| SS14 | 87.2 | 0.0021 | 99.7069 | 0.2910 | mild |
| SS15 | 106.6 | 0.0028 | 99.5918 | 0.4054 | mild |
| SS16 | 149.1 | 0.0013 | 99.6303 | 0.3684 | mild |
| SS17 | 34.3 | 0.0043 | 99.7037 | 0.2920 | mild |
| SS18 | 100.4 | 0.0025 | 99.2659 | 0.7316 | slightly fruity? |
| SS19 | 114.2 | 0.0027 | 99.3554 | 0.6419 | mild |
| SS20 | 204.1 | 0.0011 | 99.4540 | 0.5449 | mild |
| T1 | 22.9 | 0.9946 | 96.3821 | 2.6233 | |
| T2 | 24.3 | 0.3910 | 97.6079 | 2.0011 | |
| T3 | 28.0 | 0.1349 | 97.0450 | 2.8201 | |
| T4 | 13.1 | 0.1222 | 98.9421 | 0.9357 | |
| T5 | 26.7 | 0.0348 | 98.8430 | 1.1222 | |
| T6 | 58.3 | 0.0167 | 99.2596 | 0.7237 | |
| T7 | 40.1 | 0.0050 | 98.9969 | 0.9981 | |
| T8 | 22.2 | 0.0035 | 99.4025 | 0.5940 | |
| T9 | 23.2 | 0.0044 | 99.2474 | 0.7482 | |
| T10 | 33.8 | 0.0045 | 99.2555 | 0.7400 | |
| T11 | 50.8 | 0.0004 | 99.7298 | 0.2698 | |
| T12 | 50.0 | 0.0080 | 99.6148 | 0.3772 | |
| T13 | 42.4 | 0.0059 | 99.7772 | 0.2169 | |
| T14 | 20.8 | 0.0007 | 99.8123 | 0.1870 | |
| BS1 | 46.2 | | | | |
| Residues | 112.2 | n/d | 99.7989 | 0.2011 | |

* GC results normalized for water and acidity.

### Residues

The residues (112.2 g) consisted of a pale yellow colored liquid containing no solids. The residues had no significant odor.

The walls of the flask were clean once the residues were decanted, a cold water wash was used to clean the flask successfully.

### Composite Sample

A composite sample (SS 9-20, 949.8 g) was prepared and analyzed as follows:

| Test | Result | Specification | |
|---|---|---|---|
| Water | 0.02% | | |
| 1,3-Butanediol | 99.9238% * | 99.5% | min. |
| GC Lights | 0.0150% * | | |
| GC Heavies | 0.0392% * | | |
| Color | 0 Hazen | 10 Hazen max. | |
| Appearance | Clear Colorless Liquid | Clear Colorless Liquid | |
| SG (at 20° C.) | 1.005 | 1.004 to 1.006 | |
| Odor | Odorless/Low Odor | Odorless | |
| Free acid (as acetic acid) | 0.002% | 0.005% | max. |

* GC results normalized for water and acidity.

### Mass Balance

| | Weight (g) | Percentage (%) |
|---|---|---|
| Total Mass In | 1731.8 | 100.00 |
| Spec 13BD (SS) | 949.8 | 54.84 |
| Non spec 13BD (SS) | 112.3 | 6.48 |
| Lights/Tops | 456.6 | 26.37 |
| Residues | 112.2 | 6.48 |
| Base Samples | 46.2 | 2.67 |
| Losses | 54.7 | 3.16 |

### CONCLUSION

The odor of the lights fractions all had a significant odour. The odor of the early side stream fractions also had a significant odor which eventually reduced to an acceptable level. A lot of the charge was taken as tops and off-spec side stream, this resulted in a poor yield of ˜55%. Throughout the distillation, the reflux ratio at the column top had to be altered to keep the side stream purity on spec.

The yield of the distillation can be improved upon if repeated by increasing the top reflux ratio to 15:1, on the plant there is also the opportunity to recycle inters type material which would increase the yield. To improve the yield in the lab, the side stream take off should not start until the purity in the tops fractions was >99%, which can occur by slowly removing light boilers at a higher reflux ratio of ˜15:1.

### Corrosion Studies

A 321 corrosion coupon was placed in the flask during the distillation of 1,3-butanediol. The corrosion rate was deemed acceptable. The coupon showed no weight loss.

### Other Work

BS1 was taken, split, and treated with Venpure (0.7%), NaOH (10%) and NaBH₄ (0.5%) to determine whether any improvement in odor was possible. The samples were mixed and placed in the oven for a short period of time before their odor was checked using the hot water test (20 mL hot water+1 mL sample). The general opinion was that there was no odor improvement observed.

It was difficult to conclude whether any of these treatment options would provide a better odor without running through the entire distillation process.

### Analytical Details

The following equipment and conditions were used to analyze the distillation fractions:

| Column Type/Dimensions | HP-1; 30 m × 0.32 mm (film thickness | |
|---|---|---|
| | 1.0 μl) | |
| Carrier Gas/Pressure | Hydrogen/5.0 psig | |
| Injector/Detector Temps | 250° C./270° C. | |
| Sample size | 0.1 | μl |
| Temp. Programme | 80° C. to 260° C. at 20° C./min | |
| | (iso 11 mins) | |
| Total Run Time | 20 | Minutes |

| | |
|---|---|
| Split Ratio | 20:1 |
| GC | GC-3 |
| Method File | 1,2-Pentanediol |
| Method Name | CC5_HP1 |

The following equipment and conditions were used to analyze the spec composite and crude samples:

| Column Type/ | CP7834: CP-Sil 24CB; 30 m × 0.32 mm | |
|---|---|---|
| Dimensions | (film thickness 0.5 μl) | |
| Carrier Gas/Pressure | Hydrogen/5.0 psig | |
| Injector/Detector Temps | 250° C./270° C. | |
| Sample size | 1.0 | μl |
| Sample preparation | 10 drops sample: 1 mL HPLC methanol, mixed | |
| | well before injection | |
| Temp. Programme | 80° C. to 240° C. at 20° C./min | |
| | (iso 11 mins) | |
| Total Run Time | 20 | Minutes |

| | |
|---|---|
| Split Ratio | Splitless |
| Integration Notes | Lock out methanol peak |
| GC | GC-3 |
| Method File | 1,3-Butylene Glycol |
| Method Name | CIQC 210328 |

### Equipment

This distillation work used the ‘new’ side stream column which was installed in 2020. The ‘new’ and ‘old’ side stream columns were set up very similarly. Both side stream columns had an 18-plate column fitted with a side stream and a 6-plate column on top of that.

The differences between the columns are that the ‘old’ side stream used knit mesh packing and the ‘new’ side stream used Sulzer packing. The ‘old’ side stream also had column heating fitted to the lower 18-plate and upper 6-plate column. The ‘new’ side stream only had column heating fitted to the lower 18-plate column. For this reason, the boil up achieved in the upper column of the ‘new’ side stream was not as good as the ‘old’ side stream, meaning a slower reflux and distillation.

The ‘new’ side stream column was then fitted with temporary column heating on the upper column to help minimize differences between the columns.

### Specifications

The specifications of a number of suppliers were compared and an initial specification was chosen for the work described here.

The table below details the specifications for 1,3-butanediol for various suppliers and the target specification aimed to meet. The market leader is Diacel. The selected target specification was the same as Godavari's but with a tighter acidity spec of 0.005% max. as acetic acid.

| | | | | | | | Nihon | | |
|---|---|---|---|---|---|---|---|---|---|
| | Diacel | | KH NeoChem | | Godavari | | Koken | Target Spec | |
| Appearance | Clear | | Clear Colorless | | Clear | | Colorless | Clear Colorless | |
| | Colorless | | Liquid Almost | | Colorless | | Liquid | Liquid | |
| | | | Odourless | | Liquid | | | | |
| Color | 15 Pt—Co max. | | 10 APHA max. | | 10 Hazen max. | | | 10 Hazen max. | |
| SG (20/20° C.) | 1.004 to 1.007 | | 1.004 to 1.007 | | 1.004 to 1.006 | | | 1.004 to 1.006 | |
| Purity | | | 99.5% | min. | 99.50% | min. | 99.50% min. | 99.50% | min. |
| Water Content | 0.5% | max. | 0.5% | max. | | | | | |
| Distillation, | 203 | min. | 200 | min. | | | | | |
| Initial boiling | | | | | | | | | |
| point | | | | | | | | | |
| Distillation, Dry | 209 | max. | 215 | max. | | | | | |
| point | | | | | | | | | |
| Free acid (as | 0.005% | max. | 0.005% | max. | 0.010% | max. | | 0.005% | max. |

| | | |
|---|---|---|
| acetic acid) | | |
| Distillate | 95% | min. |

| | | | | |
|---|---|---|---|---|
| pH | Neutral | | | |
| Odor | | Normal | Odorless | Odorless |

### SUMMARY

Crude 1,3-butanediol was added to a laboratory distillation system equipped with a reflux head and side stream take off A vacuum of 20 mmHg was then applied. The system was brought to boil and held at total reflux for ˜15 minutes. At this point, the temperatures at the side stream and the column top were similar. The top reflux ratio was set to 1:1 to and light boilers removed for ˜45 minutes. After this time, the side stream was set to take off 1,3-butanediol at 1:1 and the reflux ratio was adjusted to ˜10:1. Under these conditions, the purity and the odor of the side stream were deemed unacceptable, and the side stream take off was stopped. Further lights were removed from the column top at a reflux ratio of ˜15:1 for the remainder of the day before being allowed to cool overnight. The column was restarted the next day under similar conditions. The side stream was set to take off 1,3-butanediol at 1:1 and the top reflux ratio was set to take off at 5:1. Small variations in the reflux ratio were made and the impact on odor and purity were observed. Generally, the odor and purity of the side stream improved with an increased reflux ratio. The rest of the distillation was run with a top reflux ratio of ˜5:1 and a side stream take off of 1:1. Towards the end of the distillation, the GC heavies in the side stream began to increase and knocked the purity of the side stream below the specification of a 99.5% minimum.

High odor bioBG was treated with a caustic solution (˜40 wt %-50 wt % NaOH), such that the final concentration of NaOH in solution was 0.5 wt %. This mixture of caustic solution in high odor bioBG was then reacted in a tank vessel or caustic reactor at 60° C. for 6 hours while stirring.

The reaction product was then fed into a distillation column where, the unreacted caustic and insoluble reaction product were removed as a bottoms waste; whereas, pure BG, referred to as a lights fraction, was distilled from the top of the column. Further distillation can be completed if required or desired.

### Example 2: Identification of Aroma Compounds in Samples

Samples of aroma compounds or off-odor compounds were identified by gas chromatography/mass spectrometry (GC/MS) and by gas chromatography/olfactory (GC/O).

Briefly, solvent extraction was performed on each sample (10 mL), which was extracted with a mixture of water (10 mL) and dichloromethane (DCM) (10 mL). The mixture (e.g., 1:1:1; 1:2:2) can be vigorously mixed and then the DCM layer collected. DCM can then be concentrated to a volume (200 μL) under nitrogen gas.

Separately, Dynamic Headspace Analysis (DHS) was performed by initially diluting the sample by 10-fold in water and then loaded to a DHS vial. The vial was incubated at 40° C. for 5 minutes. Nitrogen (300 mL) was then purged to the thermal desorption unit (TDU) trap. The trapped compounds were desorbed and injected into a GC/MS system using Gerstel cryo-focusing technology.

A panel comprising two or three trained panelists performed GC/O analysis of the odor-active regions of samples obtained from each of the methods. The chemical profiles and mass spectrometry analysis were performed.

TABLE 1 provides a description of the different samples, including a gold standard control (DSP-555-1), which is a petroleum-derived BG and the bioderived BG products of interest (DSP-555-2; DSP-555-4).

TABLE 1
Aroma Compounds Identified In Test And Control Samples

| | | | Approx | | |
|---|---|---|---|---|---|
| No. | DSP ID | Sample | Qty (g) | Solvent | FIG. |
| 1 | DSP-555-1 | petroBG Control | 10 | >99.7 wt % 1,3-BG | 2 |
| 2 | DSP-555-2 | bioBG Product 1 | 10 | >99.7 wt % 1,3-BG | 2 |
| 3 | DSP-555-3 | Commercially Available 1 | 10 | >99.7 wt % 1,3-BG | 2 |
| 4 | DSP-555-4 | bioBG Product 2 | 10 | >99.7 wt % 1,3-BG | 2 |
| 5 | DSP-555-5 | Commercially Available 2 | 10 | >99.7 wt % 1,3-BG | 3 |
| 6 | DSP-555-6 | C1 lot - MB1803 | 10 | >99.7 wt % 1,3-BG | 3 |
| 7 | DSP-555-7 | C1 lot - MB1808 | 10 | >99.7 wt % 1,3-BG | 3 |
| 8 | DSP-555-8 | C2 lot - MB1833 | 10 | >99.7 wt % 1,3-BG | 3 |
| 9 | DSP-555-9 | C2 lot - MB1836 | 10 | >99.7 wt % 1,3-BG | 4 |
| 10 | DSP-555-10 | C2 lot - MB1849 | 10 | >99.7 wt % 1,3-BG | 4 |
| 11 | DSP-555-11 | C2 lot - MB1853 | 10 | >99.7 wt % 1,3-BG | 4 |
| 12 | DSP-555-12 | C2 lot - MB1856 | 10 | >99.7 wt % 1,3-BG | 4 |
| 13 | DSP-555-13 | DSP-555-2, diluted | 10 | >10 wt % 1,3-BG in | 5 |
| | | 10 wt % | | deionized water | |
| 14 | DSP-555-14 | Pilot scale BG | 10 | >99.7 wt % 1,3-BG | 5 |
| 15 | DSP-555-15 | BDO column lights purge | 10 | >90 wt % 1,3-BG and other | 5 |
| | | from C2 lot | | impurities | |
| 16 | DSP-555-16 | DW/LT-Fraction 2 | 10 | <10 wt % 1,3-BG in water | 5 |
| | | | | with other impurities | |
| 17 | DSP-555-17 | lights waste composite | 50 | | |
| | | DSP-555-4 | | | |

### Results of GCMS/O

TABLE 2 shows a summary of the GC/MS/O chromatogram results illustrated in FIGS. 2-6. Petroleum-derived BG control sample (DSP-555-1) had an odor profile as follows: a fruity and/or sweet odor at an acquisition time of ˜9.8 mins (relative acquisition time ˜0.544 min); a solvent and/or grassy and/or floral odor at an acquisition time of ˜11.8 mins (relative acquisition time ˜0.655 min); a plastic and/or pungent and/or mushroom and/or vegetal odor at an acquisition time of ˜12.8 mins (relative acquisition time ˜0.711 min); a plastic-like and/or floral and/or fruity odor at an acquisition time of ˜13.8 mins (relative acquisition time ˜0.767 min); and a floral and/or fruity odor at an acquisition time of ˜15.7 mins (relative acquisition time ˜0.872 min). Another commercially available control sample (DSP-555-3) had an odor profile as follows: a metallic and/or waxy odor at an acquisition time of ˜11.3 mins (relative acquisition time ˜0.628 min); a fruity and/or solventy odor at an acquisition time of ˜18.3 mins (relative acquisition time ˜1.017 mins); and a vegetal and/or plastic and/or acid odor at an acquisition time of ˜20.5 mins (relative acquisition time ˜1.139 mins).

The findings demonstrated that the bioBG sample of the disclosure (DSP-555-2) uniquely had a fruity and/or solventy odor at an acquisition time of ˜10.7 mins (relative acquisition time ˜0.594 min); a citrus and/or peel odor having an acquisition time of ˜11.8 mins (relative acquisition time ˜0.655 min); and a solventy odor having an acquisition time of ˜12.8 mins (relative acquisition time ˜0.711 min).

A commercially available cosmetic grade BG sample (DSP-555-3) had toluene elute at ˜9.3 mins (relative acquisition time ˜0.517 min); a metallic and/or waxy odor having an acquisition time at ˜11.3 mins (relative acquisition time ˜0.628 min); a fruity and/or solventy odor having an acquisition time at ˜18.3 mins (relative acquisition time ˜1.017 mins); and a vegetal and/or plastic and/or acid odor having an acquisition time at ˜20.5 mins (relative acquisition time ˜1.1.39 mins).

Another bioBG sample of the disclosure (DSP-555-4) uniquely had a fruity odor having an acquisition time of ˜11.3 mins (relative acquisition time ˜0.628 min); and a roasty and/or exhaust odor which eluted at an acquisition time of ˜23.9 mins (relative acquisition time ˜1.328 mins; butyl acetate).

A commercially available BG sample (DSP-555-5 BG) had a fruity odor having an acquisition time of ˜10.8 mins (relative acquisition time ˜0.6 min); a cornchip and/or plastic odor which eluted at an acquisition time of ˜11.4 mins (relative acquisition time ˜0.633 min); and a solventy and/or fruity odor which eluted at an acquisition time of ˜18.6 mins (relative acquisition time ˜1.033 mins).

A BG sample (DSP-555-6) had a floral and/or fruity odor at an acquisition time of ˜11.8 mins (relative acquisition time ˜0.655 min); a plastic and/or mushroom and/or vegetal and/or earthy odor at an acquisition time of ˜12.8 mins (relative acquisition time ˜0.711 min); and a plastic and/or vegetal odor at an acquisition time of 14.7 mins (relative acquisition time ˜0.817 min).

Another BG sample (DSP-555-7) had a brothy and/or metallic and/or green odor at an acquisition time of ˜11.2 mins (relative acquisition time ˜0.622 min).

A BG sample (DSP-555-8) had a mushroom and/or metallic and/or earthy and/or vegetal odor at an acquisition time of ˜11.3 mins (relative acquisition time ˜0.628 min); a fruity and/or grape wine odor at an acquisition time of 12.8 mins (relative acquisition time ˜0.711 min); a cooked potato and/or roasty and/or medicinal odor at an acquisition time of ˜13.4 mins (relative acquisition time ˜0.711 min); a roasty and/or dough odor at an acquisition time of ˜13.8 mins (relative acquisition time ˜0.767 min); and a cooked pasta and/or fried odor at an acquisition time of ˜17.7 mins (relative acquisition time ˜0.983 min).

Another BG sample (DSP-555-9) had a green and/or pungent and/or cheesy odor at an acquisition time of ˜10.5 mins-˜10.9 mins (relative acquisition times ˜0.0.593 min; 0.589 min; 0.594 min; 0.6 min; 0.605 min, respectively); a medicinal and/or sulfur and/or roasty and/or nutty and/or bready odor at an acquisition time of ˜13.3 mins (relative acquisition time ˜0.739 min); and a green floral and/or burnt plastic and/or vegetal odor at an acquisition time of ˜17.5 mins (relative acquisition time ˜0.972 min).

A BG sample (DSP-555-10) had a mushroom and/or metallic odor at an acquisition time of ˜11.1 mins (relative acquisition time ˜0.616 min); and a green bell pepper and/or fruity and/or celery odor at an acquisition time of ˜21.8 mins (relative acquisition time ˜1.211 mins).

Another BG sample (DSP-555-11) had a solventy and/or fruity and/or mushroom and/or vegetal odor at an acquisition time of ˜11.4 mins (relative acquisition time ˜0.633 min); a plastic and/or fruity and/or waxy odor at an acquisition time of ˜11.8 mins (relative acquisition time ˜0.655 min); a sulfuric and/or floral (rosy) and/or vegetal and/or mushroom and/or plastic and/or acrylic odor at an acquisition time of ˜12.2 mins-˜12.8 mins (relative acquisition times ˜0.678 min, ˜0.683 min, ˜0.689 min, ˜0.694 min, ˜0.7 min, ˜0.705 min, ˜0.711 min, respectively); a nutty and/or roasty and/or brothy and/or pyrazine-like and/or metallic odor at an acquisition time of ˜13.3 mins-˜13.7 mins (relative acquisition times ˜0.739 min, ˜0.744 min, ˜0.75 min, ˜0.755 min, ˜0.761 min, respectively); a plastic and/or vegetal and/or cooked rice odor at an acquisition time of ˜14.6 mins (relative acquisition time ˜0.816 min); a cornchip and/or roasty odor at an acquisition time of ˜15 mins (relative acquisition time ˜0.833 min); a solventy and/or stinky odor at an acquisition time of ˜16.7 mins (relative acquisition time ˜0.928 min); a potato and/or pickled plum odor at and acquisition time of ˜20.5 mins (relative acquisition time ˜1.139 mins); and a lactone-like and/or solventy and/or estery and/or fruity odor at an acquisition time of ˜25.3 mins (relative acquisition time ˜1.405 mins).

A BG sample (DSP-555-12) had an earthy and/or mushroom and/or metallic odor at an acquisition time of ˜11.3 mins (relative acquisition time ˜0.628 min); a solventy odor at an acquisition time of ˜14.4 mins (relative acquisition time ˜0.8 min); and a solventy and/or plastic odor at an acquisition time of ˜20.1 mins (relative acquisition time ˜1.117 mins).

Another BG sample (DSP-555-13) had a black tea and/or solventy odor at an acquisition time of ˜18.3 mins-˜18.6 mins (relative acquisition times ˜1.017 mins, ˜1.022 mins, ˜1.028 mins, ˜1.033 mins, respectively); and a green and/or fruity and/or herbal odor at an acquisition time of ˜22.5 mins (relative acquisition time ˜1.250 mins).

A BG sample (DSP-555-14) had a fruity odor at an acquisition time of ˜9.6 mins and ˜10 mins-˜10.8 mins (relative acquisition times ˜0.533 min, ˜0.556 min, ˜0.561 min, ˜0.567 min, ˜0.572 min, ˜0.578 min, ˜0.583 min, ˜0.589 min, ˜0.594 min, ˜0.6 min, respectively); a solvent odor at an acquisition time of ˜11.8 mins (relative acquisition time ˜0.656 min); a green and/or solventy and/or plastic odor at an acquisition time of ˜13 mins (relative acquisition time ˜0.722 min); and a fruity and/or citrus and/or solvent odor at an acquisition time of ˜15.7 mins (relative acquisition time ˜0.872 min).

Another BG sample (DSP-555-15) had a green and/or perfumy and/or fruity odor at an acquisition time of ˜10.6 mins to ˜10.7 mins (relative acquisition times ˜0.589 min, ˜0.594 min, respectively); a cooked potato and/or medicinal odor at an acquisition time of ˜13.3 mins-˜13.4 mins (relative acquisition times ˜0.739 min, ˜0.744 min, respectively); a cornchip and/or medicinal and/or fruity odor at an acquisition time of ˜13.7 mins-˜13.8 mins (relative acquisition times ˜0.761 min, ˜0.767 min, respectively); a plastic and/or gasoline odor at an acquisition time of ˜17.3 mins-˜17.4 mins (relative acquisition times ˜0.961 min, ˜0.967 min, respectively); a fusal-like and/or gasoline and/or plastic odor at an acquisition time of ˜18.8 mins-˜19 mins (relative acquisition times ˜1.044 mins, ˜1.050 mins, respectively); a fruity and/or peanut odor at an acquisition time of ˜20.7 mins-˜20.9 mins (relative acquisition times ˜1.150 mins, ˜1.156 mins, ˜1.161 mins, respectively); and a floral and/or perfumy and/or plastic odor at an acquisition time of ˜22.4 mins (relative acquisition time ˜1.244 mins).

A BG sample (DSP-555-16) uniquely had a fusel and/or sulfur and/or oil odor at an acquisition time of ˜9 mins-˜9.2 mins (relative acquisition times ˜0.5 min, ˜0.506 min, ˜0.511 min, respectively); a fruity and/or plastic odor at an acquisition time of ˜10.2 mins-˜10.4 mins (relative acquisition times ˜0.567 min, ˜0.572 min, ˜0.578 min, respectively); a fruity and/or floral and/or plastic odor at an acquisition time of ˜10.5 mins-˜10.7 mins (relative acquisition times ˜0.583 mins, ˜0.589 min, ˜0.594 min, respectively); and a plastic and/or roasty odor at an acquisition time of ˜13.3 mins-˜13.5 mins (relative acquisition times ˜0.739 min, ˜0.744 min, ˜0.75 min, respectively).

TABLE 2
Results of GC/MS/O

| | | ~Acquisition | ~Acquisition | |
|---|---|---|---|---|
| | | Times | Time/Relative | |
| No. | DSP ID | (mins) | Time* | Odor Description |
| 1 | DSP-555-1 | 9.8; | 0.544; | Fruity, sweet; |
| | | 11.8; | 0.655; | Solvent, grassy, floral; |
| | | 12.8; | 0.711; | Plastic, pungent, mushroom, vegetal; |
| | | 13.8; | 0.767; | Plastic-like, floral, fruity; |
| | | 15.7; | 0.872; | Floral, fruity; |
| | | 18.0 | 1.0 | — |
| 2 | DSP-555-2 | 10.7; | 0.594; | Fruity, solventy; |
| | | 11.8; | 0.655; | citrus, peel, fruity, grassy; |
| | | 12.8; | 0.711; | solventy, acrylic, plastic, vegetal; |
| | | 18.0 | 1.0 | — |
| 3 | DSP-555-3 | 9.3 | 0.517 | (Toluene) |
| | | 11.3; | 0.628; | Metallic, waxy; |
| | | 18.0; | 1.0 | —; |
| | | 18.3; | 1.017; | Fruity, solventy; |
| | | 20.5 | 1.139 | Vegetal, plastic, acid |
| 4 | DSP-555-4 | 9.3 | 0.517 | (Toluene) |
| | | 11.3; | 0.628; | Mushroom, metallic, fruity; |
| | | 18.0; | 1.0; | —; |
| | | 23.9 | 1.328 | Roasty, exhaust |
| 5 | DSP-555-5 | 9.3 | 0.517 | (Toluene) |
| | | 10.8; | 0.6; | Fruity; |
| | | 11.4; | 0.633; | Cornchip, plastic; |
| | | 18.0; | 1.0; | —; |
| | | 18.6 | 1.033 | Solventy, fruity |
| 6 | DSP-555-6 | 11.8; | 0.655; | Floral, fruity; |
| | | 12.8; | 0.711; | Plastic, mushroom, vegetal, earthy; |
| | | 14.7; | 0.817; | Plastic, vegetal; |
| | | 18.0 | 1.0 | — |
| 7 | DSP-555-7 | 9.3 | 0.517 | (Toluene) |
| | | 11.2; | 0.622; | Brothy, metallic, green; |
| | | 18.0 | 1.0 | — |
| 8 | DSP-555-8 | 9.3 | 0.517 | (Toluene) |
| | | 11.3; | 0.628; | Mushroom, metallic, earthy, vegetal; |
| | | 12.8; | 0.711; | Fruity, grape wine; |
| | | 13.4; | 0.744; | Cooked potato, roasty, medicinal; |
| | | 13.8; | 0.767; | Roasty, dough; |
| | | 17.7; | 0.983; | Cooked pasta, fried; |
| | | 18.0 | 1.0 | — |
| 9 | DSP-555-9 | 10.5; | 0.583; | Green, pungent, cheesy; |
| | | 10.6; | 0.589; | Green, pungent, cheesy; |
| | | 10.7; | 0.594; | Green, pungent, cheesy; |
| | | 10.8; | 0.6; | Green, pungent, cheesy; |
| | | 10.9; | 0.605 | Green, pungent, cheesy; |
| | | 13.3; | 0.739; | Medicinal, sulfur, roasty, nutty, bready |
| | | 17.5; | 0.972; | Green floral, burnt plastic, vegetal; |
| | | 18.0 | 1.0 | — |
| 10 | DSP-555- | 11.1; | 0.616; | mushroom, metallic; |
| | 10 | 18.0; | 1.0; | —; |
| | | 21.8 | 1.211 | Green bell pepper, fruity, celery |
| 11 | DSP-555- | 11.4; | 0.633; | Solventy, fruity, mushroom, vegetal; |
| | 11 | 11.8; | 0.655; | Plastic, fruity, waxy; |
| | | 12.2; | 0.678; | Sulfuric, floral (rosy), vegetal, |
| | | | | mushroom, plastic, acrylic; |
| | | 12.3; | 0.683; | Sulfuric, floral (rosy), vegetal, |
| | | | | mushroom, plastic, acrylic; |
| | | 12.4; | 0.689; | Sulfuric, floral (rosy), vegetal, |
| | | | | mushroom, plastic, acrylic; |
| | | 12.5; | 0.694; | Sulfuric, floral (rosy), vegetal, |
| | | | | mushroom, plastic, acrylic; |
| | | 12.6; | 0.7; | Sulfuric, floral (rosy), vegetal, |
| | | | | mushroom, plastic, acrylic; |
| | | 12.7; | 0.705; | Sulfuric, floral (rosy), vegetal, |
| | | | | mushroom, plastic, acrylic; |
| | | 12.8; | 0.711; | Sulfuric, floral (rosy), vegetal, |
| | | | | mushroom, plastic, acrylic; |
| | | 13.3; | 0.739; | Nutty, roasty, brothy, pyrazine -like, |
| | | | | metallic; |
| | | 13.4; | 0.744; | Nutty, roasty, brothy, pyrazine-like, |
| | | | | metallic; |
| | | 13.5; | 0.75; | Nutty, roasty, brothy, pyrazine -like, |
| | | | | metallic; |
| | | 13.6; | 0.755; | Nutty, roasty, brothy, pyrazine -like, |
| | | | | metallic; |
| | | 13.7; | 0.761; | Nutty, roasty, brothy, pyrazine -like, |
| | | | | metallic; |
| | | 14.6; | 0.816; | Plastic, vegetal, cooked rice; |
| | | 15; | 0.833; | Cornchip, roasty; |
| | | 16.7; | 0.928; | Solventy, stinky; |
| | | 18.0; | 1.0; | —; |
| | | 20.5; | 1.139; | Potato, pickled plum; |
| | | 25.3 | 1.405 | Lactone-like, solventy, estery, fruity |
| 12 | DSP-555- | 11.3; | 0.628; | Earthy, mushroom, metallic; |
| | 12 | 14.4; | 0.8; | Solventy; |
| | | 18.0; | 1.0; | —; |
| | | 20.1 | 1.117 | Solventy, plastic |
| 13 | DSP-555- | 18.3; | 1.017; | Black tea, solventy; |
| | 13 | 18.4; | 1.022; | Black tea, solventy; |
| | | 18.5; | 1.028; | Black tea, solventy; |
| | | 18.6; | 1.033; | Black tea, solventy; |
| | | 22.5 | 1.250 | Green, fruity, herbal |
| 14 | DSP-555- | 9.3 | 0.517 | (Toluene) |
| | 14 | 9.6; | 0.533; | Fruity; (2,4-methyl-1,3-dioxane) |
| | | 10; | 0.556; | Fruity; |
| | | 10.1; | 0.561; | Fruity; |
| | | 10.2; | 0.567; | Fruity; |
| | | 10.3; | 0.572; | Fruity; |
| | | 10.4; | 0.578; | Fruity; |
| | | 10.5; | 0.583; | Fruity; |
| | | 10.6; | 0.589; | Fruity; |
| | | 10.7; | 0.594; | Fruity; |
| | | 10.8; | 0.600; | Fruity; |
| | | 11.8; | 0.656; | Solvent; |
| | | 13; | 0.722; | Green, solventy, plastic |
| | | 15.7 | 0.872 | Fruity, citrus, solvent |
| 15 | DSP-555- | 10.6; | 0.589; | Green, perfumy, fruity; |
| | 15 | 10.7; | 0.594; | Green, perfumy, fruity; |
| | | 13.3; | 0.739; | Cooked potato, medicinal; |
| | | 13.4; | 0.744; | Cooked potato, medicinal; |
| | | 13.7; | 0.761; | Cornchip, medicinal, fruity; |
| | | 13.8; | 0.767; | Cornchip, medicinal, fruity; |
| | | 17.3; | 0.961; | Plastic, gasoline; |
| | | 17.4; | 0.967; | Plastic, gasoline; |
| | | 18.8; | 1.044; | Fusal-like, gasoline, plastic; |
| | | 18.9; | 1.050; | Fusal-like, gasoline, plastic; |
| | | 19; | 1.056; | Fusal-like, gasoline, plastic; |
| | | 20.7; | 1.150; | Fruity, peanut; |
| | | 20.8; | 1.156; | Fruity, peanut; |
| | | 20.9; | 1.161; | Fruity, peanut; |
| | | 22.4 | 1.244 | Floral, perfumy, plastic |
| 16 | DSP-555- | 9; | 0.500; | Fusel, sulfur, oil; |
| | 16 | 9.1 | 0.506; | Fusel, sulfur, oil; |
| | | 9.2; | 0.511; | Fusel, sulfur, oil; |
| | | 9.3 | 0.517 | (Toluene) |
| | | 10.2; | 0.567; | Fruity, plastic; |
| | | 10.3; | 0.572; | Fruity, plastic; |
| | | 10.4; | 0.578; | Fruity, plastic; |
| | | 10.5; | 0.583; | Fruity, floral, plastic; |
| | | 10.6; | 0.589; | Fruity, floral, plastic; |
| | | 10.7; | 0.594; | Fruity, floral, plastic; |
| | | 13.3; | 0.739; | Plastic, roasty; |
| | | 13.4; | 0.744; | Plastic, roasty; |
| | | 13.5 | 0.750 | Plastic, roasty |

*“Clean” BG peak starts at 18 mins acquisition time with the relative time being 1.0 min.

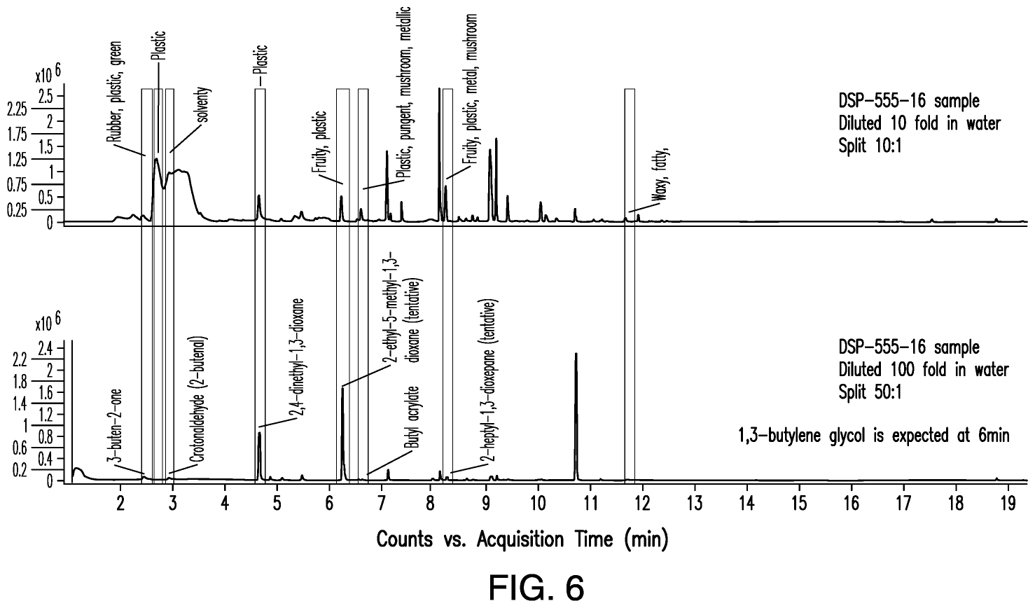

A BG sample of the disclosure where a lights fraction of impurities were removed (DSP-555-16) was diluted 10-fold in water and split 10:1 and in another sample diluted 100-fold in water and split 50:1 for GC/MS-O analysis of the sample obtained by DHS-DH5 column. The DSP-555-16 samples had an odor profile as follows: rubber and/or plastic and/or green odor at an acquisition time of ˜2.5 mins (relative acquisition time ˜0.417 min; 3-buten-2-one); a plastic odor at an acquisition time of ˜2.7 mins (relative acquisition time ˜0.450 min); a solventy odor at an acquisition time of ˜2.9 mins (relative acquisition time ˜0.483 min; 2-butenal); a plastic odor at an acquisition time of ˜4.6 mins (relative acquisition time ˜0.767 min; 2,4-dimethyl-1,3-dioxane); a fruity and/or plastic odor at an acquisition time of ˜6.2 mins (relative acquisition time ˜1.033 mins; 2-ethyl-5-methyl-1,3-dioxane); a plastic and/or pungent and/or mushroom and/or metallic odor at an acquisition time of ˜6.6 mins (relative acquisition time ˜1.1 mins; butyl acrylate); a fruity and/or plastic and/or metal and/or mushroom at an acquisition time of ˜8.2 mins (relative acquisition time ˜1.367 mins; 2-heptyl-1,3-dioxepane); and a waxy and/or fatty odor at an acquisition time of ˜11.8 mins (relative acquisition time ˜1.967 mins). See, TABLE 3; FIG. 6.

TABLE 3
GC/MS/O Analysis of DHS-DB5 Column DSP-555-16 Sample

| | | ~Acquisition | ~Acquisition | |
|---|---|---|---|---|
| | | Times | Time/Relative | |
| No. | DSP ID | (min) | Time* | Odor Description |
| 16 | DSP-555- | 2.5 | 0.417; | Rubber, plastic, green; |
| | 16 | 2.7 | 0.450; | Plastic |
| | | 2.9 | 0.483; | Solventy |
| | | 4.7 | 0.783; | Plastic |
| | | 6.2 | 1.033; | Fruity, plastic |
| | | 6.6 | 1.100; | Plastic, pungent, mushroom, metallic |
| | | 8.2 | 1.367; | Fruity, plastic, metal, mushroom |
| | | 11.8 | 1.967 | Waxy, fatty |

*BG peak starts at 6 mins acquisition time with the relative acquisition time being 1 min

### Example 3: Identification of Aroma Compounds in Test and Neutral Odor bioBG Chemical Samples

### Materials

Test (DSP-650-3) and neutral odor bioBG (DSP-650-4; bioBG product) chemical samples were provided for aroma analysis. Hexane, acetone, and sodium sulfate anhydrous were purchased from Fisher Scientific (Pittsburgh, PA). Commercial standards of 2-methyl-3-heptanone (internal standard), 1-octen-3-one, and n-alkanes (C7-C30) were purchased from Sigma Aldrich (St. Louis, MO).

### Sensory Evaluation

For sensory evaluation of test and neutral odor bioBG samples, a 5-membered sensory panel experienced in sensory analysis evaluated the test and control samples to assess differentiating aroma attributes between the samples. The samples were diluted with water to 1% v/v and 4% v/v and presented to panelists in 4-ounce plastic souffle cups.

### Volatile Aroma Extraction

For volatile aroma extraction, the test or neutral odor bioBG sample (100 mL) was spiked with 5 μg of 2-methyl-3-heptanone as an internal standard and mixed with 100 mL of nano-filtered water in a separatory funnel. The sample was then extracted three times using a 90/10 mixture of hexane/acetone (1×60 mL and 2×40 mL each). The organic layers were pooled and dried over sodium sulfate anhydrous and then concentrated to 1 mL using a BioTage TurboVap® Evaporator (1.5 L/min, 55° C.) and finally concentrated to a final volume of 200 μL with nitrogen evaporation at room temperature (20° C.). The concentrated extract was kept stored at −80° C. until analysis. The final extracts exhibited the characteristic aroma of the samples when a drop of the solution was vaporized on a strip of filter paper.

### Analysis of Aroma Compounds

Analysis of aroma compounds was performed using gas chromatography/mass spectrometry/olfactometry (GC/MS/O) and gas chromatography/quadrupole time-of-flight-mass spectrometry (GC/QTOF-MS). GC/MS/O analyses were performed using an Agilent 6890N Network Gas Chromatograph (Agilent Technologies, Santa Clara, CA) equipped with a DB-5 ms column (30 m×0.25 mm ID×0.25 μm film thickness; Agilent Technologies) that is a low bleed GC/MS non-polar phenyl arylene polymer column. The Agilent 6890N Network GC was also coupled to an Agilent 5973 capillary GC Mass Selective Detector (MSD) (Agilent Technologies) and a Gerstel sniffing port (Olfactory Detection Port; ODP2; Gerstel, Mülheim ander Ruhr, Germany), which allows compounds to be sensed by a human nose as compounds elute from the gas chromatography column. The effluent was split 1:1 after column separation to the MSD and sniffing port. Nitrogen was bubbled through distilled water and purged at the end of the heated (250° C.) sniffing arm to reduce nasal dehydration. Helium was used as a carrier at a constant flow of 1.6 mL/min. The GC oven temperature program was as follows: the initial temperature was held at 40° C. for 1 min and then increased to 240° C. at a rate of 8° C./min and held for 5 minutes. The MSD conditions were as follows: the capillary transfer line was set at 250° C.; the quadrupole temperature at 150° C.; electron ionization (EI) energy at 70 eV; EI source temperature, 230° C.; and mass scanning range, 35-300 m/z. The injection volume was 1 μL in splitless mode, which allows for all of the vaporized sample in the injector to be applied onto the column by closing the split valve, and the inlet temperature was held at 250° C.

Two panelists having experience in GC/MS/O analysis protocols were directed to record the retention time and perceived odor descriptor of detected odorant for each extract evaluated. Compounds in odor-active regions detected by both panelists were identified through further analysis of the extracts using an Agilent 7890A GC (Agilent Technologies, Santa Clara, CA) equipped with a DB-5 ms Ultra Inert column (60 m×0.250 mm×0.25 μm, Agilent Technologies) and coupled with an Agilent 7250 Quadrupole Time-of-Flight Mass Spectrometer (QTOF MS) (Agilent Technologies). The injection volume was 2 μL in splitless mode, and the inlet temperature was held at 250° C. Helium was used as a carrier at a constant flow of 1.2 mL/min. The GC oven temperature program was as follows: the initial temperature was held at 40° C. for 1 min and then increased to 240° C. at a rate of 6° C./min and held for 10 min. Mass Spectrometry conditions used were as follows: capillary transfer line temperature, 250° C.; quadrupole temperature, 150° C.; source temperature, 230° C.; ionization mode, electron ionization (EI); electron energy, 70 eV; emission current, 11.9 μA; and mass scanning range, 35-500 m/z. The software Agilent Unknowns Analysis v10.0 using the SureMass algorithm for data processing was used for compound identification and by injection of pure compound standards. Library match scores for identified compounds were reported on a 100-point scale. The linear retention index (LRI) of compounds on a DB-5 column were calculated with injection of n-alkanes from C7-C30 in the same condition as the samples. Relative concentrations of compounds in the test sample versus the neutral odor bioBG sample were calculated using internal standard normalized peak areas.

Results of Sensory Evaluation of Test and Neutral Odor bioBG Samples

The sensory evaluation of test and neutral odor bioBG samples were assessed by the panel members using aroma attributes. The test sample was evaluated and had urine-like, sweaty, and medicinal/antibiotic ointment aroma attributes. The neutral odor bioBG sample was described as having medicinal/antibiotic ointment and oily aroma attributes.

### Results of Identification and Relative Quantification of Aroma Compounds

GC/MS/O analysis detected five aroma regions in total in the sample extracts. Four aroma regions were detected only in the test sample, while one was only detected by the two panelists in the neutral odor bioBG sample (FIG. 7). The panelists identified the test sample as having solventy, plastic, musty, mushroom, earthy, sulfury, and urine-like aromas. In contrast, the aroma region in the neutral odor bioBG sample was described as fruity. Aroma 2 of the bioBG product (DSP-650-4) eluted at an acquisition time of about 6.3 mins and conveyed a fruity odor-active region. Whereas, aromas 1, 3, 4, and 5 of the test sample (DSP-650-3) eluted at acquisition times of about 6.2 mins, about 6.9 mins, about 8.9 mins, about 17.6 mins, respectively, and conveyed odor-active regions having a solventy odor, a plastic and/or musty odor, a mushroom and/or earthy odor, and a sulfury and/or urine-like odor, respectively.

Analysis of the sample extracts by GC/QTOF-MS (FIG. 8) enabled identification of the aroma compounds (TABLE 4) based on pure compound standard or accurate mass electron ionization (EI) spectra, National Institute of Standards and Technology (NIST) 17 mass spectral library matches, and linear retention indices (LRIs).

TABLE 4
Aroma Compounds Identified In Test And Neutral Odor bioBG Samples

| | | DB-5 | | | Library Match |
|---|---|---|---|---|---|
| No. | Sample | LRI¹ | Odor Description² | Compound | Score³ |
| 1 | Test | 844 | Solventy | 2-methoxymethyl-2,4,5- | 80.88 |
| | | | | trimethyl-1,3-dioxolane | |
| 2 | neutral | 856 | Fruity | 5-norbornene-2-ol | 82.42 |
| | odor | | | | |
| | bioBG | | | | |
| 3 | Test | 885 | Plastic, musty | 3-heptanone | 94.05 |
| 4 | Test | 978 | Mushroom, earthy | 1-octen-3-one | Confirmed with |
| | | | | | pure standard |
| 5 | Test | 1505 | Sulfury, urine-like | N-penty1-2-butylamine | 70.98 |

¹Experimental linear retention index on a DB-5ms column.
²Odor description from GC/MS/O analysis of same extract.
³Library match scores were calculated by Agilent Unknowns Analysis based on the NIST 17 library and are on a 100-point scale.

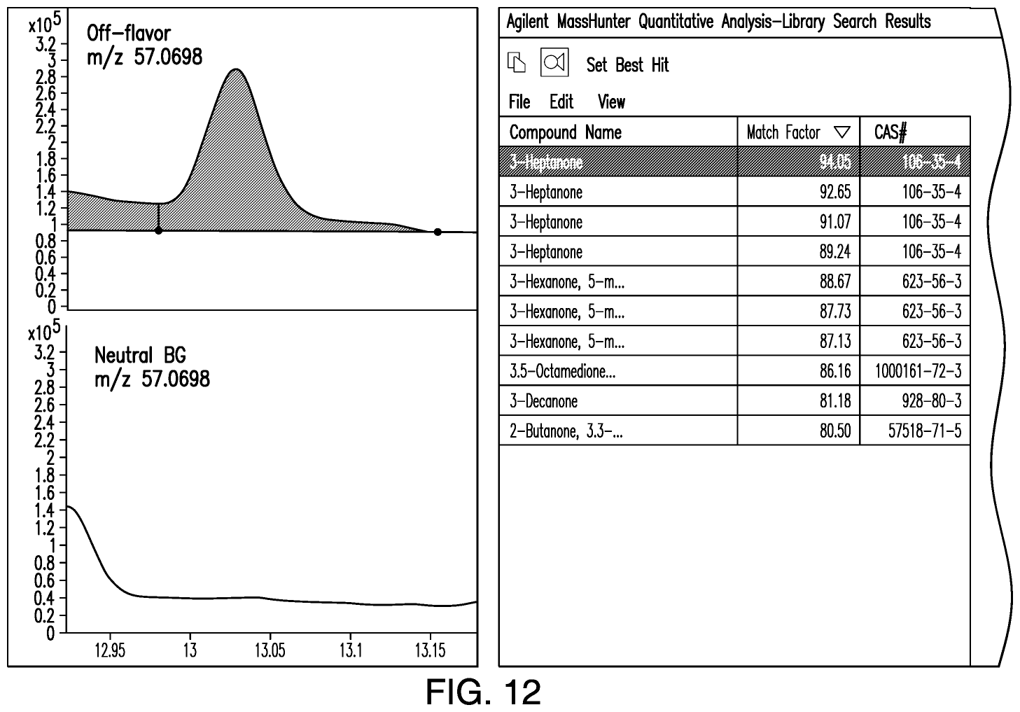

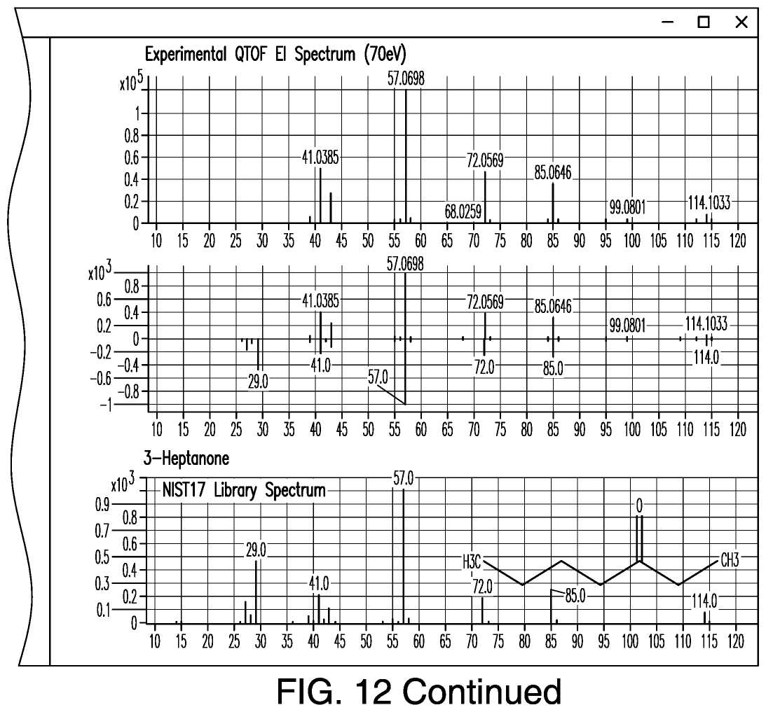

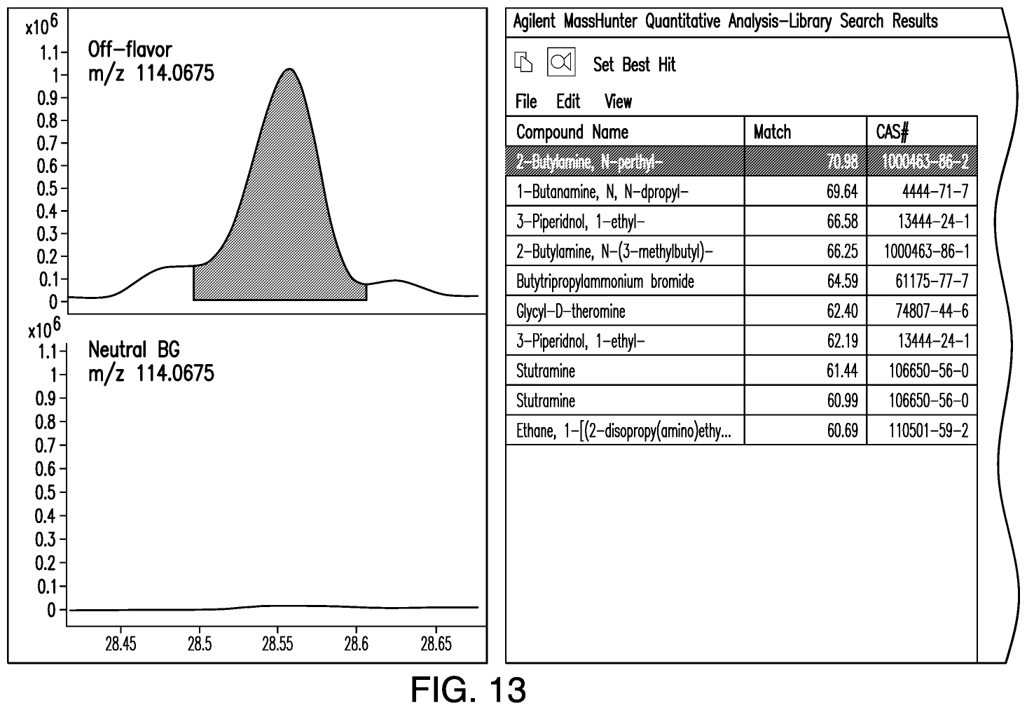

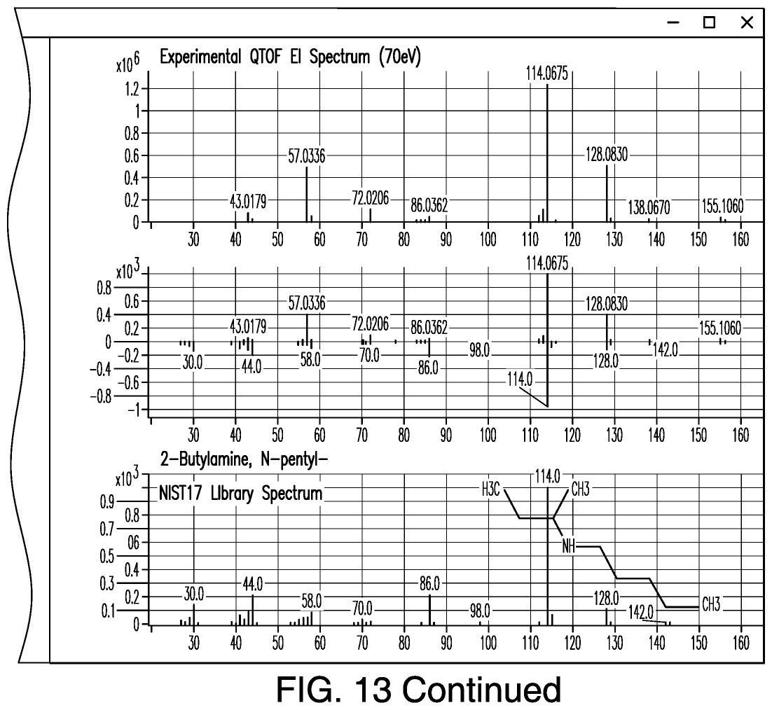

A mushroom, earthy-smelling compound, 1-octen-3-one, was positively identified as aroma 4 by injection of pure 1-octen-3-one standard (2.5 ppm) (FIG. 9), where the Test had about 0.2×10⁴-about 1×10⁴ counts and the neutral odor bioBG (neutral BG) had about 0.15×10⁴-about 0.4×10⁴ counts. Three aroma compounds were detected in the test sample and one aroma compound was detected in the neutral odor bioBG sample (FIG. 10, FIGS. 12-13; and FIG. 11, respectively). The test sample was determined to have compounds identified as 2-methoxymethyl-2,4,5-trimethyl-1,3-dioxolane (solventy) (Aroma 1—test: about 0 counts-2.6×10⁶ counts; neutral BG: about 0 counts-1.6×10⁶ counts) (FIG. 10); 3-heptanone, (plastic, musty) (Aroma 3—test: about 0.9×10⁵ counts-about 3×10⁵ counts; neutral BG: about 0.4×10⁵ counts) (FIG. 12) (80 g/L; 0.08 ppm); 1-octen-3-one (mushroom, earthy) (0.016 μg/L; 0.000016 ppm) (Aroma 4—test: about 0.2×10⁴-about 1×10⁴ counts; neutral BG: about 0.15×10⁴ counts-about 0.4×10⁴ counts), and N-pentyl-2-butylamine (sulfury, urine-like) (Aroma 5—test: about 0 counts-1×10⁶ counts; control: about 0 counts). In the neutral odor bioBG sample, 5-norbornene-2-ol (fruity) (Aroma 2—test: about 0 counts to 1.75×10⁵ counts; neutral BG: about 0 counts to about 3.75×10⁵ counts) was identified (FIG. 11).

The relative concentrations of the identified aroma compounds in the test sample compared to the neutral odor bioBG sample were calculated using internal standard normalized peak areas (TABLE 5). The relative percent increase in compound concentrations (peak area) of the four aroma compounds detected only in the test sample compared to neutral odor bioBG ranged from 124% to 279,628%. For the compound detected only in the neutral odor bioBG sample (identified as 5-norbornene-2-ol), the relative percent decrease in compound concentration in the test sample was 42%.

TABLE 5
Relative Concentration of Aroma Compounds
in Test and Neutral Odor bioBG Samples

| | | Relative Concentration |
|---|---|---|
| | Odor Threshold | Test vs. neutral odor |
| Compound | (μg/L)¹ | bioBG ² |
| 2-methoxymethyl-2,4,5- | NR | +538% |
| trimethyl-1,3-dioxolane | | |
| 5-norbornene-2-ol | NR | −42% |
| 3-heptanone³ | 80 | +29,560% |
| 1-octen-3-one | 0.016 | +124% |
| N-penty1-2-butylamine³ | NR | +279,628% |

¹Odor threshold value in water; retrieved from van Gemert, L. J. (2011). Odour Thresholds 2^{nd} Edition.
NR stands for not reported.
² Relative concentrations were calculated based on internal standard normalized peak areas.
³The noise was used as an estimate of the peak area of the compound in the neutral odor bioBG sample.

### Example 4: Chemical Analysis of Aroma Compounds in Test and Neutral Odor bioBG Chemical Samples

### Methods:

Samples were diluted 1:100 to 1:100,000 fold in water containing 20 μg/L 2-methyl-3-heptanone as an internal standard. See, TABLE 1 for details regarding the tested samples. The total volume for each sample was 5 mL. The samples were incubated at 40° C. for 10 mins under agitation at 1000 rpm. High purity nitrogen gas was then purged through the samples at a rate of 50 mL/min for 6 mins onto a Tenax TA trap. The trap was further dry-purged at 100 mL/min for 6 mins to remove any water before being eluted and injected for GC/MS analysis with Gerstel cryofocusing technology (TDU/CIS). Helium was used as a carrier gas at a flow rate of 1.2 mL/min. DB-WAX (60 m×0.250 mm×0.25 μm) was the column used for GC separation. The gas chromatography oven temperature was set as follows: initial temperature was held at 40° C. for 1 min, ramped to 230° C. at a rate of 10° C./min, and held for 10 mins.

### Results of Chemical Analysis:

Compounds 3-buten-2-one; 2-butenal; styrene; 2,4-dimethyl-1,3-dioxane, butyl acrylate and HDD were detected in some of the samples. Their levels in the samples were semi-quantified (g/L; parts per million (ppm)) based on an external calibration curve. See, TABLE 6

Compounds 3-heptanone, 1-octen-3-one and 2-(2-hydroxyethyl)-2,4-dimethyl-1,3-dioxane (HDD) were also detected in some of the samples. Their levels were quantified using dichloromethane extraction method. The levels were expressed in fold change relative to a reference or a control. See, TABLE 7. As shown in Table 7, 3-heptanone levels in low odor BG samples DSP-555-1 and DSP-555-2 were 2 and 1 in fold change, respectively, while the levels in high odor BG samples DSP-555-10 and DSP-555-12 were 4 and 95 in fold change, respectively. 3-heptanone were not detected in high odor BG samples DSP-555-14 and DSP-555-15. The 1-octen-3-one levels in low odor BG sample DSP-555-2 was 1 in fold change, while the levels in high odor BG samples DSP-555-10, DSP-555-12, and DSP-555-15 were 45, 19, and 187 in fold change, respectively. 1-octen-3-one was not detected in low odor BG sample DSP-555-1 and high odor BG sample DSP-555-14. The HDD were not detected in low odor BG samples DSP-555-1 and DSP-555-2, while the levels of HDD in high odor BG samples DSP-555-10, DSP-555-12, DSP-555-14 and DSP-555-15 were 4, 2.5, 19.3 and 215 in fold change, respectively.

TABLE 6
Semi-Quantification of Aroma Compounds

| | | | | | | | 2,4-dimethyl- | | | | | |
|---|---|---|---|---|---|---|---|---|---|---|---|---|
| | 3-buten-2-one | | 2-butenal | | Styrene | | 1,3-dioxane | | Butyl acrylate | | HDD | |
| DSP ID | μg/L | ppm | μg/L | ppm | μg/L | ppm | μg/L | ppm | μg/L | ppm | μg/L | ppm |
| DSP-555-1 | 1690.9 | 1.6909 | 254.1 | 0.2541 | 6.8 | 0.0068 | n.d. | n.d. | n.d | n.d. | n.d | n.d. |
| DSP-555-2 | 1750.1 | 1.7501 | n.d. | n.d. | 1.1 | 0.0011 | n.d. | n.d. | n.d. | n.d. | 7100 | 7.1 |
| DSP-555-3 | 2,744.8 | 2.74 | n.d. | n.d. | n.d. | n.d. | n.d. | n.d. | 574.4 | 0.5744 | | |
| DSP-555-4 | 1,807.3 | 1.81 | n.d. | n.d. | n.d. | n.d. | n.d. | n.d. | 686.6 | 0.6866 | | |
| DSP-555-5 | 2,070.7 | 2.07 | n.d. | n.d. | n.d. | n.d. | n.d. | n.d. | 156.9 | 0.1569 | | |
| DSP-555-6 | 3,254.3 | 3.25 | 162.9 | 0.1629 | 1.4 | 0.0014 | n.d. | n.d. | n.d. | n.d. | | |
| DSP-555-7 | 18,621.3 | 18.62 | 312.4 | 0.3124 | 34.8 | 0.0348 | 203,000.0 | 203 | 227.4 | 0.2274 | | |
| DSP-555-10 | 11,868.5 | 11.87 | 544.7 | 0.5447 | n.d. | n.d. | 1,182.0 | 1.182 | 530.1 | 0.5301 | 23300 | 23.3 |
| DSP-555-11 | 10,844.0 | 10.84 | 308.9 | 0.3089 | 1.3 | 0.0013 | 500.5 | 0.5005 | 0.0 | 0.0 | | |
| DSP-555-12 | 6,606.5 | 6.61 | 283.2 | 0.2832 | n.d. | n.d. | 727.6 | 0.7276 | 238.9 | 0.2389 | 18300 | 18.3 |
| DSP-555-13 | 791.9 | 0.7919 | n.d. | n.d. | 0.6 | 0.0006 | n.d. | n.d. | 252.7 | 0.2527 | | |
| DSP-555-14 | 6,740.4 | 6.74 | 1,381.8 | 1.3818 | 0.9 | 0.0009 | 1,188,764.7 | 1,188.7647 | 326.8 | 0.3268 | | |
| DSP-555-15 | 359,260.7 | 359.26 | 344.7 | 0.3447 | n.d. | n.d. | 40,483.7 | 40.4837 | 1818.4 | 1.8184 | 16100000 | 16100 |
| DSP-555-16 | 8,375,421.2 | 8,375.42 | 12,152.0 | 12.152 | 46.0 | 0.046 | 2,000,348.8 | 2,000.3488 | 176.2 | 0.1762 | | |
| DSP-555-17 | n.d. | n.d. | n.d. | n.d. | n.d. | n.d. | n.d. | n.d. | n.d. | n.d | | |

n.d. = not detected

TABLE 7
Fold Change of Aroma Compounds

| | | | Threshold | | | | | | |
|---|---|---|---|---|---|---|---|---|---|
| | | Aroma | Value^{c} | Concentration in Fold Change | | | | | |
| Compound | LRI^{a} | Attribute^{b} | (ppm) | DSP-555-1 | DSP-555-2 | DSP-555-10 | DSP-555-12 | DSP-555-14 | DSP-555-15 |
| 3-Heptanone | 1161 | fruity | 80 | 2x | 1x | 4x | 95x | n.d. | n.d. |
| 1-Octen-3-one | 1300 | mushroom | 0.016 | n.d. | 1x | 45x | 19x | n.d. | 187x |
| HDD | 1802 | — | N/A | n.d. | n.d. | 4x | 2.5x | 19.3x | 215x |

^{a}LRI = Linear Retention Index on a DB-WAX column
^{b}Main aroma descriptors detected by three panelists (GC-O)
^{c}Odor threshold in airborne concentrations
* Unknown 1, 2, 3 and 4 were analyzed using DCM extract. The data were presented as fold changes as no authentic standard was available
n.d. = not detected
** The threshold data found were in airborne samples. The ppm for airborne samples is mL/m³, and the ppm for liquid sample is mg/L.

### Example 5: Analysis of Pre- and Post-Purification Test Sample and Control Sample

Select BG product test and control samples were further analyzed. Specifically, one of the bioBG products pre-purification (Sample 1) and post-purification (neutral odor bioBG; Sample 2) was analyzed with another bioBG product of the disclosure (neutral odor bioBG; Sample 3), the gold standard petroleum-derived BG control (Sample 4), and DSP-555-17 (Sample 5).

Since solvents can contain impurities in a final product, such as the BG product of the disclosure, an analysis of purity (peak area percentage) was performed using gas chromatography with flame-ionization detection (GC-FID). Additional tests were performed, such as for stability and color (e.g., Kperm fade time) and the results of which are provided in TABLE 8 and TABLE 9.

TABLE 8
Results of Further Analyses of Samples

| Sample | | Carbonyl | Kperm fade | GC-FID % | | |
|---|---|---|---|---|---|---|
| # | UV210 nm | (ppm) | time (mins) | purity | % Lights | % Heavies |
| 1 | 0.201 | 310 | 15 | 99.88 | 0.01 | 0.11 |
| 2 | 0.048 | 57 | 19 | 99.88 | 0.05 | 0.06 |
| 3 | 0.077 | 260 | 25 | 99.92 | 0.00 | 0.08 |
| 4 | 0.025 | 140 | 30 | 99.77 | 0.01 | 0.22 |

TABLE 9
Impurities Analyses of Samples

| | Sample 1 | Sample 2 | Sample 3 | Sample 4 | Sample 5 |
|---|---|---|---|---|---|
| Compound | (ppm) | (ppm) | (ppm) | (ppm) | (ppm) |
| 1-hydroxy-2 propanone | 0 | 0 | 0 | 0 | 0 |
| 1,2 propanediol (12PDO) | 0 | 0 | 0 | 0 | 31.47 |
| 1,2,3-butanetriol | 0 | 0 | 0 | 0 | 0 |
| 1,2,3-propanetriol | 0 | 0 | 0 | 0 | 0 |
| 1,3 propanediol (13PDO) | 0 | 0 | 0 | 0 | 0 |
| 1,4-butanediol (BDO) | 0 | 0 | 0 | 339 | 0 |
| dioxane | 25 | 8 | 2 | 0 | 418.47 |
| 2,3-butanediol (23BDO) | — | 0 | 0 | 0 | 10.85 |
| 2,5-hexanedione | 0 | 0 | 0 | 0 | 0 |
| 3-buten-1-ol | 0 | 3 | 0 | 173 | 0 |
| 3-buten-2-ol | 0 | 0 | 0 | 0 | 32.22 |
| 3-hydroxy-2-butanone | 0 | 0 | 0 | 0 | 0 |
| 3-OH-butanal | 17 | 0 | 1 | 1 | 94.81 |
| 4-OH-2-butanone | 48 | 7 | 1 | 5 | 3.47 |
| cis-cryotyl alcohol | 0 | 0 | 0 | 0 | 8.91 |
| isopropyl alcohol (IPA) | 0 | 0 | 0 | 0 | — |
| methyl vinyl ketone | 0 | 0 | 0 | 0 | — |
| (MVK) | | | | | |
| n-butane (n-but) | 0 | 0 | 0 | 0 | 178.2 |
| dimethylethanolamine | 0 | 0 | 0 | 0 | 0 |
| (DMAE) | | | | | |
| trans-crotyl alcohol | 0 | 5 | 0 | 0 | 66.02 |

### Example 6: Quantification of Aroma Compounds in Test and Neutral Odor bioBG Chemical Samples

### Materials

Test (DSP-650-3; equivalent to DSP-555-6 to DSP-555-12) and neutral odor bioBG (DSP-650-4; large scale of DSP-555-4) chemical samples were provided for aroma analysis. Hexane, acetone, and sodium sulfate anhydrous were purchased from Fisher Scientific (Pittsburgh, PA). Commercial standards of 2-methyl-3-heptanone (internal standard), 1-octen-3-one, and n-alkanes (C7-C30) were purchased from Sigma Aldrich (St. Louis, MO). Compounds 3-heptanone and 2-ethyl-1-hexanol were purchased from TCI America (Portland, OR).

### Quantification of Aroma Compounds Using Dynamic Headspace And Tandem Gas Chromatography Mass Spectrometry (DHS-GC/MS-QQQ)

Aroma compounds were quantified in the test and neutral odor bioBG samples using dynamic headspace (DHS) and a standard addition method. Two of the compounds (1-octen-3-one and 3-heptanone) quantified were previously identified in the test sample through GC/MS/O analysis. Dynamic headspace and thermal desorption were performed using a MultiPurpose Sampler (MPS-2, Gerstel, Mulheim an der Ruhr, Germany) and thermal desorption unit (TDS-4, Gerstel) connected to a programmed-temperature vaporizer (PTV, Gerstel) injector/cooled injection system (CIS-4, Gerstel). GC/MS analysis was performed using an Agilent 7890B GC equipped with a DB-5 ms column (60 m×0.250 mm×0.25 m, Agilent Technologies) and coupled with an Agilent 7010B Triple Quadrupole Mass Spectrometer (Agilent Technologies Inc., Santa Clara, CA). Both samples were prepared for analysis by weighing 0.500 g of sample into a 20 mL headspace vial and mixing with 4.4 mL of ultrapure water, 10 μL of 100 μg/L 2-methyl-3-heptanone in water (used as an internal standard), and 100 μL of either ultrapure water or a mixture of standards diluted in water. The vial was then placed in an incubator at 40° C. and held for 10 min under agitation (1000 rpm). High purity nitrogen was then purged through the sample at a rate of 50 mL/min for 6 min onto an adsorbent trap packed with 180 mg of Tenax TA. The trap was further dry purged to remove water for 6 mins at a rate of 100 mL/min and then transferred to the thermal desorption unit (TDU). The volatile compounds were desorbed from the trap at 250° C. for 3 min under a flow rate of 50 mL/min. Compounds were cryo-focused on the cooled injection system (CIS) maintained at −50° C. and then desorbed at 250° C. for 3 min and introduced onto the column under split injection mode with a split ratio of 10:1. Helium was used as a carrier gas at a constant flow rate of 1.2 mL/min. The GC oven temperature program was as follows: initial temperature was held at 40° C. for 1 min, ramped to 140° C. at a rate of 5° C./min, then ramped to 240° C. at a rate of 20° C./min and held for 10 min. MS operating conditions were as follows: capillary transfer line was 250° C., source temperature 230° C., quadrupole temperature 150° C.

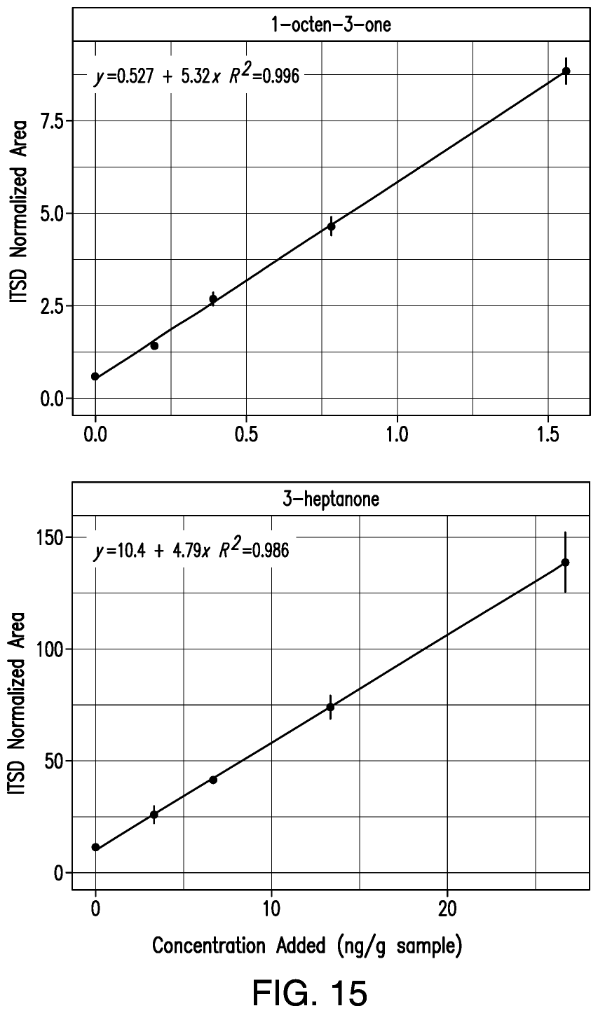

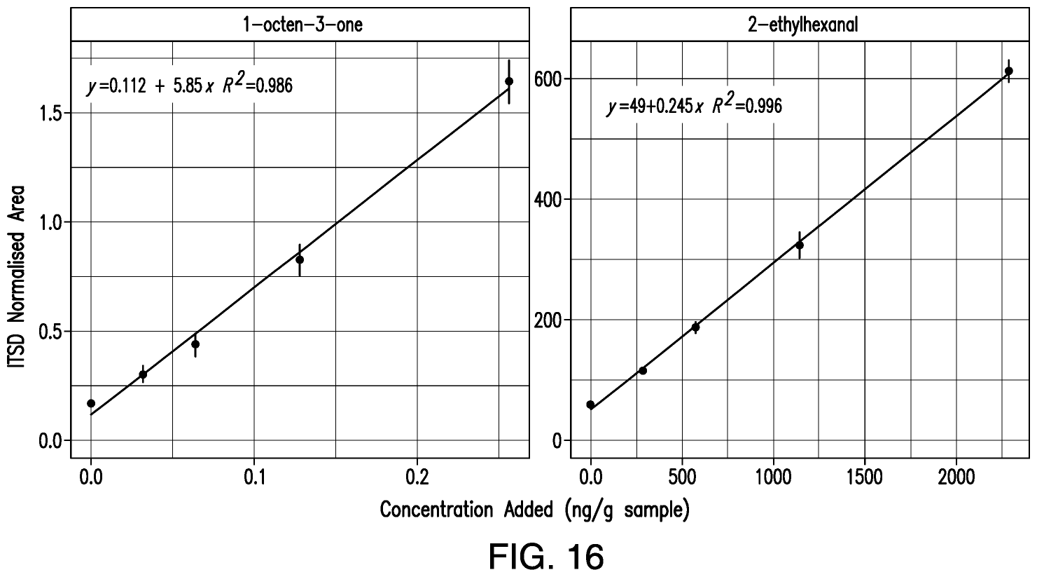

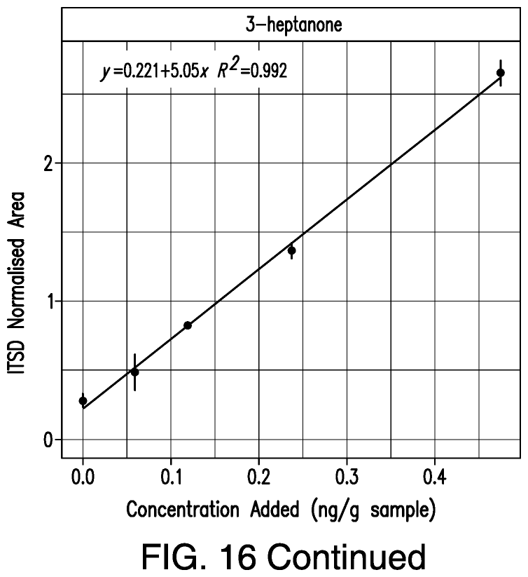

Data were collected using multiple reaction monitoring (MRM) mode with methods optimized by injection of pure standards (TABLE 10). A five-point standard addition calibration curve was built from triplicate measurements with concentrations of all the compounds in a linear range (R2 >0.98) (FIGS. 15-16). Concentrations are reported as an average±standard deviation of three biological replicates.

TABLE 10
Optimized MRM parameters for Quantification of Aroma Compounds in Samples

| | | Precursor | Product Ion (m/Z) | | Collision | |
|---|---|---|---|---|---|---|
| Compound | DB-5 LRI | Ion (m/z) | Quantifier | Qualifier | Energy (eV) | Gain |
| 3-Heptanone | 885 | 114 | 72 | 85 | 5 | 10 |
| 1-Octen-3-one | 977 | 70 | 43 | 55 | 5 | 10 |
| 2-Methyl-3-Heptanone* | | | | | | |
| 2-Ethyl-1-Hexanol | 1028 | 112 | 55 | 41 | 20 | 10 |

*internal standard (ITSD)

### Results of Quantification

In the test sample, 3-heptanone (plastic, musty, fruity) and 1-octen-3-one (mushroom, earthy) were quantified at concentrations approximately 27× and 6×, respectively, above their reported odor thresholds in water of 0.080 ng/g (0.00008 ppm) and 0.016 ng/g (0.000016 ppm), respectively. In the neutral odor bioBG, both of these compounds were quantified at levels below their reported odor thresholds and were approximately 50× and 6.6× lower, respectively, compared to the test sample. 2-ethyl-1-hexanol (citrus and/or floral and/or oily) was only detected in the neutral odor bioBG sample and was quantified at 188.2 ng/g (0.1882 ppm) of sample, which is below its reported odor threshold of 270 ng/g (0.27 ppm). See, TABLE 11.

TABLE 11
Average Concentrations (n = 3) of Aroma Compounds in Samples

| | | | | Average Concentration ± SD⁴ (ng/ g) | | | |
|---|---|---|---|---|---|---|---|
| | | | | | | Neutral odor | |
| | | Odor | | Test Sample | | bioBG Sample | |
| | Odor | Threshold³ | | (DSP-650-03) | | (DSP-650-04) | |
| Compound | Description² | ng/g | ppm | ng/g | ppm | ng/g | ppm |
| 3-heptanone¹ | Plastic, | 0.080 | 0.00008 | 2.15 ± 0.25 | 0.00215 ± 0.00025 | 0.04 ± 0.01 | 0.00004 ± 0.00001 |
| | musty, fruity | | | | | | |
| 1-octen-3-one¹ | Mushroom, | 0.016 | 0.000016 | 0.099 ± 0.017 | 0.000099 ± 0.000017 | 0.015 ± 0.002 | 0.000015 ± 0.000002 |
| | earthy | | | | | | |
| 2-ethyl-1- | Citrus, floral, | 270 | 0.27 | n.d. | n.d. | 188.2 ± 13.0 | 0.1882 ± 0.013 |
| hexanol | oily | | | | | | |

¹Compound detected in DSP-650-02 (Test) by GC/MS/O analysis.
²Odor description from GC/O analysis or literature (http://thegoodscentscompany.com/)
³Odor threshold value in water; retrieved from van Gemert, L.J. (2011). Odour Thresholds. 2nd Edition.
⁴Average ± standard deviation (SD) of three biological replicates. n.d. = not detected.

### Example 7: Gerstel Dynamic Headspace (DHS) GC/MS Analysis of DSP-722

Volatile organic compounds were extracted and concentrated from a BG sample (DSP-722; equivalent to DSP-555-6 to DSP-555-12) and placed in standard headspace vials. The sample was processed and the headspace was purged using a controlled flow of inert gas, thereby providing controlled and efficient extraction conditions. GC/MS analysis was performed using software (GERSTEL MAESTRO).

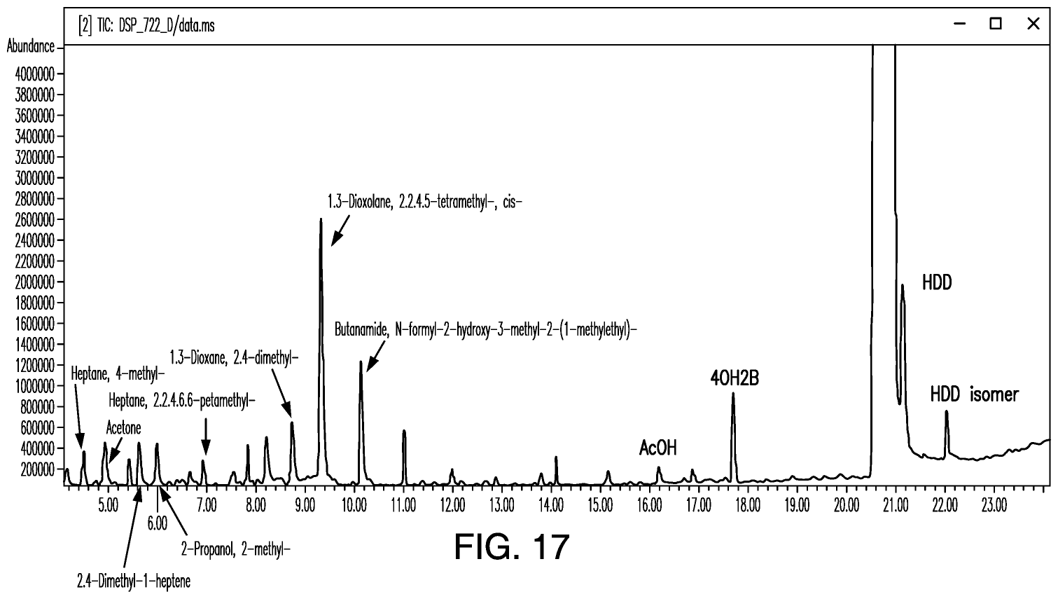

Specifically, 0.5 mL of BG sample (DSP-722) was incubated at 30° C. for 30 minutes using Crabopack B/X sorbent resin. The following compounds were identified: 4-methyl-heptane; acetone; 2,4-dimethyl-1-heptene; 2-methyl-2-propanol; 2,2,4,6,6-pentamethyl-heptane; 2,4-dimethyl-1,3-dioxane, cis-2,2,4,5-tetramethyl-1,3-dioxolane; N-formyl-2-hydroxy-3-methyl-2-(1-methylethyl)-butanamide; acetic acid; 4-hydroxy-2-butanone; 2-(2-hydroxyethyl)-2,4-dimethyl-1,3-dioxane (HDD); and HDD isomer (e.g., cis-, trans-, BG and 4-hydroxy-2-butanone reaction product). See FIG. 17.

### Example 8: Gerstel Dynamic Headspace (DHS) GC/MS Analysis of Samples

Volatile organic compounds were extracted and concentrated from various BG samples and placed in standard headspace vials. The samples were processed, and the headspace purged using a controlled flow of inert gas, thereby providing controlled and efficient extraction conditions. GC/MS analysis was performed using software (GERSTEL MAESTRO).

### Instrumentation

Samples were analyzed on the GERSTEL MPS Robotic sampler with the SHS Static Headspace, TDU 2 Thermal Desorption Unit, CIS 4 Cooled Injection System (with LN2 cooling), DHS Dynamic Headspace, and Agilent 8890 GC/5977B MSD Inert Plus.

GERSTEL's DHS system with the TDU 2 provided a completely automated approach to extracting volatile and semi-volatile compounds. DHS is a nonequilibrium-based technique that promotes more volatiles to pass into the headspace of the sample resulting in lower detection limits.

### Analysis Conditions

| | |
|---|---|
| Column: | Stabilwax-MS 30 m × 0.25 mm × 0.25 um |
| Pneumatics: | |
| CIS 4: | Glass bead liner, solvent venting with 50 mL/min |
| | Split, 10 mL/min at 0.01 min |
| TDU: | Splitless |
| GC Column: | Constant flow, 1 mL/min |
| Temperatures: | |
| CIS 4 | −120° C.; 12° C./sec; 280° C. (5 min) |
| Column | 40° C. (1.5 min); 15° C./min; 250° C. (5 min) |
| TDU | 250° C. (SHS) |
| | 40° C.; 720° C./min; 325° C. (5 min) (DHS) |
| Scan Parameters: | |
| MSD | Full Scan; 40-350 amu |

### Static Headspace (SHS) Conditions

| | | |
|---|---|---|
| Syringe | 2.5 | mL |
| Incubation Time | 20 | mins |
| Incubation Temperature | 50° | C. |
| Agitator Speed | 250 | rpm |
| Agitator On Time | 60 | sec |
| Agitator Off Time | 1 | sec |
| Syringe Temperature | 55° | C. |
| Vial Penetration | 32 | mm |
| Inject Penetration | 54 | mm |
| Inject Volume | 2 | mL |

### Dynamic Headspace (DHS) Conditions

| | | |
|---|---|---|
| Incubation Temperature | 30° | C. |
| Incubation Time | 2 | mins |
| Purge Volume | 600 | mL |
| Purge Flow | 50 | mL/min |
| Trap Temperature | 25° | C. |

| | |
|---|---|
| Trap Material | Carbopack B/X |

### Thermal Desorption System TDU2 Conditions

| | |
|---|---|
| Pneumatics Mode | Spitless |
| Temperature: | 250° C. (SHS) |
| | 40° C.; 720° C./min; |
| | 325° C. (5 mins) (DHS) |
| Transfer Line Temperature | 280° C. |

### Cooled Injection System CIS4 Conditions

| | |
|---|---|
| Liquid Nitrogen Option | |
| Liner Type | Glass bead |
| Carrier Gas | Helium |
| Pneumatics Mode | Split (10:1) or Solvent Venting |
| Vent Flow | 50 mL/min |
| Split Flow | 10 mL/min @ 0.01 min |
| Temperature | −120° C.; 12° C./sec; 280° C. (5 mins) |

### Gas Chromatograph 8890 Conditions

| | |
|---|---|
| Column | Stabilwax-MS 30 m × 0.25 mm × 0.25 μm |
| Mode | Constant flow, 1.0 mL/min |
| Temperature | 40° C. (1.5 min); 15° C./min; 250° C. (5 mins) |

### Mass Selective Detector 5977 Conditions

| EI, Scan mode | | |
|---|---|---|
| Tune | Atune | |
| EM Offset | None | |
| Mass range | 40-350 | amu |
| Gain | 1.00 | |
| Threshold | 100 | |
| Transfer line temperature | 280° | C. |
| Source temperature | 230° | C. |
| Quad temperature | 150° | C. |

### Analysis Details

### Sample Extraction

SHS: A 0.5 g aliquot of each sample was weighed into a 20 mL screw-capped vial. The vials were placed in a VT-15 tray on the MPS autosampler. The samples were incubated at 50° C. for 20 minutes with an agitation speed of 250 RPM. Then 2 mL of the headspace was sampled for injection.

DHS: A 0.5 g aliquot of each sample was weighed into a 20 mL screw-capped vial. The vials were placed in a VT-15 tray on the MPS autosampler. The samples were incubated at 30° C. for 2 minutes with an agitation speed of 500 RPM. Then the samples were extracted for 12 minutes at a helium flow of 50 mL/min. The analytes were trapped at 25° C. on a TDU tube packed with Carbopack B/X.

### Sample Introduction

SHS: The samples were injected using hot injection and trapping (MPS-HIT) through the TDU. The TDU was held isothermal at 250° C. Analytes were cold trapped in the CIS 4 inlet at −120° C. on a glass bead filled liner. When injection was complete, analytes were transferred to the column in split (10:1) mode by heating the inlet rapidly to 275° C.

DHS: The Carbopack B/X tubes were desorbed in solvent vent mode under a 50 mL/min helium flow at 325° C. for 5 minutes. Analytes were cold trapped in the CIS 4 inlet at −120° C. on a glass bead filled liner. When desorption was complete, analytes were transferred to the column in split (10:1) mode by heating the inlet rapidly to 275° C.

The following compounds were identified and quantified: crotonaldehyde, methyl vinyl ketone, and toluene. See TABLE 12. Samples DSP-555-2, DSP-555-4, and DSP-650-4 (large scale of DSP-555-4) are neutral odor bioBG samples. Sample DSP-555-1 is petro-derived BG gold standard. Samples DSP-555-16 and DSP-555-17 are lights waste samples from neutral odor bioBG products.

TABLE 12
Further Analyses and Quantification of Samples

| | | Crotonaldehyde | Methyl Vinyl Ketone | Toluene |
|---|---|---|---|---|
| Sample | Odor | (ppm) | (ppm) | (ppm) |
| DSP-555-1 | Good | 0.23 | 0.17 | 1.12 |
| DSP-555-2 | Good | 0.16 | 0.23 | 0.13 |
| DSP-555-4 | Good | 0.22 | 0.15 | 0 |
| DSP-650-4 | Good | 0.22 | 0.13 | 0.16 |
| DSP-650-3 | Medium | 0.23 | 0.37 | 0.15 |
| DSP-555-16 | Bad | 5.95 | 284.91 | 0.15 |
| DSP-555-17 | Bad | 0.63 | 1.39 | 0.14 |

Additional compounds were identified by analyzing the DHS as compared to SHS in some BG samples identified in TABLE 13, where BG eluted at ˜10 mins for all of the samples. The total ion chromatograms identified compounds in the BG samples and showed increased sensitivity or peak signal in the DHS extractions as compared to the SHS extractions.

TABLE 13
Further DHS Analyses and Compound Determination of Samples

| | Equivalent | | |
|---|---|---|---|
| Sample | Sample | Odor | Compounds (elution time) |
| DSP-651-1 | DSP-555-2 | Good | hexane (~1.8 mins); |
| | | | cyclohexane (~2 mins); |
| | | | dichloromethane (~3.1 mins) |
| DSP-651-2 | DSP-555-4 | Good | hexane (~1.8 mins); |
| | | | cyclohexane (~2 mins); |
| | | | acetone (~2.4 mins); |
| | | | dichloromethane (~3.1 mins) |
| DSP-651-3 | DSP-555-6 to -12 | Medium | hexane (~1.8 mins); |
| | | | cyclohexane (~2 mins); |
| | | | dichloromethane (~3.1 mins) |
| DSP-651-4 | DSP-555-6 to -13 | Medium | hexane (~1.8 mins); |
| | | | 2-methyl-1-pentene (~1.85 mins); |
| | | | cyclohexane (~2 mins); |
| | | | 4-methylhexane (~2.2 mins); |
| | | | acetone (~2.4 mins); |
| | | | 2,4-dimethyl-1-heptene (~2.8 mins); |
| | | | dichloromethane (~3.1 mins); |
| | | | methyl vinyl ketone (~3.3 mins); |
| | | | 2-butenal (~4.1 mins); |
| | | | 3-hydroxybutanal (~8.1 mins); |
| | | | 4-hydroxy-2-butanone (~8.6 mins) |
| DSP-651-5 | DSP-555-6 to -14 | Medium | hexane (~1.8 mins); |
| | | | cyclohexane (~2 mins); |
| | | | 2-butanone (~2.9 mins); |
| | | | dichloromethane (~3.1 mins); |
| | | | methyl vinyl ketone (~3.3 mins); |
| | | | toluene/2-butenal (~4.1 mins); |
| | | | butyl acetate (~4.3 mins); |
| | | | 4-hydroxy-2-butanone (~8.6 mins) |
| DSP-651-6 | DSP-555-6 to -15 | Medium | pentane (~1.6 mins); |
| | | | hexane (~1.8 mins); |
| | | | 2-methyl-1-pentene (~1.85 mins); |
| | | | cyclohexane (~2 mins); |
| | | | 4-methylheptane (~2.15 mins); |
| | | | acetone (~2.4 mins); |
| | | | 2,4-dimethyl-1-heptene (~2.8 mins); |
| | | | dichloromethane (~3.1 mins); |
| | | | methyl vinyl ketone (~3.3 mins); |
| | | | 2-butenal (~4.1 mins); |
| | | | 3-hydroxybutanal (~8.1 mins); |
| | | | 4-hydroxy-2-butanone (~8.6 mins) |

DHS-GC/MS was also performed to analyze and quantify compounds styrene, 2,4-dimethyl-1,3-dioxane, and butyl acrylate in DSP-744 samples (DSP-744-1; DSP-744-2; DSP-744-3; DSP-744-4; DSP-744-5; DSP-744-6; DSP-744-7). See, TABLE 14. Butyl acrylate has a fruity odor with an odor threshold of 0.035 ppm. Styrene has a sweet odor with an odor threshold of 0.016 ppm. These compounds (styrene, 2,4-dimethyl-1,3-dioxane, and butyl acrylate)

TABLE 14
Analysis and Quantification of Aroma Compounds in Samples

| | Equivalent | | Styrene | 2,4-Dimethyl-1,3-Dioxane | Butyl Acrylate |
|---|---|---|---|---|---|
| Sample | Sample | Odor | (ppm) | (ppm) | (ppm) |
| DSP-744-1 | DSP-555-2 | Good | <0.01 | <0.01 | <0.01 |
| DSP-744-2 | DSP-555-4 | Good | <0.01 | <0.01 | <0.01 |
| DSP-744-3 | DSP-555-16 | Bad | <0.01 | 500-100 | <0.01 |
| DSP-744-4 | DSP-555-17 | Bad | 0.05 | <0.01 | <0.01 |
| DSP-744-5 | DSP-555-1 | Good | 0.05 | <0.01 | <0.01 |
| DSP-744-6 | DSP-650-3 | Medium | <0.01 | <0.01 | <0.01 |
| DSP-744-7 | DSP-350-4 | Good | <0.01 | <0.01 | <0.01 |

“<0.01” means below instrument detection limits.

### Example 9: Qualitative/Quantitative Testing on Neutral Odor bioBG Sample

A comparative analysis of neutral odor bioBG samples (DSP-723-1; DSP-723-2; DSP-723-3; DSP-723-4; DSP-723-5) was performed by both GC/MS and headspace GC/MS, and a comparative analysis was also performed by GC/MS of samples of food grade 1,3-butylene glycol to the neutral odor bioBG samples in order to identify compounds that produced an off-odor or undesirable odor.

GC/MS analysis resulted in the detection of an unknown peak that was present in all of the samples and could not be identified with a high degree of certainty, but that was more prevalent in sample DSP-723-3 than in the other samples (DSP-723-1; DSP-723-2; DSP-723-4; DSP-723-5). Testing by headspace GC/MS indicated that toluene was present in all of the samples, which was believed to be the compound contributing to the off-odor characteristics that had been previously observed.

### Instrumentation

Gas Chromatography/Mass Spectrometry (GC/MS): GC/MS testing was used for the analysis of samples along multiple dimensions of chemical properties, providing specific identification of the different compounds separated during the GC analysis. The gas chromatograph separated a complex mixture into its individual components and delivered each one to the mass spectrometer. This analysis generated a chromatogram consisting of different peaks, one for each component of a mixture. The area of each peak was used to measure quantity. GC/MS analysis was used both for qualitative and quantitative determinations of chemical composition.

Solid-Phase Microextraction (SPME): SPME is a solid phase extraction sampling technique that involves the use of a fiber coated with an extracting phase, which is capable of extracting analytes from sample headspace. The fibers used included: Divinylbenzene/Carboxen/Polydimethylsiloxane (DVB/CAR/PDMS) fibers. SPME, paired with an analytical technique, was used for both identification and quantitation of sample analytes.

### Analytical Testing

GC/MS Method Designed for Analysis of Neutral Odor bioBG Samples

Sample solutions (DSP-) and a standard solution of 1,3-butylene glycol (Acros Organics, 99%) were prepared by pipetting 0.1 mL of each into individual 10-mL volumetric flasks and then each was diluted to volume with methanol. The samples were mixed well and analyzed via GC/MS using the following parameters.

### Instrument Conditions for Analysis:

Instrument: Agilent 6890N GC with Agilent 5973 Mass Selective Detector (MSD)
Column: DB-Waxetr 30 m×0.25 mm, 0.25 μm film

TABLE 15
Temperature Program

| Ramp Rate | Final Temperature | Hold Time |
|---|---|---|
| (° C./min) | (° C.) | (min) |
| 0 | 40 | 3 |
| 10 | 80 | 0 |
| 5 | 130 | 2 |
| 10 | 170 | 0 |
| 25 | 240 | 0 |

Injection Volume: 1.0 μL
Injection Mode: Split, 2:1
Inlet Temperature: 230° C.
Flow Rate: 2.8 mL/min
Detection Mode: Scan (32-800 m/z)
Detector Temperature: 240° C.
   Headspace GC/MS with SPME Method Designed for Analysis of Neutral Odor bioBG Samples

Neutral Odor bioBG samples (1-5) and a standard of 1,3-butylene glycol were prepared by pipetting 1 mL of each into individual headspace vials. Each headspace vial was heated at 80° C. for 45 minutes, and then a SPME fiber was used to collect volatiles for analysis by exposing the fiber to the headspace of the vial for two minutes. This was subsequently injected onto a GC/MS and tested using the following parameters.

### Instrument Conditions for Analysis:

Instrument: Agilent 6890N GC with Agilent 5973 MSD
Column: DB-Waxetr 30 m×0.25 mm, 0.25 μm film
Temperature Program: See, TABLE 15.
Injection Volume: 1.0 μL
Injection Mode: Split, 2:1
Inlet Temperature: 230° C.
Flow Rate: 2.8 mL/min
Detection Mode: Scan (30-300 m/z)
Detector Temperature: 240° C.
   Headspace GC/MS with SPME Method Designed for Analysis of Neutral Odor bioBG Samples

Samples (1-5), a standard of 1,3-butylene glycol, and a standard of toluene (VWR, ACS grade) were prepared by pipetting 1 mL of each into individual headspace vials. Each headspace vial was heated at 80° C. for 45 minutes, and then a SPME fiber was used to collect volatiles for analysis by exposing the fiber to the headspace of the vial for two minutes (seven seconds for toluene). This was subsequently injected onto a GC/MS and tested using the following parameters.

### Instrument Conditions for Analysis:

Instrument: Agilent 6890N GC with Agilent 5973 MSD
Column: DB-5MS 30 m×0.25 mm, 0.25 μm film

TABLE 16
Temperature Program

| Ramp Rate | Final Temperature | Hold Time |
|---|---|---|
| (° C./min) | (° C.) | (min) |
| 0 | 35 | 1 |
| 5 | 180 | 1 |
| 25 | 300 | 2 |

Injection Volume: SPME: 2 min
Injection Mode: Spitless
Inlet Temperature: 250° C.
Flow Rate: 2.8 mL/min
Detection Mode: Scan (30-300 m/z)
Detector Temperature: 280° C.

### Results of Analytical Testing

### Analysis by GC/MS: 1,3-Butylene Glycol Method

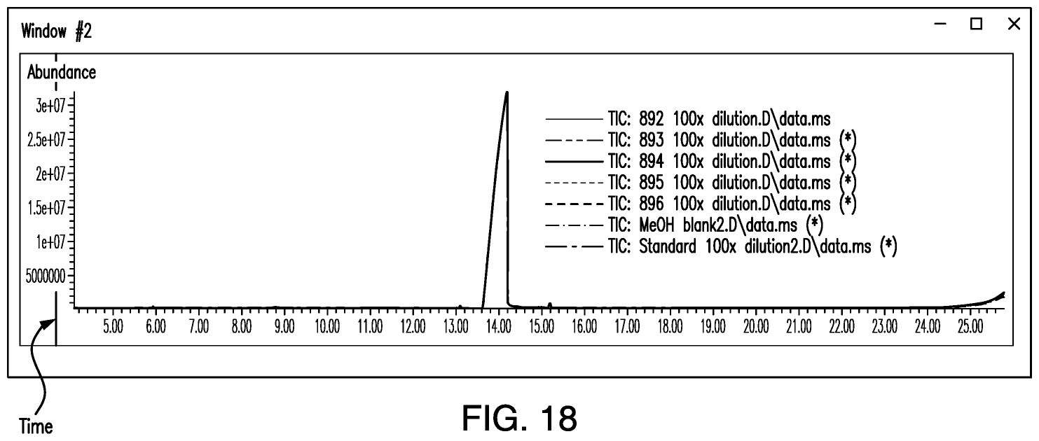

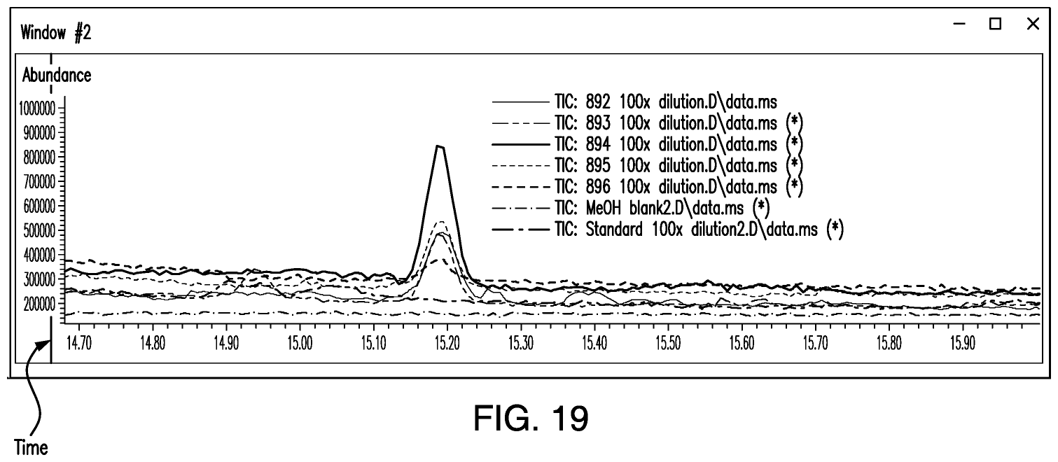

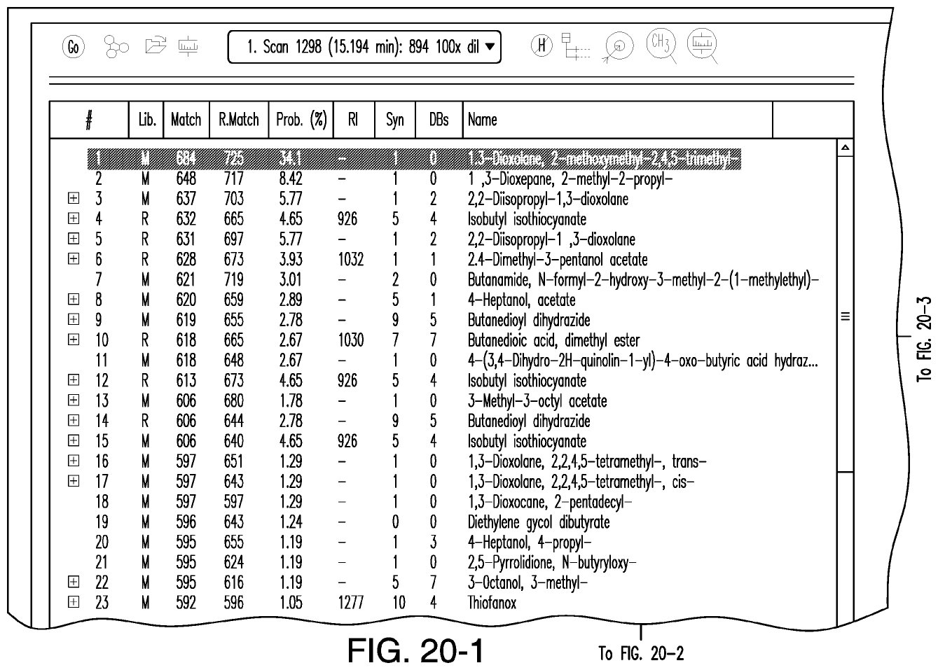

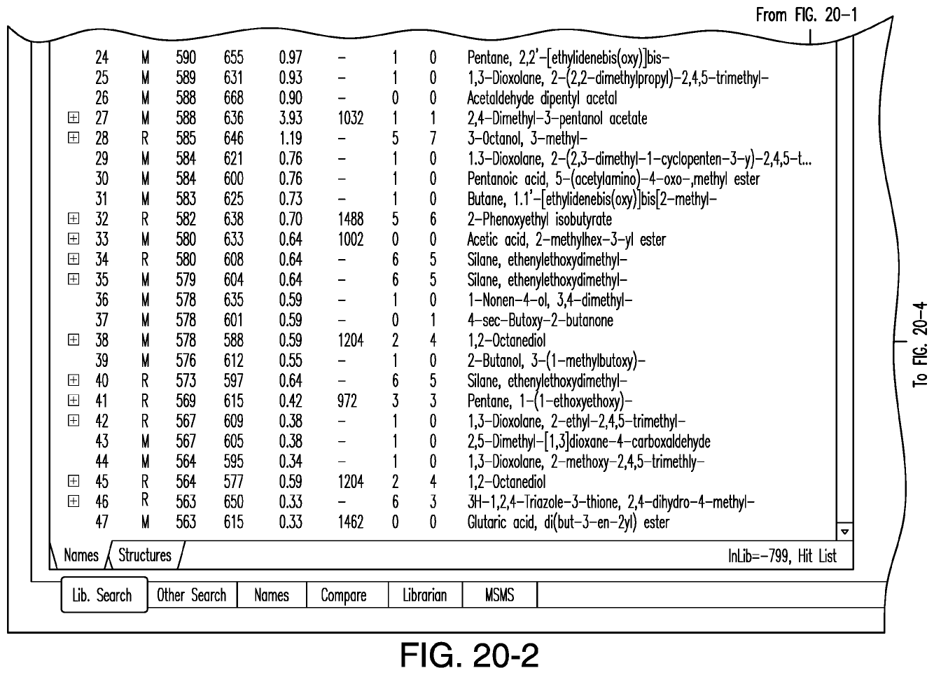

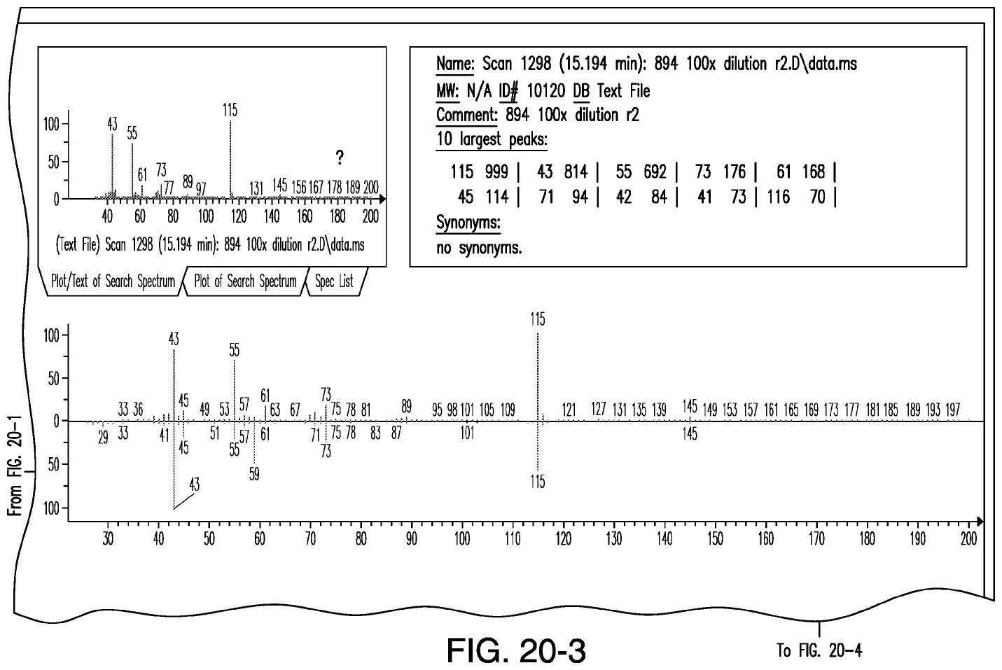

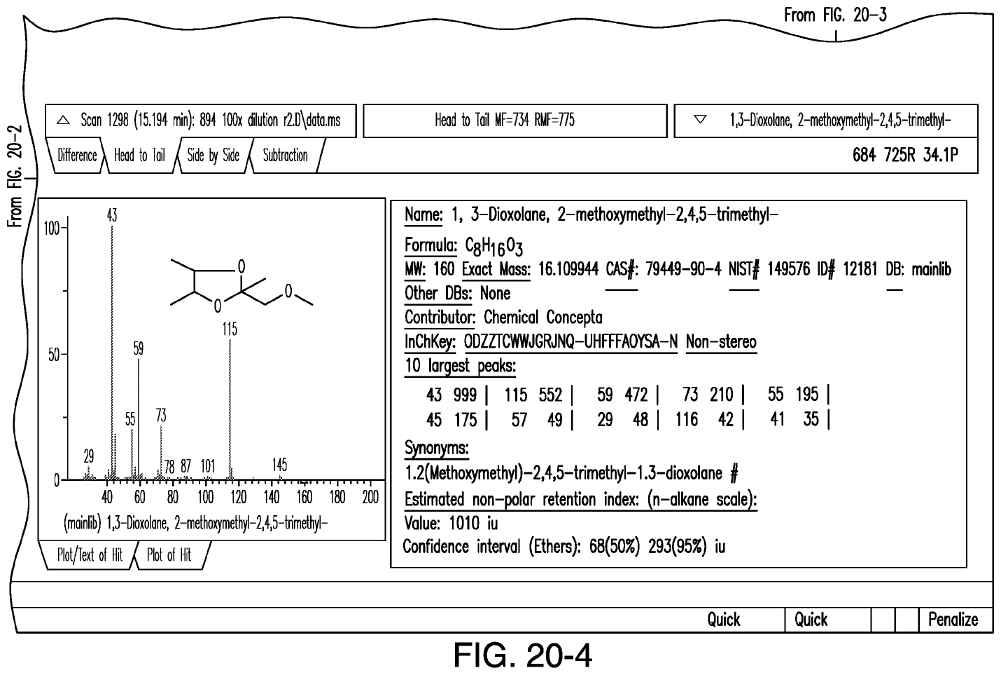

Chromatograms of the sample solutions and the 1,3-butylene glycol standard solution were compared against each other, and a peak with a retention time of 15.20 mins was observed for the samples. In particular, sample DSP-723-3 had a notably more intense peak than when compared with the other samples. No peak was detected at this retention time in the 1,3-butylene glycol standard. Using a NIST library match database, this peak was identified as being 1,3-dioxolane, 2-methoxymethyl-2,4,5-trimethyl (also known as 2-methoxymethyl-2,4,5-trimethyl-1,3-dioxolane). The results for this testing can be seen in FIGS. 18-20. This compound (2-methoxymethyl-2,4,5-trimethyl-1,3-dioxolane) can be a contributor to off-odor in a neutral odor bioBG described here.

Analysis by Headspace GC/MS with SPME: 1,3-Butylene Glycol Method

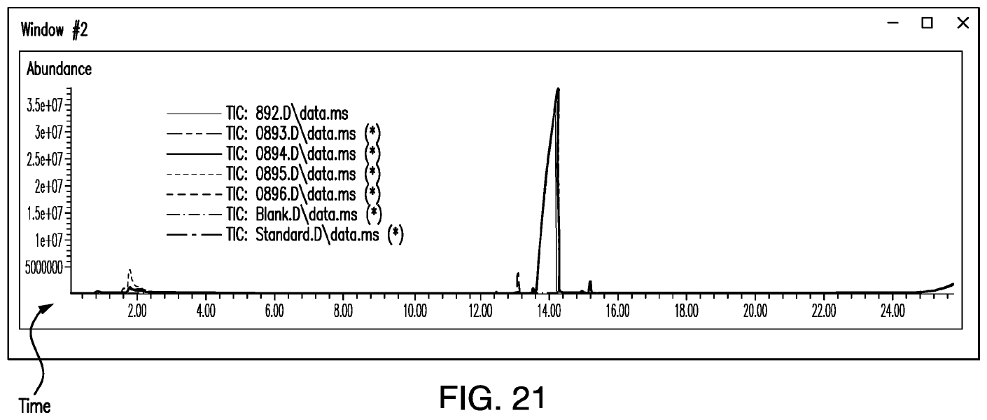

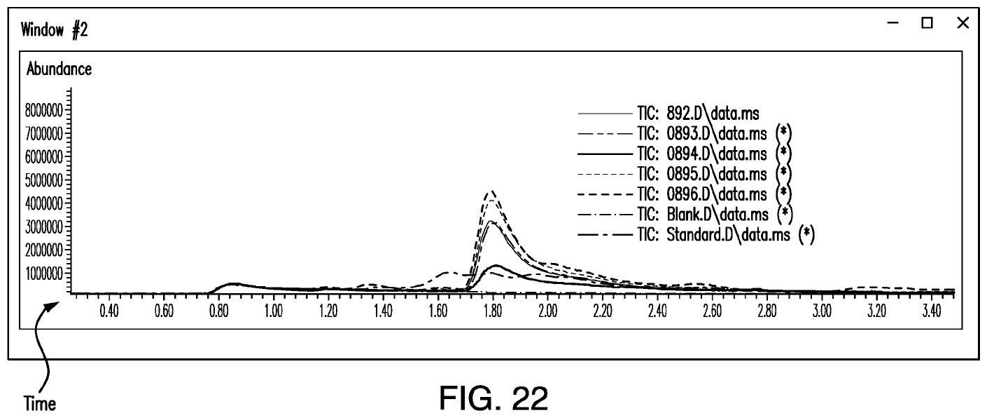

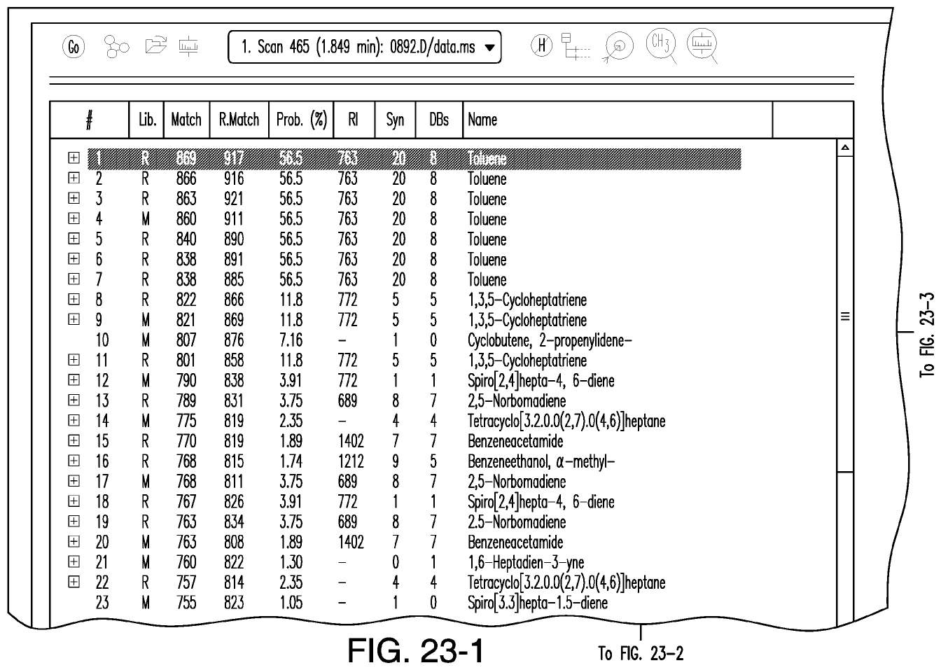

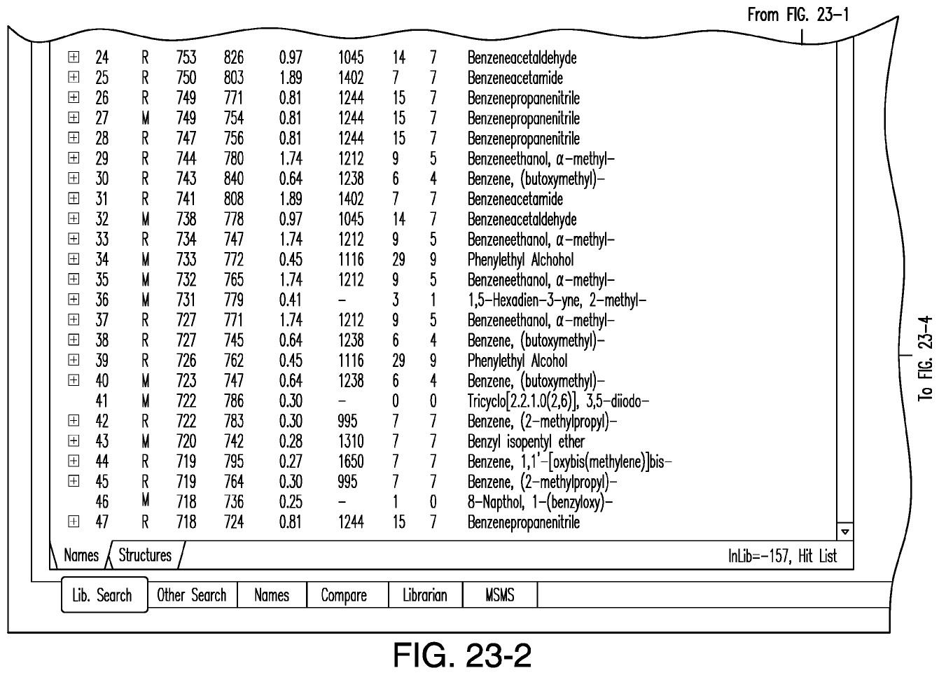

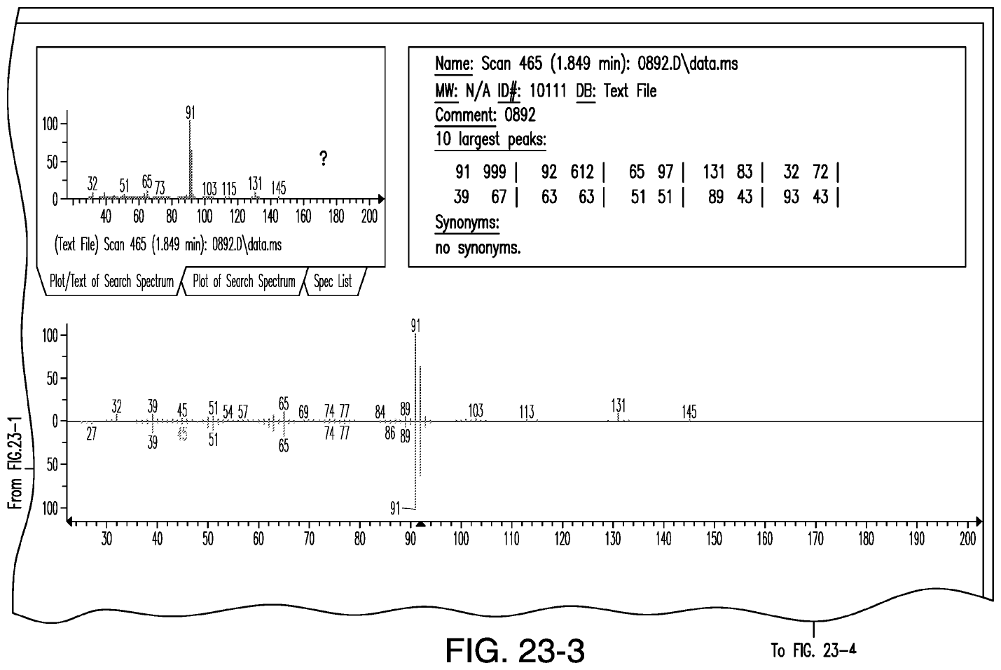

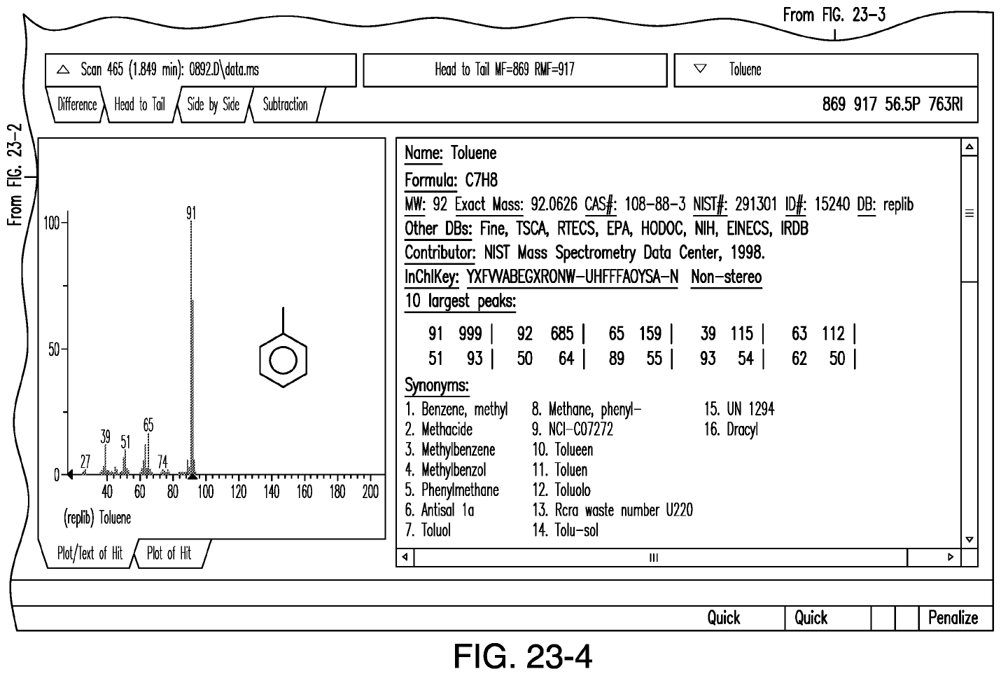

Chromatograms of the samples and the 1,3-butylene glycol standard were compared against each other, and a peak with a retention time of 1.80 min was observed in all of the samples, but was not present in the 1,3-butylene glycol standard. Using a NIST library match database, this peak was identified as being toluene. A 1,3-butylene glycol peak was also observed in all samples and in the 1,3-butylene glycol standard at a retention time of 13.7 mins. The results for this testing can be seen in FIGS. 21-23. This compound (toluene) can be a contributor to off-odor in a neutral odor bioBG described here.

Analysis by Headspace GC/MS with SPME: Volatiles Method

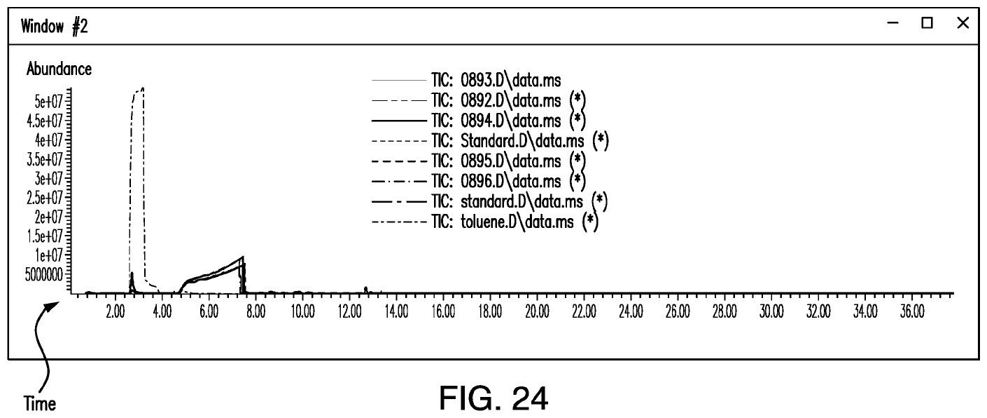

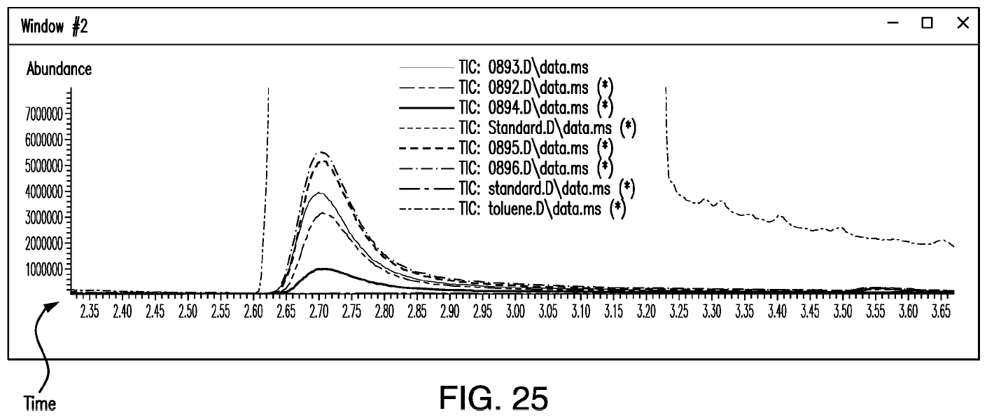

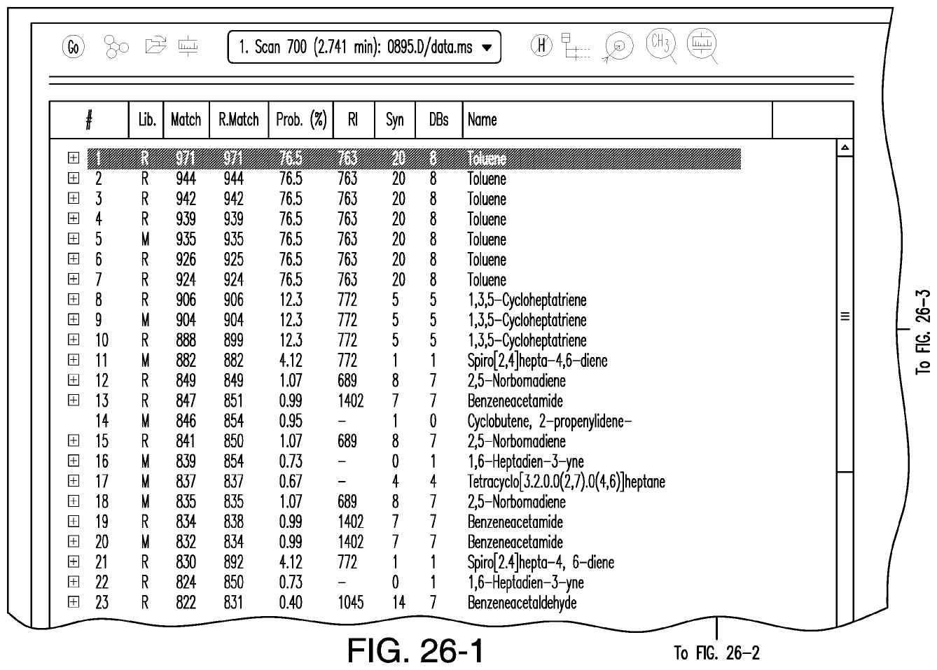

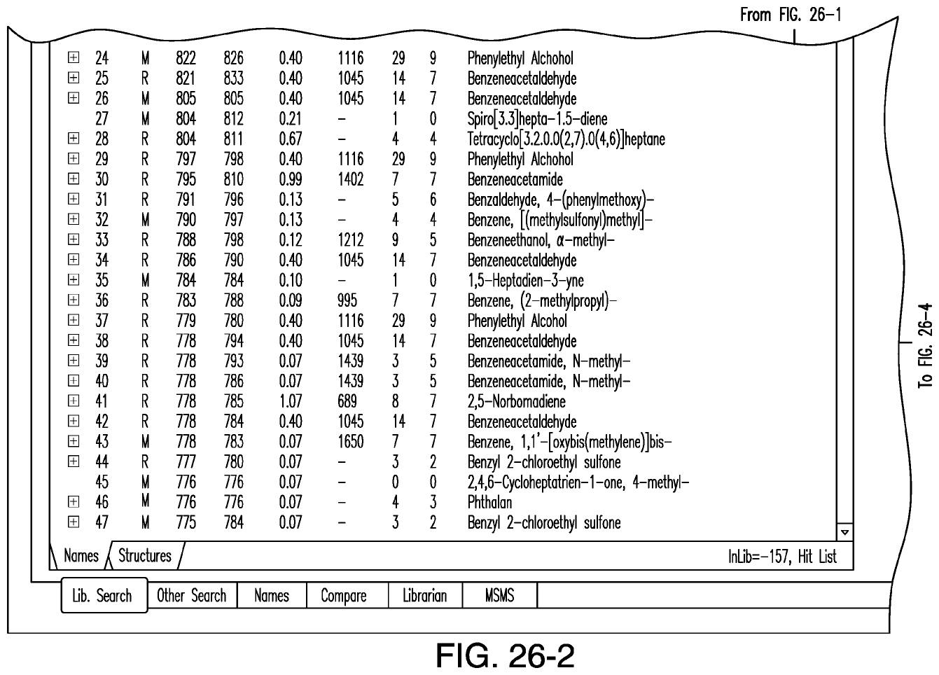

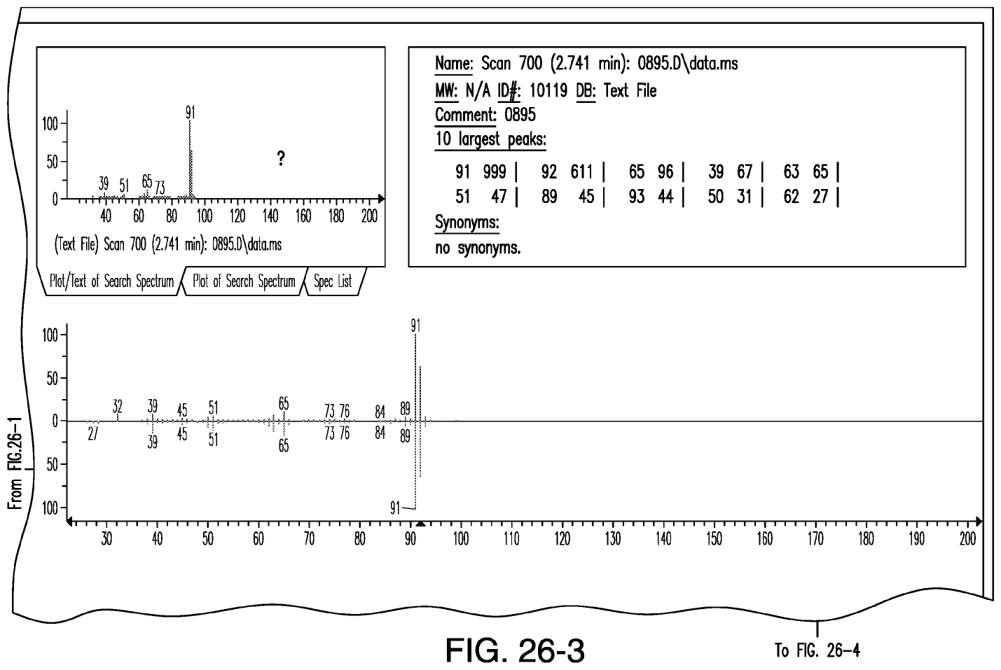

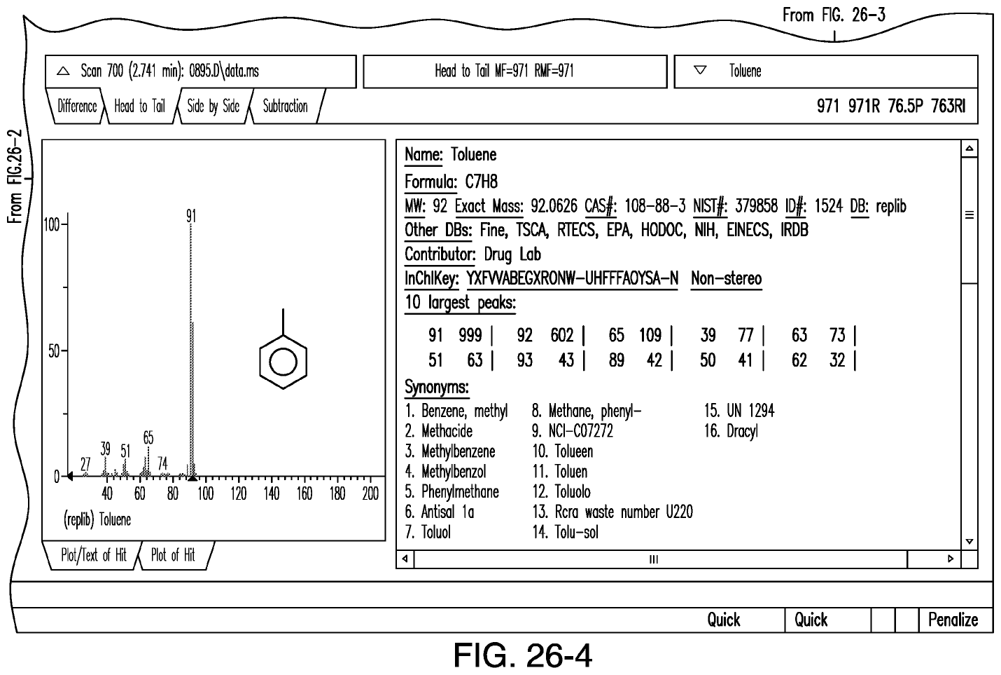

Chromatograms of the neutral odor bioBG samples, a 1,3-butylene glycol standard, and a toluene standard were compared against each other and a peak with a retention time of 2.70 min was observed in all of the samples as well as in the toluene standard. This peak was not observed in either the 1,3-butylene glycol standard or blank and was identified as toluene. This peak had the same retention time as the toluene standard peak and was a clear match to toluene through a NIST library match database search. The results for this testing can be seen in FIGS. 24-26. This compound (toluene) can be a contributor to off-odor in a neutral odor bioBG described Here.

### Specific Embodiments

Non-limiting specific embodiments are described here, each of which is considered to be within the present disclosure.

Embodiment 1. A 1,3-butylene glycol (BG) product comprising one or more compounds selected from the group consisting of: 5-norbornene-2-ol; 2-ethyl-1-hexanol; and salts, derivatives, analogs, or solvates thereof, or any combinations thereof, wherein the BG product comprises bioderived BG (bioBG).

Embodiment 2. A 1,3-butylene glycol (BG) product comprising one or more compounds characterized by an odor-active region as measured by gas chromatography/mass spectrometry/olfactory (GC/MS/O) having a relative acquisition time selected from a group consisting of: about 0.594 min having a fruity and/or solventy odor; about 0.628 min having a fruity odor; about 0.655 mins having a citrus and/or peel odor; about 0.711 mins having a solventy odor; about 1.328 mins having a roasty and/or exhaust odor; and combinations thereof, where the BG product comprises bioderived BG (bioBG), and the bioBG has a relative acquisition time of 1 min.

Embodiment 3. A 1,3-butylene glycol (BG) product comprising one or more compounds characterized by an odor-active region as measured by gas chromatography/quadrupole time-of-flight-mass spectrometry (GC/QTOF-MS), where the odor-active region elutes at an acquisition time of about 12.2 mins at 66.0463 m/z, wherein the BG product comprises bioderived BG (bioBG).

Embodiment 4. A 1,3-butylene glycol (BG) product comprising one or more compounds comprising a mass spectrum as characterized by quadrupole time-of-flight-mass spectrometry electron ionization (QTOF EI) at 70 eV, wherein the mass spectrum comprises peaks: 39.0229 m/z; 43.0707 m/z; 53.0385 m/z; 57.0169 m/z; 66.0463 m/z; 74.0156 m/z; 81.0333 m/z; 85.0284 m/z; 91.0539 m/z; 95.0489 m/z; and 110.0724 m/z; or the mass spectrum comprises peaks: 39.0229 m/z; 43.0707 m/z; 53.0385 m/z; 66.0463 m/z; 81.0333 m/z; 91.0539 m/z; 95.0489 m/z; and 110.0724 m/z, wherein the BG product comprises bioderived BG (bioBG).

Embodiment 5. A 1,3-butylene glycol (BG) product comprising one or more compounds as measured by extracted ion chromatograms selected from the group consisting of: 2-methoxymethyl-2,4,5-trimethyl-1,3-dioxolane having about 0 counts-1.6×10⁶ counts at 59.0489 m/z; 1-octen-3-one having about 0.15×10⁴-about 0.4×10⁴ counts at m/z 70.0411; 3-heptanone having about 0.4×10⁵ counts at m/z 57.0698; N-pentyl-2-butylamine having about 0 counts at m/z 114.0675; 5-norbornene-2-ol having about 0 counts to about 3.75×10⁵ counts at m/z 66.0463; and any combinations thereof, wherein the BG product comprises bioderived BG (bioBG).

Embodiment 6. The BG product according to any one of embodiments 1-5, wherein 3-hydroxy-butanal is in an amount of less than 25 ppm; 4-hydroxy-2-butanone is in an amount less than 25 ppm; 4-(3-hydroxybutoxy)butan-2-one is in an amount less than 25 ppm; 4-((4-hydroxybutan-2-yl)oxy)-butan-2-one is in an amount less than 25 ppm; 1,2-propanediol is in an amount less than 1 ppm; 1,3-propanediol is in an amount less than 10 ppm; 2,3-butanediol is in an amount less than 1 ppm; 2,4-dimethyl-6-((1-(4-methyl-1,3-dioxan-2-yl)propan-2-yl)oxy)-1,3-dioxane is in an amount less than 2 ppm; 4-(sec-butoxy)-2,6-dimethyl-1,3-dioxane is in an amount less than 4 ppm; 2,4-dimethyl-1,3-dioxane is in an amount less than 8 ppm; (E)-4-methyl-2-(prop-1-en-1-yl)-1,3-dioxane is in an amount less than 8 ppm; 3-hydroxybutyl acetate is in an amount less than 8 ppm; 1-(2-methyl-1,3-dioxan-4-yl)propan-2-ol is in an amount less than 8 ppm; 2-methyl-4-propyl-1,3-dioxane is in an amount less than 8 ppm; tri-substituted 1,3-dioxane

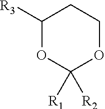

is in an amount less than 8 ppm and/or 3-hydroxybutyl carbono-substituted

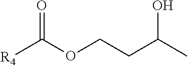

in an amount less than 8 ppm, wherein R1 to R4 are the same or different; a hydrogen atom, a C1-C4 alkyl group optionally substituted by an OH group, or a C2-C4 alkenyl group optionally substituted by OH group; 2,4-dimethyl-2-vinyl-1,3-dioxane; 2-(2,4-dimethyl-1,3-dioxan-2-yl)ethan-1-ol; 2,2,4-trimethyl-1,3-dioxane; 4-methyl-1,3-dioxane, wherein the total of 2,4-dimethyl-2-vinyl-1,3-dioxane; 2-(2,4-dimethyl-1,3-dioxan-2-yl)ethan-1-ol; 2,2,4-trimethyl-1,3-dioxane; 4-methyl-1,3-dioxane is in a total amount of less than 34 ppm; (E)-4-methyl-2-(prop-1-en-1-yl)-1,3-dioxane; 1-(4-methyl-1,3-dioxan-2-yl)propan-2-ol; 2,4-dimethyl-1,3-dioxane; 3-hydroxybutyl (E)-but-2-enoate; 3-hydroxybutyl acetate; 3-hydroxybutyl 3-hydroxybutanoate, wherein the total of (E)-4-methyl-2-(prop-1-en-1-yl)-1,3-dioxane; 1-(4-methyl-1,3-dioxan-2-yl)propan-2-ol; 2,4-dimethyl-1,3-dioxane; 3-hydroxybutyl (E)-but-2-enoate; 3-hydroxybutyl acetate; 3-hydroxybutyl 3-hydroxybutanoate is in a total amount of less than 63 ppm or wherein the total of 2,4-dimethyl-2-vinyl-1,3-dioxane; 2-(2,4-dimethyl-1,3-dioxan-2-yl)ethan-1-ol; 2,2,4-trimethyl-1,3-dioxane; 4-methyl-1,3-dioxane; (E)-4-methyl-2-(prop-1-en-1-yl)-1,3-dioxane; 1-(4-methyl-1,3-dioxan-2-yl)propan-2-ol; 2,4-dimethyl-1,3-dioxane; 3-hydroxybutyl (E)-but-2-enoate; 3-hydroxybutyl acetate; 3-hydroxybutyl 3-hydroxybutanoate is in a total amount of less than 97 ppm; methyl vinyl ketone is in an amount less than 8 ppm; 1-hydroxy-3-butanone is in an amount less than 1 ppm; acetone is in an amount less than 6 ppm; formaldehyde is in an amount less than 2 ppm; crotonaldehyde is in an amount less than 1 ppm; acetaldol is in an amount less than 1 ppm; acetaldehyde is in an amount less than 5 ppm; butyraldehyde is in an amount less than 4 ppm; 2-butanol, wherein the total of 2,4-dimethyl-2-vinyl-1,3-dioxane; 2-(2,4-dimethyl-1,3-dioxan-2-yl)ethan-1-ol; 2,2,4-trimethyl-1,3-dioxane; 4-methyl-1,3-dioxane; (E)-4-methyl-2-(prop-1-en-1-yl)-1,3-dioxane; 1-(4-methyl-1,3-dioxan-2-yl)propan-2-ol; 2,4-dimethyl-1,3-dioxane; 3-hydroxybutyl (E)-but-2-enoate; 3-hydroxybutyl acetate; 3-hydroxybutyl 3-hydroxybutanoate; and 2-butanol is in a total amount of less than 65 ppm; butyl acetate; butyl acrylate; 4-hydroxy-2-butanone; 2-methoxymethyl-2,4,5-trimethyl-1,3-dioxolane (solventy); 3-heptanone; 1-octen-3-one; N-pentyl-2-butylamine; 3-buten-2-one; 2-butenal; 2,4-dimethyl-1,3-dioxane; 2-ethyl-5-methyl-1,3-dioxane; 2-heptyl-1,3-dioxepane; 1-hydroxy-2-propanone; 1,2 propanediol (12PDO); 1,2,3-butanetriol; 1,2,3-propanetriol; 1,3 propanediol (13PDO); 1,4-butanediol (BDO); 2,3-butanediol (23BDO); 2,5-hexanedione; 3-buten-2-ol; cis-cryotyl alcohol; isopropyl alcohol (IPA); methyl vinyl ketone (MVK); n-butane (n-but); dimethylethanolamine (DMAE); toluene; HDD, and any combinations thereof.

Embodiment 7. The BG product of embodiment 6, wherein the BG product does not comprise a compound selected from the group consisting of: 3-hydroxy-butanal; 4-hydroxy-2-butanone; 4-(3-hydroxybutoxy)butan-2-one; 4-((4-hydroxybutan-2-yl)oxy)-butan-2-one; 1,2-propanediol; 1,3-propanediol; 2,3-butanediol; 2,4-dimethyl-6-((1-(4-methyl-1,3-dioxan-2-yl)propan-2-yl)oxy)-1,3-dioxane; 4-(sec-butoxy)-2,6-dimethyl-1,3-dioxane; 2,4-dimethyl-1,3-dioxane; (E)-4-methyl-2-(prop-1-en-1-yl)-1,3-dioxane; 3-hydroxybutyl acetate; 1-(2-methyl-1,3-dioxan-4-yl)propan-2-ol; 2-methyl-4-propyl-1,3-dioxane; tri-substituted 1,3-dioxane

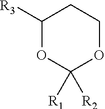

and/or 3-hydroxybutyl carbono-substituted

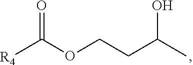

wherein R1 to R4 are the same or different; a hydrogen atom, a C1-C4 alkyl group optionally substituted by an OH group, or a C2-C4 alkenyl group optionally substituted by OH group; 2,4-dimethyl-2-vinyl-1,3-dioxane; 2-(2,4-dimethyl-1,3-dioxan-2-yl)ethan-1-ol; 2,2,4-trimethyl-1,3-dioxane; 4-methyl-1,3-dioxane; (E)-4-methyl-2-(prop-1-en-1-yl)-1,3-dioxane; 1-(4-methyl-1,3-dioxan-2-yl)propan-2-ol; 2,4-dimethyl-1,3-dioxane; 3-hydroxybutyl (E)-but-2-enoate; 3-hydroxybutyl acetate; 3-hydroxybutyl 3-hydroxybutanoate; methyl vinyl ketone; 1-hydroxy-3-butanone; acetone; formaldehyde; crotonaldehyde; acetaldol; acetaldehyde; butyraldehyde; 2-butanol; butyl acrylate; butyl acetate; 4-hydroxy-2-butanone; 2-methoxymethyl-2,4,5-trimethyl-1,3-dioxolane (solventy); 3-heptanone; 1-octen-3-one; N-pentyl-2-butylamine; 3-buten-2-one; 2-butenal; 2,4-dimethyl-1,3-dioxane; 2-ethyl-5-methyl-1,3-dioxane; 2-heptyl-1,3-dioxepane; 1-hydroxy-2-propanone; 1,2 propanediol (12PDO); 1,2,3-butanetriol; 1,2,3-propanetriol; 1,3 propanediol (13PDO); 1,4-butanediol (BDO); 2,3-butanediol (23BDO); 2,5-hexanedione; 3-buten-2-ol; cis-cryotyl alcohol; isopropyl alcohol (IPA); n-butane (n-but); dimethylethanolamine (DMAE); toluene; HDD; and or any combinations thereof, wherein the BG product comprises bioderived BG (bioBG).

Embodiment 8. A 1,3-butylene glycol (BG) product, wherein the BG product comprises a bioderived BG (bioBG), wherein the BG product further comprises one or more compounds, wherein 3-hydroxy-butanal is in an amount of less than 25 ppm; 4-hydroxy-2-butanone is in an amount less than 25 ppm; 4-(3-hydroxybutoxy)butan-2-one is in an amount less than 25 ppm; 4-((4-hydroxybutan-2-yl)oxy)-butan-2-one is in an amount less than 25 ppm; 1,2-propanediol is in an amount less than 1 ppm; 1,3-propanediol is in an amount less than 10 ppm; 2,3-butanediol is in an amount less than 1 ppm; 2,4-dimethyl-6-((1-(4-methyl-1,3-dioxan-2-yl)propan-2-yl)oxy)-1,3-dioxane is in an amount less than 2 ppm; 4-(sec-butoxy)-2,6-dimethyl-1,3-dioxane is in an amount less than 4 ppm; 2,4-dimethyl-1,3-dioxane is in an amount less than 8 ppm; (E)-4-methyl-2-(prop-1-en-1-yl)-1,3-dioxane is in an amount less than 8 ppm; 3-hydroxybutyl acetate is in an amount less than 8 ppm; 1-(2-methyl-1,3-dioxan-4-yl)propan-2-ol is in an amount less than 8 ppm; 2-methyl-4-propyl-1,3-dioxane is in an amount less than 8 ppm; tri-substituted 1,3-dioxane

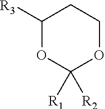

is in an amount less than 8 ppm and/or 3-hydroxybutyl carbono-substituted

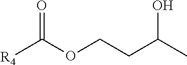

in an amount less than 8 ppm, wherein R1 to R4 are the same or different; a hydrogen atom, a C1-C4 alkyl group optionally substituted by an OH group, or a C2-C4 alkenyl group optionally substituted by OH group; 2,4-dimethyl-2-vinyl-1,3-dioxane; 2-(2,4-dimethyl-1,3-dioxan-2-yl)ethan-1-ol; 2,2,4-trimethyl-1,3-dioxane; 4-methyl-1,3-dioxane, wherein the total of 2,4-dimethyl-2-vinyl-1,3-dioxane; 2-(2,4-dimethyl-1,3-dioxan-2-yl)ethan-1-ol; 2,2,4-trimethyl-1,3-dioxane; 4-methyl-1,3-dioxane is in a total amount of less than 34 ppm; (E)-4-methyl-2-(prop-1-en-1-yl)-1,3-dioxane; 1-(4-methyl-1,3-dioxan-2-yl)propan-2-ol; 2,4-dimethyl-1,3-dioxane; 3-hydroxybutyl (E)-but-2-enoate; 3-hydroxybutyl acetate; 3-hydroxybutyl 3-hydroxybutanoate, wherein the total of (E)-4-methyl-2-(prop-1-en-1-yl)-1,3-dioxane; 1-(4-methyl-1,3-dioxan-2-yl)propan-2-ol; 2,4-dimethyl-1,3-dioxane; 3-hydroxybutyl (E)-but-2-enoate; 3-hydroxybutyl acetate; 3-hydroxybutyl 3-hydroxybutanoate is in a total amount of less than 63 ppm or wherein the total of 2,4-dimethyl-2-vinyl-1,3-dioxane; 2-(2,4-dimethyl-1,3-dioxan-2-yl)ethan-1-ol; 2,2,4-trimethyl-1,3-dioxane; 4-methyl-1,3-dioxane; (E)-4-methyl-2-(prop-1-en-1-yl)-1,3-dioxane; 1-(4-methyl-1,3-dioxan-2-yl)propan-2-ol; 2,4-dimethyl-1,3-dioxane; 3-hydroxybutyl (E)-but-2-enoate; 3-hydroxybutyl acetate; 3-hydroxybutyl 3-hydroxybutanoate is in a total amount of less than 97 ppm; methyl vinyl ketone is in an amount less than 8 ppm; 1-hydroxy-3-butanone is in an amount less than 1 ppm; acetone is in an amount less than 6 ppm; formaldehyde is in an amount less than 2 ppm; crotonaldehyde is in an amount less than 1 ppm; acetaldol is in an amount less than 1 ppm; acetaldehyde is in an amount less than 5 ppm; butyraldehyde is in an amount less than 4 ppm; 2-butanol, wherein the total of 2,4-dimethyl-2-vinyl-1,3-dioxane; 2-(2,4-dimethyl-1,3-dioxan-2-yl)ethan-1-ol; 2,2,4-trimethyl-1,3-dioxane; 4-methyl-1,3-dioxane; (E)-4-methyl-2-(prop-1-en-1-yl)-1,3-dioxane; 1-(4-methyl-1,3-dioxan-2-yl)propan-2-ol; 2,4-dimethyl-1,3-dioxane; 3-hydroxybutyl (E)-but-2-enoate; 3-hydroxybutyl acetate; 3-hydroxybutyl 3-hydroxybutanoate; and 2-butanol is in a total amount of less than 65 ppm; butyl acetate; butyl acrylate; 4-hydroxy-2-butanone; 2-methoxymethyl-2,4,5-trimethyl-1,3-dioxolane (solventy); 3-heptanone; 1-octen-3-one; N-pentyl-2-butylamine; 3-buten-2-one; 2-butenal; 2,4-dimethyl-1,3-dioxane; 2-ethyl-5-methyl-1,3-dioxane; 2-heptyl-1,3-dioxepane; 1-hydroxy-2-propanone; 1,2 propanediol (12PDO); 1,2,3-butanetriol; 1,2,3-propanetriol; 1,3 propanediol (13PDO); 1,4-butanediol (BDO); 2,3-butanediol (23BDO); 2,5-hexanedione; 3-buten-2-ol; cis-cryotyl alcohol; isopropyl alcohol (IPA); methyl vinyl ketone (MVK); n-butane (n-but); dimethylethanolamine (DMAE); toluene; HDD; and any combinations thereof.

Embodiment 9. A 1,3-butylene glycol (BG) product, wherein the BG product comprises a bioderived BG (bioBG), wherein the BG product does not comprise a compound selected from the group consisting of: 3-hydroxy-butanal; 4-hydroxy-2-butanone; 4-(3-hydroxybutoxy)butan-2-one; 4-((4-hydroxybutan-2-yl)oxy)-butan-2-one; 1,2-propanediol; 1,3-propanediol; 2,3-butanediol; 2,4-dimethyl-6-((1-(4-methyl-1,3-dioxan-2-yl)propan-2-yl)oxy)-1,3-dioxane; 4-(sec-butoxy)-2,6-dimethyl-1,3-dioxane; 2,4-dimethyl-1,3-dioxane; (E)-4-methyl-2-(prop-1-en-1-yl)-1,3-dioxane; 3-hydroxybutyl acetate; 1-(2-methyl-1,3-dioxan-4-yl)propan-2-ol; 2-methyl-4-propyl-1,3-dioxane; tri-substituted 1,3-dioxane

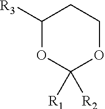

and/or 3-hydroxybutyl carbono-substituted

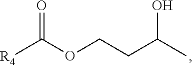

wherein R1 to R4 are the same or different; a hydrogen atom, a C1-C4 alkyl group optionally substituted by an OH group, or a C2-C4 alkenyl group optionally substituted by OH group; 2,4-dimethyl-2-vinyl-1,3-dioxane; 2-(2,4-dimethyl-1,3-dioxan-2-yl)ethan-1-ol; 2,2,4-trimethyl-1,3-dioxane; 4-methyl-1,3-dioxane; (E)-4-methyl-2-(prop-1-en-1-yl)-1,3-dioxane; 1-(4-methyl-1,3-dioxan-2-yl)propan-2-ol; 2,4-dimethyl-1,3-dioxane; 3-hydroxybutyl (E)-but-2-enoate; 3-hydroxybutyl acetate; 3-hydroxybutyl 3-hydroxybutanoate; methyl vinyl ketone; 1-hydroxy-3-butanone; acetone; formaldehyde; crotonaldehyde; acetaldol; acetaldehyde; butyraldehyde; 2-butanol; butyl acrylate; butyl acetate; 4-hydroxy-2-butanone; 2-methoxymethyl-2,4,5-trimethyl-1,3-dioxolane (solventy); 3-heptanone; 1-octen-3-one; N-pentyl-2-butylamine; 3-buten-2-one; 2-butenal; 2,4-dimethyl-1,3-dioxane; 2-ethyl-5-methyl-1,3-dioxane; 2-heptyl-1,3-dioxepane; 1-hydroxy-2-propanone; 1,2 propanediol (12PDO); 1,2,3-butanetriol; 1,2,3-propanetriol; 1,3 propanediol (13PDO); 1,4-butanediol (BDO); 2,3-butanediol (23BDO); 2,5-hexanedione; 3-buten-2-ol; cis-cryotyl alcohol; isopropyl alcohol (IPA); n-butane (n-but); dimethylethanolamine (DMAE); toluene; HDD; and or any combinations thereof, wherein the BG product comprises bioderived BG (bioBG).

Embodiment 10. A 1,3-butylene glycol (BG) product, wherein the BG product does not comprise one or more compounds having an odor-active region as measured by gas chromatography/quadrupole time-of-flight-mass spectrometry (GC/QTOF-MS), wherein the odor-active region elutes at an acquisition time selected from a group consisting of: about 12 mins at 59.0489 m/z, about 13 mins at 57.0698 m/z, about 15.5 mins at 70.0411 m/z, about 28.5 mins at 114.0675 m/z, and combinations thereof, wherein the BG product comprises bioderived BG (bioBG).

Embodiment 11. A 1,3-butylene glycol (BG) product, wherein the BG product does not comprise one or more compounds comprising a mass spectrum as characterized by quadrupole time-of-flight-mass spectrometry electron ionization (QTOF EI) at 70 eV, wherein the mass spectrum comprises an odor-active region, wherein the mass spectrum is selected from the group consisting of: (a) 43.017 m/z, 55.0541 m/z, 73.0283 m/z, 86.0723 m/z, 97.0642 m/z, and 115.0752; (b) 41.0385 m/z, 57.0698 m/z, 68.0259 m/z, 72.0569 m/z, 85.0646 m/z, 99.0801 m/z, and 114.1033 m/z; and (c) 43.0179 m/z, 57.0336 m/z, 72.0206 m/z, 86.0362 m/z, 114.0675 m/z, 128.0830 m/z, 138.0670 m/z, and 155.1060 m/z, wherein the BG product comprises bioderived BG (bioBG).

Embodiment 12. A 1,3-butylene glycol (BG) product comprising a bioderived BG (bioBG), wherein the BG product does not comprise one or more compounds comprising: an odor-active region as measured by GC/MS that elutes at an acquisition time selected from a group consisting of: about 9.3 mins (toluene); about 9.6 mins (butyl acetate); about 10.5 mins (butyl acrylate); about 16 mins (4-hydroxy-2-butanone); and any combinations thereof, where bioBG elutes at an acquisition time of about 18 mins; or
an odor-active region as measured by GC/MS that elutes at an acquisition time selected from a group consisting of: about 9.3 mins (toluene); about 9.6 mins (butyl acetate); about 10.5 mins (butyl acrylate); about 11 mins; about 11.7 mins; about 14.2 mins; about 16 mins (4-hydroxy-2-butanone); about 17.7 mins; and any combinations thereof, where bioBG elutes at an acquisition time of about 18 mins.

Embodiment 13. A 1,3-butylene glycol (BG) product, wherein the BG product does not comprise one or more compounds characterized by an odor-active region as measured by gas chromatography/mass spectrometry/olfactory (GC/MS/O) having a relative acquisition time selected from a group consisting of: about 0.517 mins (toluene); about 0.533 mins (butyl acetate); about 0.583 mins (butyl acrylate); about 0.889 mins (4-hydroxy-2-butanone); and combinations thereof, where the BG product comprises bioderived BG (bioBG), and the bioBG has a relative acquisition time of 1 min; or
about 0.517 mins (toluene); about 0.533 mins (butyl acetate); about 0.583 mins (butyl acrylate); about 0.611 mins; about 0.65 mins; about 0.789 mins; about 0.889 mins (4-hydroxy-2-butanone); about 0.983 mins; and combinations thereof, where the BG product comprises bioderived BG (bioBG), and the bioBG has a relative acquisition time of 1 min.

Embodiment 14. A 1,3-butylene glycol (BG) product, wherein the BG product does not comprise one or more compounds comprising an odor-active region as measured by GC/MS/O that elutes at an acquisition time selected from the group consisting of: ˜2.5 mins (rubber and/or plastic and/or green; 3-buten-2-one); ˜2.7 mins (plastic); ˜2.9 mins (solventy; 2-butenal); ˜4.6 mins (plastic: 2,4-dimethyl-1,3-dioxane); ˜6.2 mins (fruity and/or plastic; 2-ethyl-5-methyl-1,3-dioxane); ˜6.6 mins (plastic and/or pungent and/or mushroom and/or metallic; butyl acrylate); ˜8.2 mins (fruity and/or plastic and/or metal and/or mushroom; 2-heptyl-1,3-dioxepane); about 11.8 mins (waxy and/or fatty); and any combinations thereof, where the BG product comprises bioderived BG (bioBG).

Embodiment 15. A 1,3-butylene glycol (BG) product, wherein the BG product does not comprise one or more compounds comprising an odor-active region as measured by GC/MS/O that elutes at an acquisition time selected from the group consisting of: ˜9 mins-˜9.2 mins (relative acquisition times ˜0.5 mins, ˜0.506 mins, ˜0.511 mins, respectively) having a fusel and/or sulfur and/or oil odor; ˜9.3 mins (relative acquisition time 0.517; toluene); ˜10.2 mins-˜10.4 mins (relative acquisition times ˜0.567 mins, ˜0.572 mins, ˜0.578 mins, respectively) having a fruity and/or plastic odor; ˜10.5 mins-˜10.7 mins (relative acquisition times ˜0.583 mins, ˜0.589 mins, ˜0.594 mins, respectively) having a fruity and/or floral and/or plastic odor; and ˜13.3 mins-˜13.5 mins (relative acquisition times ˜0.739 mins, ˜0.744 mins, ˜0.75 mins, respectively) having a plastic and/or roasty odor; and any combinations thereof.

Embodiment 16. A 1,3-butylene glycol (BG) product, wherein the BG product does not comprise one or more compounds comprising an odor-active region as measured by GC/MS/O having a relative acquisition time selected from a group consisting of: about 0.417 min (rubber and/or plastic and/or green), about 0.450 min (plastic), about 0.483 min (solventy), about 0.783 min (plastic), about 1.033 mins (fruity and/or plastic), about 1.1 mins (plastic and/or pungent and/or mushroom and/or metallic), about 1.367 mins (fruity and/or plastic and/or metal and/or mushroom), about 1.967 mins (waxy and/or fatty), and combinations thereof, where the BG product comprises bioderived BG (bioBG), and the bioBG has a relative acquisition time of 1 min.

Embodiment 17. The BG product according to any one of embodiments 1-16, wherein the bioBG is selected from the group consisting of: (R)-1,3-BG; (S)-1,3-BG; and a mixture of (R)-1,3-BG and (S)-1,3-BG.

Embodiment 18. The BG product according to embodiment 17, wherein the BG comprises (R)-1,3-BG.

Embodiment 19. The BG product according to embodiment 17, wherein the BG comprises (S)-1,3-BG.

Embodiment 20. The BG product according to embodiment 17, wherein the BG comprises a mixture of (R)-1,3-BG and (S)-1,3-BG.

Embodiment 21. The BG product according to any one of embodiments 1-20, wherein the BG product comprises a neutral odor.

Embodiment 22. A composition, comprising:
the 1,3-butylene glycol (BG) product of any one of embodiments 1-21, and
a physiologically acceptable vehicle.

Embodiment 23. The composition according to embodiment 22, wherein the BG product comprises a neutral odor.

Embodiment 24. The composition according to any one of embodiments 22-23, wherein the BG product comprises 5-norbornene-2-ol; 2-ethyl-1-hexanol; and or salts, derivatives, analogs, or solvates, thereof, or any combinations thereof.

Embodiment 25. The composition according to any one of embodiments 22-24, wherein the BG product comprises a property selected from the group consisting of: humectant, fungicide, bactericide, conditioning agent, preservative, viscosity-decreasing agent, solvent, and combinations thereof.

Embodiment 26. The composition according to any one of embodiments 22-25, wherein the composition is selected from the group consisting of: cosmetics, personal care products, and cosmeceuticals.

Embodiment 27. The composition according to any one of embodiments 22-26, wherein the composition is selected from the group consisting of: eye liner, eye shadow, eye makeup, mascara, brow liner, primer, foundation, concealer, BB cream, CC cream, brighteners, blush, bronzer/highlighter, setting powder/spray, body/facial sprays, facial serums, lipstick, lip gloss, lip balm, lip liner, perfumes/fragrances, creams, lotions, moisturizers, face masks, sunscreens, sunblocks, fingernail polishes, deodorants, anti-perspirants, shampoos, conditioners, hair dyes, hair dye removers, soap, cleansers, aromatherapy compositions, massage oils, products comprising an ingredient selected from the group consisting of: vitamins, botanical or marine extracts, peptides, antioxidants, alpha-hydroxy acids, and combinations thereof.

Embodiment 28. A method, comprising applying to a subject in need thereof, a composition of any one of embodiments 22-27.

Embodiment 29. The method according to embodiment 28, wherein the composition is selected from the group consisting of: cosmetics, cosmeceutical, and personal care products.

Embodiment 30. The method of any one of embodiments 28-29, wherein the composition is selected from the group consisting of: eye liner, eye shadow, eye makeup, mascara, brow liner, primer, foundation, concealer, BB cream, CC cream, brighteners, blush, bronzer/highlighter, setting powder/spray, lipstick, lip gloss, lip balm, lip liner, perfumes/fragrances, creams, lotions, moisturizers, face masks, sunscreens, fingernail polishes, shampoos, conditioners, hair dyes, hair dye removers, deodorants, anti-perspirants, soaps, cleansers, and cosmetics with vitamins, botanical or marine extracts, peptides, antioxidants, and alpha-hydroxy acids.

Embodiment 31. Use of the 1,3-butylene glycol (BG) product of any one of embodiments 1-21 or the composition of any one of embodiments 22-27 for: improving appearance, hygiene, or skincare, or providing a property selected from the group consisting of: humectant, fungicide, bactericide, conditioning agent, preservative, viscosity-decreasing agent, solvent, and combinations thereof, to a subject in need thereof.

Embodiment 32. A bioderived 1,3-butylene glycol (BG) composition, comprising a bioderived BG (bioBG) and one or more compounds characterized by an aromatic region, an odor-active region, or a peak as measured by gas chromatography/mass spectrometry/olfactory (GC/MS/O) having (a) a fruity and/or solventy odor-active region having a relative acquisition time of 0.594 min; (b) a fruity odor-active region having a relative acquisition time of 0.628 min; (c) a citrus and/or peel odor-active region having a relative acquisition time of 0.655 min; (d) a solventy odor-active region having a relative acquisition time of 0.711 min; or any combinations thereof, as measured by GC/MS under specified conditions, wherein the relative acquisition time of bioBG is 1 min, where the composition comprises a vehicle (e.g., a carrier, a diluent, an excipient, or any combinations thereof).

Embodiment 33. A bioderived 1,3-butylene glycol (BG) composition, comprising a bioderived BG (bioBG) and a compound characterized by an aromatic region, an odor-active region, or a peak as measured by gas chromatography/mass spectrometry/olfactory (GC/MS/O) under specified conditions having an acquisition time of about 6.3 mins, where the composition comprises a vehicle (e.g., a carrier, a diluent, an excipient, or any combinations thereof).

Embodiment 34. The BG composition of embodiment 33, wherein the odor-active region comprises a fruity odor.

Embodiment 35. The BG composition of any one of embodiments 33-34, wherein the compound characterized by an odor-active region as measured by gas chromatography/mass spectrometry/olfactory (GC/MS/O) under specified conditions does not comprise, does not consist essentially of, does not consist of, or minimally comprises an acquisition time selected from the group consisting of: about 6.2 mins, about 6.9 mins, about 8.9 mins, about 17.6 mins, and any combinations thereof.

Embodiment 36. The BG composition of embodiment 35, wherein the acquisition time of about 6.2 mins corresponds to a solventy odor.

Embodiment 37. The BG composition of embodiment 35, wherein the acquisition time of about 6.9 mins corresponds to a plastic and/or musty odor.

Embodiment 38. The BG composition of embodiment 35, wherein the acquisition time of about 8.9 mins corresponds to a mushroom and/or earthy odor.

Embodiment 39. The BG composition of embodiment 35, wherein the acquisition time of about 17.6 mins corresponds to a sulfury and/or urine-like odor.

Embodiment 40. A 1,3-butylene glycol (BG) product, wherein the BG product comprises a bioderived BG (bioBG), wherein the BG product further comprises one or more compounds, wherein butyl acrylate (sharp, fruity) is in an amount of less than 1.82 mg/L, less than 0.53 mg/L, less than 0.33 mg/L, less than 0.24 mg/L, or less than 0.0001 mg/L; 3-hydroxy-butanal is in an amount of less than 25 ppm; 4-hydroxy-2-butanone is in an amount less than 25 ppm; 4-(3-hydroxybutoxy)butan-2-one is in an amount less than 25 ppm; 4-((4-hydroxybutan-2-yl)oxy)-butan-2-one is in an amount less than 25 ppm; 1,2-propanediol is in an amount less than 1 ppm; 1,3-propanediol is in an amount less than 10 ppm; 2,3-butanediol is in an amount less than 1 ppm; 2,4-dimethyl-6-((1-(4-methyl-1,3-dioxan-2-yl)propan-2-yl)oxy)-1,3-dioxane is in an amount less than 2 ppm; 4-(sec-butoxy)-2,6-dimethyl-1,3-dioxane is in an amount less than 4 ppm; 2,4-dimethyl-1,3-dioxane is in an amount less than 8 ppm; (E)-4-methyl-2-(prop-1-en-1-yl)-1,3-dioxane is in an amount less than 8 ppm; 3-hydroxybutyl acetate is in an amount less than 8 ppm; 1-(2-methyl-1,3-dioxan-4-yl)propan-2-ol is in an amount less than 8 ppm; 2-methyl-4-propyl-1,3-dioxane is in an amount less than 8 ppm; ti-substituted 1,3-dioxane

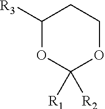

is in an amount less than 8 ppm and/or 3-hydroxybutyl carbono-substituted

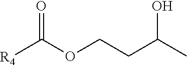

in an amount less than 8 ppm, wherein R1 to R4 are the same or different; a hydrogen atom, a C1-C4 alkyl group optionally substituted by an OH group, or a C2-C4 alkenyl group optionally substituted by OH group; 2,4-dimethyl-2-vinyl-1,3-dioxane; 2-(2,4-dimethyl-1,3-dioxan-2-yl)ethan-1-ol; 2,2,4-trimethyl-1,3-dioxane; 4-methyl-1,3-dioxane, wherein the total of 2,4-dimethyl-2-vinyl-1,3-dioxane; 2-(2,4-dimethyl-1,3-dioxan-2-yl)ethan-1-ol; 2,2,4-trimethyl-1,3-dioxane; 4-methyl-1,3-dioxane is in a total amount of less than 34 ppm; (E)-4-methyl-2-(prop-1-en-1-yl)-1,3-dioxane; 1-(4-methyl-1,3-dioxan-2-yl)propan-2-ol; 2,4-dimethyl-1,3-dioxane; 3-hydroxybutyl (E)-but-2-enoate; 3-hydroxybutyl acetate; 3-hydroxybutyl 3-hydroxybutanoate, wherein the total of (E)-4-methyl-2-(prop-1-en-1-yl)-1,3-dioxane; 1-(4-methyl-1,3-dioxan-2-yl)propan-2-ol; 2,4-dimethyl-1,3-dioxane; 3-hydroxybutyl (E)-but-2-enoate; 3-hydroxybutyl acetate; 3-hydroxybutyl 3-hydroxybutanoate is in a total amount of less than 63 ppm or wherein the total of 2,4-dimethyl-2-vinyl-1,3-dioxane; 2-(2,4-dimethyl-1,3-dioxan-2-yl)ethan-1-ol; 2,2,4-trimethyl-1,3-dioxane; 4-methyl-1,3-dioxane; (E)-4-methyl-2-(prop-1-en-1-yl)-1,3-dioxane; 1-(4-methyl-1,3-dioxan-2-yl)propan-2-ol; 2,4-dimethyl-1,3-dioxane; 3-hydroxybutyl (E)-but-2-enoate; 3-hydroxybutyl acetate; 3-hydroxybutyl 3-hydroxybutanoate is in a total amount of less than 97 ppm; methyl vinyl ketone is in an amount less than 8 ppm; 1-hydroxy-3-butanone is in an amount less than 1 ppm; acetone is in an amount less than 6 ppm; formaldehyde is in an amount less than 2 ppm; crotonaldehyde is in an amount less than 1 ppm; acetaldol is in an amount less than 1 ppm; acetaldehyde is in an amount less than 5 ppm; butyraldehyde is in an amount less than 4 ppm; 2-butanol, wherein the total of 2,4-dimethyl-2-vinyl-1,3-dioxane; 2-(2,4-dimethyl-1,3-dioxan-2-yl)ethan-1-ol; 2,2,4-trimethyl-1,3-dioxane; 4-methyl-1,3-dioxane; (E)-4-methyl-2-(prop-1-en-1-yl)-1,3-dioxane; 1-(4-methyl-1,3-dioxan-2-yl)propan-2-ol; 2,4-dimethyl-1,3-dioxane; 3-hydroxybutyl (E)-but-2-enoate; 3-hydroxybutyl acetate; 3-hydroxybutyl 3-hydroxybutanoate; and 2-butanol is in a total amount of less than 65 ppm; butyl acetate; 4-hydroxy-2-butanone; 2-methoxymethyl-2,4,5-trimethyl-1,3-dioxolane (solventy); 3-heptanone; 1-octen-3-one; N-pentyl-2-butylamine; 3-buten-2-one; 2-butenal; 2,4-dimethyl-1,3-dioxane; 2-ethyl-5-methyl-1,3-dioxane; 2-heptyl-1,3-dioxepane; 1-hydroxy-2-propanone; 1,2 propanediol (12PDO); 1,2,3-butanetriol; 1,2,3-propanetriol; 1,3 propanediol (13PDO); 1,4-butanediol (BDO); 2,3-butanediol (23BDO); 2,5-hexanedione; 3-buten-2-ol; cis-cryotyl alcohol; isopropyl alcohol (IPA); methyl vinyl ketone (MVK); n-butane (n-but); dimethylethanolamine (DMAE); toluene; HDD; and any combinations thereof.

Embodiment 41. A 1,3-butylene glycol (BG) product, wherein the BG product comprises a bioderived BG (bioBG), wherein the BG product further comprises one or more compounds, wherein 3-heptanone is in an amount of less than 95 or less than 4 in fold change relative to a reference value or a control as quantified using a dichloromethane extraction method, or 3-heptanone is in an amount of less than 80 ppm; 3-hydroxy-butanal is in an amount of less than 25 ppm; 4-hydroxy-2-butanone is in an amount less than 25 ppm; 4-(3-hydroxybutoxy)butan-2-one is in an amount less than 25 ppm; 4-((4-hydroxybutan-2-yl)oxy)-butan-2-one is in an amount less than 25 ppm; 1,2-propanediol is in an amount less than 1 ppm; 1,3-propanediol is in an amount less than 10 ppm; 2,3-butanediol is in an amount less than 1 ppm; 2,4-dimethyl-6-((1-(4-methyl-1,3-dioxan-2-yl)propan-2-yl)oxy)-1,3-dioxane is in an amount less than 2 ppm; 4-(sec-butoxy)-2,6-dimethyl-1,3-dioxane is in an amount less than 4 ppm; 2,4-dimethyl-1,3-dioxane is in an amount less than 8 ppm; (E)-4-methyl-2-(prop-1-en-1-yl)-1,3-dioxane is in an amount less than 8 ppm; 3-hydroxybutyl acetate is in an amount less than 8 ppm; 1-(2-methyl-1,3-dioxan-4-yl)propan-2-ol is in an amount less than 8 ppm; 2-methyl-4-propyl-1,3-dioxane is in an amount less than 8 ppm; tri-substituted 1,3-dioxane

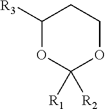

is in an amount less than 8 ppm and/or 3-hydroxybutyl carbono-substituted

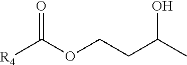

in an amount less than 8 ppm, wherein R1 to R4 are the same or different; a hydrogen atom, a C1-C4 alkyl group optionally substituted by an OH group, or a C2-C4 alkenyl group optionally substituted by OH group; 2,4-dimethyl-2-vinyl-1,3-dioxane; 2-(2,4-dimethyl-1,3-dioxan-2-yl)ethan-1-ol; 2,2,4-trimethyl-1,3-dioxane; 4-methyl-1,3-dioxane, wherein the total of 2,4-dimethyl-2-vinyl-1,3-dioxane; 2-(2,4-dimethyl-1,3-dioxan-2-yl)ethan-1-ol; 2,2,4-trimethyl-1,3-dioxane; 4-methyl-1,3-dioxane is in a total amount of less than 34 ppm; (E)-4-methyl-2-(prop-1-en-1-yl)-1,3-dioxane; 1-(4-methyl-1,3-dioxan-2-yl)propan-2-ol; 2,4-dimethyl-1,3-dioxane; 3-hydroxybutyl (E)-but-2-enoate; 3-hydroxybutyl acetate; 3-hydroxybutyl 3-hydroxybutanoate, wherein the total of (E)-4-methyl-2-(prop-1-en-1-yl)-1,3-dioxane; 1-(4-methyl-1,3-dioxan-2-yl)propan-2-ol; 2,4-dimethyl-1,3-dioxane; 3-hydroxybutyl (E)-but-2-enoate; 3-hydroxybutyl acetate; 3-hydroxybutyl 3-hydroxybutanoate is in a total amount of less than 63 ppm or wherein the total of 2,4-dimethyl-2-vinyl-1,3-dioxane; 2-(2,4-dimethyl-1,3-dioxan-2-yl)ethan-1-ol; 2,2,4-trimethyl-1,3-dioxane; 4-methyl-1,3-dioxane; (E)-4-methyl-2-(prop-1-en-1-yl)-1,3-dioxane; 1-(4-methyl-1,3-dioxan-2-yl)propan-2-ol; 2,4-dimethyl-1,3-dioxane; 3-hydroxybutyl (E)-but-2-enoate; 3-hydroxybutyl acetate; 3-hydroxybutyl 3-hydroxybutanoate is in a total amount of less than 97 ppm; methyl vinyl ketone is in an amount less than 8 ppm; 1-hydroxy-3-butanone is in an amount less than 1 ppm; acetone is in an amount less than 6 ppm; formaldehyde is in an amount less than 2 ppm; crotonaldehyde is in an amount less than 1 ppm; acetaldol is in an amount less than 1 ppm; acetaldehyde is in an amount less than 5 ppm; butyraldehyde is in an amount less than 4 ppm; 2-butanol, wherein the total of 2,4-dimethyl-2-vinyl-1,3-dioxane; 2-(2,4-dimethyl-1,3-dioxan-2-yl)ethan-1-ol; 2,2,4-trimethyl-1,3-dioxane; 4-methyl-1,3-dioxane; (E)-4-methyl-2-(prop-1-en-1-yl)-1,3-dioxane; 1-(4-methyl-1,3-dioxan-2-yl)propan-2-ol; 2,4-dimethyl-1,3-dioxane; 3-hydroxybutyl (E)-but-2-enoate; 3-hydroxybutyl acetate; 3-hydroxybutyl 3-hydroxybutanoate; and 2-butanol is in a total amount of less than 65 ppm; butyl acetate; butyl acrylate; 4-hydroxy-2-butanone; 2-methoxymethyl-2,4,5-trimethyl-1,3-dioxolane (solventy); 1-octen-3-one; N-pentyl-2-butylamine; 3-buten-2-one; 2-butenal; 2,4-dimethyl-1,3-dioxane; 2-ethyl-5-methyl-1,3-dioxane; 2-heptyl-1,3-dioxepane; 1-hydroxy-2-propanone; 1,2 propanediol (12PDO); 1,2,3-butanetriol; 1,2,3-propanetriol; 1,3 propanediol (13PDO); 1,4-butanediol (BDO); 2,3-butanediol (23BDO); 2,5-hexanedione; 3-buten-2-ol; cis-cryotyl alcohol; isopropyl alcohol (IPA); methyl vinyl ketone (MVK); n-butane (n-but); dimethylethanolamine (DMAE); toluene; HDD; and any combinations thereof.

Embodiment 42. A 1,3-butylene glycol (BG) product, wherein the BG product comprises a bioderived BG (bioBG), wherein the BG product further comprises one or more compounds, wherein 1-octen-3-one (mushroom) is in an amount of less than 80 ppm, or is in an amount of less than 187, 45 or 19 fold change relative to a reference value or a control as quantified using a dichloromethane extraction method; 3-hydroxy-butanal is in an amount of less than 25 ppm; 4-hydroxy-2-butanone is in an amount less than 25 ppm; 4-(3-hydroxybutoxy)butan-2-one is in an amount less than 25 ppm; 4-((4-hydroxybutan-2-yl)oxy)-butan-2-one is in an amount less than 25 ppm; 1,2-propanediol is in an amount less than 1 ppm; 1,3-propanediol is in an amount less than 10 ppm; 2,3-butanediol is in an amount less than 1 ppm; 2,4-dimethyl-6-((1-(4-methyl-1,3-dioxan-2-yl)propan-2-yl)oxy)-1,3-dioxane is in an amount less than 2 ppm; 4-(sec-butoxy)-2,6-dimethyl-1,3-dioxane is in an amount less than 4 ppm; 2,4-dimethyl-1,3-dioxane is in an amount less than 8 ppm; (E)-4-methyl-2-(prop-1-en-1-yl)-1,3-dioxane is in an amount less than 8 ppm; 3-hydroxybutyl acetate is in an amount less than 8 ppm; 1-(2-methyl-1,3-dioxan-4-yl)propan-2-ol is in an amount less than 8 ppm; 2-methyl-4-propyl-1,3-dioxane is in an amount less than 8 ppm; tri-substituted 1,3-dioxane

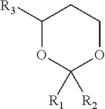

is in an amount less than 8 ppm and/or 3-hydroxybutyl carbono-substituted

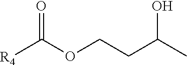

in an amount less than 8 ppm, wherein R1 to R4 are the same or different; a hydrogen atom, a C1-C4 alkyl group optionally substituted by an OH group, or a C2-C4 alkenyl group optionally substituted by OH group; 2,4-dimethyl-2-vinyl-1,3-dioxane; 2-(2,4-dimethyl-1,3-dioxan-2-yl)ethan-1-ol; 2,2,4-trimethyl-1,3-dioxane; 4-methyl-1,3-dioxane, wherein the total of 2,4-dimethyl-2-vinyl-1,3-dioxane; 2-(2,4-dimethyl-1,3-dioxan-2-yl)ethan-1-ol; 2,2,4-trimethyl-1,3-dioxane; 4-methyl-1,3-dioxane is in a total amount of less than 34 ppm; (E)-4-methyl-2-(prop-1-en-1-yl)-1,3-dioxane; 1-(4-methyl-1,3-dioxan-2-yl)propan-2-ol; 2,4-dimethyl-1,3-dioxane; 3-hydroxybutyl (E)-but-2-enoate; 3-hydroxybutyl acetate; 3-hydroxybutyl 3-hydroxybutanoate, wherein the total of (E)-4-methyl-2-(prop-1-en-1-yl)-1,3-dioxane; 1-(4-methyl-1,3-dioxan-2-yl)propan-2-ol; 2,4-dimethyl-1,3-dioxane; 3-hydroxybutyl (E)-but-2-enoate; 3-hydroxybutyl acetate; 3-hydroxybutyl 3-hydroxybutanoate is in a total amount of less than 63 ppm or wherein the total of 2,4-dimethyl-2-vinyl-1,3-dioxane; 2-(2,4-dimethyl-1,3-dioxan-2-yl)ethan-1-ol; 2,2,4-trimethyl-1,3-dioxane; 4-methyl-1,3-dioxane; (E)-4-methyl-2-(prop-1-en-1-yl)-1,3-dioxane; 1-(4-methyl-1,3-dioxan-2-yl)propan-2-ol; 2,4-dimethyl-1,3-dioxane; 3-hydroxybutyl (E)-but-2-enoate; 3-hydroxybutyl acetate; 3-hydroxybutyl 3-hydroxybutanoate is in a total amount of less than 97 ppm; methyl vinyl ketone is in an amount less than 8 ppm; 1-hydroxy-3-butanone is in an amount less than 1 ppm; acetone is in an amount less than 6 ppm; formaldehyde is in an amount less than 2 ppm; crotonaldehyde is in an amount less than 1 ppm; acetaldol is in an amount less than 1 ppm; acetaldehyde is in an amount less than 5 ppm; butyraldehyde is in an amount less than 4 ppm; 2-butanol, wherein the total of 2,4-dimethyl-2-vinyl-1,3-dioxane; 2-(2,4-dimethyl-1,3-dioxan-2-yl)ethan-1-ol; 2,2,4-trimethyl-1,3-dioxane; 4-methyl-1,3-dioxane; (E)-4-methyl-2-(prop-1-en-1-yl)-1,3-dioxane; 1-(4-methyl-1,3-dioxan-2-yl)propan-2-ol; 2,4-dimethyl-1,3-dioxane; 3-hydroxybutyl (E)-but-2-enoate; 3-hydroxybutyl acetate; 3-hydroxybutyl 3-hydroxybutanoate; and 2-butanol is in a total amount of less than 65 ppm; butyl acetate; butyl acrylate; 4-hydroxy-2-butanone; 2-methoxymethyl-2,4,5-trimethyl-1,3-dioxolane (solventy); 3-heptanone; N-pentyl-2-butylamine; 3-buten-2-one; 2-butenal; 2,4-dimethyl-1,3-dioxane; 2-ethyl-5-methyl-1,3-dioxane; 2-heptyl-1,3-dioxepane; 1-hydroxy-2-propanone; 1,2 propanediol (12PDO); 1,2,3-butanetriol; 1,2,3-propanetriol; 1,3 propanediol (13PDO); 1,4-butanediol (BDO); 2,3-butanediol (23BDO); 2,5-hexanedione; 3-buten-2-ol; cis-cryotyl alcohol; isopropyl alcohol (IPA); methyl vinyl ketone (MVK); n-butane (n-but); dimethylethanolamine (DMAE); toluene; HDD; and any combinations thereof.

Embodiment 43. A 1,3-butylene glycol (BG) product, wherein the BG product comprises a bioderived BG (bioBG), wherein the BG product further comprises one or more compounds, wherein HDD is in an amount less than 215, less than 19.3, less than 4, or less than 2.5 in fold change relative to a reference value or a control as quantified with a dichloromethane extraction method; 3-hydroxy-butanal is in an amount of less than 25 ppm; 4-hydroxy-2-butanone is in an amount less than 25 ppm; 4-(3-hydroxybutoxy)butan-2-one is in an amount less than 25 ppm; 4-((4-hydroxybutan-2-yl)oxy)-butan-2-one is in an amount less than 25 ppm; 1,2-propanediol is in an amount less than 1 ppm; 1,3-propanediol is in an amount less than 10 ppm; 2,3-butanediol is in an amount less than 1 ppm; 2,4-dimethyl-6-((1-(4-methyl-1,3-dioxan-2-yl)propan-2-yl)oxy)-1,3-dioxane is in an amount less than 2 ppm; 4-(sec-butoxy)-2,6-dimethyl-1,3-dioxane is in an amount less than 4 ppm; 2,4-dimethyl-1,3-dioxane is in an amount less than 8 ppm; (E)-4-methyl-2-(prop-1-en-1-yl)-1,3-dioxane is in an amount less than 8 ppm; 3-hydroxybutyl acetate is in an amount less than 8 ppm; 1-(2-methyl-1,3-dioxan-4-yl)propan-2-ol is in an amount less than 8 ppm; 2-methyl-4-propyl-1,3-dioxane is in an amount less than 8 ppm; tri-substituted 1,3-dioxane

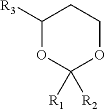

is in an amount less than 8 ppm and/or 3-hydroxybutyl carbono-substituted

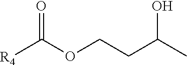

in an amount less than 8 ppm, wherein R1 to R4 are the same or different; a hydrogen atom, a C1-C4 alkyl group optionally substituted by an OH group, or a C2-C4 alkenyl group optionally substituted by OH group; 2,4-dimethyl-2-vinyl-1,3-dioxane; 2-(2,4-dimethyl-1,3-dioxan-2-yl)ethan-1-ol; 2,2,4-trimethyl-1,3-dioxane; 4-methyl-1,3-dioxane, wherein the total of 2,4-dimethyl-2-vinyl-1,3-dioxane; 2-(2,4-dimethyl-1,3-dioxan-2-yl)ethan-1-ol; 2,2,4-trimethyl-1,3-dioxane; 4-methyl-1,3-dioxane is in a total amount of less than 34 ppm; (E)-4-methyl-2-(prop-1-en-1-yl)-1,3-dioxane; 1-(4-methyl-1,3-dioxan-2-yl)propan-2-ol; 2,4-dimethyl-1,3-dioxane; 3-hydroxybutyl (E)-but-2-enoate; 3-hydroxybutyl acetate; 3-hydroxybutyl 3-hydroxybutanoate, wherein the total of (E)-4-methyl-2-(prop-1-en-1-yl)-1,3-dioxane; 1-(4-methyl-1,3-dioxan-2-yl)propan-2-ol; 2,4-dimethyl-1,3-dioxane; 3-hydroxybutyl (E)-but-2-enoate; 3-hydroxybutyl acetate; 3-hydroxybutyl 3-hydroxybutanoate is in a total amount of less than 63 ppm or wherein the total of 2,4-dimethyl-2-vinyl-1,3-dioxane; 2-(2,4-dimethyl-1,3-dioxan-2-yl)ethan-1-ol; 2,2,4-trimethyl-1,3-dioxane; 4-methyl-1,3-dioxane; (E)-4-methyl-2-(prop-1-en-1-yl)-1,3-dioxane; 1-(4-methyl-1,3-dioxan-2-yl)propan-2-ol; 2,4-dimethyl-1,3-dioxane; 3-hydroxybutyl (E)-but-2-enoate; 3-hydroxybutyl acetate; 3-hydroxybutyl 3-hydroxybutanoate is in a total amount of less than 97 ppm; methyl vinyl ketone is in an amount less than 8 ppm; 1-hydroxy-3-butanone is in an amount less than 1 ppm; acetone is in an amount less than 6 ppm; formaldehyde is in an amount less than 2 ppm; crotonaldehyde is in an amount less than 1 ppm; acetaldol is in an amount less than 1 ppm; acetaldehyde is in an amount less than 5 ppm; butyraldehyde is in an amount less than 4 ppm; 2-butanol, wherein the total of 2,4-dimethyl-2-vinyl-1,3-dioxane; 2-(2,4-dimethyl-1,3-dioxan-2-yl)ethan-1-ol; 2,2,4-trimethyl-1,3-dioxane; 4-methyl-1,3-dioxane; (E)-4-methyl-2-(prop-1-en-1-yl)-1,3-dioxane; 1-(4-methyl-1,3-dioxan-2-yl)propan-2-ol; 2,4-dimethyl-1,3-dioxane; 3-hydroxybutyl (E)-but-2-enoate; 3-hydroxybutyl acetate; 3-hydroxybutyl 3-hydroxybutanoate; and 2-butanol is in a total amount of less than 65 ppm; butyl acetate; butyl acrylate; 4-hydroxy-2-butanone; 2-methoxymethyl-2,4,5-trimethyl-1,3-dioxolane (solventy); 3-heptanone; 1-octen-3-one; N-pentyl-2-butylamine; 3-buten-2-one; 2-butenal; 2,4-dimethyl-1,3-dioxane; 2-ethyl-5-methyl-1,3-dioxane; 2-heptyl-1,3-dioxepane; 1-hydroxy-2-propanone; 1,2 propanediol (12PDO); 1,2,3-butanetriol; 1,2,3-propanetriol; 1,3 propanediol (13PDO); 1,4-butanediol (BDO); 2,3-butanediol (23BDO); 2,5-hexanedione; 3-buten-2-ol; cis-cryotyl alcohol; isopropyl alcohol (IPA); methyl vinyl ketone (MVK); n-butane (n-but); dimethylethanolamine (DMAE); toluene; HDD; and any combinations thereof.

Embodiment 44. The BG composition of any one of embodiments 40-43, wherein the BG product further comprises butyl acrylate (sharp, fruity) in an amount of less than 1.82 mg/L, less than 0.53 mg/L, less than 0.33 mg/L, less than 0.24 mg/L, or less than 0.0001 mg/L.

Embodiment 45. The BG composition of any one of embodiments 40-44, wherein the BG product further comprises 3-heptanone in an amount of less than 95 or less than 4 in fold change relative to a reference value or a control as quantified using a dichloromethane extraction method, or 3-heptanone in an amount of less than 80 ppm.

Embodiment 46. The BG composition of any one of embodiments 40-45, wherein the BG product further comprises 1-octen-3-one (mushroom) in an amount of less than 80 ppm, or is in an amount of less than 187, 45 or 19 fold change relative to a reference value or a control as quantified using a dichloromethane extraction method.

Embodiment 47. The BG composition of any one of embodiments 40-46, wherein the BG product further comprises HDD in an amount less than 215, less than 19.3, less than 4, or less than 2.5 in fold change relative to a reference value or a control as quantified with a dichloromethane extraction method.

As various changes can be made in the described subject matter without departing from the scope and spirit of the present disclosure, it is intended that all subject matter contained in the description, or defined in the appended claims, be interpreted as descriptive and illustrative of the present disclosure. Many modifications and variations of the present disclosure are possible in light of the above teachings. Accordingly, the present description is intended to embrace all such alternatives, modifications and variances which fall within the scope of the appended claims.

All documents cited or referenced herein and all documents cited or referenced in the herein cited documents, together with any manufacturer's instructions, descriptions, product specifications, and product sheets for any products mentioned herein or in any document incorporated by reference herein, are hereby incorporated by reference, and may be employed in the practice of the disclosure.

What is claimed is:

1. A 1,3-butylene glycol (BG) product comprising one or more compounds selected from the group consisting of: 5-norbornene-2-ol; 2-ethyl-1-hexanol; and salts, derivatives, analogs, or solvates thereof, or any combinations thereof, wherein the BG product comprises bioderived BG (bioBG).

2. A 1,3-butylene glycol (BG) product comprising one or more compounds characterized by an odor-active region as measured by gas chromatography/mass spectrometry/olfactory (GC/MS/O) having a relative acquisition time selected from a group consisting of: about 0.594 min having a fruity and/or solventy odor; about 0.628 min having a fruity odor; about 0.655 min having a citrus and/or peel odor; about 0.711 min having a solventy odor; about 1.328 mins having a roasty and/or exhaust odor; and combinations thereof, where the BG product comprises bioderived BG (bioBG), and the bioBG has a relative acquisition time of 1 min.

3. A 1,3-butylene glycol (BG) product comprising one or more compounds characterized by an odor-active region as measured by gas chromatography/quadrupole time-of-flight-mass spectrometry (GC/QTOF-MS), where the odor-active region elutes at an acquisition time of about 12.2 mins at 66.0463 m/z, wherein the BG product comprises bioderived BG (bioBG).

4. A 1,3-butylene glycol (BG) product comprising one or more compounds comprising a mass spectrum as characterized by quadrupole time-of-flight-mass spectrometry electron ionization (QTOF EI) at 70 eV, wherein the mass spectrum comprises peaks: 39.0229 m/z; 43.0707 m/z; 53.0385 m/z; 57.0169 m/z; 66.0463 m/z; 74.0156 m/z; 81.0333 m/z; 85.0284 m/z; 91.0539 m/z; 95.0489 m/z; and 110.0724 m/z; or the mass spectrum comprises peaks: 39.0229 m/z; 43.0707 m/z; 53.0385 m/z; 66.0463 m/z; 81.0333 m/z; 91.0539 m/z; 95.0489 m/z; and 110.0724 m/z, wherein the BG product comprises bioderived BG (bioBG).

5. A 1,3-butylene glycol (BG) product comprising one or more compounds as measured by extracted ion chromatograms selected from the group consisting of: 2-methoxymethyl-2,4,5-trimethyl-1,3-dioxolane having about 0 counts-1.6×10⁶ counts at 59.0489 m/z; 1-octen-3-one having about 0.15×10⁴-about 0.4×10⁴ counts at m/z 70.0411; 3-heptanone having about 0.4×10⁵ counts at m/z 57.0698; N-pentyl-2-butylamine having about 0 counts at m/z 114.0675; 5-norbornene-2-ol having about 0 counts to about 3.75×10⁵ counts at m/z 66.0463; and any combinations thereof, wherein the BG product comprises bioderived BG (bioBG).

6. The BG product according to any one of claims 1-5, wherein 3-hydroxy-butanal is in an amount of less than 25 ppm; 4-hydroxy-2-butanone is in an amount less than 25 ppm; 4-(3-hydroxybutoxy)butan-2-one is in an amount less than 25 ppm; 4-((4-hydroxybutan-2-yl)oxy)-butan-2-one is in an amount less than 25 ppm; 1,2-propanediol is in an amount less than 1 ppm; 1,3-propanediol is in an amount less than 10 ppm; 2,3-butanediol is in an amount less than 1 ppm; 2,4-dimethyl-6-((1-(4-methyl-1,3-dioxan-2-yl)propan-2-yl)oxy)-1,3-dioxane is in an amount less than 2 ppm; 4-(sec-butoxy)-2,6-dimethyl-1,3-dioxane is in an amount less than 4 ppm; 2,4-dimethyl-1,3-dioxane is in an amount less than 8 ppm; (E)-4-methyl-2-(prop-1-en-1-yl)-1,3-dioxane is in an amount less than 8 ppm; 3-hydroxybutyl acetate is in an amount less than 8 ppm; 1-(2-methyl-1,3-dioxan-4-yl)propan-2-ol is in an amount less than 8 ppm; 2-methyl-4-propyl-1,3-dioxane is in an amount less than 8 ppm; tri-substituted 1,3-dioxane 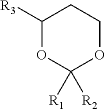
is in an amount less than 8 ppm and/or 3-hydroxybutyl carbono-substituted 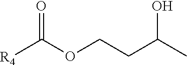
in an amount less than 8 ppm, wherein R1 to R4 are the same or different; a hydrogen atom, a C1-C4 alkyl group optionally substituted by an OH group, or a C2-C4 alkenyl group optionally substituted by OH group; 2,4-dimethyl-2-vinyl-1,3-dioxane; 2-(2,4-dimethyl-1,3-dioxan-2-yl)ethan-1-ol; 2,2,4-trimethyl-1,3-dioxane; 4-methyl-1,3-dioxane, wherein the total of 2,4-dimethyl-2-vinyl-1,3-dioxane; 2-(2,4-dimethyl-1,3-dioxan-2-yl)ethan-1-ol; 2,2,4-trimethyl-1,3-dioxane; 4-methyl-1,3-dioxane is in a total amount of less than 34 ppm; (E)-4-methyl-2-(prop-1-en-1-yl)-1,3-dioxane; 1-(4-methyl-1,3-dioxan-2-yl)propan-2-ol; 2,4-dimethyl-1,3-dioxane; 3-hydroxybutyl (E)-but-2-enoate; 3-hydroxybutyl acetate; 3-hydroxybutyl 3-hydroxybutanoate, wherein the total of (E)-4-methyl-2-(prop-1-en-1-yl)-1,3-dioxane; 1-(4-methyl-1,3-dioxan-2-yl)propan-2-ol; 2,4-dimethyl-1,3-dioxane; 3-hydroxybutyl (E)-but-2-enoate; 3-hydroxybutyl acetate; 3-hydroxybutyl 3-hydroxybutanoate is in a total amount of less than 63 ppm or wherein the total of 2,4-dimethyl-2-vinyl-1,3-dioxane; 2-(2,4-dimethyl-1,3-dioxan-2-yl)ethan-1-ol; 2,2,4-trimethyl-1,3-dioxane; 4-methyl-1,3-dioxane; (E)-4-methyl-2-(prop-1-en-1-yl)-1,3-dioxane; 1-(4-methyl-1,3-dioxan-2-yl)propan-2-ol; 2,4-dimethyl-1,3-dioxane; 3-hydroxybutyl (E)-but-2-enoate; 3-hydroxybutyl acetate; 3-hydroxybutyl 3-hydroxybutanoate is in a total amount of less than 97 ppm; methyl vinyl ketone is in an amount less than 8 ppm; 1-hydroxy-3-butanone is in an amount less than 1 ppm; acetone is in an amount less than 6 ppm; formaldehyde is in an amount less than 2 ppm; crotonaldehyde is in an amount less than 1 ppm; acetaldol is in an amount less than 1 ppm; acetaldehyde is in an amount less than 5 ppm; butyraldehyde is in an amount less than 4 ppm; 2-butanol, wherein the total of 2,4-dimethyl-2-vinyl-1,3-dioxane; 2-(2,4-dimethyl-1,3-dioxan-2-yl)ethan-1-ol; 2,2,4-trimethyl-1,3-dioxane; 4-methyl-1,3-dioxane; (E)-4-methyl-2-(prop-1-en-1-yl)-1,3-dioxane; 1-(4-methyl-1,3-dioxan-2-yl)propan-2-ol; 2,4-dimethyl-1,3-dioxane; 3-hydroxybutyl (E)-but-2-enoate; 3-hydroxybutyl acetate; 3-hydroxybutyl 3-hydroxybutanoate; and 2-butanol is in a total amount of less than 65 ppm; butyl acetate; butyl acrylate; 4-hydroxy-2-butanone; 2-methoxymethyl-2,4,5-trimethyl-1,3-dioxolane (solventy); 3-heptanone; 1-octen-3-one; N-pentyl-2-butylamine; 3-buten-2-one; 2-butenal; 2,4-dimethyl-1,3-dioxane; 2-ethyl-5-methyl-1,3-dioxane; butyl acrylate; 2-heptyl-1,3-dioxepane; 1-hydroxy-2-propanone; 1,2 propanediol (12PDO); 1,2,3-butanetriol; 1,2,3-propanetriol; 1,3 propanediol (13PDO); 1,4-butanediol (BDO); 2,3-butanediol (23BDO); 2,5-hexanedione; 3-buten-2-ol; cis-cryotyl alcohol; isopropyl alcohol (IPA); methyl vinyl ketone (MVK); n-butane (n-but); dimethylethanolamine (DMAE); toluene; and any combinations thereof.

7. The BG product of claim 6, wherein the BG product does not comprise a compound selected from the group consisting of: 3-hydroxy-butanal; 4-hydroxy-2-butanone; 4-(3-hydroxybutoxy)butan-2-one; 4-((4-hydroxybutan-2-yl)oxy)-butan-2-one; 1,2-propanediol; 1,3-propanediol; 2,3-butanediol; 2,4-dimethyl-6-((1-(4-methyl-1,3-dioxan-2-yl)propan-2-yl)oxy)-1,3-dioxane; 4-(sec-butoxy)-2,6-dimethyl-1,3-dioxane; 2,4-dimethyl-1,3-dioxane; (E)-4-methyl-2-(prop-1-en-1-yl)-1,3-dioxane; 3-hydroxybutyl acetate; 1-(2-methyl-1,3-dioxan-4-yl)propan-2-ol; 2-methyl-4-propyl-1,3-dioxane; tri-substituted 1,3-dioxane 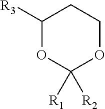
and/or 3-hydroxybutyl carbono-substituted 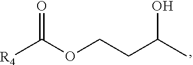
wherein R1 to R4 are the same or different; a hydrogen atom, a C1-C4 alkyl group optionally substituted by an OH group, or a C2-C4 alkenyl group optionally substituted by OH group; 2,4-dimethyl-2-vinyl-1,3-dioxane; 2-(2,4-dimethyl-1,3-dioxan-2-yl)ethan-1-ol; 2,2,4-trimethyl-1,3-dioxane; 4-methyl-1,3-dioxane; (E)-4-methyl-2-(prop-1-en-1-yl)-1,3-dioxane; 1-(4-methyl-1,3-dioxan-2-yl)propan-2-ol; 2,4-dimethyl-1,3-dioxane; 3-hydroxybutyl (E)-but-2-enoate; 3-hydroxybutyl acetate; 3-hydroxybutyl 3-hydroxybutanoate; methyl vinyl ketone; 1-hydroxy-3-butanone; acetone; formaldehyde; crotonaldehyde; acetaldol; acetaldehyde; butyraldehyde; 2-butanol; butyl acrylate; butyl acetate; 4-hydroxy-2-butanone; 2-methoxymethyl-2,4,5-trimethyl-1,3-dioxolane (solventy); 3-heptanone; 1-octen-3-one; N-pentyl-2-butylamine; 3-buten-2-one; 2-butenal; 2,4-dimethyl-1,3-dioxane; 2-ethyl-5-methyl-1,3-dioxane; butyl acrylate; 2-heptyl-1,3-dioxepane; 1-hydroxy-2-propanone; 1,2 propanediol (12PDO); 1,2,3-butanetriol; 1,2,3-propanetriol; 1,3 propanediol (13PDO); 1,4-butanediol (BDO); 2,3-butanediol (23BDO); 2,5-hexanedione; 3-buten-2-ol; cis-cryotyl alcohol; isopropyl alcohol (IPA); n-butane (n-but); dimethylethanolamine (DMAE); toluene; and or any combinations thereof, wherein the BG product comprises bioderived BG (bioBG).

8. A 1,3-butylene glycol (BG) product, wherein the BG product comprises a bioderived BG (bioBG), wherein the BG product further comprises one or more compounds, wherein 3-hydroxy-butanal is in an amount of less than 25 ppm; 4-hydroxy-2-butanone is in an amount less than 25 ppm; 4-(3-hydroxybutoxy)butan-2-one is in an amount less than 25 ppm; 4-((4-hydroxybutan-2-yl)oxy)-butan-2-one is in an amount less than 25 ppm; 1,2-propanediol is in an amount less than 1 ppm; 1,3-propanediol is in an amount less than 10 ppm; 2,3-butanediol is in an amount less than 1 ppm; 2,4-dimethyl-6-((1-(4-methyl-1,3-dioxan-2-yl)propan-2-yl)oxy)-1,3-dioxane is in an amount less than 2 ppm; 4-(sec-butoxy)-2,6-dimethyl-1,3-dioxane is in an amount less than 4 ppm; 2,4-dimethyl-1,3-dioxane is in an amount less than 8 ppm; (E)-4-methyl-2-(prop-1-en-1-yl)-1,3-dioxane is in an amount less than 8 ppm; 3-hydroxybutyl acetate is in an amount less than 8 ppm; 1-(2-methyl-1,3-dioxan-4-yl)propan-2-ol is in an amount less than 8 ppm; 2-methyl-4-propyl-1,3-dioxane is in an amount less than 8 ppm; tri-substituted 1,3-dioxane 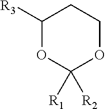
is in an amount less than 8 ppm and/or 3-hydroxybutyl carbono-substituted 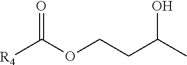
in an amount less than 8 ppm, wherein R1 to R4 are the same or different; a hydrogen atom, a C1-C4 alkyl group optionally substituted by an OH group, or a C2-C4 alkenyl group optionally substituted by OH group; 2,4-dimethyl-2-vinyl-1,3-dioxane; 2-(2,4-dimethyl-1,3-dioxan-2-yl)ethan-1-ol; 2,2,4-trimethyl-1,3-dioxane; 4-methyl-1,3-dioxane, wherein the total of 2,4-dimethyl-2-vinyl-1,3-dioxane; 2-(2,4-dimethyl-1,3-dioxan-2-yl)ethan-1-ol; 2,2,4-trimethyl-1,3-dioxane; 4-methyl-1,3-dioxane is in a total amount of less than 34 ppm; (E)-4-methyl-2-(prop-1-en-1-yl)-1,3-dioxane; 1-(4-methyl-1,3-dioxan-2-yl)propan-2-ol; 2,4-dimethyl-1,3-dioxane; 3-hydroxybutyl (E)-but-2-enoate; 3-hydroxybutyl acetate; 3-hydroxybutyl 3-hydroxybutanoate, wherein the total of (E)-4-methyl-2-(prop-1-en-1-yl)-1,3-dioxane; 1-(4-methyl-1,3-dioxan-2-yl)propan-2-ol; 2,4-dimethyl-1,3-dioxane; 3-hydroxybutyl (E)-but-2-enoate; 3-hydroxybutyl acetate; 3-hydroxybutyl 3-hydroxybutanoate is in a total amount of less than 63 ppm or wherein the total of 2,4-dimethyl-2-vinyl-1,3-dioxane; 2-(2,4-dimethyl-1,3-dioxan-2-yl)ethan-1-ol; 2,2,4-trimethyl-1,3-dioxane; 4-methyl-1,3-dioxane; (E)-4-methyl-2-(prop-1-en-1-yl)-1,3-dioxane; 1-(4-methyl-1,3-dioxan-2-yl)propan-2-ol; 2,4-dimethyl-1,3-dioxane; 3-hydroxybutyl (E)-but-2-enoate; 3-hydroxybutyl acetate; 3-hydroxybutyl 3-hydroxybutanoate is in a total amount of less than 97 ppm; methyl vinyl ketone is in an amount less than 8 ppm; 1-hydroxy-3-butanone is in an amount less than 1 ppm; acetone is in an amount less than 6 ppm; formaldehyde is in an amount less than 2 ppm; crotonaldehyde is in an amount less than 1 ppm; acetaldol is in an amount less than 1 ppm; acetaldehyde is in an amount less than 5 ppm; butyraldehyde is in an amount less than 4 ppm; 2-butanol, wherein the total of 2,4-dimethyl-2-vinyl-1,3-dioxane; 2-(2,4-dimethyl-1,3-dioxan-2-yl)ethan-1-ol; 2,2,4-trimethyl-1,3-dioxane; 4-methyl-1,3-dioxane; (E)-4-methyl-2-(prop-1-en-1-yl)-1,3-dioxane; 1-(4-methyl-1,3-dioxan-2-yl)propan-2-ol; 2,4-dimethyl-1,3-dioxane; 3-hydroxybutyl (E)-but-2-enoate; 3-hydroxybutyl acetate; 3-hydroxybutyl 3-hydroxybutanoate; and 2-butanol is in a total amount of less than 65 ppm; butyl acetate; butyl acrylate; 4-hydroxy-2-butanone; 2-methoxymethyl-2,4,5-trimethyl-1,3-dioxolane (solventy); 3-heptanone; 1-octen-3-one; N-pentyl-2-butylamine; 3-buten-2-one; 2-butenal; 2,4-dimethyl-1,3-dioxane; 2-ethyl-5-methyl-1,3-dioxane; butyl acrylate; 2-heptyl-1,3-dioxepane; 1-hydroxy-2-propanone; 1,2 propanediol (12PDO); 1,2,3-butanetriol; 1,2,3-propanetriol; 1,3 propanediol (13PDO); 1,4-butanediol (BDO); 2,3-butanediol (23BDO); 2,5-hexanedione; 3-buten-2-ol; cis-cryotyl alcohol; isopropyl alcohol (IPA); methyl vinyl ketone (MVK); n-butane (n-but); dimethylethanolamine (DMAE); toluene; and any combinations thereof.

9. A 1,3-butylene glycol (BG) product, wherein the BG product comprises a bioderived BG (bioBG), wherein the BG product does not comprise a compound selected from the group consisting of: 3-hydroxy-butanal; 4-hydroxy-2-butanone; 4-(3-hydroxybutoxy)butan-2-one; 4-((4-hydroxybutan-2-yl)oxy)-butan-2-one; 1,2-propanediol; 1,3-propanediol; 2,3-butanediol; 2,4-dimethyl-6-((1-(4-methyl-1,3-dioxan-2-yl)propan-2-yl)oxy)-1,3-dioxane; 4-(sec-butoxy)-2,6-dimethyl-1,3-dioxane; 2,4-dimethyl-1,3-dioxane; (E)-4-methyl-2-(prop-1-en-1-yl)-1,3-dioxane; 3-hydroxybutyl acetate; 1-(2-methyl-1,3-dioxan-4-yl)propan-2-ol; 2-methyl-4-propyl-1,3-dioxane; tri-substituted 1,3-dioxane 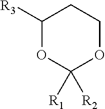
and/or 3-hydroxybutyl carbono-substituted 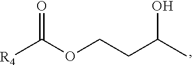
wherein R1 to R4 are the same or different; a hydrogen atom, a C1-C4 alkyl group optionally substituted by an OH group, or a C2-C4 alkenyl group optionally substituted by OH group; 2,4-dimethyl-2-vinyl-1,3-dioxane; 2-(2,4-dimethyl-1,3-dioxan-2-yl)ethan-1-ol; 2,2,4-trimethyl-1,3-dioxane; 4-methyl-1,3-dioxane; (E)-4-methyl-2-(prop-1-en-1-yl)-1,3-dioxane; 1-(4-methyl-1,3-dioxan-2-yl)propan-2-ol; 2,4-dimethyl-1,3-dioxane; 3-hydroxybutyl (E)-but-2-enoate; 3-hydroxybutyl acetate; 3-hydroxybutyl 3-hydroxybutanoate; methyl vinyl ketone; 1-hydroxy-3-butanone; acetone; formaldehyde; crotonaldehyde; acetaldol; acetaldehyde; butyraldehyde; 2-butanol; butyl acrylate; butyl acetate; 4-hydroxy-2-butanone; 2-methoxymethyl-2,4,5-trimethyl-1,3-dioxolane (solventy); 3-heptanone; 1-octen-3-one; N-pentyl-2-butylamine; 3-buten-2-one; 2-butenal; 2,4-dimethyl-1,3-dioxane; 2-ethyl-5-methyl-1,3-dioxane; butyl acrylate; 2-heptyl-1,3-dioxepane; 1-hydroxy-2-propanone; 1,2 propanediol (12PDO); 1,2,3-butanetriol; 1,2,3-propanetriol; 1,3 propanediol (13PDO); 1,4-butanediol (BDO); 2,3-butanediol (23BDO); 2,5-hexanedione; 3-buten-2-ol; cis-cryotyl alcohol; isopropyl alcohol (IPA); n-butane (n-but); dimethylethanolamine (DMAE); toluene; and or any combinations thereof, wherein the BG product comprises bioderived BG (bioBG).

10. A 1,3-butylene glycol (BG) product, wherein the BG product does not comprise one or more compounds having an odor-active region as measured by gas chromatography/quadrupole time-of-flight-mass spectrometry (GC/QTOF-MS), wherein the odor-active region elutes at an acquisition time selected from a group consisting of: about 12 mins at 59.0489 m/z, about 13 mins at 57.0698 m/z, about 15.5 mins at 70.0411 m/z, about 28.5 mins at 114.0675 m/z, and combinations thereof, wherein the BG product comprises bioderived BG (bioBG).

11. A 1,3-butylene glycol (BG) product, wherein the BG product does not comprise one or more compounds comprising a mass spectrum as characterized by quadrupole time-of-flight-mass spectrometry electron ionization (QTOF EI) at 70 eV, wherein the mass spectrum comprises an odor-active region, wherein the mass spectrum is selected from the group consisting of: (a) 43.017 m/z, 55.0541 m/z, 73.0283 m/z, 86.0723 m/z, 97.0642 m/z, and 115.0752; (b) 41.0385 m/z, 57.0698 m/z, 68.0259 m/z, 72.0569 m/z, 85.0646 m/z, 99.0801 m/z, and 114.1033 m/z; and (c) 43.0179 m/z, 57.0336 m/z, 72.0206 m/z, 86.0362 m/z, 114.0675 m/z, 128.0830 m/z, 138.0670 m/z, and 155.1060 m/z, wherein the BG product comprises bioderived BG (bioBG).

12. A 1,3-butylene glycol (BG) product, wherein the BG product comprising bioderived BG (bioBG) does not comprise one or more compounds comprising: an odor-active region as measured by GC/MS that elutes at an acquisition time selected from a group consisting of: about 9.3 mins (toluene); about 9.6 mins (butyl acetate); about 10.5 mins (butyl acrylate); about 16 mins (4-hydroxy-2-butanone); and any combinations thereof, where bioBG elutes at an acquisition time of about 18 mins; or an odor-active region as measured by GC/MS that elutes at an acquisition time selected from a group consisting of: about 9.3 mins (toluene); about 9.6 mins (butyl acetate); about 10.5 mins (butyl acrylate); about 11 mins; about 11.7 mins; about 14.2 mins; about 16 mins (4-hydroxy-2-butanone); about 17.7 mins; and any combinations thereof, where bioBG elutes at an acquisition time of about 18 mins.

13. A 1,3-butylene glycol (BG) product, wherein the BG product comprising bioderived BG (bioBG) does not comprise one or more compounds characterized by an odor-active region as measured by gas chromatography/mass spectrometry/olfactory (GC/MS/O) having a relative acquisition time selected from a group consisting of: about 0.517 min (toluene); about 0.533 min (butyl acetate); about 0.583 min (butyl acrylate); about 0.889 min (4-hydroxy-2-butanone); and combinations thereof, where the BG product comprises bioderived BG (bioBG), and the bioBG has a relative acquisition time of 1 min; or
about 0.517 mins (toluene); about 0.533 mins (butyl acetate); about 0.583 mins (butyl acrylate); about 0.611 mins; about 0.65 mins; about 0.789 mins; about 0.889 mins (4-hydroxy-2-butanone); about 0.983 mins; and combinations thereof, where the BG product comprises bioderived BG (bioBG), and the bioBG has a relative acquisition time of 1 min.

14. A 1,3-butylene glycol (BG) product, wherein the BG product does not comprise one or more compounds comprising an odor-active region as measured by GC/MS/O that elutes at an acquisition time selected from the group consisting of: ˜2.5 mins (rubber and/or plastic and/or green; 3-buten-2-one); ˜2.7 mins (plastic); ˜2.9 mins (solventy; 2-butenal); ˜4.6 mins (plastic: 2,4-dimethyl-1,3-dioxane); ˜6.2 mins (fruity and/or plastic; 2-ethyl-5-methyl-1,3-dioxane); ˜6.6 mins (plastic and/or pungent and/or mushroom and/or metallic; butyl acrylate); ˜8.2 mins (fruity and/or plastic and/or metal and/or mushroom; 2-heptyl-1,3-dioxepane); about 11.8 mins (waxy and/or fatty); and any combinations thereof, where the BG product comprises bioderived BG (bioBG).

15. A 1,3-butylene glycol (BG) product, wherein the BG product does not comprise one or more compounds comprising an odor-active region as measured by GC/MS/O that elutes at an acquisition time selected from the group consisting of: ˜9 mins-˜9.2 mins (relative acquisition times ˜0.5 min, ˜0.506 min, ˜0.511 min, respectively) having a fusel and/or sulfur and/or oil odor; ˜9.3 mins (relative acquisition time 0.517 min; toluene); ˜10.2 mins-˜10.4 mins (relative acquisition times ˜0.567 min, ˜0.572 min, ˜0.578 min, respectively) having a fruity and/or plastic odor; ˜10.5 mins-˜10.7 mins (relative acquisition times ˜0.583 min, ˜0.589 min, ˜0.594 min, respectively) having a fruity and/or floral and/or plastic odor; and ˜13.3 mins-˜13.5 mins (relative acquisition times ˜0.739 min, ˜0.744 min, ˜0.75 min, respectively) having a plastic and/or roasty odor; and any combinations thereof, where the BG product comprises bioderived BG (bioBG).

16. A 1,3-butylene glycol (BG) product, wherein the BG product does not comprise one or more compounds comprising an odor-active region as measured by GC/MS/O having a relative acquisition time selected from a group consisting of: about 0.417 min (rubber and/or plastic and/or green), about 0.450 min (plastic), about 0.483 min (solventy), about 0.783 min (plastic), about 1.033 min (fruity and/or plastic), about 1.1 mins (plastic and/or pungent and/or mushroom and/or metallic), about 1.367 mins (fruity and/or plastic and/or metal and/or mushroom), about 1.967 mins (waxy and/or fatty), and combinations thereof, where the BG product comprises bioderived BG (bioBG), and the bioBG has a relative acquisition time of 1 min.

17. The BG product according to any one of claims 1-16, wherein the bioBG is selected from the group consisting of: (R)-1,3-BG; (S)-1,3-BG; and a mixture of (R)-1,3-BG and (S)-1,3-BG.

18. The BG product according to claim 17, wherein the BG comprises (R)-1,3-BG.

19. The BG product according to claim 17, wherein the BG comprises (S)-1,3-BG.

20. The BG product according to claim 17, wherein the BG comprises a mixture of (R)-1,3-BG and (S)-1,3-BG.

21. The BG product according to any one of claims 1-20, wherein the BG product comprises a neutral odor.

22. A composition, comprising:
the 1,3-butylene glycol (BG) product of any one of claims 1-21, and
a physiologically acceptable vehicle.

23. The composition according to claim 22, wherein the BG product comprises a neutral odor.

24. The composition according to any one of claims 22-23, wherein the BG product comprises 5-norbornene-2-ol; 2-ethyl-1-hexanol; and salts, derivatives, analogs, or solvates, thereof; or any combinations thereof.

25. The composition according to any one of claims 22-24, wherein the BG product comprises a property selected from the group consisting of: humectant, fungicide, bactericide, conditioning agent, preservative, viscosity-decreasing agent, solvent, and combinations thereof.

26. The composition according to any one of claims 22-25, wherein the composition is selected from the group consisting of: cosmetics, personal care products, and cosmeceuticals.

27. The composition according to any one of claims 22-26, wherein the composition is selected from the group consisting of: eye liner, eye shadow, eye makeup, mascara, brow liner, primer, foundation, concealer, BB cream, CC cream, brighteners, blush, bronzer/highlighter, setting powder/spray, body/facial sprays, facial serums, lipstick, lip gloss, lip balm, lip liner, perfumes/fragrances, creams, lotions, moisturizers, face masks, sunscreens, sunblocks, fingernail polishes, deodorants, anti-perspirants, shampoos, conditioners, hair dyes, hair dye removers, soap, cleansers, aromatherapy compositions, massage oils, products comprising an ingredient selected from the group consisting of: vitamins, botanical or marine extracts, peptides, antioxidants, alpha-hydroxy acids, and combinations thereof.

28. A method, comprising applying to a subject in need thereof, a composition of any one of claims 22-27.

29. The method according to claim 28, wherein the composition is selected from the group consisting of: cosmetics, cosmeceutical, and personal care products.

30. The method of any one of claims 28-29, wherein the composition is selected from the group consisting of: eye liner, eye shadow, eye makeup, mascara, brow liner, primer, foundation, concealer, BB cream, CC cream, brighteners, blush, bronzer/highlighter, setting powder/spray, lipstick, lip gloss, lip balm, lip liner, perfumes/fragrances, creams, lotions, moisturizers, face masks, sunscreens, fingernail polishes, shampoos, conditioners, hair dyes, hair dye removers, deodorants, anti-perspirants, soaps, cleansers, and cosmetics with vitamins, botanical or marine extracts, peptides, antioxidants, and alpha-hydroxy acids.

31. Use of the 1,3-butylene glycol (BG) product of any one of claims 1-21 or the composition of any one of claims 22-27 for: improving appearance, hygiene, or skincare, or providing a property selected from the group consisting of: humectant, fungicide, bactericide, conditioning agent, preservative, viscosity-decreasing agent, solvent, and combinations thereof, to a subject in need thereof.
